(19) United States
(12) Patent Application Publication
(10) Pub. No.: US 2024/0327838 A1
(43) Pub. Date: Oct. 3, 2024

(54) MODULATORS OF ENaC EXPRESSION

(71) Applicant: IONIS PHARMACEUTICALS, INC., Carlsbad, CA (US)

(72) Inventors: Jeffrey R. Crosby, Carlsbad, CA (US); Shuling Guo, Carlsbad, CA (US); Huynh-Hoa Bui, Carlsbad, CA (US); Andrew T. Watt, Carlsbad, CA (US); Susan M. Freier, Carlsbad, CA (US)

(21) Appl. No.: 18/492,683

(22) Filed: Oct. 23, 2023

Related U.S. Application Data
(63) Continuation of application No. 16/759,908, filed on Apr. 28, 2020, now abandoned.
(60) Provisional application No. 62/743,669, filed on Oct. 10, 2018.
(60) Provisional application No. 62/579,640, filed on Oct. 31, 2017.

(51) Int. Cl. C12N 15/113; A61K 9/00; A61K 31/404; A61K 31/47; A61K 31/7105; A61K 31/713; A61M 11/00; A61P 11/00

(52) U.S. Cl. CPC C12N 15/113; A61K 9/0019; A61K 9/0075; A61K 9/0078; A61K 31/404; A61K 31/47; A61K 31/7105; A61K 31/713; A61P 11/00; A61M 11/005; C12N 2310/14; C12N 2310/315; C12N 2310/321; C12N 2310/3231; C12N 2310/3341; C12N 2310/341; C12N 2310/345; C12N 2310/346; C12N 2320/31; C12N 2320/32; C12N 2320/35

(57) ABSTRACT

The present embodiments provide methods, compounds, and compositions useful for inhibiting ENaC expression, which may be useful for treating, preventing, or ameliorating a disease associated with ENaC.

### SEQUENCE LISTING

The present application is being filed along with a Sequence Listing in electronic format. The Sequence Listing is provided as a file entitled BIOL0315SEQ.xml created Oct. 10, 2023, which is 1,828 kb in size. The information in the electronic format of the sequence listing is incorporated herein by reference in its entirety.

### FIELD

The present embodiments provide methods, compounds, and compositions useful for inhibiting ENaC expression, which can be useful for treating, preventing, or ameliorating a disease associated with ENaC.

### BACKGROUND

The epithelial sodium channel (ENaC) is a channel made up of three subunits (typically α-ENaC, (β-ENaC, and γ-ENaC; or SCNN1A, SCNN1B, and SCNN1G, respectively) that is expressed in several tissues, including the lungs. It allows passage of sodium ions across the epithelial cell membrane and is negatively regulated by chloride ions. In cystic fibrosis patients, the inhibition of ENaC is reduced due to decreased function of the chloride transporter, CFTR.

### SUMMARY

Certain embodiments provided herein are directed to potent and tolerable compounds and compositions useful for inhibiting ENaC expression, which can be useful for treating, preventing, ameliorating, or slowing progression of lung disorders, e.g., cystic fibrosis, chronic obstructive pulmonary disease (COPD), chronic bronchitis, and asthma. Certain embodiments provided herein comprise modified oligonucleotides complementary to an α-ENaC nucleic acid that potently reduce α-ENaC expression in animals.

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase.

As used herein, “2′-deoxynucleoside” means a nucleoside comprising 2′-H(H) ribosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” or “2-modified nucleoside” means a nucleoside comprising a 2′-substituted or 2′-modified sugar moiety. As used herein, “2′-substituted” or “2-modified” in reference to a furanosyl sugar moiety means a sugar moiety comprising at least one 2′-substituent group other than H or OH.

As used herein, “administration” or “administering” refers to routes of introducing a compound or composition provided herein to an individual to perform its intended function. An example of a route of administration that can be used includes, but is not limited to, administration by inhalation.

As used herein, “administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel or sequentially.

As used herein, “animal” refers to a human or non-human animal, including, but not limited to, mice, rats, rabbits, dogs, cats, pigs, and non-human primates, including, but not limited to, monkeys and chimpanzees.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means a compound comprising an antisense oligonucleotide and optionally one or more additional features, such as a conjugate group or terminal group.

As used herein, “antisense oligonucleotide” means an oligonucleotide having a nucleobase sequence that is at least partially complementary to a target nucleic acid.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cEt” or “constrained ethyl” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the β-D configuration.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more sterorandom chiral centers. In certain embodiments, the molecules are modified oligonucleotides. In certain embodiments, the molecules are compounds comprising modified oligonucleotides.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “effective amount” means the amount of compound sufficient to effectuate a desired physiological outcome in an individual in need of the compound. The effective amount may vary among individuals depending on the health and physical condition of the individual to be treated, the taxonomic group of the individuals to be treated, the formulation of the composition, assessment of the individual's medical condition, and other relevant factors.

As used herein, “efficacy” means the ability to produce a desired effect.

As used herein “ENaC” means any ENaC (epithelial sodium channel) nucleic acid or protein. “ENaC nucleic acid” means any nucleic acid encoding an ENaC subunit. For example, in certain embodiments, an ENaC nucleic acid includes a DNA chromosomal region encoding ENaC, an RNA transcribed from DNA encoding ENaC (e.g., a pre-mRNA transcript), and an mRNA transcript encoding ENaC. In certain embodiments, an ENaC nucleic acid or protein is an α-ENaC or SCNN1A (sodium channel epithelial 1 alpha subunit) nucleic acid or protein. Herein, α-ENaC and SCNN1A are used interchangeably and have the same meaning.

As used herein, “expression” includes all the functions by which a gene's coded information is converted into structures present and operating in a cell. Such structures include, but are not limited to, the products of transcription and translation.

As used herein, “gapmer” means an oligonucleotide, such as an antisense oligonucleotide, comprising an internal segment having a plurality of nucleosides that support RNase H cleavage positioned between external segments, each having one or more nucleosides, wherein the nucleosides comprising the internal segment are chemically distinct from the immediately adjacent nucleoside or nucleosides comprising the external segments. The internal segment may be referred to as the “gap” or “gap segment” and the external segments may be referred to as the “wings” or “wing segments”.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “individual” means a human or non-human animal selected for treatment or therapy.

As used herein, “inhibiting the expression or activity” refers to a reduction or blockade of the expression or activity relative to the expression or activity in an untreated or control sample and does not necessarily indicate a total elimination of expression or activity.

As used herein, the terms “internucleoside linkage” means a group or bond that forms a covalent linkage between adjacent nucleosides in an oligonucleotide. As used herein “modified internucleoside linkage” means any internucleoside linkage other than a naturally occurring, phosphate internucleoside linkage. Non-phosphate linkages are referred to herein as modified internucleoside linkages. “Phosphorothioate linkage” means a modified phosphate linkage in which one of the non-bridging oxygen atoms is replaced with a sulfur atom. A phosphorothioate internucleoside linkage is a modified internucleoside linkage. Modified internucleoside linkages include linkages that comprise abasic nucleosides.

As used herein, “abasic nucleoside” means a sugar moiety in an oligonucleotide or oligomeric compound that is not directly connected to a nucleobase. In certain embodiments, an abasic nucleoside is adjacent to one or two nucleosides in an oligonucleotide.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar” or “non-bicyclic modified sugar moiety” means a modified sugar moiety that comprises a modification, such as a substitutent, that does not form a bridge between two atoms of the sugar to form a second ring.

As used herein, “linked nucleosides” are nucleosides that are connected in a continuous sequence (i.e. no additional nucleosides are present between those that are linked).

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ or organism. For example, modulating ENaC expression can mean to increase or decrease the level of an ENaC RNA and/or an ENaC protein in a cell, tissue, organ or organism. A “modulator” effects the change in the cell, tissue, organ or organism. For example, a compound that modulates ENaC expression can be a modulator that decreases the amount of an ENaC RNA and/or an ENaC protein in a cell, tissue, organ or organism.

As used herein, “MOE” means methoxyethyl. “2′-MOE” or “2′-O-methoxyethyl” means a 2′-OCH₂CH₂OCH₃ group in place of the 2′-OH group of a ribosyl ring.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), and guanine (G). As used herein, a modified nucleobase is a group of atoms capable of pairing with at least one unmodified nucleobase. A universal base is a nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of an oligonucleotide and optionally one or more additional features, such as a conjugate group or terminal group.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a different form within the body or cells thereof. Typically conversion of a prodrug within the body is facilitated by the action of an enzymes (e.g., endogenous or viral enzyme) or chemicals present in cells or tissues and/or by physiologic conditions.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “standard cell assay” means the assay described in Example 3 and reasonable variations thereof.

As used herein, “standard in vivo experiment” means the procedure described in Example 4, 6, or 7, and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) moiety, as found in DNA (an “unmodified DNA sugar moiety”). As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” mean a nucleic acid that an antisense compound is designed to affect.

As used herein, “target region” means a portion of a target nucleic acid to which an antisense compound is designed to hybridize.

As used herein, “terminal group” means a chemical group or group of atoms that is covalently linked to a terminus of an oligonucleotide.

As used herein, “terminal wing nucleoside” means a nucleoside that is located at the terminus of a wing segment of a gapmer. Any wing segment that comprises or consists of at least two nucleosides has two termini: one that is immediately adjacent to the gap segment; and one that is at the end opposite the gap segment. Thus, any wing segment that comprises or consists of at least two nucleosides has two terminal nucleosides, one at each terminus.

As used herein, “therapeutically effective amount” means an amount of a compound, pharmaceutical agent, or composition that provides a therapeutic benefit to an individual.

As used herein, “treat” refers to administering a compound or pharmaceutical composition to an animal in order to effect an alteration or improvement of a disease, disorder, or condition in the animal.

### Certain Embodiments

Certain embodiments provide methods, compounds and compositions for inhibiting ENaC expression.

Certain embodiments provide compounds comprising or consisting of oligonucleotides complementary to an α-ENaC or SCNN1A nucleic acid. In certain embodiments, the α-ENaC or SCNN1A nucleic acid has the sequence set forth in RefSeq or GenBank Accession No. NM_001038.5 (disclosed herein as SEQ ID NO: 1), the complement of NC_000012.12 truncated from nucleosides 6343001 to 6380000 (disclosed herein as SEQ ID NO: 2), or NG_011945.1 (disclosed herein as SEQ ID NO: 1957). In certain embodiments, the compound is an antisense compound or oligomeric compound. In certain embodiments, the compound is single-stranded. In certain embodiments, the compound is double-stranded.

Certain embodiments provide a compound comprising a modified oligonucleotide 8 to 50 linked nucleosides in length and having a nucleobase sequence comprising at least 8 contiguous nucleobases of any of the nucleobase sequences of SEQ ID NOs: 6-1954. In certain embodiments, the compound is an antisense compound or oligomeric compound. In certain embodiments, the compound is single-stranded. In certain embodiments, the compound is double-stranded. In certain embodiments, the modified oligonucleotide is 10 to 30 linked nucleosides in length.

Certain embodiments provide a compound comprising a modified oligonucleotide 8 to 50 linked nucleosides in length and having a nucleobase sequence comprising at least 8 contiguous nucleobases of any of the nucleobase sequences of SEQ ID NOs: 6 to 1954. For example, the nucleobase sequence of the modified oligonucleotide comprises or consists of any one of SEQ ID NOs 6, 7, etc. . . . or 1954. In certain embodiments, the nucleobase sequence of the modified oligonucleotide comprises or consists of SEQ ID NO: 167. In certain embodiments, the nucleobase sequence of the modified oligonucleotide comprises or consists of SEQ ID NO: 244. In certain embodiments, the nucleobase sequence of the modified oligonucleotide comprises or consists of SEQ ID NO: 399. In certain embodiments, the nucleobase sequence of the modified oligonucleotide comprises or consists of SEQ ID NO: 428. In certain embodiments, the nucleobase sequence of the modified oligonucleotide comprises or consists of SEQ ID NO: 431. In certain embodiments, the nucleobase sequence of the modified oligonucleotide comprises or consists of SEQ ID NO: 438. In certain embodiments, the nucleobase sequence of the modified oligonucleotide comprises or consists of SEQ ID NO: 590. In certain embodiments, the nucleobase sequence of the modified oligonucleotide comprises or consists of SEQ ID NO: 824. In certain embodiments, the nucleobase sequence of the modified oligonucleotide comprises or consists of SEQ ID NO: 935. In certain embodiments, the nucleobase sequence of the modified oligonucleotide comprises or consists of SEQ ID NO: 1049. In certain embodiments, the nucleobase sequence of the modified oligonucleotide comprises or consists of SEQ ID NO: 1114. In certain embodiments, the nucleobase sequence of the modified oligonucleotide comprises or consists of SEQ ID NO: 1124. In certain embodiments, the nucleobase sequence of the modified oligonucleotide comprises or consists of SEQ ID NO: 1134. In certain embodiments, the nucleobase sequence of the modified oligonucleotide comprises or consists of SEQ ID NO: 1139. In certain embodiments, the nucleobase sequence of the modified oligonucleotide comprises or consists of SEQ ID NO: 1145. In certain embodiments, the nucleobase sequence of the modified oligonucleotide comprises or consists of SEQ ID NO: 1170. In certain embodiments, the nucleobase sequence of the modified oligonucleotide comprises or consists of SEQ ID NO: 1530. In certain embodiments, the nucleobase sequence of the modified oligonucleotide comprises or consists of SEQ ID NO: 1532. In certain embodiments, the nucleobase sequence of the modified oligonucleotide comprises or consists of SEQ ID NO: 1672. In certain embodiments, the nucleobase sequence of the modified oligonucleotide comprises or consists of SEQ ID NO: 1730. In certain embodiments, the nucleobase sequence of the modified oligonucleotide comprises or consists of SEQ ID NO: 1802. In certain embodiments, the nucleobase sequence of the modified oligonucleotide comprises or consists of SEQ ID NO: 1832. In certain embodiments, the compound is an antisense compound or oligomeric compound. In certain embodiments, the compound is single-stranded. In certain embodiments, the compound is double-stranded. In certain embodiments, the modified oligonucleotide is 10 to 30 linked nucleosides in length. In certain embodiments, the modified oligonucleotide has a nucleobase sequence comprising at least 12 contiguous nucleobases of any of SEQ ID Numbers from 6 to 1954.

Certain embodiments provide a compound comprising a modified oligonucleotide 10 to 50 linked nucleosides in length and having a nucleobase sequence comprising at least 10 contiguous nucleobases of any of the nucleobase sequences of SEQ ID NOs: 6-1954. In certain embodiments, the compound is an antisense compound or oligomeric compound. In certain embodiments, the compound is single-stranded. In certain embodiments, the compound is double-stranded. In certain embodiments, the modified oligonucleotide is 10 to 30 linked nucleosides in length.

Certain embodiments provide a compound comprising a modified oligonucleotide 11 to 50 linked nucleosides in length and having a nucleobase sequence comprising at least 11 contiguous nucleobases of any of the nucleobase sequences of SEQ ID NOs: 6-1954. In certain embodiments, the compound is an antisense compound or oligomeric compound. In certain embodiments, the compound is single-stranded. In certain embodiments, the compound is double-stranded. In certain embodiments, the modified oligonucleotide is 11 to 30 linked nucleosides in length.

Certain embodiments provide a compound comprising a modified oligonucleotide 12 to 50 linked nucleosides in length and having a nucleobase sequence comprising at least 12 contiguous nucleobases of any of the nucleobase sequences of SEQ ID NOs: 6-1954. In certain embodiments, the compound is an antisense compound or oligomeric compound. In certain embodiments, the compound is single-stranded. In certain embodiments, the compound is double-stranded. In certain embodiments, the modified oligonucleotide is 12 to 30 linked nucleosides in length.

In certain embodiments, the compound comprises a modified oligonucleotide 30 linked nucleosides in length. In certain embodiments, the compound is an antisense compound or oligomeric compound.

Certain embodiments provide a compound comprising a modified oligonucleotide 16 to 50 linked nucleosides in length and having a nucleobase sequence comprising the nucleobase sequence of any one of SEQ ID NOs: 6-1954. In certain embodiments, the compound is an antisense compound or oligomeric compound. In certain embodiments, the compound is single-stranded. In certain embodiments, the compound is double-stranded. In certain embodiments, the modified oligonucleotide is 16 to 30 linked nucleosides in length.

Certain embodiments provide a compound comprising a modified oligonucleotide consisting of the nucleobase sequence of any one of SEQ ID NOs: 6-1954. In certain embodiments, the compound is an antisense compound or oligomeric compound. In certain embodiments, the compound is single-stranded. In certain embodiments, the compound is double-stranded.

In certain embodiments, compounds comprise or consist of modified oligonucleotides complementary to an intron of an α-ENaC nucleic acid transcript. In certain embodiments, modified oligonucleotides are complementary to intron 1, intron 2, intron 3, intron 4, intron 5, intron 6, intron 7, intron 8, intron 9, intron 10, intron 11, or intron 12 of an α-ENaC nucleic acid transcript. In certain such embodiments, modified oligonucleotides are complementary to a sequence within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, compounds comprise or consist of oligonucleotides having at least an 8, 9, 10, 11, 12, 13, 14, 15, or 16 contiguous nucleobase portion complementary to an equal length portion of intron 1, intron 2, intron 3, intron 4, intron 5, intron 6, intron 7, intron 8, intron 9, intron 10, intron 11, or intron 12 of an α-ENaC nucleic acid transcript. In certain embodiments, such oligonucleotides have at least an 8, 9, 10, 11, 12, 13, 14, 15, or 16 contiguous nucleobase portion complementary to an equal length portion within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, these compounds are antisense compounds or oligomeric compounds. Compounds comprising modified oligonucleotide complementary to nearly any portion of certain introns of an α-ENaC nucleic acid transcript, e.g., intron 4 of an α-ENaC pre-mRNA, are generally especially potent and tolerable. Thus, such certain introns can be considered hot spot regions for targeting an α-ENaC nucleic acid transcript.

In certain embodiments, compounds comprise or consist of modified oligonucleotides complementary to intron 4 or the 3′-UTR of an α-ENaC nucleic acid transcript. In certain embodiments, modified oligonucleotides are complementary to a sequence within nucleotides 17,951-24,120; or 32,129-33,174 of SEQ ID NO: 2. In certain embodiments, compounds comprise or consist of oligonucleotides having at least an 8, 9, 10, 11, 12, 13, 14, 15, or 16 contiguous nucleobase portion complementary to an equal length portion of intron 4 or the 3′-UTR of an α-ENaC nucleic acid transcript. In certain embodiments, such oligonucleotides have at least an 8, 9, 10, 11, 12, 13, 14, 15, or 16 contiguous nucleobase portion complementary to an equal length portion within nucleotides 17,951-24,120; or 32,129-33,174 of SEQ ID NO: 2. In certain embodiments, these compounds are antisense compounds or oligomeric compounds.

In certain embodiments, a compound comprises a modified oligonucleotide 8 to 50 linked nucleosides in length and having at least an 8, 9, 10, 11, 12, 13, 14, 15, or 16 contiguous nucleobase portion complementary to an equal length portion within nucleotides 19,022-19,037; 20,415-20,430; 21,750-21,766; 32,844-32,859; or 32,989-33,004 of SEQ ID NO: 2. In certain embodiments, the modified oligonucleotide is 10 to 30 linked nucleosides in length.

In certain embodiments, a compound comprises a modified oligonucleotide 8 to 50 linked nucleosides in length and complementary within nucleotides 19,022-19,037; 20,415-20,430; 21,750-21,766; 32,844-32,859; or 32,989-33,004 of SEQ ID NO: 2. In certain embodiments, the modified oligonucleotide is 10 to 30 linked nucleosides in length.

In certain embodiments, a compound comprises a modified oligonucleotide 8 to 50 linked nucleosides in length and having a nucleobase sequence comprising at least an 8, 9, 10, 11, 12, 13, 14, 15, or 16 contiguous nucleobase portion of the nucleobase sequence of any one of compound numbers 797308, 797495, 826763, 827307, 827359, or 827392 (SEQ ID NOs: 239, 426, 1541, 1812, 1113, or 593). In certain embodiments, the modified oligonucleotide is 10 to 30 linked nucleosides in length. In certain embodiments, the nucleobase sequence of the modified oligonucleotide comprises or consists of SEQ ID NO: 239. In certain embodiments, the nucleobase sequence of the modified oligonucleotide comprises or consists of SEQ ID NO: 426. In certain embodiments, the nucleobase sequence of the modified oligonucleotide comprises or consists of SEQ ID NO: 1541. In certain embodiments, the nucleobase sequence of the modified oligonucleotide comprises or consists of SEQ ID NO: 1812. In certain embodiments, the nucleobase sequence of the modified oligonucleotide comprises or consists of SEQ ID NO: 1113. In certain embodiments, the nucleobase sequence of the modified oligonucleotide comprises or consists of SEQ ID NO: 593.

In certain embodiments, a compound comprises a modified oligonucleotide 8 to 50 linked nucleosides in length and having a nucleobase sequence comprising the nucleobase sequence of any one of compound numbers 797308, 797495, 826763, 827307, 827359, or 827392 (SEQ ID NOs: 239, 426, 1541, 1812, 1113, or 593). In certain embodiments, the modified oligonucleotide is 10 to 30 linked nucleosides in length.

In certain embodiments, a compound comprises a modified oligonucleotide having a nucleobase sequence consisting of the nucleobase sequence of any one of compound numbers 797308, 797495, 826763, 827307, 827359, or 827392 (SEQ ID NOs: 239, 426, 1541, 1812, 1113, or 593).

In certain embodiments, a compound comprising or consisting of a modified oligonucleotide complementary to α-ENaC is compound number 827359. Out of over 1,900 compounds that were screened as described in the Examples section below, compound numbers 797308, 797495, 826763, 827307, 827359, and 827392 emerged as the top lead compounds. In particular, compound number 827359 exhibited the best combination of properties in terms of potency and tolerability out of over 1,900 compounds.

Any of the foregoing oligonucleotides is a modified oligonucleotide comprising at least one modified internucleoside linkage, at least one modified sugar, and/or at least one modified nucleobase.

In certain embodiments, any of the foregoing modified oligonucleotides comprises at least one modified sugar. In certain embodiments, at least one modified sugar comprises a 2′-MOE modification. In certain embodiments, at least one modified sugar is a bicyclic sugar, such as a cEt bicyclic sugar, an LNA bicyclic sugar, or an ENA bicyclic sugar.

In certain embodiments, the modified oligonucleotide comprises at least one modified internucleoside linkage, such as a phosphorothioate internucleoside linkage.

In certain embodiments, any of the foregoing modified oligonucleotides comprises at least one modified nucleobase, such as 5-methylcytosine.

In certain embodiments, any of the foregoing modified oligonucleotides comprises: a gap segment consisting of linked 2′-deoxynucleosides;
a 5′ wing segment consisting of linked nucleosides; and
a 3′ wing segment consisting of linked nucleosides;
wherein the gap segment is positioned between the 5′ wing segment and the 3′ wing segment and
wherein each nucleoside of each wing segment comprises a modified sugar. In certain embodiments, the modified oligonucleotide is 16 to 50 linked nucleosides in length having a nucleobase sequence comprising the sequence recited in any one of SEQ ID NO: 239, 426, 1541, 1812, 1113, or 593. In certain embodiments, the modified oligonucleotide is 10 to 30 linked nucleosides in length having a nucleobase sequence comprising the sequence recited in any one of SEQ ID NOs: 239, 426, 1541, 1812, 1113, or 593. In certain embodiments, the modified oligonucleotide is 16 linked nucleosides in length having a nucleobase sequence consisting of the sequence recited in any one of SEQ ID NOs: 239, 426, 1541, 1812, 1113, or 593.

In certain embodiments, a compound comprises or consists of a modified oligonucleotide 20-80 linked nucleobases in length having a nucleobase sequence comprising the sequence recited in any one of SEQ ID NOs: 239, 426, 1541, 1812, 1113, or 593, wherein the modified oligonucleotide comprises a gap segment consisting of ten linked 2′-deoxynucleosides;
a 5′ wing segment consisting of five linked nucleosides; and
a 3′ wing segment consisting of five linked nucleosides;
wherein the gap segment is positioned between the 5′ wing segment and the 3′ wing segment, wherein each nucleoside of each wing segment comprises a 2′-O-methoxyethyl sugar; wherein each internucleoside linkage is a phosphorothioate linkage and wherein each cytosine is a 5-methylcytosine. In certain embodiments, the modified oligonucleotide consists of 20-30 linked nucleosides. In certain embodiments, the modified oligonucleotide consists of 20 linked nucleosides.

In certain embodiments, a compound comprises or consists of a modified oligonucleotide 16-80 linked nucleobases in length having a nucleobase sequence comprising the sequence recited in any one of SEQ ID NOs: 239, 426, 1541, 1812, 1113, or 593, wherein the modified oligonucleotide comprises a gap segment consisting of ten linked 2′-deoxynucleosides;
a 5′ wing segment consisting of three linked nucleosides; and
a 3′ wing segment consisting of three linked nucleosides;
wherein the gap segment is positioned between the 5′ wing segment and the 3′ wing segment; wherein the nucleosides of the 5′ wing segment each comprise a cEt bicyclic sugar; wherein the nucleosides of the 3′ wing segment each comprises a cEt bicyclic sugar; wherein each internucleoside linkage is a phosphorothioate linkage; and wherein each cytosine is a 5-methylcytosine. In certain embodiments, the modified oligonucleotide is 16-80 linked nucleosides in length. In certain embodiments, the modified oligonucleotide is 16-30 linked nucleosides in length.

In certain embodiments, a compound comprises or consists of a modified oligonucleotide according to one of the following formulas:

(SEQ ID NO: 1963)
mCks mCks mCks Gds Ads Tds Ads Gds mCds Tds Gds
Gds Tds Tks Gks Tk;
(SEQ ID NO: 1964)
Aks Aks Gks Tds Ads Tds Gds Gds Tds Gds mCds Ads
Ads mCks Aks Gk;
(SEQ ID NO: 1965)
Aks mCks Gks Ads Tds Tds Ads mCds Ads Gds Gds
Gds Ads Tks Tks mCk;
(SEQ ID NO: 1966)
Tks Gks mCks Ads Tds Ads Gds Gds Ads Gds Tds Tds
mCds Tks mCks Tk;
(SEQ ID NO: 1967)
Aks Gks Aks Gds Tds Ads Ads Tds Gds Ads Ads Ads
mCds mCks mCks Ak;
(SEQ ID NO: 1968)
mCks Gks Aks Tds Tds Ads mCds Ads Gds Gds Gds
Ads Tds Tks mCks Ak;

wherein A=an adenine, mC=a 5-methylcytosine, G=a guanine, T=a thymine, k=a cEt sugar moiety, d=a 2′-deoxyribosyl sugar moiety, and s=a phosphorothioate internucleoside linkage.

In certain embodiments, a compound comprises or consists of compound 827359 or salt thereof, a modified oligonucleotide having the following chemical structure:

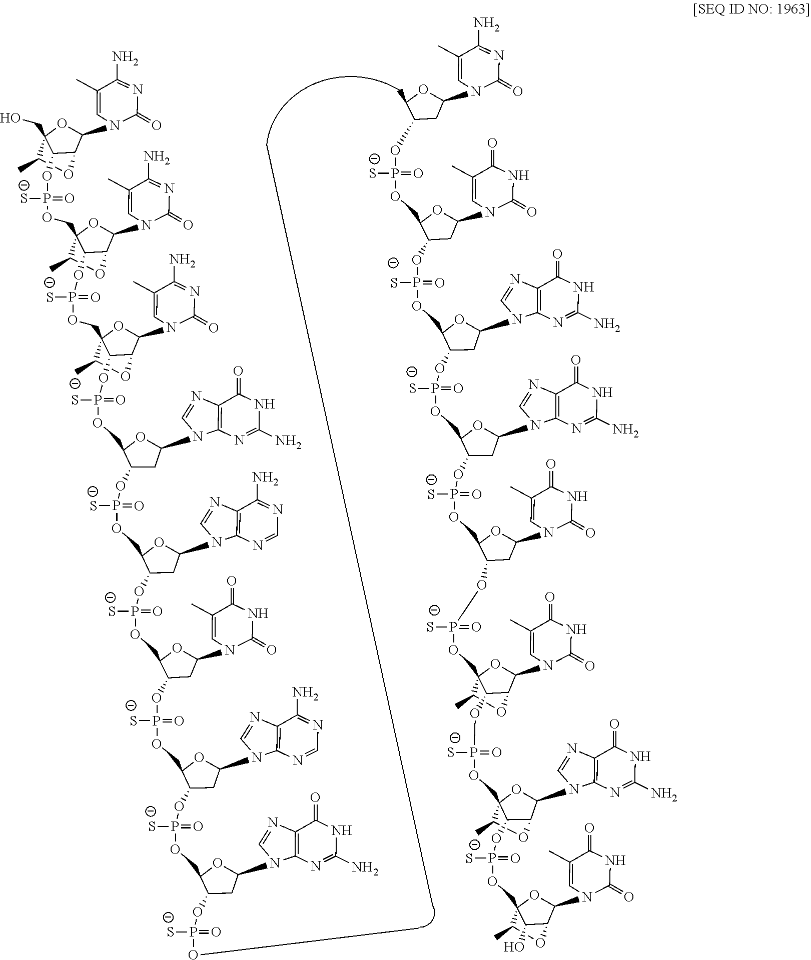

In certain embodiments, a compound comprises or consists of the sodium salt of compound 827359, having the following chemical structure:

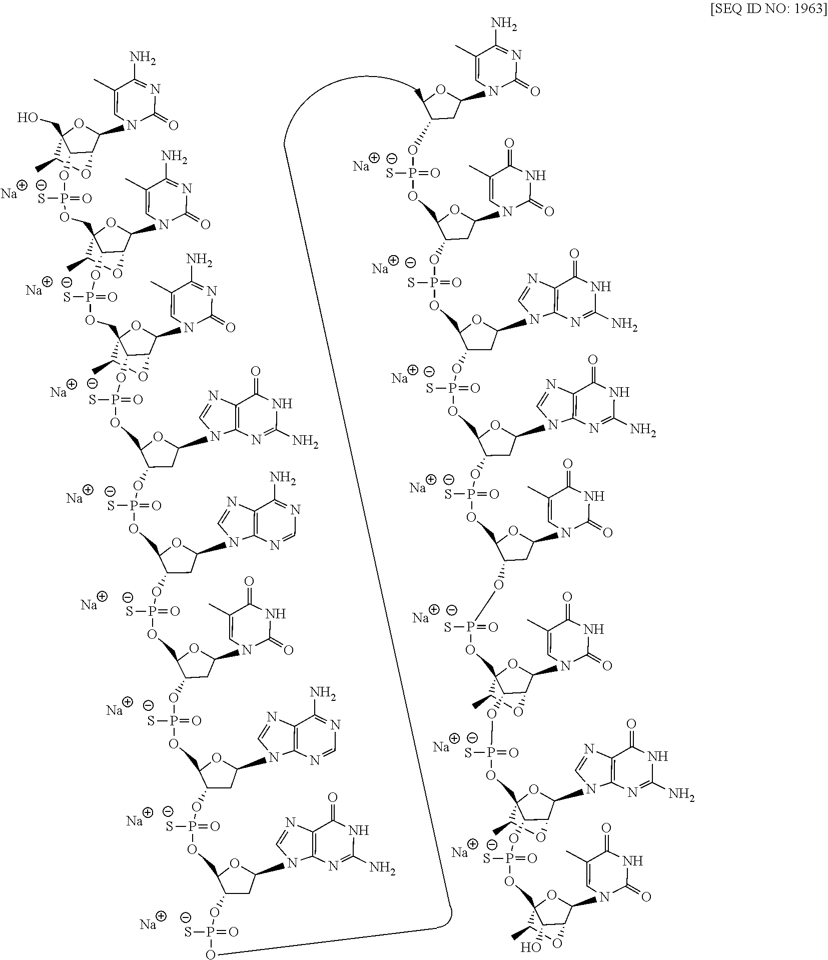

In any of the foregoing embodiments, the compound or oligonucleotide can be at least 85%, at least 90%, at least 95%, at least 98%, at least 99%, or 100% complementary to a nucleic acid encoding α-ENaC.

In any of the foregoing embodiments, the compound can be single-stranded. In certain embodiments, the compound comprises 2′-deoxyribonucleosides. In certain embodiments, the compound is double-stranded. In certain embodiments, the compound is double-stranded and comprises ribonucleosides. In any of the foregoing embodiments, the compound can be an antisense compound or oligomeric compound.

In any of the foregoing embodiments, the compound can be 8 to 80, 10 to 30, 12 to 50, 13 to 30, 13 to 50, 14 to 30, 14 to 50, 15 to 30, 15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked nucleosides in length. In certain embodiments, the compound comprises or consists of an oligonucleotide.

In certain embodiments, a compound comprises a modified oligonucleotide described herein and a conjugate group. In certain embodiments, the conjugate group is linked to the modified oligonucleotide at the 5′ end of the modified oligonucleotide. In certain embodiments, the conjugate group is linked to the modified oligonucleotide at the 3′ end of the modified oligonucleotide.

In certain embodiments, compounds or compositions provided herein comprise a salt of the modified oligonucleotide. In certain embodiments, the salt is a sodium salt. In certain embodiments, the salt is a potassium salt.

In certain embodiments, the compounds or compositions as described herein are active by virtue of having at least one of an in vitro IC₅₀ of less than 250 nM, less than 200 nM, less than 150 nM, less than 100 nM, less than 90 nM, less than 80 nM, less than 70 nM, less than 65 nM, less than 60 nM, less than 55 nM, less than 50 nM, less than 45 nM, less than 40 nM, less than 35 nM, less than 30 nM, less than 25 nM, less than 20 nM, or less than 15 nM in a standard cell assay.

In certain embodiments, the compounds or compositions as described herein are highly tolerable as demonstrated by having at least one of an increase an alanine transaminase (ALT) or aspartate transaminase (AST) value of no more than 4 fold, 3 fold, 2 fold, or 1.5 fold over saline treated animals or an increase in liver, spleen, or kidney weight of no more than 30%, 20%, 15%, 12%, 10%, 5%, or 2% compared to control treated animals. In certain embodiments, the compounds or compositions as described herein are highly tolerable as demonstrated by having no increase of ALT or AST over control treated animals. In certain embodiments, the compounds or compositions as described herein are highly tolerable as demonstrated by having no increase in liver, spleen, or kidney weight over control animals.

Certain embodiments provide a composition comprising the compound of any of the aforementioned embodiments or salt thereof and at least one of a pharmaceutically acceptable carrier or diluent. In certain embodiments, the composition has a viscosity less than about 40 centipoise (cP), less than about 30 cP, less than about 20 cP, less than about 15 cP, less than about 10 cP, less than about 5 cP, or less than about 3 cP, or less than about 1.5 cP. In certain embodiments, the composition having any of the aforementioned viscosities comprises a compound provided herein at a concentration of about 15 mg/mL, 20 mg/mL, 25 mg/mL, or about 50 mg/mL. In certain embodiments, the composition having any of the aforementioned viscosities and/or compound concentrations has a temperature of room temperature or about 20° C., about 21° C., about 22° C., about 23° C., about 24° C., about 25° C., about 26° C., about 27° C., about 28° C., about 29° C., or about 30° C.

Any of the foregoing compounds can be used for treating, preventing, or ameliorating a disease associated with ENaC as further described herein.

### Certain Indications

Certain embodiments provided herein relate to methods of inhibiting ENaC expression, which can be useful for treating, preventing, or ameliorating a disease associated with ENaC in an individual, by administration of a compound that targets α-ENaC. In certain embodiments, the compound can be an α-ENaC inhibitor. In certain embodiments, the compound can be an antisense compound, oligomeric compound, or oligonucleotide complementary to α-ENaC. In certain embodiments, the compound can be any of the compounds described herein.

Examples of diseases associated with ENaC that are treatable, preventable, and/or ameliorable with the methods provided herein include cystic fibrosis, COPD, asthma, and chronic bronchitis.

In certain embodiments, a method of treating, preventing, or ameliorating a disease associated with α-ENaC in an individual comprises administering to the individual a compound comprising an α-ENaC inhibitor, thereby treating, preventing, or ameliorating the disease. In certain embodiments, the compound comprises an antisense compound targeted to α-ENaC. In certain embodiments, the compound comprises an oligonucleotide complementary to an α-ENaC nucleic acid transcript. In certain embodiments, the oligonucleotide is a modified oligonucleotide. In certain embodiments, the compound comprise a modified oligonucleotide complementary to an intron of an α-ENaC nucleic acid transcript. In certain embodiments, the modified oligonucleotide is complementary to intron 1, intron 2, intron 3, intron 4, intron 5, intron 6, intron 7, intron 8, intron 9, intron 10, intron 11, or intron 12 of an α-ENaC nucleic acid transcript. In certain such embodiments, the oligonucleotide is complementary to a sequence within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, the compound comprises a modified oligonucleotide 8 to 50 linked nucleosides in length and having a nucleobase sequence comprising at least 8 contiguous nucleobases of any of the nucleobase sequences of SEQ ID NOs: 6-1954. In certain embodiments, the compound comprises a modified oligonucleotide 12 to 50 linked nucleosides in length and having a nucleobase sequence comprising the nucleobase sequence of any one of SEQ ID NOs: 6-1954. In certain embodiments, the compound comprises a modified oligonucleotide consisting of the nucleobase sequence of any one of SEQ ID NOs: 6-1954. In certain embodiments, the compound comprises a modified oligonucleotide 16 to 50 linked nucleosides in length having a nucleobase sequence comprising any one of SEQ ID NOs: 239, 426, 1541, 1812, 1113, or 593. In certain embodiments, the compound comprises a modified oligonucleotide having a nucleobase sequence consisting of any one of SEQ ID NOs: 239, 426, 1541, 1812, 1113, or 593. In any of the foregoing embodiments, the modified oligonucleotide can be 10 to 30 linked nucleosides in length. In certain embodiments, the compound is compound number 797308, 797495, 826763, 827307, 827359, or 827392. In any of the foregoing embodiments, the compound can be single-stranded or double-stranded. In any of the foregoing embodiments, the compound can be an antisense compound or oligomeric compound. In certain embodiments, the compound is administered to the individual via inhalation. In certain embodiments, administering the compound improves or preserves spirometry or mucociliary clearance.

In certain embodiments, a method of treating, preventing, or ameliorating cystic fibrosis, COPD, asthma, or chronic bronchitis comprises administering to the individual a compound comprising a modified oligonucleotide complementary to an α-ENaC nucleic acid, thereby treating, preventing, or ameliorating cystic fibrosis, COPD, asthma, or chronic bronchitis. In certain embodiments, the compound is an antisense compound targeted to α-ENaC. In certain embodiments, the oligonucleotide is a modified oligonucleotide. In certain embodiments, the compound comprise a modified oligonucleotide complementary to an intron of an α-ENaC nucleic acid transcript. In certain embodiments, the modified oligonucleotide is complementary to intron 1, intron 2, intron 3, intron 4, intron 5, intron 6, intron 7, intron 8, intron 9, intron 10, intron 11, or intron 12 of an α-ENaC nucleic acid transcript. In certain such embodiments, the oligonucleotide is complementary to a sequence within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, the compound comprises a modified oligonucleotide 8 to 50 linked nucleosides in length and having a nucleobase sequence comprising at least 8 contiguous nucleobases of any of the nucleobase sequences of SEQ ID NOs: 6-1954. In certain embodiments, the compound comprises a modified oligonucleotide 12 to 50 linked nucleosides in length and having a nucleobase sequence comprising the nucleobase sequence of any one of SEQ ID NOs: 6-1954. In certain embodiments, the compound comprises a modified oligonucleotide consisting of the nucleobase sequence of any one of SEQ ID NOs: 6-1954. In certain embodiments, the compound comprises a modified oligonucleotide of 16 to 50 linked nucleosides in length having a nucleobase sequence comprising any one of SEQ ID NOs: 239, 426, 1541, 1812, 1113, or 593. In certain embodiments, the compound comprises a modified oligonucleotide having a nucleobase sequence consisting of any one of SEQ ID NOs: 239, 426, 1541, 1812, 1113, or 593. In any of the foregoing embodiments, the modified oligonucleotide can be 10 to 30 linked nucleosides in length. In certain embodiments, the compound is compound number 797308, 797495, 826763, 827307, 827359, or 827392. In any of the foregoing embodiments, the compound can be single-stranded or double-stranded. In any of the foregoing embodiments, the compound can be an antisense compound or oligomeric compound. In certain embodiments, the compound is administered to the individual via inhalation. In certain embodiments, administering the compound improves or preserves lung function. In certain such embodiments, spirometry or mucociliary clearance is improved or preserved. In certain such embodiments, forced expiratory volume in one second (FEV₁), FVC, or FEF₂₅₋₇₅ is increased. In certain embodiments, pulmonary exacerbations, hospitalization rate or frequency, or antibiotic use is decreased. In certain embodiments, quality of life is improved, as measured by the respiratory questionnaire, CFQ-R. In certain embodiments, the individual is identified as having or at risk of having a disease associated with ENaC.

In certain embodiments, a method of inhibiting expression of α-ENaC in an individual having, or at risk of having, a disease associated with ENaC comprises administering to the individual a compound comprising an α-ENaC inhibitor, thereby inhibiting expression of α-ENaC in the individual. In certain embodiments, administering the compound inhibits expression of α-ENaC in the lung. In certain embodiments, the individual has, or is at risk of having cystic fibrosis, COPD, asthma, or chronic bronchitis. In certain embodiments, the compound comprises an antisense compound targeted to α-ENaC. In certain embodiments, the compound comprises an oligonucleotide complementary to an α-ENaC nucleic acid transcript. In certain embodiments, the oligonucleotide is a modified oligonucleotide. In certain embodiments, the compound comprise a modified oligonucleotide complementary to an intron of an α-ENaC nucleic acid transcript. In certain embodiments, the modified oligonucleotide is complementary to intron 1, intron 2, intron 3, intron 4, intron 5, intron 6, intron 7, intron 8, intron 9, intron 10, intron 11, or intron 12 of an α-ENaC nucleic acid transcript. In certain such embodiments, the oligonucleotide is complementary to a sequence within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, the compound comprises a modified oligonucleotide 8 to 50 linked nucleosides in length and having a nucleobase sequence comprising at least 8 contiguous nucleobases of any of the nucleobase sequences of SEQ ID NOs: 6-1954. In certain embodiments, the compound comprises a modified oligonucleotide 12 to 50 linked nucleosides in length and having a nucleobase sequence comprising the nucleobase sequence of any one of SEQ ID NOs: 6-1954. In certain embodiments, the compound comprises a modified oligonucleotide consisting of the nucleobase sequence of any one of SEQ ID NOs: 6-1954. In certain embodiments, the compound comprises a modified oligonucleotide 16 to 50 linked nucleosides in length having a nucleobase sequence comprising any one of SEQ ID NOs: 239, 426, 1541, 1812, 1113, or 593. In certain embodiments, the compound comprises a modified oligonucleotide having a nucleobase sequence consisting of any one of SEQ ID NOs: 239, 426, 1541, 1812, 1113, or 593. In any of the foregoing embodiments, the modified oligonucleotide can be 10 to 30 linked nucleosides in length. In certain embodiments, the compound is compound number 797308, 797495, 826763, 827307, 827359, or 827392. In any of the foregoing embodiments, the compound can be single-stranded or double-stranded. In any of the foregoing embodiments, the compound can be an antisense compound or oligomeric compound. In certain embodiments, the compound is administered to the individual via inhalation. In certain embodiments, administering the compound improves or preserves spirometry or mucociliary clearance. In certain embodiments, the individual is identified as having or at risk of having a disease associated with ENaC.

In certain embodiments, a method of inhibiting expression of α-ENaC in a cell comprises contacting the cell with a compound comprising an α-ENaC inhibitor, thereby inhibiting expression of α-ENaC in the cell. In certain embodiments, the cell is a lung cell. In certain embodiments, the cell is in the lung. In certain embodiments, the cell is in the lung of an individual who has, or is at risk of having cystic fibrosis, COPD, asthma, or chronic bronchitis. In certain embodiments, the compound comprises an antisense compound targeted to α-ENaC. In certain embodiments, the compound comprises an oligonucleotide complementary to an α-ENaC nucleic acid transcript. In certain embodiments, the oligonucleotide is a modified oligonucleotide. In certain embodiments, the compound comprise a modified oligonucleotide complementary to an intron of an α-ENaC nucleic acid transcript. In certain embodiments, the modified oligonucleotide is complementary to intron 1, intron 2, intron 3, intron 4, intron 5, intron 6, intron 7, intron 8, intron 9, intron 10, intron 11, or intron 12 of an α-ENaC nucleic acid transcript. In certain such embodiments, the oligonucleotide is complementary to a sequence within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, the compound comprises a modified oligonucleotide 8 to 50 linked nucleosides in length and having a nucleobase sequence comprising at least 8 contiguous nucleobases of any of the nucleobase sequences of SEQ ID NOs: 6-1954. In certain embodiments, the compound comprises a modified oligonucleotide 12 to 50 linked nucleosides in length and having a nucleobase sequence comprising the nucleobase sequence of any one of SEQ ID NOs: 6-1954. In certain embodiments, the compound comprises a modified oligonucleotide consisting of the nucleobase sequence of any one of SEQ ID NOs: 6-1954. In certain embodiments, the compound comprises a modified oligonucleotide 16 to 50 linked nucleosides in length having a nucleobase sequence comprising any one of SEQ ID NOs: 239, 426, 1541, 1812, 1113, or 593. In certain embodiments, the compound comprises a modified oligonucleotide having a nucleobase sequence consisting of any one of SEQ ID NOs: 239, 426, 1541, 1812, 1113, or 593. In any of the foregoing embodiments, the modified oligonucleotide can be 10 to 30 linked nucleosides in length. In certain embodiments, the compound is compound number 797308, 797495, 826763, 827307, 827359, or 827392. In any of the foregoing embodiments, the compound can be single-stranded or double-stranded. In any of the foregoing embodiments, the compound can be an antisense compound or oligomeric compound.

In certain embodiments, a method of increasing or improving spirometry or mucociliary clearance in the lung of an individual having, or at risk of having, a disease associated with ENaC comprises administering to the individual a compound comprising an α-ENaC inhibitor, thereby increasing or improving spirometry or mucociliary clearance in the lung of the individual. In certain such embodiments, forced expiratory volume in one second (FEV₁), FVC, or FEF₂₅₋₇₅ is increased. In certain embodiments, pulmonary exacerbations, hospitalization rate or frequency, or antibiotic use is decreased. In certain embodiments, quality of life is improved, as measured by the respiratory questionnaire, CFQ-R. In certain embodiments, the individual has, or is at risk of having, cystic fibrosis, COPD, asthma, or chronic bronchitis. In certain embodiments, the compound comprises an antisense compound targeted to α-ENaC. In certain embodiments, the compound comprises an oligonucleotide complementary to an α-ENaC nucleic acid transcript. In certain embodiments, the oligonucleotide is a modified oligonucleotide. In certain embodiments, the compound comprise a modified oligonucleotide complementary to an intron of an α-ENaC nucleic acid transcript. In certain embodiments, the modified oligonucleotide is complementary to intron 1, intron 2, intron 3, intron 4, intron 5, intron 6, intron 7, intron 8, intron 9, intron 10, intron 11, or intron 12 of an α-ENaC nucleic acid transcript. In certain such embodiments, the oligonucleotide is complementary to a sequence within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, the compound comprises a modified oligonucleotide 8 to 50 linked nucleosides in length and having a nucleobase sequence comprising at least 8 contiguous nucleobases of any of the nucleobase sequences of SEQ ID NOs: 6-1954. In certain embodiments, the compound comprises a modified oligonucleotide 12 to 50 linked nucleosides in length and having a nucleobase sequence comprising the nucleobase sequence of any one of SEQ ID NOs: 6-1954. In certain embodiments, the compound comprises a modified oligonucleotide consisting of the nucleobase sequence of any one of SEQ ID NOs: 6-1954. In certain embodiments, the compound comprises a modified oligonucleotide 16 to 50 linked nucleosides in length having a nucleobase sequence comprising any one of SEQ ID NOs: 239, 426, 1541, 1812, 1113, or 593. In certain embodiments, the compound comprises a modified oligonucleotide having a nucleobase sequence consisting of any one of SEQ ID NOs: 239, 426, 1541, 1812, 1113, or 593. In any of the foregoing embodiments, the modified oligonucleotide can be 10 to 30 linked nucleosides in length. In certain embodiments, the compound is compound number 797308, 797495, 826763, 827307, 827359, or 827392. In any of the foregoing embodiments, the compound can be single-stranded or double-stranded. In any of the foregoing embodiments, the compound can be an antisense compound or oligomeric compound. In certain embodiments, the compound is administered to the individual via inhalation. In certain embodiments, the individual is identified as having or at risk of having a disease associated with ENaC.

Certain embodiments are drawn to a compound comprising an α-ENaC inhibitor for use in treating a disease associated with ENaC. In certain embodiments, the disease is cystic fibrosis, COPD, asthma, or chronic bronchitis. In certain embodiments, the compound comprises an antisense compound targeted to α-ENaC. In certain embodiments, the compound comprises an oligonucleotide complementary to an α-ENaC nucleic acid transcript. In certain embodiments, the oligonucleotide is a modified oligonucleotide. In certain embodiments, the compound comprise a modified oligonucleotide complementary to an intron of an α-ENaC nucleic acid transcript. In certain embodiments, the modified oligonucleotide is complementary to intron 1, intron 2, intron 3, intron 4, intron 5, intron 6, intron 7, intron 8, intron 9, intron 10, intron 11, or intron 12 of an α-ENaC nucleic acid transcript. In certain such embodiments, the oligonucleotide is complementary to a sequence within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, the compound comprises a modified oligonucleotide 8 to 50 linked nucleosides in length and having a nucleobase sequence comprising at least 8 contiguous nucleobases of any of the nucleobase sequences of SEQ ID NOs: 6-1954. In certain embodiments, the compound comprises a modified oligonucleotide 12 to 50 linked nucleosides in length and having a nucleobase sequence comprising the nucleobase sequence of any one of SEQ ID NOs: 6-1954. In certain embodiments, the compound comprises a modified oligonucleotide consisting of the nucleobase sequence of any one of SEQ ID NOs: 6-1954. In certain embodiments, the compound comprises a modified oligonucleotide 16 to 50 linked nucleosides in length having a nucleobase sequence comprising any one of SEQ ID NOs: 239, 426, 1541, 1812, 1113, or 593. In certain embodiments, the compound comprises a modified oligonucleotide having a nucleobase sequence consisting of any one of SEQ ID NOs: 239, 426, 1541, 1812, 1113, or 593. In any of the foregoing embodiments, the modified oligonucleotide can be 10 to 30 linked nucleosides in length. In certain embodiments, the compound is compound number 797308, 797495, 826763, 827307, 827359, or 827392. In any of the foregoing embodiments, the compound can be single-stranded or double-stranded. In any of the foregoing embodiments, the compound can be an antisense compound or oligomeric compound.

Certain embodiments are drawn to a compound comprising an α-ENaC inhibitor for use in increasing or improving spirometry or mucociliary clearance of an individual having or at risk of having cystic fibrosis, COPD, asthma, or chronic bronchitis. In certain embodiments, the compound comprises an antisense compound targeted to α-ENaC. In certain embodiments, the compound comprises an oligonucleotide complementary to an α-ENaC nucleic acid transcript. In certain embodiments, the oligonucleotide is a modified oligonucleotide. In certain embodiments, the compound comprise a modified oligonucleotide complementary to an intron of an α-ENaC nucleic acid transcript. In certain embodiments, the modified oligonucleotide is complementary to intron 1, intron 2, intron 3, intron 4, intron 5, intron 6, intron 7, intron 8, intron 9, intron 10, intron 11, or intron 12 of an α-ENaC nucleic acid transcript. In certain such embodiments, the oligonucleotide is complementary to a sequence within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, the compound comprises a modified oligonucleotide 8 to 50 linked nucleosides in length and having a nucleobase sequence comprising at least 8 contiguous nucleobases of any of the nucleobase sequences of SEQ ID NOs: 6-1954. In certain embodiments, the compound comprises a modified oligonucleotide 12 to 50 linked nucleosides in length and having a nucleobase sequence comprising the nucleobase sequence of any one of SEQ ID NOs: 6-1954. In certain embodiments, the compound comprises a modified oligonucleotide consisting of the nucleobase sequence of any one of SEQ ID NOs: 6-1954. In certain embodiments, the compound comprises a modified oligonucleotide 16 to 50 linked nucleosides in length having a nucleobase sequence comprising any one of SEQ ID NOs: 239, 426, 1541, 1812, 1113, or 593. In certain embodiments, the compound comprises a modified oligonucleotide having a nucleobase sequence consisting of any one of SEQ ID NOs: 239, 426, 1541, 1812, 1113, or 593. In any of the foregoing embodiments, the modified oligonucleotide can be 10 to 30 linked nucleosides in length. In certain embodiments, the compound is compound number 797308, 797495, 826763, 827307, 827359, or 827392. In any of the foregoing embodiments, the compound can be single-stranded or double-stranded. In any of the foregoing embodiments, the compound can be an antisense compound or oligomeric compound.

Certain embodiments are drawn to use of a compound comprising an α-ENaC inhibitor for the manufacture or preparation of a medicament for treating a disease associated with ENaC. Certain embodiments are drawn to use of a compound comprising an α-ENaC inhibitor for the preparation of a medicament for treating a disease associated with ENaC. In certain embodiments, the disease is cystic fibrosis, COPD, asthma, or chronic bronchitis. In certain embodiments, the compound comprises an antisense compound targeted to α-ENaC. In certain embodiments, the compound comprises an oligonucleotide complementary to an α-ENaC nucleic acid transcript. In certain embodiments, the oligonucleotide is a modified oligonucleotide. In certain embodiments, the compound comprise a modified oligonucleotide complementary to an intron of an α-ENaC nucleic acid transcript. In certain embodiments, the modified oligonucleotide is complementary to intron 1, intron 2, intron 3, intron 4, intron 5, intron 6, intron 7, intron 8, intron 9, intron 10, intron 11, or intron 12 of an α-ENaC nucleic acid transcript. In certain such embodiments, the oligonucleotide is complementary to a sequence within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, the compound comprises a modified oligonucleotide 8 to 50 linked nucleosides in length and having a nucleobase sequence comprising at least 8 contiguous nucleobases of any of the nucleobase sequences of SEQ ID NOs: 6-1954. In certain embodiments, the compound comprises a modified oligonucleotide 12 to 50 linked nucleosides in length and having a nucleobase sequence comprising the nucleobase sequence of any one of SEQ ID NOs: 6-1954. In certain embodiments, the compound comprises a modified oligonucleotide consisting of the nucleobase sequence of any one of SEQ ID NOs: 6-1954. In certain embodiments, the compound comprises a modified oligonucleotide 16 to 50 linked nucleosides in length having a nucleobase sequence comprising any one of SEQ ID NOs: 239, 426, 1541, 1812, 1113, or 593. In certain embodiments, the compound comprises a modified oligonucleotide having a nucleobase sequence consisting of any one of SEQ ID NOs: 239, 426, 1541, 1812, 1113, or 593. In any of the foregoing embodiments, the modified oligonucleotide can be 10 to 30 linked nucleosides in length. In certain embodiments, the compound is compound number 797308, 797495, 826763, 827307, 827359, or 827392. In any of the foregoing embodiments, the compound can be single-stranded or double-stranded. In any of the foregoing embodiments, the compound can be an antisense compound or oligomeric compound.

Certain embodiments are drawn to use of a compound comprising an α-ENaC inhibitor for the manufacture or preparation of a medicament for increasing or improving spirometry or mucociliary clearance in an individual having or at risk of having cystic fibrosis, COPD, asthma, or chronic bronchitis. In certain embodiments, the compound comprises an antisense compound targeted to α-ENaC. In certain embodiments, the compound comprises an oligonucleotide complementary to an α-ENaC nucleic acid transcript. In certain embodiments, the oligonucleotide is a modified oligonucleotide. In certain embodiments, the compound comprise a modified oligonucleotide complementary to an intron of an α-ENaC nucleic acid transcript. In certain embodiments, the modified oligonucleotide is complementary to intron 1, intron 2, intron 3, intron 4, intron 5, intron 6, intron 7, intron 8, intron 9, intron 10, intron 11, or intron 12 of an α-ENaC nucleic acid transcript. In certain such embodiments, the oligonucleotide is complementary to a sequence within nucleotides 4,497-5,163; 5,634-16,290; 16,559-17,759; 17,951-24,120; 24,225-24,565; 24,730-25,152; 25,252-25,445; 25,564-30,595; 30,675-30,779; 30,838-30,995; 31,052-31,198; or 31,275-31,747 of SEQ ID NO: 2. In certain embodiments, the compound comprises a modified oligonucleotide 8 to 50 linked nucleosides in length and having a nucleobase sequence comprising at least 8 contiguous nucleobases of any of the nucleobase sequences of SEQ ID NOs: 6-1954. In certain embodiments, the compound comprises a modified oligonucleotide 12 to 50 linked nucleosides in length and having a nucleobase sequence comprising the nucleobase sequence of any one of SEQ ID NOs: 6-1954. In certain embodiments, the compound comprises a modified oligonucleotide consisting of the nucleobase sequence of any one of SEQ ID NOs: 6-1954. In certain embodiments, the compound comprises a modified oligonucleotide 16 to 50 linked nucleosides in length having a nucleobase sequence comprising any one of SEQ ID NOs: 239, 426, 1541, 1812, 1113, or 593. In certain embodiments, the compound comprises a modified oligonucleotide having a nucleobase sequence consisting of any one of SEQ ID NOs: 239, 426, 1541, 1812, 1113, or 593. In any of the foregoing embodiments, the modified oligonucleotide can be 10 to 30 linked nucleosides in length. In certain embodiments, the compound is compound number 797308, 797495, 826763, 827307, 827359, or 827392. In any of the foregoing embodiments, the compound can be single-stranded or double-stranded. In any of the foregoing embodiments, the compound can be an antisense compound or oligomeric compound.

In any of the foregoing methods or uses, the compound can be targeted to α-ENaC. In certain embodiments, the compound comprises or consists of a modified oligonucleotide, for example a modified oligonucleotide 8 to 50 linked nucleosides in length, 10 to 30 linked nucleosides in length, 12 to 30 linked nucleosides in length, or 20 linked nucleosides in length. In certain embodiments, the modified oligonucleotide is at least 80%, 85%, 90%, 95% or 100% complementary to any of the nucleobase sequences recited in SEQ ID NOs: 1, 2, or 1957. In certain embodiments, the modified oligonucleotide comprises at least one modified internucleoside linkage, at least one modified sugar, and at least one modified nucleobase. In certain such embodiments, the at least one modified internucleoside linkage is a phosphorothioate internucleoside linkage, the at least one modified sugar is a bicyclic sugar or a 2′-MOE sugar, and the at least one modified nucleobase is a 5-methylcytosine. In certain embodiments, the modified oligonucleotide comprises a gap segment consisting of linked 2′-deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each terminal wing nucleoside comprises a modified sugar.

In any of the foregoing embodiments, the modified oligonucleotide is 12 to 30, 15 to 30, 15 to 25, 15 to 24, 16 to 24, 17 to 24, 18 to 24, 19 to 24, 20 to 24, 19 to 22, 20 to 22, 16 to 20, or 17 or 20 linked nucleosides in length. In certain embodiments, the modified oligonucleotide is at least 80%, 85%, 90%, 95% or 100% complementary to any of the nucleobase sequences recited in SEQ ID NOs: 1, 2, or 1957. In certain embodiments, the modified oligonucleotide comprises at least one modified intemucleoside linkage, at least one modified sugar, and at least one modified nucleobase. In certain embodiments, the at least one modified internucleoside linkage is a phosphorothioate intemucleoside linkage, the at least one modified sugar is a bicyclic sugar or a 2′-MOE sugar, and the at least one modified nucleobase is a 5-methylcytosine. In certain embodiments, the modified oligonucleotide comprises a gap segment consisting of linked 2′-deoxynucleosides; a 5′ wing segment consisting of linked nucleosides; and a 3′ wing segment consisting of linked nucleosides, wherein the gap segment is positioned immediately adjacent to and between the 5′ wing segment and the 3′ wing segment and wherein each terminal wing nucleoside comprises a modified sugar.

In any of the foregoing methods or uses, the compound comprises or consists of a modified oligonucleotide 16 to 30 linked nucleosides in length and having a nucleobase sequence comprising any one of SEQ ID NOs: 6-1954, wherein the modified oligonucleotide comprises:
a gap segment consisting of linked 2′-deoxynucleosides;
a 5′ wing segment consisting of linked nucleosides; and
a 3′ wing segment consisting of linked nucleosides;
wherein the gap segment is positioned between the 5′ wing segment and the 3′ wing segment and wherein each nucleoside of each wing segment comprises a modified sugar.

In any of the foregoing methods or uses, the compound comprises or consists of a modified oligonucleotide 16 to 30 linked nucleosides in length and having a nucleobase sequence comprising any one of SEQ ID NOs: 6-1954, wherein the modified oligonucleotide comprises: a gap segment consisting of linked 2′-deoxynucleosides;
a 5′ wing segment consisting of linked nucleosides; and a 3′ wing segment consisting of linked nucleosides;
wherein the gap segment is positioned between the 5′ wing segment and the 3′ wing segment, wherein each terminal wing nucleoside comprises a modified sugar.

In any of the foregoing methods or uses, the compound comprises or consists a modified oligonucleotide 20 linked nucleosides in length having a nucleobase sequence comprising the sequence recited in any one of SEQ ID NOs: 6-1954, wherein the modified oligonucleotide comprises
a gap segment consisting of ten linked 2′-deoxynucleosides;
a 5′ wing segment consisting of five linked nucleosides; and
a 3′ wing segment consisting of five linked nucleosides;
wherein the gap segment is positioned between the 5′ wing segment and the 3′ wing segment, wherein each nucleoside of each wing segment comprises a 2′-O-methoxyethyl sugar; wherein each internucleoside linkage is a phosphorothioate linkage and wherein each cytosine is a 5-methylcytosine. In certain embodiments, the modified oligonucleotide consists of 20-30 linked nucleosides. In certain embodiments, the modified oligonucleotide consists of 20 linked nucleosides.

In any of the foregoing methods or uses, the compound comprises or consists a modified oligonucleotide 16 to 50 linked nucleobases in length having a nucleobase sequence comprising or consisting of the sequence recited in any one of SEQ ID NOs: 6-1954, wherein the modified oligonucleotide comprises
a gap segment consisting of 10 linked 2′-deoxynucleosides;
a 5′ wing segment consisting of 3 linked nucleosides; and
a 3′ wing segment consisting of 3 linked nucleosides;
wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine. In certain embodiments, the modified oligonucleotide consists of 16-30 linked nucleosides. In certain embodiments, the modified oligonucleotide consists of 16 linked nucleosides.

In any of the foregoing methods or uses, the compound comprises or consists a modified oligonucleotide 16 to 50 linked nucleobases in length having a nucleobase sequence comprising or consisting of the sequence recited in any one of SEQ ID NOs: 239, 426, 593, 1113, 1541, or 1812, wherein the modified oligonucleotide comprises
a gap segment consisting of 10 linked 2′-deoxynucleosides;
a 5′ wing segment consisting of 3 linked nucleosides; and
a 3′ wing segment consisting of 3 linked nucleosides;
wherein the gap segment is positioned between the 5′ wing segment and the 3′ wing segment, wherein each nucleoside of each wing segment comprises a cEt sugar; wherein each internucleoside linkage is a phosphorothioate linkage and wherein each cytosine is a 5-methylcytosine. In certain embodiments, the modified oligonucleotide consists of 16-30 linked nucleosides. In certain embodiments, the modified oligonucleotide consists of 16 linked nucleosides.

In any of the foregoing methods or uses, the compound has the following chemical structure:

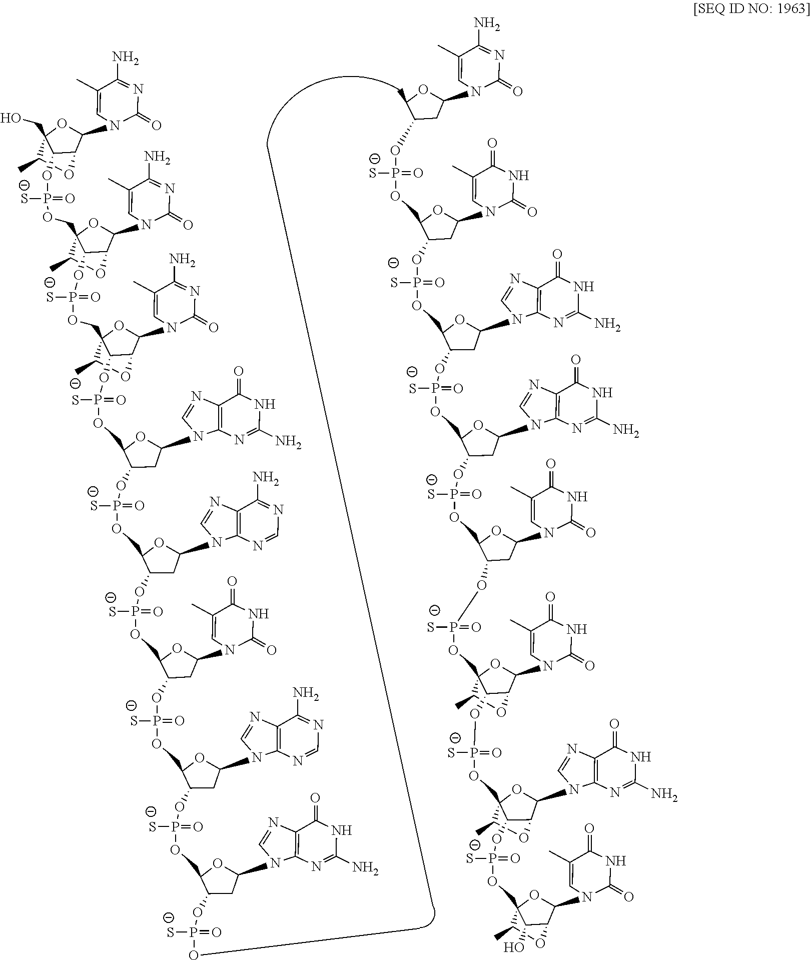

In any of the foregoing methods or uses, the compound can be administered via inhalation. In certain embodiments, the compound of any of the foregoing methods or uses can be administered through injection or infusion. In certain embodiments, the compound of any of the foregoing methods or uses can be administered via subcutaneous administration, intravenous administration, intramuscular administration, intraarterial administration, intraperitoneal administration, or intracranial administration, e.g. intrathecal or intracerebroventricular administration. In certain embodiments, the compound of any of the foregoing methods or uses can be administered systemically. In certain embodiments, the compound of any of the foregoing methods or uses can be administered orally.

### Certain Combinations and Combination Therapies

In certain embodiments, a first agent comprising the compound described herein is co-administered with one or more secondary agents. In certain embodiments, such second agents are designed to treat the same disease, disorder, or condition as the first agent described herein. In certain embodiments, such second agents are designed to treat a different disease, disorder, or condition as the first agent described herein. In certain embodiments, a first agent is designed to treat an undesired side effect of a second agent. In certain embodiments, second agents are co-administered with the first agent to treat an undesired effect of the first agent. In certain embodiments, such second agents are designed to treat an undesired side effect of one or more pharmaceutical compositions as described herein. In certain embodiments, second agents are co-administered with the first agent to produce a combinational effect. In certain embodiments, second agents are co-administered with the first agent to produce a synergistic effect. In certain embodiments, the co-administration of the first and second agents permits use of lower dosages than would be required to achieve a therapeutic or prophylactic effect if the agents were administered as independent therapy.

In certain embodiments, one or more compounds or compositions provided herein are co-administered with one or more secondary agents. In certain embodiments, one or more compounds or compositions provided herein and one or more secondary agents, are administered at different times. In certain embodiments, one or more compounds or compositions provided herein and one or more secondary agents, are prepared together in a single formulation. In certain embodiments, one or more compounds or compositions provided herein and one or more secondary agents, are prepared separately. In certain embodiments, a secondary agent is a bronchodilator, a corticosteroid, an antibiotic, a second compound comprising or consisting of a modified oligonucleotide, and/or a chloride channel (CFTR) modulator. In certain embodiments, a secondary agent is selected from: hypertonic saline, dornase alfa, ivacaftor, tezacaftor, and lumacaftor.

Certain embodiments are directed to the use of a compound comprising a modified oligonucleotide complementary to an α-ENaC nucleic acid transcript as described herein in combination with a secondary agent. In particular embodiments such use is in a method of treating a patient suffering from cystic fibrosis, COPD, asthma, or chronic bronchitis or in the preparation or manufacture of a medicament for treating cystic fibrosis, COPD, asthma, or chronic bronchitis. In certain embodiments, a secondary agent is a bronchodilator, a corticosteroid, an antibiotic, or a chloride channel (CFTR) modulator. In certain embodiments, a secondary agent is selected from: hypertonic saline, dornase alfa, ivacaftor, tezacaftor, and lumacaftor.

Certain embodiments are directed to the use of a compound comprising a modified oligonucleotide complementary to an α-ENaC nucleic acid transcript as described herein in combination with two or more secondary agents. In particular embodiments such use is in a method of treating a patient suffering from cystic fibrosis, COPD, asthma, or chronic bronchitis or in the preparation or manufacture of a medicament for treating cystic fibrosis, COPD, asthma, or chronic bronchitis. In certain embodiments, two or more secondary agents are selected from bronchodilators, corticosteroids, antibiotics, and chloride channel (CFTR) modulators. In certain embodiments, two or more secondary agents are selected from: hypertonic saline, dornase alfa, ivacaftor, tezacaftor, and lumacaftor.

Certain embodiments are drawn to a combination of a compound comprising a modified oligonucleotide complemetary to an α-ENaC nucleic acid transcript as described herein and a secondary agent, such as a secondary agent selected from: hypertonic saline, dornase alfa, ivacaftor, tezacaftor, and lumacaftor. In certain embodiments, such a combination of a compound comprising a modified oligonucleotide complemetary to an α-ENaC nucleic acid transcript as described herein and a secondary agent, such as a secondary agent selected from: hypertonic saline, dornase alfa, ivacaftor, tezacaftor, and lumacaftor is useful for improving or preserving spirometry or mucociliary clearance and/or treating cystic fibrosis, COPD, asthma, or chronic bronchitis.

Certain embodiments are drawn to a combination of a compound comprising a modified oligonucleotide complemetary to an α-ENaC nucleic acid transcript as described herein and two or more secondary agents, such as secondary agents selected from: hypertonic saline, dornase alfa, ivacaftor, tezacaftor, and lumacaftor. In certain embodiments, such a combination of a compound comprising a modified oligonucleotide complemetary to an α-ENaC nucleic acid transcript as described herein and two ore more secondary agents, such as secondary agents selected from: hypertonic saline, dornase alfa, ivacaftor, tezacaftor, and lumacaftor is useful for improving or preserving spirometry or mucociliary clearance and/or treating cystic fibrosis, COPD, asthma, or chronic bronchitis.

In certain embodiments the compound comprising a modified oligonucleotide complemetary to an α-ENaC nucleic acid transcript as described herein and the secondary agent are used in combination treatment by administering the two agents simultaneously, separately or sequentially. In certain embodiments the two agents are formulated as a fixed dose combination product. In other embodiments the two agents are provided to the patient as separate units which can then either be taken simultaneously or serially (sequentially).

In certain embodiments the compound comprising a modified oligonucleotide complemetary to an α-ENaC nucleic acid transcript as described herein and two or more secondary agents are used in combination treatment by administering the three or more agents simultaneously, separately or sequentially. In certain embodiments the three or more agents are formulated as a fixed dose combination product. In other embodiments the three or more agents are provided to the patient as separate units which can then either be taken simultaneously or serially (sequentially).

### Certain Compounds

In certain embodiments, compounds described herein can be antisense compounds. In certain embodiments, the antisense compound comprises or consists of an oligomeric compound. In certain embodiments, the oligomeric compound comprises or consists of a modified oligonucleotide. In certain embodiments, the modified oligonucleotide has a nucleobase sequence complementary to that of a target nucleic acid.

In certain embodiments, a compound described herein comprises or consists of a modified oligonucleotide. In certain embodiments, the modified oligonucleotide has a nucleobase sequence complementary to that of a target nucleic acid.

In certain embodiments, a compound or antisense compound is single-stranded. Such a single-stranded compound or antisense compound comprises or consists of an oligomeric compound. In certain embodiments, such an oligomeric compound comprises or consists of an oligonucleotide and optionally a conjugate group. In certain embodiments, the oligonucleotide is an antisense oligonucleotide. In certain embodiments, the oligonucleotide is modified. In certain embodiments, the oligonucleotide of a single-stranded antisense compound or oligomeric compound comprises a self-complementary nucleobase sequence.

In certain embodiments, a compound or antisense compound is double-stranded. Such double-stranded compounds comprise a first oligomeric compound comprising or consisting of a first modified oligonucleotide having a region complementary to a target nucleic acid and a second oligomeric compound comprising or consisting of a second oligonucleotide having a region complementary to the first modified oligonucleotide. In certain embodiments, the first oligonucleotide is 100% complementary to the second oligonucleotide. In certain embodiments, the first and second oligonucleotides include non-complementary, overhanging nucleosides. In certain embodiments, the first modified oligonucleotide comprises unmodified ribosyl sugar moieties as those found in RNA. In such embodiments, thymine nucleobases in the first and/or second oligonucleotide are replaced by uracil nucleobases. In certain embodiments, the first and/or second oligomeric compound comprises a conjugate group. In certain embodiments, the first modified oligonucleotide is 12-30 linked nucleosides in length and the second oligonucleotide is 12-30 linked nucleosides in length. In certain embodiments, the second oligonucleotide is modified. In certain embodiments, the first modified oligonucleotide has a nucleobase sequence comprising at least 8 contiguous nucleobases of any of SEQ ID NOs: 6-1954.

Examples of single-stranded and double-stranded compounds include but are not limited to oligonucleotides, siRNAs, microRNA targeting oligonucleotides, and single-stranded RNAi compounds, such as small hairpin RNAs (shRNAs), single-stranded siRNAs (ssRNAs), and microRNA mimics.

In certain embodiments, a compound described herein has a nucleobase sequence that, when written in the 5′ to 3′ direction, comprises the reverse complement of the target segment of a target nucleic acid to which it is targeted.

In certain embodiments, a compound described herein comprises an oligonucleotide 10 to 30 linked subunits in length. In certain embodiments, a compound described herein comprises an oligonucleotide 12 to 30 linked subunits in length. In certain embodiments, a compound described herein comprises an oligonucleotide 12 to 22 linked subunits in length. In certain embodiments, compound described herein comprises an oligonucleotide 14 to 30 linked subunits in length. In certain embodiments, compound described herein comprises an oligonucleotide 14 to 20 linked subunits in length. In certain embodiments, a compound described herein comprises an oligonucleotide 15 to 30 linked subunits in length. In certain embodiments, a compound described herein comprises an oligonucleotide 15 to 20 linked subunits in length. In certain embodiments, a compound described herein comprises an oligonucleotide 16 to 30 linked subunits in length. In certain embodiments, a compound described herein comprises an oligonucleotide 16 to 20 linked subunits in length. In certain embodiments, a compound described herein comprises an oligonucleotide 17 to 30 linked subunits in length. In certain embodiments, a compound described herein comprises an oligonucleotide 17 to 20 linked subunits in length. In certain embodiments, a compound described herein comprises an oligonucleotide 18 to 30 linked subunits in length. In certain embodiments, a compound described herein comprises an oligonucleotide 18 to 21 linked subunits in length. In certain embodiments, a compound described herein comprises an oligonucleotide 18 to 20 linked subunits in length. In certain embodiments, a compound described herein comprises an oligonucleotide 20 to 30 linked subunits in length. In other words, such oligonucleotides are 12 to 30 linked subunits, 14 to 30 linked subunits, 14 to 20 subunits, 15 to 30 subunits, 15 to 20 subunits, 16 to 30 subunits, 16 to 20 subunits, 17 to 30 subunits, 17 to 20 subunits, 18 to 30 subunits, 18 to 20 subunits, 18 to 21 subunits, 20 to 30 subunits, or 12 to 22 linked subunits in length, respectively. In certain embodiments, a compound described herein comprises an oligonucleotide 14 linked subunits in length. In certain embodiments, a compound described herein comprises an oligonucleotide 16 linked subunits in length. In certain embodiments, a compound described herein comprises an oligonucleotide 17 linked subunits in length. In certain embodiments, compound described herein comprises an oligonucleotide 18 linked subunits in length. In certain embodiments, a compound described herein comprises an oligonucleotide 19 linked subunits in length. In certain embodiments, a compound described herein comprises an oligonucleotide 20 linked subunits in length. In other embodiments, a compound described herein comprises an oligonucleotide 8 to 80, 12 to 50, 13 to 30, 13 to 50, 14 to 30, 14 to 50, 15 to 30,15 to 50, 16 to 30, 16 to 50, 17 to 30, 17 to 50, 18 to 22, 18 to 24, 18 to 30, 18 to 50, 19 to 22, 19 to 30, 19 to 50, or 20 to 30 linked subunits. In certain such embodiments, the compound described herein comprises an oligonucleotide 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 linked subunits in length, or a range defined by any two of the above values. In some embodiments the linked subunits are nucleotides, nucleosides, or nucleobases.

In certain embodiments, the compound may further comprise additional features or elements, such as a conjugate group, that are attached to the oligonucleotide. In certain embodiments, such compounds are antisense compounds. In certain embodiments, such compounds are oligomeric compounds. In embodiments where a conjugate group comprises a nucleoside (i.e. a nucleoside that links the conjugate group to the oligonucleotide), the nucleoside of the conjugate group is not counted in the length of the oligonucleotide.

In certain embodiments, compounds may be shortened or truncated. For example, a single subunit may be deleted from the 5′ end (5′ truncation), or alternatively from the 3′ end (3′ truncation). A shortened or truncated compound targeted to an α-ENaC nucleic acid may have two subunits deleted from the 5′ end, or alternatively may have two subunits deleted from the 3′ end, of the compound. Alternatively, the deleted nucleosides may be dispersed throughout the compound.

When a single additional subunit is present in a lengthened compound, the additional subunit may be located at the 5′ or 3′ end of the compound. When two or more additional subunits are present, the added subunits may be adjacent to each other, for example, in a compound having two subunits added to the 5′ end (5′ addition), or alternatively to the 3′ end (3′ addition), of the compound. Alternatively, the added subunits may be dispersed throughout the compound.

It is possible to increase or decrease the length of a compound, such as an oligonucleotide, and/or introduce mismatch bases without eliminating activity (Woolf et al. Proc. Natl. Acad. Sci. USA 1992, 89:7305-7309; Gautschi et al. J Natl. Cancer Inst. March 2001, 93:463-471; Maher and Dolnick Nuc. Acid. Res. 1998, 16:3341-3358). However, seemingly small changes in oligonucleotide sequence, chemistry and motif can make large differences in one or more of the many properties required for clinical development (Seth et al. J. Med. Chem. 2009, 52, 10; Egli et al. J Am. Chem. Soc. 2011, 133, 16642).

In certain embodiments, compounds described herein are interfering RNA compounds (RNAi), which include double-stranded RNA compounds (also referred to as short-interfering RNA or siRNA) and single-stranded RNAi compounds (or ssRNA). Such compounds work at least in part through the RISC pathway to degrade and/or sequester a target nucleic acid (thus, include microRNA/microRNA-mimic compounds). As used herein, the term siRNA is meant to be equivalent to other terms used to describe nucleic acid molecules that are capable of mediating sequence specific RNAi, for example short interfering RNA (siRNA), double-stranded RNA (dsRNA), micro-RNA (miRNA), short hairpin RNA (shRNA), short interfering oligonucleotide, short interfering nucleic acid, short interfering modified oligonucleotide, chemically modified siRNA, post-transcriptional gene silencing RNA (ptgsRNA), and others. In addition, as used herein, the term “RNAi” is meant to be equivalent to other terms used to describe sequence specific RNA interference, such as post transcriptional gene silencing, translational inhibition, or epigenetics.

In certain embodiments, a compound described herein can comprise any of the oligonucleotide sequences targeted to an α-ENaC nucleic acid transcript described herein. In certain embodiments, the compound can be double-stranded. In certain embodiments, the compound comprises a first strand comprising at least an 8, 9, 10, 11, 12, 13, 14, 15, 16, 17, 18, 19, or 20 contiguous nucleobase portion of any one of SEQ ID NOs: 6-1954 and a second strand. In certain embodiments, the compound comprises a first strand comprising the nucleobase sequence of any one of SEQ ID NOs: 6-1954 and a second strand. In certain embodiments, the compound comprises ribonucleotides in which the first strand has uracil (U) in place of thymine (T) in any one of SEQ ID NOs: 6-1954. In certain embodiments, the compound comprises (i) a first strand comprising a nucleobase sequence complementary to the site on an α-ENaC nucleic acid to which any of SEQ ID NOs: 6-1954 is complementary, and (ii) a second strand. In certain embodiments, the compound comprises one or more modified nucleotides in which the 2′ position in the sugar contains a halogen (such as fluorine group; 2′-F) or contains an alkoxy group (such as a methoxy group; 2′-OMe). In certain embodiments, the compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the dsRNA compound. In certain embodiments, the compound comprises one or more linkages between adjacent nucleotides other than a naturally-occurring phosphodiester linkage. Examples of such linkages include phosphoramide, phosphorothioate, and phosphorodithioate linkages. The compounds may also be chemically modified nucleic acid molecules as taught in U.S. Pat. No. 6,673,661. In other embodiments, the compound contains one or two capped strands, as disclosed, for example, by WO 00/63364, filed Apr. 19, 2000.

In certain embodiments, the first strand of the compound is an siRNA guide strand and the second strand of the compound is an siRNA passenger strand. In certain embodiments, the second strand of the compound is complementary to the first strand. In certain embodiments, each strand of the compound is 16, 17, 18, 19, 20, 21, 22, or 23 linked nucleosides in length. In certain embodiments, the first or second strand of the compound can comprise a conjugate group.

In certain embodiments, a compound described herein can comprise any of the oligonucleotide sequences targeted to an α-ENaC nucleic acid described herein. In certain embodiments, the compound is single-stranded. In certain embodiments, such a compound is a single-stranded RNAi (ssRNAi) compound. In certain embodiments, the compound comprises at least an 8, 9, 10, 11, 12, 13, 14, 15, 16, 17, 18, 19, or 20 contiguous nucleobase portion of any one of SEQ ID NOs: 6-1954. In certain embodiments, the compound comprises the nucleobase sequence of any one of SEQ ID NOs: 6-1954. In certain embodiments, the compound comprises ribonucleotides in which uracil (U) is in place of thymine (T) in any one of SEQ ID NOs: 6-1954. In certain embodiments, the compound comprises a nucleobase sequence complementary to the site on α-ENaC to which any of SEQ ID NOs: 6-1954 is targeted. In certain embodiments, the compound comprises one or more modified nucleotides in which the 2′ position in the sugar contains a halogen (such as fluorine group; 2′-F) or contains an alkoxy group (such as a methoxy group; 2′-OMe). In certain embodiments, the compound comprises at least one 2′-F sugar modification and at least one 2′-OMe sugar modification. In certain embodiments, the at least one 2′-F sugar modification and at least one 2′-OMe sugar modification are arranged in an alternating pattern for at least 2, 3, 4, 5, 6, 7, 8, 9, 10, 11, 12, 13, 14, 15, 16, 17, 18, 19, or 20 contiguous nucleobases along a strand of the compound. In certain embodiments, the compound comprises one or more linkages between adjacent nucleotides other than a naturally-occurring phosphodiester linkage. Examples of such linkages include phosphoramide, phosphorothioate, and phosphorodithioate linkages. The compounds may also be chemically modified nucleic acid molecules as taught in U.S. Pat. No. 6,673,661. In other embodiments, the compound contains a capped strand, as disclosed, for example, by WO 00/63364, filed Apr. 19, 2000. In certain embodiments, the compound consists of 16, 17, 18, 19, 20, 21, 22, or 23 linked nucleosides. In certain embodiments, the compound can comprise a conjugate group.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β, such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### Certain Mechanisms

In certain embodiments, compounds described herein comprise or consist of modified oligonucleotides. In certain embodiments, compounds described herein are antisense compounds. In certain embodiments, compounds comprise oligomeric compounds. In certain embodiments, compounds described herein are capable of hybridizing to an α-ENaC target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Further, in certain embodiments, one or more non-DNA-like nucleoside in the gap of a gapmer is tolerated.

In certain antisense activities, compounds described herein or a portion of the compound is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi compounds. RNAi compounds may be double-stranded (siRNA) or single-stranded (ssRNA).

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Target Nucleic Acids, Target Regions and Nucleotide Sequences

In certain embodiments, compounds described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain embodiments, a pre-mRNA and corresponding mRNA are both target nucleic acids of a single compound. In certain such embodiments, the target region is entirely within an intron of a target pre-mRNA. In certain embodiments, the target region spans an intron/exon junction. In certain embodiments, the target region is at least 50% within an intron. Target nucleic acid sequences that encode α-ENaC include, without limitation, the following: RefSEQ No. NM_001038.5; the complement of NC_000012.12 truncated from nucleosides 6343001 to 6380000; and NG_011945.1 (SEQ ID Nos: 1, 2, and 1957, respectively).

### Hybridization

In some embodiments, hybridization occurs between a compound disclosed herein and an α-ENaC nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Hybridization conditions are sequence-dependent and are determined by the nature and composition of the nucleic acid molecules to be hybridized.

Methods of determining whether a sequence is specifically hybridizable to a target nucleic acid are well known in the art. In certain embodiments, the compounds provided herein are specifically hybridizable with an α-ENaC nucleic acid.

### Complementarity

In certain embodiments, compounds described herein comprise or consist of modified oligonucleotides. In certain embodiments, compounds described herein are antisense compounds. In certain embodiments, compounds comprise oligomeric compounds. In certain embodiments, oligonucleotides complementary to an α-ENaC nucleic acid comprise nucleobase that are non-complementary with the α-ENaC nucleic acid, yet may be tolerated provided that the compound remains able to specifically hybridize to a target nucleic acid. Moreover, a compound may hybridize over one or more segments of an α-ENaC nucleic acid such that intervening or adjacent segments are not involved in the hybridization event (e.g., a loop structure, mismatch or hairpin structure).

In certain embodiments, the compounds provided herein, or a specified portion thereof, are, are at least, or are up to 70%, 80%, 85%, 86%, 87%, 88%, 89%, 90%, 91%, 92%, 93%, 94%, 95%, 96%, 97%, 98%, 99%, or 100% complementary to an α-ENaC nucleic acid, a target region, target segment, or specified portion thereof. In certain embodiments, the compounds provided herein, or a specified portion thereof, are 70% to 75%, 75% to 80%, 80% to 85%, 85% to 90%, 90% to 95%, 95% to 100%, or any number in between these ranges, complementary to an α-ENaC nucleic acid, a target region, target segment, or specified portion thereof. Percent complementarity of a compound with a target nucleic acid can be determined using routine methods.

For example, a compound in which 18 of 20 nucleobases of the compound are complementary to a target region, and would therefore specifically hybridize, would represent 90 percent complementarity. In this example, the remaining non-complementary nucleobases may be clustered or interspersed with complementary nucleobases and need not be contiguous to each other or to complementary nucleobases. As such, a compound which is 18 nucleobases in length having four non-complementary nucleobases which are flanked by two regions of complete complementarity with the target nucleic acid would have 77.8% overall complementarity with the target nucleic acid. Percent complementarity of a compound with a region of a target nucleic acid can be determined routinely using BLAST programs (basic local alignment search tools) and PowerBLAST programs known in the art (Altschul et al., J Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, compounds described herein, or specified portions thereof, are fully complementary (i.e. 100% complementary) to a target nucleic acid, or specified portion thereof. For example, a compound may be 100% complementary to an α-ENaC nucleic acid, or a target region, or a target segment or target sequence thereof. As used herein, “fully complementary” means each nucleobase of a compound is complementary to the corresponding nucleobase of a target nucleic acid. For example, a 20 nucleobase compound is fully complementary to a target sequence that is 400 nucleobases long, so long as there is a corresponding 20 nucleobase portion of the target nucleic acid that is fully complementary to the compound. Fully complementary can also be used in reference to a specified portion of the first and/or the second nucleic acid. For example, a 20 nucleobase portion of a 30 nucleobase compound can be “fully complementary” to a target sequence that is 400 nucleobases long. The 20 nucleobase portion of the 30 nucleobase compound is fully complementary to the target sequence if the target sequence has a corresponding 20 nucleobase portion wherein each nucleobase is complementary to the 20 nucleobase portion of the compound. At the same time, the entire 30 nucleobase compound may or may not be fully complementary to the target sequence, depending on whether the remaining 10 nucleobases of the compound are also complementary to the target sequence.

In certain embodiments, compounds described herein comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap segment. In certain such embodiments, the mismatch is at position 9, 8, 7, 6, 5, 4, 3, 2, 1 from the 3′-end of the gap segment. In certain such embodiments, the mismatch is at position 1, 2, 3, or 4 from the 5′-end of the wing segment. In certain such embodiments, the mismatch is at position 4, 3, 2, or 1 from the 3′-end of the wing segment. In certain embodiments, the mismatch is specifically positioned within an oligonucleotide not having a gapmer motif. In certain such embodiments, the mismatch is at position 1, 2, 3, 4, 5, 6, 7, 8, 9, 10, 11, or 12 from the 5′-end of the oligonucleotide. In certain such embodiments, the mismatch is at position, 2, 3, 4, 5, 6, 7, 8, 9, 10, 11, or 12 from the 3′-end of the oligonucleotide.

The location of a non-complementary nucleobase may be at the 5′ end or 3′ end of the compound. Alternatively, the non-complementary nucleobase or nucleobases may be at an internal position of the compound. When two or more non-complementary nucleobases are present, they may be contiguous (i.e. linked) or non-contiguous. In one embodiment, a non-complementary nucleobase is located in the wing segment of a gapmer oligonucleotide.

In certain embodiments, compounds described herein that are, or are up to 11, 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as an α-ENaC nucleic acid, or specified portion thereof.

In certain embodiments, compounds described herein that are, or are up to 11, 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as an α-ENaC nucleic acid, or specified portion thereof.

In certain embodiments, compounds described herein also include those which are complementary to a portion (a defined number of contiguous nucleobases within a region or segment) of a target nucleic acid. In certain embodiments, the compounds, are complementary to at least an 8 nucleobase portion of a target segment. In certain embodiments, the compounds are complementary to at least a 9 nucleobase portion of a target segment. In certain embodiments, the compounds are complementary to at least a 10 nucleobase portion of a target segment. In certain embodiments, the compounds are complementary to at least an 11 nucleobase portion of a target segment. In certain embodiments, the compounds are complementary to at least a 12 nucleobase portion of a target segment. In certain embodiments, the compounds are complementary to at least a 13 nucleobase portion of a target segment. In certain embodiments, the compounds are complementary to at least a 14 nucleobase portion of a target segment. In certain embodiments, the compounds are complementary to at least a 15 nucleobase portion of a target segment. In certain embodiments, the compounds are complementary to at least a 16 nucleobase portion of a target segment. Also contemplated are compounds that are complementary to at least a 9, 10, 11, 12, 13, 14, 15, 16, 17, 18, 19, 20, or more nucleobase portion of a target segment, or a range defined by any two of these values.

### Certain Compounds

In certain embodiments, compounds described herein comprise or consist of oligonucleotides consisting of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

### I. Modifications

### A. Modified Nucleosides

Modified nucleosides comprise a modified sugar moiety or a modified nucleobase or both a modified sugar moiety and a modified nucleobase.

### 1. Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including but not limited to substituents at the 2′, 4′, and/or 5′ positions. In certain embodiments, the furanosyl sugar moiety is a ribosyl sugar moiety. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted nucleoside or 2′-non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a non-bridging 2′-substituent group selected from: F, OCH₃, and OCH₂CH₂OCH₃.

Nucleosides comprising modified sugar moieties, such as non-bicyclic modified sugar moieties, may be referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂-O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—O—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂-, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al., Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

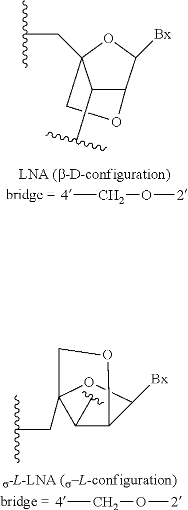

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, CJ. Bioorg. & Med. Chem. 2002, 10, 841-854), fluoro HNA:

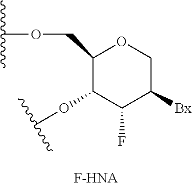

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3-fluoro tetrahydropyran), and nucleosides comprising additional modified THP compounds having the formula:

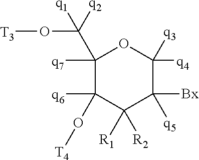

wherein, independently, for each of said modified THP nucleoside:
Bx is a nucleobase moiety;
T₃ and T₄ are each, independently, an intemucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3-terminal group; q₁, q₂, q₃, q₄, q₅, q₆ and q are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and g₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

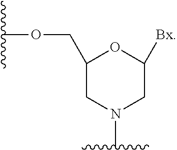

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

### 2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds comprise or consist of a modified oligonucleotide complementary to an α-ENaC nucleic acid comprising one or more modified nucleobases. In certain embodiments, the modified nucleobase is 5-methylcytosine. In certain embodiments, each cytosine is a 5-methylcytosine.

### B. Modified Internucleoside Linkages

In certain embodiments, compounds described herein having one or more modified internucleoside linkages are selected over compounds having only phosphodiester internucleoside linkages because of desirable properties such as, for example, enhanced cellular uptake, enhanced affinity for target nucleic acids, and increased stability in the presence of nucleases.

In certain embodiments, compounds comprise or consist of a modified oligonucleotide complementary to an α-ENaC nucleic acid comprising one or more modified internucleoside linkages. In certain embodiments, the modified internucleoside linkages are phosphorothioate linkages. In certain embodiments, each internucleoside linkage of an antisense compound is a phosphorothioate internucleoside linkage.

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

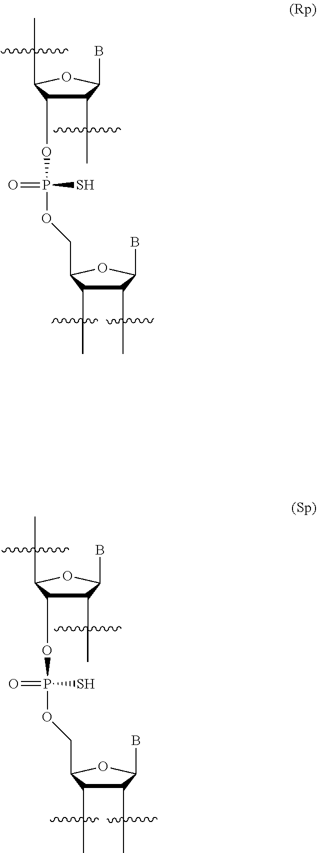

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

### II. Certain Motifs

In certain embodiments, compounds described herein comprise or consist of oligonucleotides. Oligonucleotides can have a motif, e.g. a pattern of unmodified and/or modified sugar moieties, nucleobases, and/or internucleoside linkages. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### A. Certain Sugar Motifs

In certain embodiments, compounds described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external segments or “wings” and a central or internal segment or “gap.” The three segments of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are immediately adjacent to the gap (the 3′-terminal wing nucleoside of the 5′-wing and the 5′-terminal wing nucleoside of the 3′-wing) differ from the sugar moiety of the adjacent gap nucleosides. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer each comprise 1-5 nucleosides. In certain embodiments, the wings of a gapmer each comprise 2-5 nucleosides. In certain embodiments, the wings of a gapmer each comprise 3-5 nucleosides. In certain embodiments, the nucleosides of the wings of a gapmer are all modified nucleosides. In certain such embodiments, the sugar moieties of the wings of a gapmer are all modified sugar moieties.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is a 2′-deoxynucleoside.

In certain embodiments, the gapmer is a deoxy gapmer. In such embodiments, the nucleosides on the gap side of each wing/gap junction are 2′-deoxynucleosides and the terminal wing nucleosides immediately adjacent to the gap comprise modified sugar moieties. In certain such embodiments, each nucleoside of the gap is a 2′-deoxynucleoside. In certain such embodiments, each nucleoside of each wing comprises a modified sugar moiety.

In certain embodiments, a modified oligonucleotide has a fully modified sugar motif wherein each nucleoside of the modified oligonucleotide comprises a modified sugar moiety. In certain embodiments, modified oligonucleotides comprise or consist of a region having a fully modified sugar motif wherein each nucleoside of the region comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

In certain embodiments, a modified oligonucleotide can comprise a sugar motif described in Swayze et al., US2010/0197762; Freier et al., US2014/0107330; Freier et al., US2015/0184153; and Seth et al., US2015/0267195.

### B. Certain Nucleobase Motifs

In certain embodiments, compounds described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

### C. Certain Internucleoside Linkage Motifs

In certain embodiments, compounds described herein comprise or consist of oligonucleotides. In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

In certain embodiments, oligonucleotides comprise a region having an alternating internucleoside linkage motif. In certain embodiments, oligonucleotides comprise a region of uniformly modified internucleoside linkages. In certain such embodiments, the internucleoside linkages are phosphorothioate internucleoside linkages. In certain embodiments, all of the internucleoside linkages of the oligonucleotide are phosphorothioate internucleoside linkages. In certain embodiments, each internucleoside linkage of the oligonucleotide is selected from phosphodiester or phophate and phosphorothioate. In certain embodiments, each internucleoside linkage of the oligonucleotide is selected from phosphodiester or phosphate and phosphorothioate and at least one internucleoside linkage is phosphorothioate.

In certain embodiments, the oligonucleotide comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, oligonucleotides comprise one or more methylphosponate linkages. In certain embodiments, oligonucleotides having a gapmer nucleoside motif comprise a linkage motif comprising all phosphorothioate linkages except for one or two methylphosponate linkages. In certain embodiments, one methylphosponate linkage is in the gap of an oligonucleotide having a gapmer sugar motif.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

### III. Certain Modified Oligonucleotides

In certain embodiments, compounds described herein comprise or consist of modified oligonucleotides. In certain embodiments, the above modifications (sugar, nucleobase, internucleoside linkage) are incorporated into a modified oligonucleotide. In certain embodiments, modified oligonucleotides are characterized by their modifications, motifs, and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. Likewise, such gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar motif, region or segment B consists of 6-10 linked nucleosides having a specified sugar motif, and region or segment C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence.

In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30,13 to 14,13 to 15,13 to 16,13 to 17,13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30,15 to 16,15 to 17,15 to 18,15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

### IV. Certain Conjugated Compounds

In certain embodiments, the compounds described herein comprise or consist of an oligonucleotide (modified or unmodified) and optionally one or more conjugate groups and/or terminal groups. Conjugate groups consist of one or more conjugate moiety and a conjugate linker that links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EM1BO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J Pharmacol. Exp. Ther., 1996, i, 923-937), a tocopherol group (Nishina et al., Molecular Therapy Nucleic Acids, 2015, 4, e220; doi:10.1038/mtna. 2014.72 and Nishina et al., Molecular Therapy, 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate linkers

Conjugate moieties are attached to oligonucleotides through conjugate linkers. In certain compounds, a conjugate group is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which a compound comprises an oligonucleotide consisting of a specified number or range of linked nucleosides and/or a specified percent complementarity to a reference nucleic acid and the compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, a compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, an compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances compounds comprising a particular conjugate moiety are better taken up by a particular cell type, but once the compound has been taken up, it is desirable that the conjugate group be cleaved to release the unconjugated or parent oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Certain Cell-Targeting Conjugate Moieties

In certain embodiments, a conjugate group comprises a cell-targeting conjugate moiety. In certain embodiments, a conjugate group has the general formula:

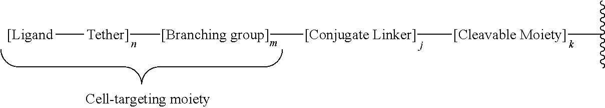

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments compounds described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Korilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Compositions and Methods for Formulating Pharmaceutical Compositions

Compounds described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more compounds or a salt thereof. In certain embodiments, the compounds are antisense compounds or oligomeric compounds. In certain embodiments, the compounds comprise or consist of a modified oligonucleotide. In certain such embodiments, the pharmaceutical composition comprises a suitable pharmaceutically acceptable diluent or carrier. In certain embodiments, a pharmaceutical composition comprises a sterile saline solution and one or more compound. In certain embodiments, such pharmaceutical composition consists of a sterile saline solution and one or more compound. In certain embodiments, the sterile saline is pharmaceutical grade saline. In certain embodiments, a pharmaceutical composition comprises one or more compound and sterile water. In certain embodiments, a pharmaceutical composition consists of one compound and sterile water. In certain embodiments, the sterile water is pharmaceutical grade water. In certain embodiments, a pharmaceutical composition comprises or consists of one or more compound and phosphate-buffered saline (PBS). In certain embodiments, a pharmaceutical composition consists of one or more compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

Certain embodiments provide pharmaceutical compositions suitable for aerosolization and/or dispersal by a nebulizer or inhaler. Such devices are well known in the art. In certain such embodiments, the pharmaceutical composition is a solid comprising particles of compounds that are of respirable size. A solid particulate composition can optionally contain a dispersant which serves to facilitate the formation of an aerosol, e.g., lactose. Solid pharmaceutical compositions comprising an oligonucleotide can also be aerosolized using any solid particulate medicament aerosol generator known in the art, e.g., a dry powder inhaler. In certain embodiments, the powder employed in the inhaler consists of the compound comprising the active compound or of a powder blend comprising the active compound, a suitable powder diluent, and an optional surfactant.

In certain embodiments, the pharmaceutical composition is a liquid. In certain such embodiments, the liquid is administered as an aerosol that is produced by any suitable means, such as with a nebulizer or inhaler. See, e.g., U.S. Pat. No. 4,501,729. Nebulizers are devices that transform solutions or suspensions into an aerosol mist and are well known in the art. Suitable nebulizers include jet nebulizers, ultrasonic nebulizers, electronic mesh nebulizers, and vibrating mesh nebulizers. Companies such as PARI and Vectura sell some types of such suitable nebulizers. In certain embodiments, the aerosol is produced by a metered dose inhaler, which typically contains a suspension or solution formulation of the active compound in a liquefied propellant. Inhalers suitable for dispensing liquid aerosol also include certain inhalers sold by Respimat (See, e.g., Anderson, Int J Chron Obstruct Pulmon Dis. 1, 251 (2006).) Pharmaceutical compositions suitable for aerosolization can comprise propellants, surfactants, co-solvents, dispersants, preservatives, and/or other additives or excipients.

A compound described herein complementary to an α-ENaC nucleic acid can be utilized in pharmaceutical compositions by combining the compound with a suitable pharmaceutically acceptable diluent or carrier and/or additional components such that the pharmaceutical composition is suitable for aerosolization by a nebulizer. In certain embodiments, a pharmaceutically acceptable diluent is phosphate buffered saline. Accordingly, in one embodiment, employed in the methods described herein is a pharmaceutical composition comprising a compound complementary to an α-ENaC nucleic acid and a pharmaceutically acceptable diluent. In certain embodiments, the pharmaceutically acceptable diluent is phosphate buffered saline. In certain embodiments, the compound comprises or consists of a modified oligonucleotide provided herein.

Pharmaceutical compositions comprising compounds provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the compounds are antisense compounds or oligomeric compounds. In certain embodiments, the compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of a compound which are cleaved by endogenous nucleases within the body, to form the active compound.

In certain embodiments, the compounds or compositions further comprise a pharmaceutically acceptable carrier or diluent.

### EXAMPLES

The Examples below describe the screening process used to identify lead compounds targeted to α-ENaC. Out of over 1,900 oligonucleotides that were screened, many potent and tolerable oligonucleotides were identified, and compounds 797308, 797495, 826763, 827307, 827359, and 827392 emerged as the top lead compounds. In particular, compound 827359 exhibited the best combination of properties in terms of potency and tolerability.

### Non-Limiting Disclosure and Incorporation by Reference

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine nucleobase could be described as a DNA having an RNA sugar, or as an RNA having a DNA nucleobase.

Accordingly, nucleic acid sequences provided herein, including, but not limited to those in the sequence listing, are intended to encompass nucleic acids containing any combination of unmodified or modified RNA and/or DNA, including, but not limited to such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and compounds having other modified nucleobases, such as “AT^{m}CGAUCG,” wherein ^{m}C indicates a cytosine base comprising a methyl group at the 5-position.

While certain compounds, compositions and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Effect of Modified Oligonucleotides Complementary to α-ENaC In Vitro

Modified oligonucleotides complementary to one or more human α-ENaC nucleic acids were designed and tested for their effect on α-ENaC mRNA in vitro. The modified oligonucleotides were tested in a series of experiments that had similar culture conditions.

Cultured Hep3B cells at a density of 20,000 cells per well were transfected using electroporation with 2,000 nM of modified oligonucleotide or no modified oligonucleotide for untreated controls. After approximately 24 hours, RNA was isolated from the cells and α-ENaC mRNA levels were measured by quantitative real-time PCR. Human primer probe set hSCNN1A_LTS01170 (forward sequence ACATCCCAGGAATGGGTCTTC, designated herein as SEQ ID NO: 3; reverse sequence ACTTTGGCCACTCCATTTCTCTT, designated herein as SEQ ID NO: 4; probe sequence TGCTATCGCGACAGAACAATTACACCGTC, designated herein as SEQ ID: 5) was used to measure mRNA levels. α-ENaC mRNA levels were normalized to total RNA content, as measured by RIBOGREEN®. Results are presented in the tables below as normalized α-ENaC mRNA level, relative to untreated control cells (these conditions describe a “Standard Cell Assay”).

The modified oligonucleotides in the tables below each have a 3-10-3 phosphothiorate cEt gapmer motif. The modified oligonucleotides are 16 nuceobases in length, wherein the central gap segment contains ten 2′-deoxynucleosides and is flanked by wing segments on the 3′ and 5′ ends, each containing three cEt nucleosides. All cytosine residues throughout each modified oligonucletoide are 5-methyl cytosines. The intermucleoside linkages are all phosphorothioate intermucleoside linkages.

Each modified oligonucleotide listed in the tables below is 10000 complementary to the human α-ENaC nucleic acid sequence of GenBank Number NM_001038.5 (designated herein as SEQ ID NO: 1), the complement of GenBank Number NC_000012.12, truncated from nucleosides 6343001 to 6380000 (designated herein as SEQ ID NO: 2), and/or GenBank Number NG_011945.1 (designated herein as SEQ ID NO: 1957). “Start Site” indicates the 5′-most nucleoside of the designated α-ENaC nucleic acid to which the oligonucleotide is complementary. “Stop Site” indicates the 3′-most nucleoside of the human α-ENaC nucleic acid to which the oligonucleotide is complementary. ‘N/A’ indicates that the modified oligonucleotide is not complementary to that particular nucleic acid with 100% complementarity. Several oligonucleotides match two or more sites on the mRNA, as shown in the tables below. As shown below, modified oligonucleotides complementary to human α-ENaC reduced the amount of human α-ENaC mRNA in vitro.

TABLE 1
Percent level of human α-ENaC mRNA

| | SEQ | SEQ | | | SEQ | SEQ | |
|---|---|---|---|---|---|---|---|
| | ID: 1 | ID: 1 | | α-ENaC | ID: 2 | ID 2: | SEQ |
| Compound | Start | Stop | | (% | Start | Stop | ID |
| Number | Site | Site | Sequence | control) | Site | Site | NO |
| 668181 | 523 | 538 | CTTCATGCGGTTGTGC | 37 | 5451 | 5466 | 6 |
| 668248 | 1240 | 1255 | AGGCATGGAAGACATC | 94 | 24196 | 24211 | 7 |
| 668279 | 1575 | 1590 | ATGTAGGCACAGCCAC | 30 | 25489 | 25504 | 8 |
| 668280 | 1580 | 1595 | AGAAGATGTAGGCACA | 27 | 25494 | 25509 | 9 |
| 668324 | 1930 | 1945 | GCCCAGGTTGGACAGG | 54 | 31759 | 31774 | 10 |
| 668325 | 1954 | 1969 | GCCGAACCACAGGCTC | 72 | 31783 | 31798 | 11 |
| 668358 | 2599 | 2614 | TGTCAAAGCTCCAAGT | 43 | 32428 | 32443 | 12 |
| 668364 | 2766 | 2781 | ACCCAAGTTCAAGAGG | 76 | 32595 | 32610 | 13 |
| 797074 | 4 | 19 | TTAGACGCAGACAGGC | 73 | 4265 | 4280 | 14 |
| 797075 | 30 | 45 | GAGAAGGCGGACTCTG | 88 | 4291 | 4306 | 15 |
| 797076 | 43 | 58 | GAGTACTGGACCTGAG | 64 | 4304 | 4319 | 16 |
| 797077 | 51 | 66 | TGAACTGGGAGTACTG | 71 | 4312 | 4327 | 17 |
| 797078 | 63 | 78 | CCCGAGGGCAGGTGAA | 97 | 4324 | 4339 | 18 |
| 797079 | 75 | 90 | GGAAGGAGGGCTCCCG | 98 | 4336 | 4351 | 19 |
| 797080 | 82 | 97 | TTCCGAAGGAAGGAGG | 123 | 4343 | 4358 | 20 |
| 797081 | 90 | 105 | CGGGAGTTTTCCGAAG | 135 | 4351 | 4366 | 21 |
| 797082 | 97 | 112 | TCAGAGCCGGGAGTTT | 64 | 4358 | 4373 | 22 |
| 797083 | 110 | 125 | GGCTGAGGAGGAGTCA | 86 | 4371 | 4386 | 23 |
| 797084 | 135 | 150 | TAAAGGTGAGCAGGGC | 107 | 4396 | 4411 | 24 |
| 797085 | 137 | 152 | ATTAAAGGTGAGCAGG | 96 | 4398 | 4413 | 25 |
| 797086 | 139 | 154 | CAATTAAAGGTGAGCA | 137 | 4400 | 4415 | 26 |
| 797087 | 141 | 156 | CTCAATTAAAGGTGAG | 150 | 4402 | 4417 | 27 |
| 797088 | 142 | 157 | TCTCAATTAAAGGTGA | 73 | 4403 | 4418 | 28 |
| 797089 | 147 | 162 | TAGCATCTCAATTAAA | 61 | 4408 | 4423 | 29 |
| 797090 | 151 | 166 | TCATTAGCATCTCAAT | 104 | 4412 | 4427 | 30 |
| 797091 | 158 | 173 | AGGAATCTCATTAGCA | 70 | 4419 | 4434 | 31 |
| 797092 | 168 | 183 | TGGAAGCGACAGGAAT | 94 | 4429 | 4444 | 32 |
| 797093 | 177 | 192 | GGCCAGGGATGGAAGC | 100 | 4438 | 4453 | 33 |
| 797094 | 213 | 228 | GTGCAGCGGCCTGGCT | 73 | 4474 | 4489 | 34 |
| 797095 | 22 | 236 | CCTGACAGGTGCAGCG | 71 | 4482 | 4497 | 35 |
| 797096 | 235 | 250 | CTCCAGCTTGTTCCCC | 41 | 5163 | 5178 | 36 |
| | 295 | 310 | | | 5223 | 5238 | |
| 797097 | 237 | 252 | TCCTCCAGCTTGTTCC | 49 | 5165 | 5180 | 37 |
| | 297 | 312 | | | 5225 | 5240 | |
| 797098 | 238 | 253 | CTCCTCCAGCTTGTTC | 58 | 5166 | 5181 | 38 |
| | 298 | 313 | | | 5226 | 5241 | |
| 797099 | 240 | 255 | TGCTCCTCCAGCTTGT | 26 | 5168 | 5183 | 39 |
| | 300 | 315 | | | 5228 | 5243 | |
| 797100 | 242 | 257 | CCTGCTCCTCCAGCTT | 19 | 5170 | 5185 | 40 |
| | 302 | 317 | | | 5230 | 5245 | |
| 797101 | 244 | 259 | GTCCTGCTCCTCCAGC | 21 | 5172 | 5187 | 41 |
| | 304 | 319 | | | 5232 | 5247 | |
| 797102 | 251 | 266 | GTCTAGGGTCCTGCTC | 41 | 5179 | 5194 | 42 |
| 797103 | 258 | 273 | TGCAGAGGTCTAGGGT | 27 | 5186 | 5201 | 43 |
| 797104 | 268 | 283 | TGGTATGGGCTGCAGA | 18 | 5196 | 5211 | 44 |
| 797105 | 277 | 292 | CATGAGACCTGGTATG | 85 | 5205 | 5220 | 45 |
| 797106 | 311 | 326 | GGCTAGAGTCCTGCTC | 78 | 5239 | 5254 | 46 |
| 797107 | 329 | 344 | CTGGAGTGGACTGTGG | 73 | 5257 | 5272 | 47 |
| 797108 | 403 | 418 | CGCCGTGGGCTGCTGG | 60 | 5331 | 5346 | 48 |
| 797109 | 424 | 439 | CTCGATCAGGGCCTCC | 33 | 5352 | 5367 | 49 |
| 797110 | 438 | 453 | TAGGAGCGGTGGAACT | 51 | 5366 | 5381 | 50 |
| 797111 | 440 | 455 | GGTAGGAGCGGTGGAA | 32 | 5368 | 5383 | 51 |
| 797112 | 447 | 462 | AGCTCTCGGTAGGAGC | 84 | 5375 | 5390 | 52 |
| 797113 | 454 | 469 | CTCGAAGAGCTCTCGG | 34 | 5382 | 5397 | 53 |
| 797114 | 462 | 477 | CAGAAGAACTCGAAGA | 48 | 5390 | 5405 | 54 |
| 797115 | 534 | 549 | CAGAAGGCCGTCTTCA | 30 | 5462 | 5477 | 55 |
| 797116 | 537 | 552 | GCCCAGAAGGCCGTCT | 41 | 5465 | 5480 | 56 |
| 797117 | 554 | 569 | TGCAGAGCCACAGCAC | 47 | 5482 | 5497 | 57 |
| 797118 | 561 | 576 | CCAAAGGTGCAGAGCC | 17 | 5489 | 5504 | 58 |
| 797119 | 568 | 583 | CATCATGCCAAAGGTG | 26 | 5496 | 5511 | 59 |
| 797120 | 576 | 591 | TGCCAGTACATCATGC | 45 | 5504 | 5519 | 60 |
| 797121 | 583 | 598 | GCCGAATTGCCAGTAC | 20 | 5511 | 5526 | 61 |
| 797122 | 592 | 607 | GAAAAGCAGGCCGAAT | 49 | 5520 | 5535 | 62 |
| 797123 | 604 | 619 | GAAGTACTCTCCGAAA | 36 | 5532 | 5547 | 63 |
| 797124 | 642 | 657 | TCCGAGTTGAGGTTGA | 25 | 5570 | 5585 | 64 |
| 797125 | 651 | 666 | ACGAGCTTGTCCGAGT | 23 | 5579 | 5594 | 65 |
| 797126 | 682 | 697 | ATTGAGGGTGCAGATG | 58 | 5610 | 5625 | 66 |
| 797127 | 704 | 719 | TAATTTCCGGGTACCT | 30 | 16289 | 16304 | 67 |
| 797128 | 736 | 751 | TGTGATGCGGTCCAGC | 24 | 16321 | 16336 | 68 |
| 797129 | 760 | 775 | GTACAGGTCAAAGAGC | 20 | 16345 | 16360 | 69 |
| 797130 | 765 | 780 | TATTTGTACAGGTCAA | 20 | 16350 | 16365 | 70 |
| 797131 | 767 | 782 | TGTATTTGTACAGGTC | 13 | 16352 | 16367 | 71 |
| 797132 | 778 | 793 | GGTGAAGGAGCTGTAT | 35 | 16363 | 16378 | 72 |
| 797133 | 785 | 800 | CGAGAGTGGTGAAGGA | 72 | 16370 | 16385 | 73 |
| 797134 | 793 | 808 | GCCGGCCACGAGAGTG | 101 | 16378 | 16393 | 74 |
| 797135 | 802 | 817 | GCTGCGGGAGCCGGCC | 59 | 16387 | 16402 | 75 |
| 797136 | 809 | 824 | CGCGACGGCTGCGGGA | 38 | 16394 | 16409 | 76 |
| 797137 | 817 | 832 | CCGCAGGTCGCGACGG | 74 | 16402 | 16417 | 77 |
| 797138 | 880 | 895 | TCGACGGGCCCCGTGA | 41 | 16465 | 16480 | 78 |
| 797139 | 890 | 905 | CGCTACGGGCTCGACG | 20 | 16475 | 16490 | 79 |
| 797140 | 901 | 916 | GCTGGAGGCCACGCTA | 21 | 16486 | 16501 | 80 |
| 797141 | 942 | 957 | TTCCAGTCCTTCCAGT | 45 | 16527 | 16542 | 81 |
| 797142 | 950 | 965 | AGCCGATCTTCCAGTC | 44 | 16535 | 16550 | 82 |
| 797143 | 961 | 976 | GCACAGCTGGAAGCCG | 30 | N/A | N/A | 83 |
| 797144 | 977 | 992 | CCGATTTGTTCTGGTT | 19 | 17763 | 17778 | 84 |
| 797145 | 984 | 999 | AAGCAGTCCGATTTGT | 74 | 17770 | 17785 | 85 |
| 797146 | 1002 | 1017 | GATGAGTATGTCTGGT | 18 | 17788 | 17803 | 86 |
| 797147 | 1016 | 1031 | CCGCATCCACCCCTGA | 27 | 17802 | 17817 | 87 |
| 797148 | 1044 | 1059 | ATGTAGTGGAAGCGGT | 59 | 17830 | 17845 | 88 |
| 797149 | 1057 | 1072 | CGACAGGATGTTGATG | 45 | 17843 | 17858 | 89 |
| 797150 | 1066 | 1081 | TGGCAGCCTCGACAGG | 24 | 17852 | 17867 | 90 |
| 797151 | 1077 | 1092 | GGCAGAGTCTCTGGCA | 39 | 17863 | 17878 | 91 |
| 797152 | 1087 | 1102 | CTCCAGGGATGGCAGA | 60 | 17873 | 17888 | 92 |
| 797153 | 1111 | 1126 | GATGAAGTTGCCCAGC | 24 | 17897 | 17912 | 93 |
| 797154 | 1118 | 1133 | AGGCGAAGATGAAGTT | 51 | 17904 | 17919 | 94 |
| 797155 | 1128 | 1143 | TTGAAGCGGCAGGCGA | 48 | 17914 | 17929 | 95 |
| 797156 | 1143 | 1158 | TTGCAGGAGACCTGGT | 45 | 17929 | 17944 | 96 |
| 797157 | 1156 | 1171 | GTAATTCGCCTGGTTG | 102 | N/A | N/A | 97 |
| 797158 | 1163 | 1178 | AGTGAGAGTAATTCGC | 53 | N/A | N/A | 98 |
| 797159 | 1227 | 1242 | ATCCAGAGGTTGGAGT | 105 | 24183 | 24198 | 99 |
| 797160 | 1235 | 1250 | TGGAAGACATCCAGAG | 50 | 24191 | 24206 | 100 |
| 797161 | 1244 | 1259 | TTCCAGGCATGGAAGA | 68 | 24200 | 24215 | 101 |
| 797162 | 1256 | 1271 | GACCGTTGTTGATTCC | 28 | N/A | N/A | 102 |
| 797163 | 1264 | 1279 | CAGGGACAGACCGTTG | 22 | N/A | N/A | 103 |
| 797164 | 1272 | 1287 | CGCAGCATCAGGGACA | 14 | 24569 | 24584 | 104 |
| 797165 | 1289 | 1304 | AGTCATTCTGCTCTGC | 9 | 24586 | 24601 | 105 |
| 797166 | 1293 | 1308 | ATGAAGTCATTCTGCT | 27 | 24590 | 24605 | 106 |
| 797167 | 1297 | 1312 | GGGAATGAAGTCATTC | 37 | 24594 | 24609 | 107 |
| 797168 | 1315 | 1330 | AGTCACTGTGGACAGC | 20 | 24612 | 24627 | 108 |
| 797169 | 1330 | 1345 | CATTACCCGGGCCCCA | 53 | 24627 | 24642 | 109 |
| 797170 | 1355 | 1370 | AGGCAGGTTCATCCTG | 54 | 24652 | 24667 | 110 |
| 797171 | 1362 | 1377 | TCCATAAAGGCAGGTT | 48 | 24659 | 24674 | 111 |
| 797172 | 1370 | 1385 | CACCATCATCCATAAA | 48 | 24667 | 24682 | 112 |
| 797173 | 1373 | 1388 | AGCCACCATCATCCAT | 31 | 24670 | 24685 | 113 |
| 797174 | 1377 | 1392 | TTAAAGCCACCATCAT | 39 | 24674 | 24689 | 114 |
| 797175 | 1384 | 1399 | CCGCAAGTTAAAGCCA | 24 | 24681 | 24696 | 115 |
| 797176 | 1391 | 1406 | CGCCAGGCCGCAAGTT | 18 | 24688 | 24703 | 116 |
| 797177 | 1414 | 1429 | CCTCATGCTGATGGAG | 40 | 24711 | 24726 | 117 |
| 797178 | 1429 | 1444 | GTCCAGGGTTTCCTTC | 48 | N/A | N/A | 118 |
| 797179 | 1439 | 1454 | CCCCAAGTCTGTCCAG | 49 | 25159 | 25174 | 119 |
| 797180 | 1449 | 1464 | CCATAATCGCCCCCAA | 15 | 25169 | 25184 | 120 |
| 797181 | 1465 | 1480 | ATTCTTGGTGCAGTCG | 18 | 25185 | 25200 | 121 |
| 797182 | 1475 | 1490 | CATCACTGCCATTCTT | 30 | 25195 | 25210 | 122 |
| 797183 | 1482 | 1497 | ACAGGAACATCACTGC | 22 | 25202 | 25217 | 123 |
| 797184 | 1495 | 1510 | GTAAAGGTTCTCAACA | 84 | 25215 | 25230 | 124 |
| 797185 | 1502 | 1517 | TTGAAGGGTAAAGGTT | 57 | 25222 | 25237 | 125 |
| 797186 | 1524 | 1539 | ATACACACCTGCTGTG | 87 | N/A | N/A | 126 |
| 797187 | 1533 | 1548 | CAGGAGTGAATACACA | 65 | 25447 | 25462 | 127 |
| 797188 | 1552 | 1567 | GATCATGCTCTCCTGG | 13 | 25466 | 25481 | 128 |
| 797189 | 1563 | 1578 | CCACACTCCTTGATCA | 36 | 25477 | 25492 | 129 |
| 797190 | 1570 | 1585 | GGCACAGCCACACTCC | 29 | 25484 | 25499 | 130 |
| 797191 | 1573 | 1588 | GTAGGCACAGCCACAC | 18 | 25487 | 25502 | 131 |
| 797192 | 1578 | 1593 | AAGATGTAGGCACAGC | 17 | 25492 | 25507 | 132 |
| 797193 | 1586 | 1601 | GCGGATAGAAGATGTA | 20 | 25500 | 25515 | 133 |
| 797194 | 1613 | 1628 | AGTCACAGTACTCCAC | 40 | 25527 | 25542 | 134 |
| 797195 | 1623 | 1638 | TGCTTTCTGTAGTCAC | 39 | 25537 | 25552 | 135 |
| 797196 | 1631 | 1646 | AGGAACTGTGCTTTCT | 48 | 25545 | 25560 | 136 |
| 797197 | 1644 | 1659 | TAGCAGTACCCCCAGG | 51 | N/A | N/A | 137 |
| 797198 | 1651 | 1666 | CTTATAGTAGCAGTAC | 42 | 30597 | 30612 | 138 |
| 797199 | 1663 | 1678 | GTCAACCTGGAGCTTA | 14 | 30609 | 30624 | 139 |
| 797200 | 1671 | 1686 | GAGGAGAAGTCAACCT | 66 | 30617 | 30632 | 140 |
| 797201 | 1684 | 1699 | GCCCAGGTGGTCTGAG | 43 | 30630 | 30645 | 141 |
| 797202 | 1692 | 1707 | GTGAAACAGCCCAGGT | 55 | 30638 | 30653 | 142 |
| 797203 | 1700 | 1715 | GGCACTTGGTGAAACA | 77 | 30646 | 30661 | 143 |
| 797204 | 1707 | 1722 | GGCTTCCGGCACTTGG | 22 | 30653 | 30668 | 144 |
| 797205 | 1715 | 1730 | CGCTGCATGGCTTCCG | 23 | N/A | N/A | 145 |
| 797206 | 1731 | 1746 | AGCTGGTAGCTGGTCA | 49 | 30782 | 30797 | 146 |
| 797207 | 1739 | 1754 | CAGCAGAGAGCTGGTA | 52 | 30790 | 30805 | 147 |
| 797208 | 1746 | 1761 | GAGTAACCAGCAGAGA | 47 | 30797 | 30812 | 148 |
| 797218 | 1875 | 1890 | TTGTAGTTCAGCTCCT | 41 | 31231 | 31246 | 149 |
| 797219 | 1885 | 1900 | AGAATTGGTTTTGTAG | 55 | 31241 | 31256 | 150 |
| 797220 | 1895 | 1910 | AGGGAGACTCAGAATT | 101 | 31251 | 31266 | 151 |
| 797221 | 1919 | 1934 | ACAGGAGGGTGACCAT | 41 | 31748 | 31763 | 152 |
| 797222 | 1941 | 1956 | CTCCACTGGCTGCCCA | 56 | 31770 | 31785 | 153 |
| 797223 | 1948 | 1963 | CCACAGGCTCCACTGG | 69 | 31777 | 31792 | 154 |
| 797224 | 1950 | 1965 | AACCACAGGCTCCACT | 64 | 31779 | 31794 | 155 |
| 797225 | 1955 | 1970 | AGCCGAACCACAGGCT | 139 | 31784 | 31799 | 156 |
| 797226 | 1963 | 1978 | CACCGAGGAGCCGAAC | 76 | 31792 | 31807 | 157 |
| 797227 | 1971 | 1986 | ACAGACAACACCGAGG | 24 | 31800 | 31815 | 158 |
| 797228 | 1978 | 1993 | CTCCACCACAGACAAC | 46 | 31807 | 31822 | 159 |
| 797229 | 1988 | 2003 | GCTCAGCCATCTCCAC | 63 | 31817 | 31832 | 160 |
| 797230 | 1997 | 2012 | CAAAGACGAGCTCAGC | 99 | 31826 | 31841 | 161 |
| 797231 | 2005 | 2020 | CAGCAGGTCAAAGACG | 96 | 31834 | 31849 | 162 |
| 797232 | 2017 | 2032 | GAACATGATGACCAGC | 22 | 31846 | 31861 | 163 |
| 797233 | 2024 | 2039 | GCATGAGGAACATGAT | 41 | 31853 | 31868 | 164 |
| 797234 | 2037 | 2052 | AACCTTCGGAGCAGCA | 18 | 31866 | 31881 | 165 |
| 797235 | 2045 | 2060 | GGCTTCGGAACCTTCG | 19 | 31874 | 31889 | 166 |
| 797236 | 2052 | 2067 | CAGTATCGGCTTCGGA | 19 | 31881 | 31896 | 167 |
| 797237 | 2060 | 2075 | CTGGAGACCAGTATCG | 35 | 31889 | 31904 | 168 |
| 797238 | 2104 | 2119 | TGCCAGGGTGGAGGCT | 81 | 31933 | 31948 | 169 |
| 797239 | 2127 | 2142 | CAGAAGTGGGAAGGAG | 63 | 31956 | 31971 | 170 |
| 797240 | 2151 | 2166 | AAGGACAGAGACATGG | 33 | 31980 | 31995 | 171 |
| 797241 | 2187 | 2202 | GTCAAGGCTGGAGAGG | 33 | 32016 | 32031 | 172 |
| 797242 | 2218 | 2233 | GCCCAGGGTGGCATAG | 76 | 32047 | 32062 | 173 |
| 797243 | 2259 | 2274 | GAGGAACTGGCCCCTG | 58 | 32088 | 32103 | 174 |
| 797244 | 2268 | 2283 | GGACAGGTGGAGGAAC | 76 | 32097 | 32112 | 175 |
| 797245 | 2275 | 2290 | CCCCAGAGGACAGGTG | 73 | 32104 | 32119 | 176 |
| 797246 | 2304 | 2319 | GAGAAACCTCTCCTTC | 64 | 32133 | 32148 | 177 |
| 797247 | 2329 | 2344 | ACCAGAGGAGCATCTG | 34 | 32158 | 32173 | 178 |
| 797248 | 2350 | 2365 | TGCCAGGGCCAGCACC | 50 | 32179 | 32194 | 179 |
| 797249 | 2358 | 2373 | TTCAATCTTGCCAGGG | 28 | 32187 | 32202 | 180 |
| 797250 | 2366 | 2381 | GCACATCCTTCAATCT | 36 | 32195 | 32210 | 181 |
| 797251 | 2377 | 2392 | GAGGAAGCCCTGCACA | 51 | 32206 | 32221 | 182 |
| 797252 | 2394 | 2409 | AGTTTGGGCGGCTCTG | 54 | 32223 | 32238 | 183 |
| 797253 | 2402 | 2417 | TCAACGGCAGTTTGGG | 22 | 32231 | 32246 | 184 |
| 797254 | 2409 | 2424 | CCACACATCAACGGCA | 23 | 32238 | 32253 | 185 |
| 797255 | 2427 | 2442 | ACCCATCTTGCTTCCC | 22 | 32256 | 32271 | 186 |
| 797256 | 2446 | 2461 | AGCAACTTCCTGAGCC | 36 | 32275 | 32290 | 187 |
| 797257 | 2461 | 2476 | AGCTACTGTTCTTGGA | 23 | 32290 | 32305 | 188 |
| 797258 | 2481 | 2496 | CACTTCTGGGCAGCTT | 14 | 32310 | 32325 | 189 |
| 797259 | 2488 | 2503 | GCCAAGGCACTTCTGG | 41 | 32317 | 32332 | 190 |
| 797260 | 2501 | 2516 | GTACAGGGCTGGAGCC | 65 | 32330 | 32345 | 191 |
| 797261 | 2522 | 2537 | GTTCAGAGGCAGTACC | 24 | 32351 | 32366 | 192 |
| 797262 | 2529 | 2544 | CCAGAGTGTTCAGAGG | 17 | 32358 | 32373 | 193 |
| 797263 | 2550 | 2565 | AGCCGCAGTTGGGTGG | 28 | 32379 | 32394 | 194 |
| 797264 | 2558 | 2573 | AGAGACTTAGCCGCAG | 12 | 32387 | 32402 | 195 |
| 797265 | 2565 | 2580 | GGGAAAAAGAGACTTA | 21 | 32394 | 32409 | 196 |
| 797266 | 2576 | 2591 | GGCTGATCCAAGGGAA | 14 | 32405 | 32420 | 197 |
| 797267 | 2590 | 2605 | TCCAAGTTTCGCTTGG | 85 | 32419 | 32434 | 198 |
| 797268 | 2598 | 2613 | GTCAAAGCTCCAAGTT | 26 | 32427 | 32442 | 199 |
| 797269 | 2611 | 2626 | AGGAAAGTTCCTTGTC | 21 | 32440 | 32455 | 200 |
| 797270 | 2626 | 2641 | TATCAGCGGTTTCTTA | 52 | 32455 | 32470 | 201 |
| 797271 | 2675 | 2690 | GGAAACCCGTGCATGC | 27 | 32504 | 32519 | 202 |
| 797272 | 2684 | 2699 | CGCTGGGCAGGAAACC | 25 | 32513 | 32528 | 203 |
| 797273 | 2694 | 2709 | CTTAAGCCGTCGCTGG | 31 | 32523 | 32538 | 204 |
| 797274 | 2716 | 2731 | GGCCAGGCCAGTCGGG | 64 | 32545 | 32560 | 205 |
| 797275 | 2725 | 2740 | GAGCAGTGTGGCCAGG | 18 | 32554 | 32569 | 206 |
| 797276 | 2732 | 2747 | TACTGGAGAGCAGTGT | 24 | 32561 | 32576 | 207 |
| 797277 | 2744 | 2759 | AGACATCTGTGCTACT | 10 | 32573 | 32588 | 208 |
| 797278 | 2757 | 2772 | CAAGAGGAGGAGCAGA | 46 | 32586 | 32601 | 209 |
| 797279 | 2765 | 2780 | CCCAAGTTCAAGAGGA | 68 | 32594 | 32609 | 210 |
| 797280 | 2803 | 2818 | CCTAAGTAACAAAGGG | 44 | 32632 | 32647 | 211 |
| 797281 | 2811 | 2826 | GGGAATTGCCTAAGTA | 44 | 32640 | 32655 | 212 |
| 797282 | 2857 | 2872 | ACTTACCCGGGTCTGC | 77 | 32686 | 32701 | 213 |
| 797283 | 2864 | 2879 | TGCCTTTACTTACCCG | 34 | 32693 | 32708 | 214 |
| 797284 | 2883 | 2898 | GCTAGAGGAGCCCTGG | 60 | 32712 | 32727 | 215 |
| 797285 | 2890 | 2905 | GTATGAGGCTAGAGGA | 70 | 32719 | 32734 | 216 |
| 797286 | 2897 | 2912 | GGCACGGGTATGAGGC | 71 | 32726 | 32741 | 217 |
| 797287 | 2914 | 2929 | GGGCATGGCTCTGTGA | 34 | 32743 | 32758 | 218 |
| 797288 | 2937 | 2952 | AAAGACACAGGGCAGA | 27 | 32766 | 32781 | 219 |
| 797289 | 2944 | 2959 | AGGTATGAAAGACACA | 17 | 32773 | 32788 | 220 |
| 797290 | 2952 | 2967 | ACATGTAGAGGTATGA | 17 | 32781 | 32796 | 221 |
| 797291 | 2962 | 2977 | TCTCAAGCAGACATGT | 20 | 32791 | 32806 | 222 |
| 797292 | 2969 | 2984 | GGAAATATCTCAAGCA | 15 | 32798 | 32813 | 223 |
| 797293 | 2983 | 2998 | AACTTTCAGGCTGAGG | 15 | 32812 | 32827 | 224 |
| 797294 | 3013 | 3028 | CATAGGAGTTCTCTGG | 10 | 32842 | 32857 | 225 |
| 797295 | 3020 | 3035 | AGGGATGCATAGGAGT | 19 | 32849 | 32864 | 226 |
| 797296 | 3033 | 3048 | GAGCAGGGTTCTAAGG | 60 | 32862 | 32877 | 227 |
| 797297 | 3046 | 3061 | AGTAATGGTGTCTGAG | 12 | 32875 | 32890 | 228 |
| 797298 | 3053 | 3068 | TCACAAAAGTAATGGT | 26 | 32882 | 32897 | 229 |
| 797299 | 3072 | 3087 | CAAGATGTGGCAGAAG | 58 | 32901 | 32916 | 230 |
| 797300 | 3079 | 3094 | GGGAAGACAAGATGTG | 22 | 32908 | 32923 | 231 |
| 797301 | 3093 | 3108 | AGTGATCAATTTTGGG | 22 | 32922 | 32937 | 232 |
| 797302 | 3103 | 3118 | GAGAAGGCGGAGTGAT | 61 | 32932 | 32947 | 233 |
| 797303 | 3118 | 3133 | GCTACGGGAGCCCAGG | 27 | 32947 | 32962 | 234 |
| 797304 | 3127 | 3142 | TTATAGTGTGCTACGG | 13 | 32956 | 32971 | 235 |
| 797305 | 3134 | 3149 | GCAGATGTTATAGTGT | 21 | 32963 | 32978 | 236 |
| 797306 | 3144 | 3159 | CAACACTCCAGCAGAT | 53 | 32973 | 32988 | 237 |
| 797307 | 3151 | 3166 | GCAACAGCAACACTCC | 6 | 32980 | 32995 | 238 |
| 797308 | 3160 | 3175 | AAGTATGGTGCAACAG | 9 | 32989 | 33004 | 239 |
| 797309 | 3167 | 3182 | TACAAGAAAGTATGGT | 11 | 32996 | 33011 | 240 |
| 797310 | 3180 | 3195 | GGAGACACAAATGTAC | 37 | 33009 | 33024 | 241 |
| 797311 | 3199 | 3214 | TTACAGTCTAGTTGGG | 20 | 33028 | 33043 | 242 |
| 797312 | 3209 | 3224 | CGCAAGGCACTTACAG | 13 | 33038 | 33053 | 243 |
| 797313 | 3227 | 3242 | CAAGATTCAGTCCCTG | 15 | 33056 | 33071 | 244 |
| 797314 | 3234 | 3249 | AAACGGGCAAGATTCA | 21 | 33063 | 33078 | 245 |
| 797315 | 3241 | 3256 | CATACATAAACGGGCA | 19 | 33070 | 33085 | 246 |
| 797316 | 3248 | 3263 | CATGGAGCATACATAA | 52 | 33077 | 33092 | 247 |
| 797317 | 3255 | 3270 | GGCTAGACATGGAGCA | 39 | 33084 | 33099 | 248 |
| 797318 | 3263 | 3278 | GGATGATGGGCTAGAC | 18 | 33092 | 33107 | 249 |
| 797319 | 3271 | 3286 | TCCAAGCAGGATGATG | 82 | 33100 | 33115 | 250 |
| 797320 | 3282 | 3297 | TGCCTACTTGCTCCAA | 51 | 33111 | 33126 | 251 |
| 797321 | 3291 | 3306 | TTGAGCTCCTGCCTAC | 21 | 33120 | 33135 | 252 |
| 797322 | 3293 | 3308 | TATTGAGCTCCTGCCT | 52 | 33122 | 33137 | 253 |
| 797323 | 3294 | 3309 | TTATTGAGCTCCTGCC | 35 | 33123 | 33138 | 254 |
| 797324 | N/A | N/A | AATCAGTTTTCTGAGG | 80 | 2644 | 2659 | 255 |
| 797325 | N/A | N/A | AAGGATAAATCAGTTT | 98 | 2651 | 2666 | 256 |
| 797326 | N/A | N/A | AGAAACTGACCCTTCC | 75 | 2668 | 2683 | 257 |
| 797327 | N/A | N/A | CCTAATGAAGAAACTG | 78 | 2676 | 2691 | 258 |
| 797328 | N/A | N/A | CTTCATTGTCCTAATG | 103 | 2685 | 2700 | 259 |
| 797329 | N/A | N/A | AGCTGGATTTTTCTTC | 68 | 2697 | 2712 | 260 |
| 797330 | N/A | N/A | GAAGGGACAGCTGGAT | 64 | 2705 | 2720 | 261 |
| 797331 | N/A | N/A | CATGATACCTCCCCTT | 47 | 2722 | 2737 | 262 |
| 797332 | N/A | N/A | TGATACTGCTCATGAT | 73 | 2732 | 2747 | 263 |
| 797333 | N/A | N/A | TCCTAGCACCTCCCTT | 57 | 4867 | 4882 | 264 |
| 797334 | N/A | N/A | ACCTTTCGAGTTTTGT | 26 | 4882 | 4897 | 265 |
| 797335 | N/A | N/A | TGATAGGGCCACCTTT | 47 | 4892 | 4907 | 266 |
| 797336 | N/A | N/A | CTAGAACGGCCTCTCC | 48 | 4918 | 4933 | 267 |
| 797337 | N/A | N/A | CCGGAGCTGGGCTTCC | 69 | 4934 | 4949 | 268 |
| 797338 | N/A | N/A | CAAAAGTGCCGGAGCT | 36 | 4942 | 4957 | 269 |
| 797339 | N/A | N/A | CAGCAGACCTGCGGGA | 33 | 4966 | 4981 | 270 |
| 797340 | N/A | N/A | CTGGAGCCAGCAGACC | 16 | 4973 | 4988 | 271 |
| 797341 | N/A | N/A | CCACATTCTCCCACTC | 50 | 5011 | 5026 | 272 |
| 797342 | N/A | N/A | TCCCACCCTGCGCCCA | 82 | 5024 | 5039 | 273 |
| 797343 | N/A | N/A | GGCCATGCCCATGTCC | 65 | 5037 | 5052 | 274 |
| 797344 | N/A | N/A | CCCGAGTGAGGCTGCC | 22 | 5056 | 5071 | 275 |
| 797345 | N/A | N/A | GGCTGAGCTCTGGGCC | 72 | 5101 | 5116 | 276 |
| 797346 | N/A | N/A | GGTCAGGGTCAAGGCT | 50 | 5113 | 5128 | 277 |
| 797347 | N/A | N/A | CCCCGGAGTGGATTGG | 71 | 5139 | 5154 | 278 |
| 797348 | N/A | N/A | GGCCTTTCGGCTTGAG | 72 | 15860 | 15875 | 279 |
| 797349 | N/A | N/A | GGCGAGTGTCGGTGGC | 43 | 15873 | 15888 | 280 |
| 797350 | N/A | N/A | GGTCATCCCGCCCTGA | 40 | 15897 | 15912 | 281 |
| 797351 | N/A | N/A | CGGTACCCAGGTCATC | 51 | 15906 | 15921 | 282 |
| 797352 | N/A | N/A | CGTGACCGCGGTACCC | 57 | 15914 | 15929 | 283 |
| 797353 | N/A | N/A | GGTGAGGGCCTCGGCC | 88 | 15934 | 15949 | 284 |
| 797354 | N/A | N/A | CGGAACTTGTCTGCCC | 45 | 15968 | 15983 | 285 |
| 797355 | N/A | N/A | GGGAACCCGGAACTTG | 85 | 15975 | 15990 | 286 |
| 797356 | N/A | N/A | CCTAGAAGGGAACCCG | 38 | 15982 | 15997 | 287 |
| 797357 | N/A | N/A | GCCCGGACCTAGAAGG | 82 | 15989 | 16004 | 288 |
| 797358 | N/A | N/A | AGAGAGGCAGGAGGCG | 104 | 16034 | 16049 | 289 |
| 797359 | N/A | N/A | TTGAAGGAGAGAGGCA | 52 | 16041 | 16056 | 290 |
| 797360 | N/A | N/A | GTGGACTGTTTATTGA | 67 | 16053 | 16068 | 291 |
| 797361 | N/A | N/A | GGACACTGTGGACTGT | 51 | 16060 | 16075 | 292 |
| 797362 | N/A | N/A | GCCCAGCCGGGACACT | 71 | 16069 | 16084 | 293 |
| 797363 | N/A | N/A | CCACGGCGAGCCCAGC | 69 | 16078 | 16093 | 294 |
| 797364 | N/A | N/A | GCGGAGGCCACGGCGA | 83 | 16085 | 16100 | 295 |
| 797365 | N/A | N/A | CGGGAAGGCGGAGGCC | 78 | 16092 | 16107 | 296 |
| 797366 | N/A | N/A | AAACAGGTGTGTCCGC | 77 | 16108 | 16123 | 297 |
| 797367 | N/A | N/A | GTGAAGGGTAAACAGG | 77 | 16117 | 16132 | 298 |
| 797368 | N/A | N/A | GGCCGTCCGGCGGTGA | 55 | 16129 | 16144 | 299 |
| 797369 | N/A | N/A | GGAGAAGCCTGGGCGG | 83 | 16170 | 16185 | 300 |
| 797370 | N/A | N/A | GTCCATCCCGGAGAAG | 63 | 16179 | 16194 | 301 |
| 797371 | N/A | N/A | CGGGAGGCCGGTCCAT | 52 | 16189 | 16204 | 302 |
| 797372 | N/A | N/A | GGTCAGGGTCCTCATC | 79 | 16212 | 16227 | 303 |
| 797373 | N/A | N/A | AGCGAGTGTCTGGCCC | 94 | 16234 | 16249 | 304 |
| 797374 | N/A | N/A | GAAGAGGGTCAGGCCA | 52 | 16260 | 16275 | 305 |
| 797375 | N/A | N/A | GAGTAATTCCTGGTTG | 103 | N/A | N/A | 306 |
| 797376 | N/A | N/A | TGTCTTTAAAACGGAC | 112 | 3040 | 3055 | 307 |
| 797377 | N/A | N/A | GAGGAGATAGGCCTGC | 42 | 3098 | 3113 | 308 |
| 797378 | N/A | N/A | AAAAAGGGCTGGAGGA | 113 | 3291 | 3306 | 309 |
| 797379 | N/A | N/A | AATCAGACCCAAAAAG | 134 | 3301 | 3316 | 310 |
| 797380 | N/A | N/A | GAGGAGGGTCAGAGAA | 69 | 3315 | 3330 | 311 |
| 797381 | N/A | N/A | TTGCAGGAATGTGGGC | 80 | 3593 | 3608 | 312 |
| 797382 | N/A | N/A | GGCCAGGAATGTGTAA | 103 | 3632 | 3647 | 313 |
| 797383 | N/A | N/A | GGACATTCTGTTCTTT | 97 | 3667 | 3682 | 314 |
| 797384 | N/A | N/A | GACAATAGAGAGGGAC | 93 | 4090 | 4105 | 315 |
| 797385 | N/A | N/A | GCAGAAAGAGGGAGAC | 85 | 4103 | 4118 | 316 |
| 797386 | 346 | 361 | GTTCCCCTTCATGAGC | 26 | 5154 | 5169 | 317 |
| | | | | | 5274 | 5289 | |
| 797387 | 349 | 364 | CTTGTTCCCCTTCATG | 15 | 5157 | 5172 | 318 |
| | | | | | 5277 | 5292 | |
| 797388 | N/A | N/A | AGTAAGCTGGAGGCTC | 69 | 5784 | 5799 | 319 |
| 797389 | N/A | N/A | TGCAAGCACTCCCTCC | 40 | 5932 | 5947 | 320 |
| 797390 | N/A | N/A | GAAAAGGGATGCAGCT | 81 | 5967 | 5982 | 321 |
| 797391 | N/A | N/A | AGCATTTTAGCCTGGG | 20 | 6265 | 6280 | 322 |
| 797392 | N/A | N/A | TATACAAAAGCACTCA | 62 | 6422 | 6437 | 323 |
| 797393 | N/A | N/A | GAATTATTCATGAATC | 42 | 6523 | 6538 | 324 |
| 797394 | N/A | N/A | TGGAATATACGAAGGG | 24 | 6782 | 6797 | 325 |
| 797395 | N/A | N/A | CATATTTTCAACCACA | 25 | 7349 | 7364 | 326 |
| 797396 | N/A | N/A | TAATACTGCCCACCTC | 76 | 7746 | 7761 | 327 |
| 797397 | N/A | N/A | TTGATTTAGATTCATT | 61 | 8239 | 8254 | 328 |
| 797398 | N/A | N/A | ACTTAAAGTGTAATGG | 89 | 8562 | 8577 | 329 |
| 797399 | N/A | N/A | TGCCTGATCCCTACTT | 59 | 8574 | 8589 | 330 |
| 797400 | N/A | N/A | GAAAAATATGTCTGTG | 53 | 9080 | 9095 | 331 |
| 797401 | N/A | N/A | AGCCGTGGGAGCCGCC | 31 | 9458 | 9473 | 332 |
| 797402 | N/A | N/A | GTCCAGGACGGAGCAG | 32 | 9587 | 9602 | 333 |
| 797403 | N/A | N/A | AAGACATCCGATCTTG | 92 | 10021 | 10036 | 334 |
| 797404 | N/A | N/A | CACTAAACAGAAAGCA | 38 | 10042 | 10057 | 335 |
| 797405 | N/A | N/A | GCATAAGATAAGACGG | 15 | 10454 | 10469 | 336 |
| 797406 | N/A | N/A | CACAAACCTGTGACAA | 48 | 10544 | 10559 | 337 |
| 797407 | N/A | N/A | CTCCTGCCACCCTACG | 66 | 10638 | 10653 | 338 |
| | | | | | 10661 | 10676 | |
| | | | | | 10684 | 10699 | |
| 797408 | N/A | N/A | CTCCCTCCTGCCACCC | 50 | 10642 | 10657 | 339 |
| | | | | | 10665 | 10680 | |
| | | | | | 10688 | 10703 | |
| | | | | | 10711 | 10726 | |
| 797409 | N/A | N/A | CACCTCCCTCCTGCCA | 43 | 10645 | 10660 | 340 |
| | | | | | 10668 | 10683 | |
| | | | | | 10691 | 10706 | |
| 797410 | N/A | N/A | ACGCACCTCCCTCCTG | 30 | 10648 | 10663 | 341 |
| | | | | | 10671 | 10686 | |
| 797411 | N/A | N/A | CCTACGCACCTCCCTC | 54 | 10651 | 10666 | 342 |
| | | | | | 10674 | 10689 | |
| 797412 | N/A | N/A | CACCCTACGCACCTCC | 59 | 10654 | 10669 | 343 |
| | | | | | 10677 | 10692 | |
| 797413 | N/A | N/A | TGCCACCCTACGCACC | 29 | 10657 | 10672 | 344 |
| | | | | | 10680 | 10695 | |
| 797414 | N/A | N/A | GAAGAATCCAGATCCC | 56 | 10944 | 10959 | 345 |
| 797415 | N/A | N/A | AGGAAGAATCCAGATC | 118 | 10946 | 10961 | 346 |
| 797416 | N/A | N/A | GCGAATTTGCCTTTCT | 40 | 10973 | 10988 | 347 |
| 797417 | N/A | N/A | TGAGAAAATACTCAGT | 31 | 11051 | 11066 | 348 |
| 797418 | N/A | N/A | TCTGAGATGTAGGGCC | 41 | 11208 | 11223 | 349 |
| 797419 | N/A | N/A | CTCCAACCACCACACT | 73 | 11307 | 11322 | 350 |
| 797420 | N/A | N/A | TGGCACAGCTAGCAAA | 65 | 11337 | 11352 | 351 |
| 797421 | N/A | N/A | CTTTAGGCTAAAACTT | 101 | 11469 | 11484 | 352 |
| 797422 | N/A | N/A | CACTATGCATGAAGAA | 40 | 11521 | 11536 | 353 |
| 797423 | N/A | N/A | GACAAGTGGGCTGCCT | 27 | 11611 | 11626 | 354 |
| 797424 | N/A | N/A | TTAATTGTTAAAAGAA | 72 | 11860 | 11875 | 355 |
| | | | | | 12660 | 12675 | |
| 797425 | N/A | N/A | ATTTAATTGTTAAAAG | 103 | 11862 | 11877 | 356 |
| | | | | | 12662 | 12677 | |
| 797426 | N/A | N/A | TATTTAATTGTTAAAA | 113 | 11863 | 11878 | 357 |
| | | | | | 12663 | 12678 | |
| 797427 | N/A | N/A | CATTATTTAATTGTTA | 127 | 11866 | 11881 | 358 |
| | | | | | 12666 | 12681 | |
| 797428 | N/A | N/A | AAAGAATGGCAAGCAT | 25 | 11937 | 11952 | 359 |
| 797429 | N/A | N/A | GGTTGAAGGTGTGTTT | 36 | 11988 | 12003 | 360 |
| 797430 | N/A | N/A | TGTCAAACCTGAGTGG | 76 | 12309 | 12324 | 361 |
| 797431 | N/A | N/A | CAACATCTCGACTGTC | 25 | 12321 | 12336 | 362 |
| 797432 | N/A | N/A | GATAGAGATAGCATTC | 71 | 12401 | 12416 | 363 |
| 797433 | N/A | N/A | ACAGACAAAACCAGTT | 85 | 12417 | 12432 | 364 |
| 797434 | N/A | N/A | TGGCAATCATAGCTAG | 38 | 12731 | 12746 | 365 |
| 797435 | N/A | N/A | CGACGAAACCTTGTAT | 53 | 12931 | 12946 | 366 |
| 797436 | N/A | N/A | AAGAATGGTAATCTGC | 57 | 13042 | 13057 | 367 |
| 797437 | N/A | N/A | GCCAAAAAGCCTGAAG | 28 | 13125 | 13140 | 368 |
| 797438 | N/A | N/A | TCATAGCCATTTTATT | 83 | 13148 | 13163 | 369 |
| 797439 | N/A | N/A | AAAGATTTGTACATGA | 23 | 13168 | 13183 | 370 |
| | | | | | 13483 | 13498 | |
| 797440 | N/A | N/A | ATATTAAGAAGGAATG | 71 | 13566 | 13581 | 371 |
| 797441 | N/A | N/A | TACGATCATTTTGGAA | 42 | 13770 | 13785 | 372 |
| 797442 | N/A | N/A | GAACAGACCTACATTT | 74 | 13832 | 13847 | 373 |
| | | | | | 14129 | 14144 | |
| 797443 | N/A | N/A | CAGACCTACATTTTTT | 48 | 14126 | 14141 | 374 |
| 797444 | N/A | N/A | ACCGTATGTAGTAGGC | 10 | 14152 | 14167 | 375 |
| 797445 | N/A | N/A | AACAAATAATCCCTAG | 88 | 14172 | 14187 | 376 |
| 797446 | N/A | N/A | TCTAGAAGATGGAAGA | 56 | 14238 | 14253 | 377 |
| 797447 | N/A | N/A | GAACCATAAACACTCT | 19 | 14251 | 14266 | 378 |
| 797448 | N/A | N/A | CTATACAGGCAAAAAT | 80 | 14304 | 14319 | 379 |
| 797449 | N/A | N/A | CAAAAGTGTGCCACCA | 31 | 14828 | 14843 | 380 |
| 797450 | N/A | N/A | ATGGATTCAACACAAT | 26 | 14993 | 15008 | 381 |
| 797451 | N/A | N/A | AAATTAATTGCATTTC | 103 | 15184 | 15199 | 382 |
| | | | | | 15212 | 15227 | |
| 797452 | N/A | N/A | TTGCATTTCCGTCTCA | 23 | 15205 | 15220 | 383 |
| 797453 | N/A | N/A | AAAAATTAATTGCATT | 86 | 15214 | 15229 | 384 |
| | | | | | 15530 | 15545 | |
| 797454 | N/A | N/A | AAAAAAATTAATTGCA | 106 | 15216 | 15231 | 385 |
| | | | | | 15532 | 15547 | |
| 797455 | N/A | N/A | AAAAAAAATTAATTGC | 67 | 15217 | 15232 | 386 |
| | | | | | 15533 | 15548 | |
| 797456 | N/A | N/A | TAACAAACTGAACAAG | 73 | 15574 | 15589 | 387 |
| 797457 | N/A | N/A | TGTTTCGGGTGCGGCC | 62 | 15731 | 15746 | 388 |
| 797458 | N/A | N/A | CCAAAGACTGTTCTAA | 44 | 15749 | 15764 | 389 |
| 797459 | N/A | N/A | ACCCACCCCGCCTCCC | 100 | 15769 | 15784 | 390 |
| 797460 | N/A | N/A | TTAGAATCTCCAACTC | 52 | 15804 | 15819 | 391 |
| 797461 | N/A | N/A | GGTCAGGAAAGGAGCG | 22 | 16629 | 16644 | 392 |
| 797462 | N/A | N/A | GGTGTTATTTTAATTA | 84 | 16730 | 16745 | 393 |
| 797463 | N/A | N/A | AAAAGCTTGGGCACCA | 27 | 16749 | 16764 | 394 |
| 797464 | N/A | N/A | CAAGAGCTGGGACTAG | 65 | 17403 | 17418 | 395 |
| 797465 | N/A | N/A | AAAGAATGAGTGATCT | 65 | 17676 | 17691 | 396 |
| 797466 | N/A | N/A | GTCAACAAGCATTTCC | 15 | 18034 | 18049 | 397 |
| 797467 | N/A | N/A | GGCATTTTTTTAGTCA | 59 | 18046 | 18061 | 398 |
| 797468 | N/A | N/A | CAAGATCCATGCTTCC | 14 | 18218 | 18233 | 399 |
| 797469 | N/A | N/A | GGATGATGTGATACAT | 9 | 18734 | 18749 | 400 |
| 797470 | N/A | N/A | CAATCTAAGAAATAGG | 24 | 18757 | 18772 | 401 |
| 797471 | N/A | N/A | TCCAAATGCCTAGAAC | 16 | 18859 | 18874 | 402 |
| 797472 | N/A | N/A | GGCGGACTCAGGCTTA | 75 | 19484 | 19499 | 403 |
| 797473 | N/A | N/A | TGACAGTTAGAGGAAC | 30 | 19515 | 19530 | 404 |
| 797474 | N/A | N/A | GGAAAGCACAGGTGTC | 29 | 19535 | 19550 | 405 |
| | | | | | 19617 | 19632 | |
| 797475 | N/A | N/A | AGGGAAAGCACAGGTG | 41 | 19537 | 19552 | 406 |
| | | | | | 19619 | 19634 | |
| 797476 | N/A | N/A | GCATAGGGAAAGCACA | 33 | 19541 | 19556 | 407 |
| | | | | | 19623 | 19638 | |
| 797477 | N/A | N/A | GGGCATAGGGAAAGCA | 33 | 19543 | 19558 | 408 |
| | | | | | 19625 | 19640 | |
| 797478 | N/A | N/A | GAGGGCATAGGGAAAG | 60 | 19545 | 19560 | 409 |
| | | | | | 19627 | 19642 | |
| 797479 | N/A | N/A | GGTGAGGGCATAGGGA | 40 | 19548 | 19563 | 410 |
| | | | | | 19630 | 19645 | |
| 797480 | N/A | N/A | ATCGGGTGAGGGCATA | 34 | 19552 | 19567 | 411 |
| | | | | | 19634 | 19649 | |
| 797481 | N/A | N/A | ACAGAGCAAAGGGAGG | 24 | 19569 | 19584 | 412 |
| | | | | | 19652 | 19667 | |
| 797482 | N/A | N/A | ACACAGAGCAAAGGGA | 45 | 19571 | 19586 | 413 |
| | | | | | 19654 | 19669 | |
| 797483 | N/A | N/A | GCACACAGAGCAAAGG | 19 | 19573 | 19588 | 414 |
| | | | | | 19656 | 19671 | |
| 797484 | N/A | N/A | AGGCACACAGAGCAAA | 25 | 19575 | 19590 | 415 |
| | | | | | 19658 | 19673 | |
| 797485 | N/A | N/A | AGGCAGGCACACAGAG | 26 | 19579 | 19594 | 416 |
| | | | | | 19662 | 19677 | |
| 797486 | N/A | N/A | GGGGAGGCAGGCACAC | 46 | 19583 | 19598 | 417 |
| | | | | | 19666 | 19681 | |
| 797487 | N/A | N/A | GCACAGGTGTCCATGG | 34 | 19612 | 19627 | 418 |
| 797488 | N/A | N/A | GGAATAGTTACATGTG | 10 | 19866 | 19881 | 419 |
| 797489 | N/A | N/A | ACCCGATAGCTGGTTG | 19 | 20416 | 20431 | 420 |
| 797490 | N/A | N/A | TCACACTATTAATTAG | 89 | 20435 | 20450 | 421 |
| 797491 | N/A | N/A | ACTGAACGATTTTAAA | 64 | 20606 | 20621 | 422 |
| 797492 | N/A | N/A | CCATGCTAAGGAGTAC | 30 | 20650 | 20665 | 423 |
| 797493 | N/A | N/A | GTACAGGGTTTCTTTT | 62 | 20864 | 20879 | 424 |
| | | | | | 27928 | 27943 | |
| 797494 | N/A | N/A | TTAAATGGTGTGACCA | 10 | 21648 | 21663 | 425 |
| 797495 | N/A | N/A | ACGATTACAGGGATTC | 9 | 21751 | 21766 | 426 |
| 797496 | N/A | N/A | AGCTGTATTAGCTCAC | 71 | 21771 | 21786 | 427 |
| 797497 | N/A | N/A | GTTACTTACTTAATCT | 17 | 21895 | 21910 | 428 |
| 797498 | N/A | N/A | AACAAGTATTAGATGT | 93 | 21923 | 21938 | 429 |
| 797499 | N/A | N/A | GCACAGACTCCAGAAT | 41 | 21970 | 21985 | 430 |
| 797500 | N/A | N/A | CAGTATAATGTGATGG | 5 | 22302 | 22317 | 431 |
| 797501 | N/A | N/A | GAGATACACACTAAGC | 5 | 22344 | 22359 | 432 |
| 797502 | N/A | N/A | CAGCGGTGGAGAAACA | 31 | 22750 | 22765 | 433 |
| 797503 | N/A | N/A | ATGGAAAGCAGGCACA | 8 | 22774 | 22789 | 434 |
| 797504 | N/A | N/A | AAGTATAATGGTGGGT | 32 | 22799 | 22814 | 435 |
| 797505 | N/A | N/A | TAAAATGTAGGATGAT | 86 | 23033 | 23048 | 436 |
| 797506 | N/A | N/A | ACTAAAGAGAAGAGGG | 68 | 23136 | 23151 | 437 |
| 797507 | N/A | N/A | AGCAAATCACAGGTTC | 5 | 23303 | 23318 | 438 |
| 797508 | N/A | N/A | CAGTACCAAGTGTTTC | 17 | 23442 | 23457 | 439 |
| 797509 | N/A | N/A | AGGAGATAGAGGAGAT | 39 | 23589 | 23604 | 440 |
| 797510 | N/A | N/A | CTTAAACTCCTACAGG | 59 | 24017 | 24032 | 441 |
| 797511 | N/A | N/A | AGCTCAAGGTAAGTAC | 98 | 24277 | 24292 | 442 |
| 797512 | N/A | N/A | TCCCAGTCAGCATCCT | 89 | 24733 | 24748 | 443 |
| 797513 | N/A | N/A | CCCCAGGGTCCACCCT | 75 | 24902 | 24917 | 444 |
| 797514 | N/A | N/A | CTCACATACACAACCC | 64 | 25321 | 25336 | 445 |
| 797515 | N/A | N/A | CTGCAGGTTGTTTTTA | 26 | 26098 | 26113 | 446 |
| | | | | | 26434 | 26449 | |
| 797516 | N/A | N/A | AATGGATACAGTATGT | 98 | 26688 | 26703 | 447 |
| 797517 | N/A | N/A | AGTCAAAAGGAAAGAG | 25 | 26848 | 26863 | 448 |
| 797518 | N/A | N/A | CAAACAACTCAACTGT | 24 | 26864 | 26879 | 449 |
| 797519 | N/A | N/A | AGCATGAACTCCAGGA | 11 | 26959 | 26974 | 450 |
| 797520 | N/A | N/A | AAATGCAACAGACTGT | 30 | 27542 | 27557 | 451 |
| 797521 | N/A | N/A | ACCCAAATCCCTACCA | 46 | 27624 | 27639 | 452 |
| 797522 | N/A | N/A | GATATAAATTATCTCA | 88 | 27780 | 27795 | 453 |
| 797523 | N/A | N/A | GCTAATGAGTACAAGG | 9 | 28243 | 28258 | 454 |
| 797524 | N/A | N/A | ACCATACGGATGAACC | 13 | 28742 | 28757 | 455 |
| 797525 | N/A | N/A | TCAAAAAAGGTTAAAC | 90 | 28996 | 29011 | 456 |
| 797526 | N/A | N/A | ATGTACAATGGTGATG | 34 | 29040 | 29055 | 457 |
| 797527 | N/A | N/A | AAAGAAAAATGGGAAC | 87 | 29535 | 29550 | 458 |
| | | | | | 29852 | 29867 | |
| 797528 | N/A | N/A | TTATAATGATGCCTTA | 78 | 30466 | 30481 | 459 |
| 797529 | N/A | N/A | CCCAAGAGGTCTCCCA | 58 | 30522 | 30537 | 460 |
| 797530 | N/A | N/A | CATAATACCCAGAGAA | 57 | 30895 | 30910 | 461 |
| 797531 | N/A | N/A | CACTCATACACATAAT | 68 | 30905 | 30920 | 462 |
| 797532 | N/A | N/A | TGTCACCCAGAGAAAA | 66 | 31564 | 31579 | 463 |
| 797533 | N/A | N/A | AGGTACATTGACGATG | 85 | 31720 | 31735 | 464 |

TABLE 2
Percent level of human α-ENaC mRNA

| | SEQ | SEQ | | | SEQ | SEQ | |
|---|---|---|---|---|---|---|---|
| | ID: 1 | ID: 1 | | α-ENaC | ID: 2 | ID 2: | SEQ |
| Compound | Start | Stop | | (% | Start | Stop | ID |
| Number | Site | Site | Sequence | control) | Site | Site | NO |
| 797192 | 1578 | 1593 | AAGATGTAGGCACAGC | 30 | 25492 | 25507 | 132 |
| 797235 | 2045 | 2060 | GGCTTCGGAACCTTCG | 18 | 31874 | 31889 | 166 |
| 797507 | N/A | N/A | AGCAAATCACAGGTTC | 20 | 23303 | 23318 | 438 |
| 826070 | 1 | 16 | GACGCAGACAGGCAAG | 76 | 4262 | 4277 | 465 |
| 826071 | 5 | 20 | TTTAGACGCAGACAGG | 67 | 4266 | 4281 | 466 |
| 826090 | 52 | 67 | GTGAACTGGGAGTACT | 91 | 4313 | 4328 | 467 |
| 826091 | 53 | 68 | GGTGAACTGGGAGTAC | 57 | 4314 | 4329 | 468 |
| 826110 | 145 | 160 | GCATCTCAATTAAAGG | 86 | 4406 | 4421 | 469 |
| 826111 | 163 | 178 | GCGACAGGAATCTCAT | 66 | 4424 | 4439 | 470 |
| 826130 | 195 | 210 | GGAGCCCGCCCGCTGG | 60 | 4456 | 4471 | 471 |
| 826131 | 216 | 231 | CAGGTGCAGCGGCCTG | 79 | 4477 | 4492 | 472 |
| 826150 | 282 | 29 | CCCTCCATGAGACCTG | 30 | 5210 | 5225 | 473 |
| 826151 | 283 | 298 | CCCCTCCATGAGACCT | 19 | 5211 | 5226 | 474 |
| 826170 | 354 | 369 | TCACGCTTGTTCCCCT | 25 | 5282 | 5297 | 475 |
| 826171 | 355 | 370 | CTCACGCTTGTTCCCC | 17 | 5283 | 5298 | 476 |
| 826190 | 439 | 454 | GTAGGAGCGGTGGAAC | 88 | 5367 | 5382 | 477 |
| 826191 | 441 | 456 | CGGTAGGAGCGGTGGA | 64 | 5369 | 5384 | 478 |
| 826209 | 565 | 580 | CATGCCAAAGGTGCAG | 43 | 5493 | 5508 | 479 |
| 826210 | 566 | 581 | TCATGCCAAAGGTGCA | 45 | 5494 | 5509 | 480 |
| 826229 | 606 | 62 | CTGAAGTACTCTCCGA | 23 | 5534 | 5549 | 481 |
| 826230 | 607 | 622 | GCTGAAGTACTCTCCG | 26 | 5535 | 5550 | 482 |
| 826249 | 702 | 717 | ATTTCCGGGTACCTGT | 33 | N/A | N/A | 483 |
| 826250 | 703 | 718 | AATTTCCGGGTACCTG | 40 | 16288 | 16303 | 484 |
| 826267 | 791 | 806 | CGGCCACGAGAGTGGT | 62 | 16376 | 16391 | 485 |
| 826268 | 792 | 807 | CCGGCCACGAGAGTGG | 58 | 16377 | 16392 | 486 |
| 826287 | 828 | 843 | GGCAGAGTCCCCCGCA | 35 | 16413 | 16428 | 487 |
| 826288 | 830 | 845 | GCGGCAGAGTCCCCCG | 38 | 16415 | 16430 | 488 |
| 826307 | 914 | 929 | TGTTGTCCCGCAAGCT | 42 | 16499 | 16514 | 489 |
| 826308 | 916 | 931 | GTTGTTGTCCCGCAAG | 26 | 16501 | 16516 | 490 |
| 826325 | 979 | 994 | GTCCGATTTGTTCTGG | 31 | 17765 | 17780 | 491 |
| 826326 | 980 | 995 | AGTCCGATTTGTTCTG | 58 | 17766 | 17781 | 492 |
| 826345 | 1017 | 1032 | ACCGCATCCACCCCTG | 42 | 17803 | 17818 | 493 |
| 826346 | 1018 | 1033 | CACCGCATCCACCCCT | 41 | 17804 | 17819 | 494 |
| 826365 | 1113 | 1128 | AAGATGAAGTTGCCCA | 41 | 17899 | 17914 | 495 |
| 826366 | 1115 | 1130 | CGAAGATGAAGTTGCC | 55 | 17901 | 17916 | 496 |
| 826385 | 1162 | 1177 | GTGAGAGTAATTCGCC | 65 | N/A | N/A | 497 |
| 826386 | 1164 | 1179 | AAGTGAGAGTAATTCG | 29 | N/A | N/A | 498 |
| 826405 | 1281 | 1296 | TGCTCTGCGCGCAGCA | 62 | 24578 | 24593 | 499 |
| 826406 | 1282 | 1297 | CTGCTCTGCGCGCAGC | 58 | 24579 | 24594 | 500 |
| 826425 | 1354 | 1369 | GGCAGGTTCATCCTGC | 140 | 24651 | 24666 | 50 |
| 826426 | 1356 | 1371 | AAGGCAGGTTCATCCT | 63 | 24653 | 24668 | 502 |
| 826445 | 1405 | 1420 | GATGGAGGTCTCCACG | 67 | 24702 | 24717 | 503 |
| 826446 | 1416 | 1431 | TTCCTCATGCTGATGG | 24 | 24713 | 24728 | 504 |
| 826465 | 1497 | 1512 | GGGTAAAGGTTCTCAA | 25 | 25217 | 25232 | 505 |
| 826466 | 1500 | 1515 | GAAGGGTAAAGGTTCT | 63 | 25220 | 25235 | 506 |
| 826485 | 1579 | 1594 | GAAGATGTAGGCACAG | 32 | 25493 | 25508 | 507 |
| 826504 | 1656 | 1671 | TGGAGCTTATAGTAGC | 30 | 30602 | 30617 | 508 |
| 826505 | 1661 | 1676 | CAACCTGGAGCTTATA | 42 | 30607 | 30622 | 509 |
| 826524 | 1733 | 1748 | AGAGCTGGTAGCTGGT | 67 | 30784 | 30799 | 510 |
| 826525 | 1736 | 1751 | CAGAGAGCTGGTAGCT | 75 | 30787 | 30802 | 511 |
| 826564 | 1992 | 2007 | ACGAGCTCAGCCATCT | 57 | 31821 | 31836 | 512 |
| 826565 | 1993 | 2008 | GACGAGCTCAGCCATC | 16 | 31822 | 31837 | 513 |
| 826584 | 2046 | 2061 | CGGCTTCGGAACCTTC | 23 | 31875 | 31890 | 514 |
| 826603 | 2216 | 2231 | CCAGGGTGGCATAGGC | 28 | 32045 | 32060 | 515 |
| 826604 | 2217 | 2232 | CCCAGGGTGGCATAGG | 42 | 32046 | 32061 | 516 |
| 826623 | 2360 | 2375 | CCTTCAATCTTGCCAG | 31 | 32189 | 32204 | 517 |
| 826624 | 2390 | 2405 | TGGGCGGCTCTGAGAG | 49 | 32219 | 32234 | 518 |
| 826643 | 2464 | 2479 | ATCAGCTACTGTTCTT | 32 | 32293 | 32308 | 519 |
| 826644 | 2476 | 2491 | CTGGGCAGCTTCATCA | 33 | 32305 | 32320 | 520 |
| 826662 | 2575 | 2590 | GCTGATCCAAGGGAAA | 35 | 32404 | 32419 | 521 |
| 826681 | 2610 | 2625 | GGAAAGTTCCTTGTCA | 40 | 32439 | 32454 | 522 |
| 826682 | 2612 | 2627 | TAGGAAAGTTCCTTGT | 32 | 32441 | 32456 | 523 |
| 826701 | 2679 | 2694 | GGCAGGAAACCCGTGC | 74 | 32508 | 32523 | 524 |
| 826702 | 2681 | 2696 | TGGGCAGGAAACCCGT | 64 | 32510 | 32525 | 525 |
| 826721 | 2832 | 2847 | AGCCCTCGGGAGTCAG | 27 | 32661 | 32676 | 526 |
| 826722 | 2835 | 2850 | CCTAGCCCTCGGGAGT | 62 | 32664 | 32679 | 527 |
| 826741 | 2896 | 2911 | GCACGGGTATGAGGCT | 69 | 32725 | 32740 | 528 |
| 826742 | 2949 | 2964 | TGTAGAGGTATGAAAG | 31 | 32778 | 32793 | 529 |
| 826761 | 3012 | 3027 | ATAGGAGTTCTCTGGC | 16 | 32841 | 32856 | 530 |
| 826780 | 3101 | 3116 | GAAGGCGGAGTGATCA | 65 | 32930 | 32945 | 531 |
| 826781 | 3117 | 3132 | CTACGGGAGCCCAGGA | 22 | 32946 | 32961 | 532 |
| 826799 | 3143 | 3158 | AACACTCCAGCAGATG | 40 | 32972 | 32987 | 533 |
| 826800 | 3145 | 3160 | GCAACACTCCAGCAGA | 19 | 32974 | 32989 | 534 |
| 826817 | 3212 | 3227 | GACCGCAAGGCACTTA | 51 | 33041 | 33056 | 535 |
| 826818 | 3213 | 3228 | TGACCGCAAGGCACTT | 19 | 33042 | 33057 | 536 |
| 826836 | 3235 | 3250 | TAAACGGGCAAGATTC | 41 | 33064 | 33079 | 537 |
| 826837 | 3236 | 3251 | ATAAACGGGCAAGATT | 71 | 33065 | 33080 | 538 |
| 826856 | N/A | N/A | TTGTCCTAATGAAGAA | 95 | 2680 | 2695 | 539 |
| 826857 | N/A | N/A | TCCCCTTGGAAGGGAC | 66 | 2713 | 2728 | 540 |
| 826876 | N/A | N/A | AGGATCTGTGTCTCAG | 19 | 4815 | 4830 | 541 |
| 826877 | N/A | N/A | GTCCTAGCACCTCCCT | 15 | 4868 | 4883 | 542 |
| 826896 | N/A | N/A | CCCTAGAACGGCCTCT | 27 | 4920 | 4935 | 543 |
| 826897 | N/A | N/A | CTTCCCTAGAACGGCC | 19 | 4923 | 4938 | 544 |
| 826916 | N/A | N/A | ACCCGAGTGAGGCTGC | 13 | 5057 | 5072 | 545 |
| 826917 | N/A | N/A | GAACCCGAGTGAGGCT | 60 | 5059 | 5074 | 546 |
| 826936 | N/A | N/A | CCACACAGAGCCCGTG | 66 | 2463 | 2478 | 547 |
| 826937 | N/A | N/A | AGCCGGGAAGGCCTCC | 72 | 2486 | 2501 | 548 |
| 826956 | N/A | N/A | CCCAAAAAGGGCTGGA | 66 | 3294 | 3309 | 549 |
| 826957 | N/A | N/A | GCCAAGTGGTGAGCAA | 74 | 3338 | 3353 | 550 |
| 826976 | N/A | N/A | GTTGAATCTGGCAGCC | 64 | 4517 | 4532 | 551 |
| 826977 | N/A | N/A | TGGGACTGGTTCCTTT | 109 | 4536 | 4551 | 552 |
| 826996 | N/A | N/A | TGGCAAAGAGCACCGA | 51 | 6348 | 6363 | 553 |
| 826997 | N/A | N/A | CCAGACCCAACATTGG | 87 | 6361 | 6376 | 554 |
| 827016 | N/A | N/A | GTCCGTAACGCACCTT | 36 | 6807 | 6822 | 555 |
| 827017 | N/A | N/A | CTTTCTTAGTCCGTAA | 41 | 6815 | 6830 | 556 |
| 827036 | N/A | N/A | GACTACAAGGTCAAGT | 77 | 7660 | 7675 | 557 |
| 827056 | N/A | N/A | ATTCACTGCGCTCCCG | 44 | 8755 | 8770 | 558 |
| 827057 | N/A | N/A | TCGGTAGGAGTCATTC | 15 | 8767 | 8782 | 559 |
| 827076 | N/A | N/A | ACAGAAGAGCCCATGC | 55 | 9484 | 9499 | 560 |
| 827077 | N/A | N/A | TCTTACCCCGGTGGCC | 53 | 9507 | 9522 | 561 |
| 827096 | N/A | N/A | CCCTACGCGCCTCCCT | 92 | 10629 | 10644 | 562 |
| 827097 | N/A | N/A | CTGCCACCCTACGCGC | 53 | 10635 | 10650 | 563 |
| 827116 | N/A | N/A | GCCTACCGCATGAAGC | 42 | 11082 | 11097 | 564 |
| 827117 | N/A | N/A | GGGCATAACACTAGAT | 42 | 11100 | 11115 | 565 |
| 827136 | N/A | N/A | TGGACAAGGTTTGACA | 65 | 11623 | 11638 | 566 |
| 827137 | N/A | N/A | GGTTACACCCCCGGCG | 60 | 11650 | 11665 | 567 |
| 827156 | N/A | N/A | GCTACATTAACCACCG | 31 | 12134 | 12149 | 568 |
| 827157 | N/A | N/A | TTGAAAGAGCCCCCAC | 26 | 12166 | 12181 | 569 |
| 827176 | N/A | N/A | CAGGAACTATGGTATT | 18 | 12690 | 12705 | 570 |
| 827177 | N/A | N/A | GCAATCATAGCTAGCA | 116 | 12729 | 12744 | 571 |
| 827196 | N/A | N/A | TTTATGAACAGACCTA | 19 | 14134 | 14149 | 572 |
| 827197 | N/A | N/A | GTAGGCACTTTATGAA | 28 | 14142 | 14157 | 573 |
| 827215 | N/A | N/A | GCATAGATGGTCAACT | 43 | 14438 | 14453 | 574 |
| 827216 | N/A | N/A | GGAGAGACAATAGATC | 31 | 14488 | 14503 | 575 |
| 827235 | N/A | N/A | GGCTTGAGTGCCGCTT | 17 | 15852 | 15867 | 576 |
| 827236 | N/A | N/A | CCGCAGGCGAGTGTCG | 63 | 15878 | 15893 | 577 |
| 827255 | N/A | N/A | ACTAATGGGAACTTCC | 47 | 17363 | 17378 | 578 |
| 827256 | N/A | N/A | CAAGAGATTTGTCCCA | 39 | 17420 | 17435 | 579 |
| 827275 | N/A | N/A | GAGCAGCAGGAGTTCG | 45 | 18126 | 18141 | 580 |
| 827276 | N/A | N/A | CCTCAGATCCAGCAGT | 50 | 18147 | 18162 | 581 |
| 827294 | N/A | N/A | ATACATCCAGAGTCAC | 25 | 18724 | 18739 | 582 |
| 827295 | N/A | N/A | GATACATCCAGAGTCA | 31 | 18725 | 18740 | 583 |
| 827313 | N/A | N/A | CTCCGGAAAATAAACG | 25 | 19270 | 19285 | 584 |
| 827314 | N/A | N/A | GGAGATGGCTCCGGAA | 55 | 19278 | 19293 | 585 |
| 827333 | N/A | N/A | ACCATGGACTTTCTGT | 18 | 19686 | 19701 | 586 |
| 827334 | N/A | N/A | AACCATGGACTTTCTG | 42 | 19687 | 19702 | 587 |
| 827352 | N/A | N/A | CTAACTGGAATAGTTA | 89 | 19872 | 19887 | 588 |
| 827353 | N/A | N/A | ACTAACTGGAATAGTT | 60 | 19873 | 19888 | 589 |
| 827372 | N/A | N/A | ATCATAGTATTCAGCC | 18 | 21087 | 21102 | 590 |
| 827373 | N/A | N/A | AAAAAGGTGGTGTATC | 50 | 21111 | 21126 | 591 |
| 827391 | N/A | N/A | ATTACAGGGATTCATT | 26 | 21748 | 21763 | 592 |
| 827392 | N/A | N/A | CGATTACAGGGATTCA | 20 | 21750 | 21765 | 593 |
| 827409 | N/A | N/A | GGCCAGTAAGAGTGAA | 90 | 22013 | 22028 | 594 |
| 827410 | N/A | N/A | GAATCAGTATAATGTG | 11 | 22306 | 22321 | 595 |
| 827428 | N/A | N/A | TGCCCCCATGGAAAGC | 54 | 22781 | 22796 | 596 |
| 827429 | N/A | N/A | CTGCCCCCATGGAAAG | 32 | 22782 | 22797 | 597 |
| 827448 | N/A | N/A | CTGCAGTAGGACTGCA | 80 | 23326 | 23341 | 598 |
| 827467 | N/A | N/A | AAGCAGGGAGCTTCTC | 80 | 24227 | 24242 | 599 |
| 827468 | N/A | N/A | CGCCATGGAGCAAGCA | 95 | 24238 | 24253 | 600 |
| 827487 | N/A | N/A | TGCAACACTGAGAGGG | 45 | 26035 | 26050 | 601 |
| 827488 | N/A | N/A | GGACAATTCCTTGACA | 38 | 26078 | 26093 | 602 |
| 827507 | N/A | N/A | GACAATCCGCTGCCTT | 15 | 27116 | 27131 | 603 |
| 827508 | N/A | N/A | AGGTAGGGATGGACGC | 15 | 27147 | 27162 | 604 |
| 827527 | N/A | N/A | GGGACTTGCTAATGAG | 50 | 28250 | 28265 | 605 |
| 827528 | N/A | N/A | TGGGACTTGCTAATGA | 48 | 28251 | 28266 | 606 |
| 827546 | N/A | N/A | GTCCACTAACTGATAA | 52 | 28839 | 28854 | 607 |
| 827547 | N/A | N/A | GGTGATGTCACTTCGG | 12 | 29031 | 29046 | 608 |
| 827566 | N/A | N/A | CACATAATACCCAGAG | 61 | 30897 | 30912 | 609 |
| 827567 | N/A | N/A | GGATAGGGTTGTGTCA | 57 | 30925 | 30940 | 610 |

TABLE 3
Percent level of human α-ENaC mRNA

| | SEQ | SEQ | | | | | |
|---|---|---|---|---|---|---|---|
| | ID: | ID: | | | SEQ | SEQ | |
| | 1 | 1 | | α-ENaC | ID: 2 | ID 2: | SEQ |
| Compound | Start | Stop | | (% | Start | Stop | ID |
| Number | Site | Site | Sequence | control) | Site | Site | NO |
| 797494 | N/A | N/A | TTAAATGGTGTGACCA | 12 | 21648 | 21663 | 425 |
| 797524 | N/A | N/A | ACCATACGGATGAACC | 37 | 28742 | 28757 | 455 |
| 826074 | 25 | 40 | GGCGGACTCTGGGCAG | 135 | 4286 | 4301 | 611 |
| 826075 | 28 | 43 | GAAGGCGGACTCTGGG | 121 | 4289 | 4304 | 612 |
| 826076 | 29 | 44 | AGAAGGCGGACTCTGG | 119 | 4290 | 4305 | 613 |
| 826077 | 31 | 46 | TGAGAAGGCGGACTCT | 75 | 4292 | 4307 | 614 |
| 826078 | 32 | 47 | CTGAGAAGGCGGACTC | 70 | 4293 | 4308 | 615 |
| 826079 | 33 | 48 | CCTGAGAAGGCGGACT | 163 | 4294 | 4309 | 616 |
| 826081 | 36 | 51 | GGACCTGAGAAGGCGG | 46 | 4297 | 4312 | 617 |
| 826094 | 73 | 88 | AAGGAGGGCTCCCGAG | 111 | 4334 | 4349 | 618 |
| 826095 | 74 | 89 | GAAGGAGGGCTCCCGA | 196 | 4335 | 4350 | 619 |
| 826096 | 81 | 96 | TCCGAAGGAAGGAGGG | 197 | 4342 | 4357 | 620 |
| 826097 | 83 | 98 | TTTCCGAAGGAAGGAG | 89 | 4344 | 4359 | 621 |
| 826098 | 85 | 100 | GTTTTCCGAAGGAAGG | 125 | 4346 | 4361 | 622 |
| 826099 | 88 | 103 | GGAGTTTTCCGAAGGA | 149 | 4349 | 4364 | 623 |
| 826100 | 91 | 106 | CCGGGAGTTTTCCGAA | 59 | 4352 | 4367 | 624 |
| 826101 | 92 | 107 | GCCGGGAGTTTTCCGA | 98 | 4353 | 4368 | 625 |
| 826114 | 167 | 182 | GGAAGCGACAGGAATC | 162 | 4428 | 4443 | 626 |
| 826115 | 169 | 184 | ATGGAAGCGACAGGAA | 118 | 4430 | 4445 | 627 |
| 826116 | 170 | 185 | GATGGAAGCGACAGGA | 145 | 4431 | 4446 | 628 |
| 826117 | 171 | 186 | GGATGGAAGCGACAGG | 143 | 4432 | 4447 | 629 |
| 826118 | 172 | 187 | GGGATGGAAGCGACAG | 82 | 4433 | 4448 | 630 |
| 826119 | 175 | 190 | CCAGGGATGGAAGCGA | 95 | 4436 | 4451 | 631 |
| 826120 | 176 | 191 | GCCAGGGATGGAAGCG | 54 | 4437 | 4452 | 632 |
| 826121 | 180 | 195 | GCCGGCCAGGGATGGA | 74 | 4441 | 4456 | 633 |
| 826134 | 226 | 241 | GTTCCCCTGACAGGTG | 74 | N/A | N/A | 634 |
| 826135 | 228 | 243 | TTGTTCCCCTGACAGG | 68 | N/A | N/A | 635 |
| 826136 | 229 | 244 | CTTGTTCCCCTGACAG | 102 | N/A | N/A | 636 |
| 826137 | 230 | 245 | GCTTGTTCCCCTGACA | 15 | N/A | N/A | 637 |
| 826138 | 232 | 247 | CAGCTTGTTCCCCTGA | 30 | N/A | N/A | 638 |
| 826139 | 233 | 248 | CCAGCTTGTTCCCCTG | 77 | N/A | N/A | 639 |
| 826140 | 249 | 264 | CTAGGGTCCTGCTCCT | 55 | 5177 | 5192 | 640 |
| 826141 | 254 | 269 | GAGGTCTAGGGTCCTG | 44 | 5182 | 5197 | 641 |
| 826154 | 292 | 307 | CAGCTTGTTCCCCTCC | 33 | 5220 | 5235 | 642 |
| 826155 | 310 | 325 | GCTAGAGTCCTGCTCC | 82 | 5238 | 5253 | 643 |
| 826156 | 312 | 327 | GGGCTAGAGTCCTGCT | 134 | 5240 | 5255 | 644 |
| 826157 | 315 | 330 | GGAGGGCTAGAGTCCT | 91 | 5243 | 5258 | 645 |
| 826158 | 320 | 335 | ACTGTGGAGGGCTAGA | 50 | 5248 | 5263 | 646 |
| 826159 | 321 | 336 | GACTGTGGAGGGCTAG | 59 | 5249 | 5264 | 647 |
| 826160 | 322 | 337 | GGACTGTGGAGGGCTA | 34 | 5250 | 5265 | 648 |
| 826161 | 331 | 346 | CCCTGGAGTGGACTGT | 50 | 5259 | 5274 | 649 |
| 826174 | 360 | 375 | TGCTCCTCACGCTTGT | 27 | 5288 | 5303 | 650 |
| 826175 | 362 | 377 | CCTGCTCCTCACGCTT | 33 | 5290 | 5305 | 651 |
| 826176 | 363 | 378 | CCCTGCTCCTCACGCT | 51 | 5291 | 5306 | 652 |
| 826177 | 386 | 401 | GCGCCGCAGGTTCGGG | 51 | 5314 | 5329 | 653 |
| 826178 | 405 | 420 | TCCGCCGTGGGCTGCT | 52 | 5333 | 5348 | 654 |
| 826179 | 407 | 422 | CCTCCGCCGTGGGCTG | 50 | 5335 | 5350 | 655 |
| 826180 | 411 | 426 | TCCTCCTCCGCCGTGG | 31 | 5339 | 5354 | 656 |
| 826181 | 422 | 437 | CGATCAGGGCCTCCTC | 33 | 5350 | 5365 | 657 |
| 826194 | 446 | 461 | GCTCTCGGTAGGAGCG | 44 | 5374 | 5389 | 658 |
| 826195 | 448 | 463 | GAGCTCTCGGTAGGAG | 94 | 5376 | 5391 | 659 |
| 826196 | 451 | 466 | GAAGAGCTCTCGGTAG | 114 | 5379 | 5394 | 660 |
| 826197 | 453 | 468 | TCGAAGAGCTCTCGGT | 41 | 5381 | 5396 | 661 |
| 826198 | 456 | 471 | AACTCGAAGAGCTCTC | 30 | 5384 | 5399 | 662 |
| 826199 | 457 | 472 | GAACTCGAAGAGCTCT | 95 | 5385 | 5400 | 663 |
| 826200 | 466 | 481 | GTTGCAGAAGAACTCG | 27 | 5394 | 5409 | 664 |
| 826201 | 467 | 482 | TGTTGCAGAAGAACTC | 44 | 5395 | 5410 | 665 |
| 826213 | 575 | 590 | GCCAGTACATCATGCC | 34 | 5503 | 5518 | 666 |
| 826214 | 577 | 592 | TTGCCAGTACATCATG | 58 | 5505 | 5520 | 667 |
| 826215 | 580 | 595 | GAATTGCCAGTACATC | 46 | 5508 | 5523 | 668 |
| 826216 | 581 | 596 | CGAATTGCCAGTACAT | 27 | 5509 | 5524 | 669 |
| 826217 | 582 | 597 | CCGAATTGCCAGTACA | 26 | 5510 | 5525 | 670 |
| 826218 | 585 | 600 | AGGCCGAATTGCCAGT | 102 | 5513 | 5528 | 671 |
| 826219 | 587 | 602 | GCAGGCCGAATTGCCA | 34 | 5515 | 5530 | 672 |
| 826220 | 589 | 604 | AAGCAGGCCGAATTGC | 51 | 5517 | 5532 | 673 |
| 826233 | 626 | 641 | TGTTGAGGCTGACGGG | 14 | 5554 | 5569 | 674 |
| 826234 | 628 | 643 | GATGTTGAGGCTGACG | 28 | 5556 | 5571 | 675 |
| 826235 | 639 | 654 | GAGTTGAGGTTGATGT | 64 | 5567 | 5582 | 676 |
| 826236 | 641 | 656 | CCGAGTTGAGGTTGAT | 32 | 5569 | 5584 | 677 |
| 826237 | 643 | 658 | GTCCGAGTTGAGGTTG | 28 | 5571 | 5586 | 678 |
| 826238 | 644 | 659 | TGTCCGAGTTGAGGTT | 65 | 5572 | 5587 | 679 |
| 826239 | 645 | 660 | TTGTCCGAGTTGAGGT | 40 | 5573 | 5588 | 680 |
| 826240 | 647 | 662 | GCTTGTCCGAGTTGAG | 19 | 5575 | 5590 | 681 |
| 826253 | 731 | 746 | TGCGGTCCAGCTCCTC | 42 | 16316 | 16331 | 682 |
| 826254 | 734 | 749 | TGATGCGGTCCAGCTC | 88 | 16319 | 16334 | 683 |
| 826255 | 737 | 752 | CTGTGATGCGGTCCAG | 38 | 16322 | 16337 | 684 |
| 826256 | 739 | 754 | CTCTGTGATGCGGTCC | 20 | 16324 | 16339 | 685 |
| 826257 | 740 | 755 | GCTCTGTGATGCGGTC | 43 | 16325 | 16340 | 686 |
| 826258 | 759 | 774 | TACAGGTCAAAGAGCG | 52 | 16344 | 16359 | 687 |
| 826259 | 761 | 776 | TGTACAGGTCAAAGAG | 18 | 16346 | 16361 | 688 |
| 826260 | 762 | 777 | TTGTACAGGTCAAAGA | 41 | 16347 | 16362 | 689 |
| 826271 | 798 | 813 | CGGGAGCCGGCCACGA | 55 | 16383 | 16398 | 690 |
| 826272 | 800 | 815 | TGCGGGAGCCGGCCAC | 23 | 16385 | 16400 | 691 |
| 826273 | 803 | 818 | GGCTGCGGGAGCCGGC | 147 | 16388 | 16403 | 692 |
| 826274 | 804 | 819 | CGGCTGCGGGAGCCGG | 70 | 16389 | 16404 | 693 |
| 826275 | 805 | 820 | ACGGCTGCGGGAGCCG | 104 | 16390 | 16405 | 694 |
| 826276 | 807 | 822 | CGACGGCTGCGGGAGC | 101 | 16392 | 16407 | 695 |
| 826277 | 808 | 823 | GCGACGGCTGCGGGAG | 52 | 16393 | 16408 | 696 |
| 826278 | 810 | 825 | TCGCGACGGCTGCGGG | 52 | 16395 | 16410 | 697 |
| 826291 | 834 | 849 | GGGTGCGGCAGAGTCC | 46 | 16419 | 16434 | 698 |
| 826292 | 858 | 873 | GGCGGGACCCTCAGGC | 37 | 16443 | 16458 | 699 |
| 826293 | 877 | 892 | ACGGGCCCCGTGAGGC | 85 | 16462 | 16477 | 700 |
| 826294 | 879 | 894 | CGACGGGCCCCGTGAG | 49 | 16464 | 16479 | 701 |
| 826295 | 882 | 897 | GCTCGACGGGCCCCGT | 27 | 16467 | 16482 | 702 |
| 826296 | 883 | 898 | GGCTCGACGGGCCCCG | 64 | 16468 | 16483 | 703 |
| 826297 | 889 | 904 | GCTACGGGCTCGACGG | 33 | 16474 | 16489 | 704 |
| 826298 | 891 | 906 | ACGCTACGGGCTCGAC | 35 | 16476 | 16491 | 705 |
| 826311 | 952 | 967 | GAAGCCGATCTTCCAG | 43 | 16537 | 16552 | 706 |
| 826312 | 953 | 968 | GGAAGCCGATCTTCCA | 64 | 16538 | 16553 | 707 |
| 826313 | 954 | 969 | TGGAAGCCGATCTTCC | 161 | 16539 | 16554 | 708 |
| 826314 | 956 | 971 | GCTGGAAGCCGATCTT | 80 | 16541 | 16556 | 709 |
| 826315 | 958 | 973 | CAGCTGGAAGCCGATC | 41 | 16543 | 16558 | 710 |
| 826316 | 968 | 983 | TCTGGTTGCACAGCTG | 38 | N/A | N/A | 711 |
| 826317 | 969 | 984 | TTCTGGTTGCACAGCT | 53 | N/A | N/A | 712 |
| 826318 | 970 | 985 | GTTCTGGTTGCACAGC | 19 | N/A | N/A | 713 |
| 826329 | 983 | 998 | AGCAGTCCGATTTGTT | 45 | 17769 | 17784 | 714 |
| 826330 | 985 | 1000 | GAAGCAGTCCGATTTG | 72 | 17771 | 17786 | 715 |
| 826331 | 986 | 1001 | AGAAGCAGTCCGATTT | 92 | 17772 | 17787 | 716 |
| 826332 | 987 | 1002 | TAGAAGCAGTCCGATT | 124 | 17773 | 17788 | 717 |
| 826333 | 988 | 1003 | GTAGAAGCAGTCCGAT | 50 | 17774 | 17789 | 718 |
| 826334 | 989 | 1004 | GGTAGAAGCAGTCCGA | 18 | 17775 | 17790 | 719 |
| 826335 | 994 | 1009 | TGTCTGGTAGAAGCAG | 25 | 17780 | 17795 | 720 |
| 826336 | 995 | 1010 | ATGTCTGGTAGAAGCA | 35 | 17781 | 17796 | 721 |
| 826349 | 1022 | 1037 | CCCTCACCGCATCCAC | 32 | 17808 | 17823 | 722 |
| 826350 | 1025 | 1040 | ACTCCCTCACCGCATC | 68 | 17811 | 17826 | 723 |
| 826351 | 1026 | 1041 | CACTCCCTCACCGCAT | 128 | 17812 | 17827 | 724 |
| 826352 | 1028 | 1043 | ACCACTCCCTCACCGC | 33 | 17814 | 17829 | 725 |
| 826353 | 1032 | 1047 | CGGTACCACTCCCTCA | 49 | 17818 | 17833 | 726 |
| 826354 | 1033 | 1048 | GCGGTACCACTCCCTC | 69 | 17819 | 17834 | 727 |
| 826355 | 1034 | 1049 | AGCGGTACCACTCCCT | 24 | 17820 | 17835 | 728 |
| 826356 | 1045 | 1060 | GATGTAGTGGAAGCGG | 36 | 17831 | 17846 | 729 |
| 826369 | 1123 | 1138 | GCGGCAGGCGAAGATG | 49 | 17909 | 17924 | 730 |
| 826370 | 1126 | 1141 | GAAGCGGCAGGCGAAG | 55 | 17912 | 17927 | 731 |
| 826371 | 1129 | 1144 | GTTGAAGCGGCAGGCG | 97 | 17915 | 17930 | 732 |
| 826372 | 1130 | 1145 | GGTTGAAGCGGCAGGC | 30 | 17916 | 17931 | 733 |
| 826373 | 1134 | 1149 | ACCTGGTTGAAGCGGC | 20 | 17920 | 17935 | 734 |
| 826374 | 1136 | 1151 | AGACCTGGTTGAAGCG | 44 | 17922 | 17937 | 735 |
| 826375 | 1138 | 1153 | GGAGACCTGGTTGAAG | 68 | 17924 | 17939 | 736 |
| 826376 | 1146 | 1161 | TGGTTGCAGGAGACCT | 143 | 17932 | 17947 | 737 |
| 826389 | 1232 | 1247 | AAGACATCCAGAGGTT | 80 | 24188 | 24203 | 738 |
| 826390 | 1250 | 1265 | TGTTGATTCCAGGCAT | 53 | 24206 | 24221 | 739 |
| 826391 | 1251 | 1266 | TTGTTGATTCCAGGCA | 43 | 24207 | 24222 | 740 |
| 826392 | 1252 | 1267 | GTTGTTGATTCCAGGC | 31 | 24208 | 24223 | 741 |
| 826393 | 1254 | 1269 | CCGTTGTTGATTCCAG | 15 | 24210 | 24225 | 742 |
| 826394 | 1255 | 1270 | ACCGTTGTTGATTCCA | 15 | 24211 | 24226 | 743 |
| 826395 | 1257 | 1272 | AGACCGTTGTTGATTC | 30 | N/A | N/A | 744 |
| 826396 | 1259 | 1274 | ACAGACCGTTGTTGAT | 33 | N/A | N/A | 745 |
| 826409 | 1285 | 1300 | ATTCTGCTCTGCGCGC | 39 | 24582 | 24597 | 746 |
| 826410 | 1286 | 1301 | CATTCTGCTCTGCGCG | 36 | 24583 | 24598 | 747 |
| 826411 | 1287 | 1302 | TCATTCTGCTCTGCGC | 80 | 24584 | 24599 | 748 |
| 826412 | 1323 | 1338 | CGGGCCCCAGTCACTG | 118 | 24620 | 24635 | 749 |
| 826413 | 1325 | 1340 | CCCGGGCCCCAGTCAC | 82 | 24622 | 24637 | 750 |
| 826414 | 1327 | 1342 | TACCCGGGCCCCAGTC | 31 | 24624 | 24639 | 751 |
| 826415 | 1329 | 1344 | ATTACCCGGGCCCCAG | 56 | 24626 | 24641 | 752 |
| 826416 | 1331 | 1346 | CCATTACCCGGGCCCC | 37 | 24628 | 24643 | 753 |
| 826429 | 1366 | 1381 | ATCATCCATAAAGGCA | 75 | 24663 | 24678 | 754 |
| 826430 | 1379 | 1394 | AGTTAAAGCCACCATC | 57 | 24676 | 24691 | 755 |
| 826431 | 1383 | 1398 | CGCAAGTTAAAGCCAC | 70 | 24680 | 24695 | 756 |
| 826432 | 1385 | 1400 | GCCGCAAGTTAAAGCC | 33 | 24682 | 24697 | 757 |
| 826433 | 1387 | 1402 | AGGCCGCAAGTTAAAG | 32 | 24684 | 24699 | 758 |
| 826434 | 1388 | 1403 | CAGGCCGCAAGTTAAA | 47 | 24685 | 24700 | 759 |
| 826435 | 1389 | 1404 | CCAGGCCGCAAGTTAA | 40 | 24686 | 24701 | 760 |
| 826436 | 1390 | 1405 | GCCAGGCCGCAAGTTA | 40 | 24687 | 24702 | 761 |
| 826449 | 1446 | 1461 | TAATCGCCCCCAAGTC | 35 | 25166 | 25181 | 762 |
| 826450 | 1447 | 1462 | ATAATCGCCCCCAAGT | 56 | 25167 | 25182 | 763 |
| 826451 | 1448 | 1463 | CATAATCGCCCCCAAG | 53 | 25168 | 25183 | 764 |
| 826452 | 1450 | 1465 | GCCATAATCGCCCCCA | 23 | 25170 | 25185 | 765 |
| 826453 | 1451 | 1466 | CGCCATAATCGCCCCC | 22 | 25171 | 25186 | 766 |
| 826454 | 1453 | 1468 | GTCGCCATAATCGCCC | 43 | 25173 | 25188 | 767 |
| 826455 | 1457 | 1472 | TGCAGTCGCCATAATC | 36 | 25177 | 25192 | 768 |
| 826456 | 1458 | 1473 | GTGCAGTCGCCATAAT | 38 | 25178 | 25193 | 769 |
| 826469 | 1528 | 1543 | GTGAATACACACCTGC | 98 | N/A | N/A | 770 |
| 826470 | 1530 | 1545 | GAGTGAATACACACCT | 47 | 25444 | 25459 | 771 |
| 826471 | 1531 | 1546 | GGAGTGAATACACACC | 128 | 25445 | 25460 | 772 |
| 826472 | 1534 | 1549 | GCAGGAGTGAATACAC | 106 | 25448 | 25463 | 773 |
| 826473 | 1553 | 1568 | TGATCATGCTCTCCTG | 25 | 25467 | 25482 | 774 |
| 826474 | 1554 | 1569 | TTGATCATGCTCTCCT | 31 | 25468 | 25483 | 775 |
| 826475 | 1556 | 1571 | CCTTGATCATGCTCTC | 28 | 25470 | 25485 | 776 |
| 826476 | 1557 | 1572 | TCCTTGATCATGCTCT | 23 | 25471 | 25486 | 777 |
| 826488 | 1583 | 1598 | GATAGAAGATGTAGGC | 38 | 25497 | 25512 | 778 |
| 826489 | 1584 | 1599 | GGATAGAAGATGTAGG | 36 | 25498 | 25513 | 779 |
| 826490 | 1585 | 1600 | CGGATAGAAGATGTAG | 101 | 25499 | 25514 | 780 |
| 826491 | 1587 | 1602 | CGCGGATAGAAGATGT | 33 | 25501 | 25516 | 781 |
| 826492 | 1588 | 1603 | CCGCGGATAGAAGATG | 69 | 25502 | 25517 | 782 |
| 826493 | 1589 | 1604 | GCCGCGGATAGAAGAT | 64 | 25503 | 25518 | 783 |
| 826494 | 1591 | 1606 | GGGCCGCGGATAGAAG | 48 | 25505 | 25520 | 784 |
| 826495 | 1612 | 1627 | GTCACAGTACTCCACG | 27 | 25526 | 25541 | 785 |
| 826508 | 1669 | 1684 | GGAGAAGTCAACCTGG | 21 | 30615 | 30630 | 786 |
| 826509 | 1675 | 1690 | GTCTGAGGAGAAGTCA | 43 | 30621 | 30636 | 787 |
| 826510 | 1696 | 1711 | CTTGGTGAAACAGCCC | 159 | 30642 | 30657 | 788 |
| 826511 | 1702 | 1717 | CCGGCACTTGGTGAAA | 123 | 30648 | 30663 | 789 |
| 826512 | 1708 | 1723 | TGGCTTCCGGCACTTG | 36 | 30654 | 30669 | 790 |
| 826513 | 1709 | 1724 | ATGGCTTCCGGCACTT | 26 | 30655 | 30670 | 791 |
| 826514 | 1711 | 1726 | GCATGGCTTCCGGCAC | 15 | 30657 | 30672 | 792 |
| 826515 | 1716 | 1731 | ACGCTGCATGGCTTCC | 44 | N/A | N/A | 793 |
| 826555 | 1876 | 1891 | TTTGTAGTTCAGCTCC | 67 | 31232 | 31247 | 794 |
| 826568 | 2001 | 2016 | AGGTCAAAGACGAGCT | 76 | 31830 | 31845 | 795 |
| 826569 | 2002 | 2017 | CAGGTCAAAGACGAGC | 28 | 31831 | 31846 | 796 |
| 826570 | 2003 | 2018 | GCAGGTCAAAGACGAG | 103 | 31832 | 31847 | 797 |
| 826571 | 2009 | 2024 | TGACCAGCAGGTCAAA | 148 | 31838 | 31853 | 798 |
| 826572 | 2011 | 2026 | GATGACCAGCAGGTCA | 170 | 31840 | 31855 | 799 |
| 826573 | 2032 | 2047 | TCGGAGCAGCATGAGG | 61 | 31861 | 31876 | 800 |
| 826574 | 2034 | 2049 | CTTCGGAGCAGCATGA | 52 | 31863 | 31878 | 801 |
| 826575 | 2035 | 2050 | CCTTCGGAGCAGCATG | 44 | 31864 | 31879 | 802 |
| 826587 | 2049 | 2064 | TATCGGCTTCGGAACC | 28 | 31878 | 31893 | 803 |
| 826588 | 2050 | 2065 | GTATCGGCTTCGGAAC | 50 | 31879 | 31894 | 804 |
| 826589 | 2051 | 2066 | AGTATCGGCTTCGGAA | 28 | 31880 | 31895 | 805 |
| 826590 | 2053 | 2068 | CCAGTATCGGCTTCGG | 37 | 31882 | 31897 | 806 |
| 826591 | 2054 | 2069 | ACCAGTATCGGCTTCG | 23 | 31883 | 31898 | 807 |
| 826592 | 2055 | 2070 | GACCAGTATCGGCTTC | 32 | 31884 | 31899 | 808 |
| 826593 | 2056 | 2071 | AGACCAGTATCGGCTT | 20 | 31885 | 31900 | 809 |
| 826594 | 2058 | 2073 | GGAGACCAGTATCGGC | 25 | 31887 | 31902 | 810 |
| 826607 | 2282 | 2297 | AGGGCCCCCCCAGAGG | 90 | 32111 | 32126 | 811 |
| 826608 | 2284 | 2299 | TCAGGGCCCCCCCAGA | 55 | 32113 | 32128 | 812 |
| 826609 | 2308 | 2323 | GTGTGAGAAACCTCTC | 64 | 32137 | 32152 | 813 |
| 826610 | 2310 | 2325 | TGGTGTGAGAAACCTC | 81 | 32139 | 32154 | 814 |
| 826611 | 2313 | 2328 | CCTTGGTGTGAGAAAC | 93 | 32142 | 32157 | 815 |
| 826612 | 2314 | 2329 | GCCTTGGTGTGAGAAA | 52 | 32143 | 32158 | 816 |
| 826613 | 2315 | 2330 | TGCCTTGGTGTGAGAA | 31 | 32144 | 32159 | 817 |
| 826614 | 2316 | 2331 | CTGCCTTGGTGTGAGA | 30 | 32145 | 32160 | 818 |
| 826627 | 2399 | 2414 | ACGGCAGTTTGGGCGG | 34 | 32228 | 32243 | 819 |
| 826628 | 2400 | 2415 | AACGGCAGTTTGGGCG | 93 | 32229 | 32244 | 820 |
| 826629 | 2401 | 2416 | CAACGGCAGTTTGGGC | 87 | 32230 | 32245 | 821 |
| 826630 | 2403 | 2418 | ATCAACGGCAGTTTGG | 60 | 32232 | 32247 | 822 |
| 826631 | 2405 | 2420 | ACATCAACGGCAGTTT | 25 | 32234 | 32249 | 823 |
| 826632 | 2407 | 2422 | ACACATCAACGGCAGT | 19 | 32236 | 32251 | 824 |
| 826633 | 2408 | 2423 | CACACATCAACGGCAG | 38 | 32237 | 32252 | 825 |
| 826634 | 2410 | 2425 | TCCACACATCAACGGC | 49 | 32239 | 32254 | 826 |
| 826647 | 2491 | 2506 | GGAGCCAAGGCACTTC | 30 | 32320 | 32335 | 827 |
| 826648 | 2492 | 2507 | TGGAGCCAAGGCACTT | 31 | 32321 | 32336 | 828 |
| 826649 | 2502 | 2517 | GGTACAGGGCTGGAGC | 117 | 32331 | 32346 | 829 |
| 826650 | 2520 | 2535 | TCAGAGGCAGTACCAA | 48 | 32349 | 32364 | 830 |
| 826651 | 2523 | 2538 | TGTTCAGAGGCAGTAC | 38 | 32352 | 32367 | 831 |
| 826652 | 2533 | 2548 | GAAACCAGAGTGTTCA | 52 | 32362 | 32377 | 832 |
| 826653 | 2551 | 2566 | TAGCCGCAGTTGGGTG | 49 | 32380 | 32395 | 833 |
| 826654 | 2552 | 2567 | TTAGCCGCAGTTGGGT | 28 | 32381 | 32396 | 834 |
| 826665 | 2579 | 2594 | CTTGGCTGATCCAAGG | 33 | 32408 | 32423 | 835 |
| 826666 | 2580 | 2595 | GCTTGGCTGATCCAAG | 69 | 32409 | 32424 | 836 |
| 826667 | 2581 | 2596 | CGCTTGGCTGATCCAA | 66 | 32410 | 32425 | 837 |
| 826668 | 2582 | 2597 | TCGCTTGGCTGATCCA | 10 | 32411 | 32426 | 838 |
| 826669 | 2583 | 2598 | TTCGCTTGGCTGATCC | 33 | 32412 | 32427 | 839 |
| 826670 | 2584 | 2599 | TTTCGCTTGGCTGATC | 43 | 32413 | 32428 | 840 |
| 826671 | 2585 | 2600 | GTTTCGCTTGGCTGAT | 24 | 32414 | 32429 | 841 |
| 826672 | 2586 | 2601 | AGTTTCGCTTGGCTGA | 39 | 32415 | 32430 | 842 |
| 826685 | 2623 | 2638 | CAGCGGTTTCTTAGGA | 27 | 32452 | 32467 | 843 |
| 826686 | 2625 | 2640 | ATCAGCGGTTTCTTAG | 45 | 32454 | 32469 | 844 |
| 826687 | 2627 | 2642 | TTATCAGCGGTTTCTT | 18 | 32456 | 32471 | 845 |
| 826688 | 2629 | 2644 | GGTTATCAGCGGTTTC | 33 | 32458 | 32473 | 846 |
| 826689 | 2632 | 2647 | CCTGGTTATCAGCGGT | 29 | 32461 | 32476 | 847 |
| 826690 | 2634 | 2649 | GTCCTGGTTATCAGCG | 19 | 32463 | 32478 | 848 |
| 826691 | 2636 | 2651 | TTGTCCTGGTTATCAG | 36 | 32465 | 32480 | 849 |
| 826692 | 2652 | 2667 | TACCCTTGGTTGTGTT | 39 | 32481 | 32496 | 850 |
| 826705 | 2692 | 2707 | TAAGCCGTCGCTGGGC | 51 | 32521 | 32536 | 851 |
| 826706 | 2693 | 2708 | TTAAGCCGTCGCTGGG | 63 | 32522 | 32537 | 852 |
| 826707 | 2696 | 2711 | GGCTTAAGCCGTCGCT | 58 | 32525 | 32540 | 853 |
| 826708 | 2698 | 2713 | CTGGCTTAAGCCGTCG | 31 | 32527 | 32542 | 854 |
| 826709 | 2700 | 2715 | GGCTGGCTTAAGCCGT | 131 | 32529 | 32544 | 855 |
| 826710 | 2701 | 2716 | GGGCTGGCTTAAGCCG | 92 | 32530 | 32545 | 856 |
| 826711 | 2734 | 2749 | GCTACTGGAGAGCAGT | 20 | 32563 | 32578 | 857 |
| 826712 | 2735 | 2750 | TGCTACTGGAGAGCAG | 53 | 32564 | 32579 | 858 |
| 826725 | 2846 | 2861 | TCTGCTCTAGCCCTAG | 53 | 32675 | 32690 | 859 |
| 826726 | 2847 | 2862 | GTCTGCTCTAGCCCTA | 32 | 32676 | 32691 | 860 |
| 826727 | 2850 | 2865 | CGGGTCTGCTCTAGCC | 107 | 32679 | 32694 | 861 |
| 826728 | 2852 | 2867 | CCCGGGTCTGCTCTAG | 66 | 32681 | 32696 | 862 |
| 826729 | 2854 | 2869 | TACCCGGGTCTGCTCT | 92 | 32683 | 32698 | 863 |
| 826730 | 2855 | 2870 | TTACCCGGGTCTGCTC | 52 | 32684 | 32699 | 864 |
| 826731 | 2856 | 2871 | CTTACCCGGGTCTGCT | 51 | 32685 | 32700 | 865 |
| 826732 | 2858 | 2873 | TACTTACCCGGGTCTG | 38 | 32687 | 32702 | 866 |
| 826745 | 2954 | 2969 | AGACATGTAGAGGTAT | 16 | 32783 | 32798 | 867 |
| 826746 | 2955 | 2970 | CAGACATGTAGAGGTA | 8 | 32784 | 32799 | 868 |
| 826747 | 2959 | 2974 | CAAGCAGACATGTAGA | 32 | 32788 | 32803 | 869 |
| 826748 | 2960 | 2975 | TCAAGCAGACATGTAG | 25 | 32789 | 32804 | 870 |
| 826749 | 2961 | 2976 | CTCAAGCAGACATGTA | 22 | 32790 | 32805 | 871 |
| 826750 | 2963 | 2978 | ATCTCAAGCAGACATG | 50 | 32792 | 32807 | 872 |
| 826751 | 2964 | 2979 | TATCTCAAGCAGACAT | 26 | 32793 | 32808 | 873 |
| 826752 | 2965 | 2980 | ATATCTCAAGCAGACA | 27 | 32794 | 32809 | 874 |
| 826764 | 3016 | 3031 | ATGCATAGGAGTTCTC | 17 | 32845 | 32860 | 875 |
| 826765 | 3017 | 3032 | GATGCATAGGAGTTCT | 23 | 32846 | 32861 | 876 |
| 826766 | 3019 | 3034 | GGGATGCATAGGAGTT | 42 | 32848 | 32863 | 877 |
| 826767 | 3021 | 3036 | AAGGGATGCATAGGAG | 61 | 32850 | 32865 | 878 |
| 826768 | 3022 | 3037 | TAAGGGATGCATAGGA | 41 | 32851 | 32866 | 879 |
| 826769 | 3023 | 3038 | CTAAGGGATGCATAGG | 37 | 32852 | 32867 | 880 |
| 826770 | 3024 | 3039 | TCTAAGGGATGCATAG | 34 | 32853 | 32868 | 881 |
| 826771 | 3026 | 3041 | GTTCTAAGGGATGCAT | 25 | 32855 | 32870 | 882 |
| 826784 | 3121 | 3136 | TGTGCTACGGGAGCCC | 17 | 32950 | 32965 | 883 |
| 826785 | 3122 | 3137 | GTGTGCTACGGGAGCC | 10 | 32951 | 32966 | 884 |
| 826786 | 3123 | 3138 | AGTGTGCTACGGGAGC | 59 | 32952 | 32967 | 885 |
| 826787 | 3124 | 3139 | TAGTGTGCTACGGGAG | 12 | 32953 | 32968 | 886 |
| 826788 | 3125 | 3140 | ATAGTGTGCTACGGGA | 44 | 32954 | 32969 | 887 |
| 826789 | 3126 | 3141 | TATAGTGTGCTACGGG | 46 | 32955 | 32970 | 888 |
| 826790 | 3128 | 3143 | GTTATAGTGTGCTACG | 23 | 32957 | 32972 | 889 |
| 826803 | 3153 | 3168 | GTGCAACAGCAACACT | 67 | 32982 | 32997 | 890 |
| 826804 | 3154 | 3169 | GGTGCAACAGCAACAC | 37 | 32983 | 32998 | 891 |
| 826805 | 3155 | 3170 | TGGTGCAACAGCAACA | 64 | 32984 | 32999 | 892 |
| 826806 | 3156 | 3171 | ATGGTGCAACAGCAAC | 10 | 32985 | 33000 | 893 |
| 826807 | 3157 | 3172 | TATGGTGCAACAGCAA | 20 | 32986 | 33001 | 894 |
| 826808 | 3158 | 3173 | GTATGGTGCAACAGCA | 20 | 32987 | 33002 | 895 |
| 826809 | 3159 | 3174 | AGTATGGTGCAACAGC | 4 | 32988 | 33003 | 896 |
| 826821 | 3216 | 3231 | CCCTGACCGCAAGGCA | 16 | 33045 | 33060 | 897 |
| 826822 | 3217 | 3232 | TCCCTGACCGCAAGGC | 51 | 33046 | 33061 | 898 |
| 826823 | 3218 | 3233 | GTCCCTGACCGCAAGG | 35 | 33047 | 33062 | 899 |
| 826824 | 3219 | 3234 | AGTCCCTGACCGCAAG | 34 | 33048 | 33063 | 900 |
| 826825 | 3220 | 3235 | CAGTCCCTGACCGCAA | 33 | 33049 | 33064 | 901 |
| 826826 | 3222 | 3237 | TTCAGTCCCTGACCGC | 23 | 33051 | 33066 | 902 |
| 826827 | 3223 | 3238 | ATTCAGTCCCTGACCG | 69 | 33052 | 33067 | 903 |
| 826828 | 3225 | 3240 | AGATTCAGTCCCTGAC | 14 | 33054 | 33069 | 904 |
| 826840 | 3239 | 3254 | TACATAAACGGGCAAG | 50 | 33068 | 33083 | 905 |
| 826841 | 3242 | 3257 | GCATACATAAACGGGC | 61 | 33071 | 33086 | 906 |
| 826842 | 3244 | 3259 | GAGCATACATAAACGG | 59 | 33073 | 33088 | 907 |
| 826843 | 3249 | 3264 | ACATGGAGCATACATA | 94 | 33078 | 33093 | 908 |
| 826844 | 3250 | 3265 | GACATGGAGCATACAT | 42 | 33079 | 33094 | 909 |
| 826845 | 3251 | 3266 | AGACATGGAGCATACA | 81 | 33080 | 33095 | 910 |
| 826846 | 3253 | 3268 | CTAGACATGGAGCATA | 69 | 33082 | 33097 | 911 |
| 826847 | 3254 | 3269 | GCTAGACATGGAGCAT | 47 | 33083 | 33098 | 912 |
| 826860 | N/A | N/A | TGATACCTCCCCTTGG | 78 | 2720 | 2735 | 913 |
| 826861 | N/A | N/A | ATGATACCTCCCCTTG | 84 | 2721 | 2736 | 914 |
| 826862 | N/A | N/A | GCTCATGATACCTCCC | 176 | 2725 | 2740 | 915 |
| 826863 | N/A | N/A | ACTGCTCATGATACCT | 95 | 2728 | 2743 | 916 |
| 826864 | N/A | N/A | ATACTGCTCATGATAC | 59 | 2730 | 2745 | 917 |
| 826865 | N/A | N/A | GATACTGCTCATGATA | 87 | 2731 | 2746 | 918 |
| 826866 | N/A | N/A | CTTGATACTGCTCATG | 96 | 2734 | 2749 | 919 |
| 826867 | N/A | N/A | CCTTGATACTGCTCAT | 79 | 2735 | 2750 | 920 |
| 826880 | N/A | N/A | CGAGTTTTGTCCTAGC | 7 | 4876 | 4891 | 921 |
| 826881 | N/A | N/A | TCGAGTTTTGTCCTAG | 30 | 4877 | 4892 | 922 |
| 826882 | N/A | N/A | CTTTCGAGTTTTGTCC | 86 | 4880 | 4895 | 923 |
| 826883 | N/A | N/A | CACCTTTCGAGTTTTG | 30 | 4883 | 4898 | 924 |
| 826884 | N/A | N/A | GCCACCTTTCGAGTTT | 50 | 4885 | 4900 | 925 |
| 826885 | N/A | N/A | GGGCCACCTTTCGAGT | 21 | 4887 | 4902 | 926 |
| 826886 | N/A | N/A | TAGGGCCACCTTTCGA | 20 | 4889 | 4904 | 927 |
| 826887 | N/A | N/A | ATAGGGCCACCTTTCG | 27 | 4890 | 4905 | 928 |
| 826900 | N/A | N/A | GCCGGAGCTGGGCTTC | 55 | 4935 | 4950 | 929 |
| 826901 | N/A | N/A | TGCCGGAGCTGGGCTT | 82 | 4936 | 4951 | 930 |
| 826902 | N/A | N/A | GTGCCGGAGCTGGGCT | 179 | 4937 | 4952 | 931 |
| 826903 | N/A | N/A | AAGTGCCGGAGCTGGG | 31 | 4939 | 4954 | 932 |
| 826904 | N/A | N/A | AAAAGTGCCGGAGCTG | 44 | 4941 | 4956 | 933 |
| 826905 | N/A | N/A | CCAAAAGTGCCGGAGC | 34 | 4943 | 4958 | 934 |
| 826906 | N/A | N/A | GCCAAAAGTGCCGGAG | 19 | 4944 | 4959 | 935 |
| 826907 | N/A | N/A | GGCCAAAAGTGCCGGA | 38 | 4945 | 4960 | 936 |
| 826920 | N/A | N/A | CCCCTGGAACCCGAGT | 31 | 5065 | 5080 | 937 |
| 826921 | N/A | N/A | CACCCCTGGAACCCGA | 38 | 5067 | 5082 | 938 |
| 826922 | N/A | N/A | CCCGGAGTGGATTGGG | 185 | 5138 | 5153 | 939 |
| 826923 | N/A | N/A | GCCCCGGAGTGGATTG | 76 | 5140 | 5155 | 940 |
| 826924 | N/A | N/A | GAGCCCCGGAGTGGAT | 64 | 5142 | 5157 | 941 |
| 826925 | N/A | N/A | ATGAGCCCCGGAGTGG | 28 | 5144 | 5159 | 942 |
| 826926 | N/A | N/A | TTCATGAGCCCCGGAG | 42 | 5147 | 5162 | 943 |
| 826927 | N/A | N/A | CCTTCATGAGCCCCGG | 29 | 5149 | 5164 | 944 |
| 826940 | N/A | N/A | TGCTTACCTTGATACT | 152 | 2741 | 2756 | 945 |
| 826941 | N/A | N/A | CCAAACCAGGTTCCCT | 104 | 2757 | 2772 | 946 |
| 826942 | N/A | N/A | AGCCGGTGTCAACCAG | 187 | 2777 | 2792 | 947 |
| 826943 | N/A | N/A | AAAGTGAAAGCCGGTG | 138 | 2785 | 2800 | 948 |
| 826944 | N/A | N/A | TGCGACTTCTTAAAGT | 97 | 2796 | 2811 | 949 |
| 826945 | N/A | N/A | GCTCAGGGTCCAACCT | 79 | 2844 | 2859 | 950 |
| 826946 | N/A | N/A | AGCAAGGAGTTTAGCA | 95 | 2889 | 2904 | 951 |
| 826947 | N/A | N/A | CATAAGAGCCAAGGGC | 142 | 2983 | 2998 | 952 |
| 826960 | N/A | N/A | CGTTGATGGGCTATAT | 168 | 3408 | 3423 | 953 |
| 826961 | N/A | N/A | CGCCTAGACAGGCCCT | 61 | 3440 | 3455 | 954 |
| 826962 | N/A | N/A | ACGCAGGACACTGTGG | 90 | 3555 | 3570 | 955 |
| 826963 | N/A | N/A | AGGCAGCGCGAGGGCC | 109 | 3571 | 3586 | 956 |
| 826964 | N/A | N/A | GTGTAATCGCCCCTGC | 84 | 3622 | 3637 | 957 |
| 826965 | N/A | N/A | GGCCCTAGGACATTCT | 73 | 3674 | 3689 | 958 |
| 826966 | N/A | N/A | GTCCAGACCCGGGAGG | 66 | 3718 | 3733 | 959 |
| 826967 | N/A | N/A | GGGAGCAGCGCACTCA | 106 | 3804 | 3819 | 960 |
| 826980 | N/A | N/A | GGGACTAACCGACCTG | 146 | 5631 | 5646 | 961 |
| 826981 | N/A | N/A | TTCCAGGCGCAGGCAC | 76 | 5662 | 5677 | 962 |
| 826982 | N/A | N/A | CAGTAAGCTGGAGGCT | 181 | 5785 | 5800 | 963 |
| 826983 | N/A | N/A | CGCCAGTCCAGTAAGC | 137 | 5793 | 5808 | 964 |
| 826984 | N/A | N/A | GCTAGGATGGCTCCAC | 59 | 5819 | 5834 | 965 |
| 826985 | N/A | N/A | CCACACTCTGGGTGAG | 42 | 5843 | 5858 | 966 |
| 826986 | N/A | N/A | GGGCAATGCTGCTCCA | 76 | 5863 | 5878 | 967 |
| 826987 | N/A | N/A | TCCCACTTGCAGGAGG | 91 | 5919 | 5934 | 968 |
| 827000 | N/A | N/A | TCCCAAGGTGTGGCAT | 44 | 6462 | 6477 | 969 |
| 827001 | N/A | N/A | TTGAAGCAGGTGTTCC | 60 | 6475 | 6490 | 970 |
| 827002 | N/A | N/A | TGCCAGGTGCCTAGCC | 55 | 6502 | 6517 | 971 |
| 827003 | N/A | N/A | CAATAAAGGGCTTATG | 94 | 6538 | 6553 | 972 |
| 827004 | N/A | N/A | AACTACCTGGCCTTCA | 63 | 6552 | 6567 | 973 |
| 827005 | N/A | N/A | GGCTTATATGCCTGTC | 77 | 6605 | 6620 | 974 |
| 827006 | N/A | N/A | TGCCACAGTTACTGGC | 80 | 6618 | 6633 | 975 |
| 827007 | N/A | N/A | ACTTATCCCAGTGTGC | 30 | 6631 | 6646 | 976 |
| 827020 | N/A | N/A | AGGAAATGGTCCCTAC | 70 | 6912 | 6927 | 977 |
| 827021 | N/A | N/A | GTGCACACGGCAGCTT | 77 | 6932 | 6947 | 978 |
| 827022 | N/A | N/A | CCCAAGACACCTTCGC | 55 | 6955 | 6970 | 979 |
| 827023 | N/A | N/A | TAGCACCGGGCTTGTA | 62 | 6994 | 7009 | 980 |
| 827024 | N/A | N/A | AACAGGATGAGTCACA | 26 | 7088 | 7103 | 981 |
| 827025 | N/A | N/A | AGTTTTGGGATTAGGC | 51 | 7107 | 7122 | 982 |
| 827026 | N/A | N/A | GGCGGAAGCCACATCT | 61 | 7178 | 7193 | 983 |
| 827027 | N/A | N/A | TGAAATGAGGCGGAAG | 117 | 7186 | 7201 | 984 |
| 827040 | N/A | N/A | GGGAATAATACTGCCC | 115 | 7751 | 7766 | 985 |
| 827041 | N/A | N/A | AATGTATGTTCCCTTG | 34 | 7816 | 7831 | 986 |
| 827042 | N/A | N/A | GTAAAAAGTCTGGCCC | 34 | 8222 | 8237 | 987 |
| 827043 | N/A | N/A | TCCAAGGTGTGTTGTG | 35 | 8283 | 8298 | 988 |
| 827044 | N/A | N/A | CATGAGACCTACTTCC | 30 | 8296 | 8311 | 989 |
| 827045 | N/A | N/A | ATAAGAGTCATCATGA | 54 | 8307 | 8322 | 990 |
| 827046 | N/A | N/A | GGTGAGGGTGGACGGT | 86 | 8444 | 8459 | 991 |
| 827047 | N/A | N/A | GGCTTTCCATTGGAGC | 64 | 8483 | 8498 | 992 |
| 827060 | N/A | N/A | CCTCAGCAGGTAGGCA | 45 | 8836 | 8851 | 993 |
| 827061 | N/A | N/A | TCGGACTCAGCACTTC | 75 | 8961 | 8976 | 994 |
| 827062 | N/A | N/A | CTGCAGTGGCCAACCC | 98 | 8983 | 8998 | 995 |
| 827063 | N/A | N/A | CTGTAGGTATGACTGG | 31 | 9047 | 9062 | 996 |
| 827064 | N/A | N/A | TTCCATGACTGTAGGT | 24 | 9055 | 9070 | 997 |
| 827065 | N/A | N/A | GCCTAAACCGTTCCTG | 53 | 9105 | 9120 | 998 |
| 827066 | N/A | N/A | TTCAAGAACCCCAAGT | 50 | 9132 | 9147 | 999 |
| 827067 | N/A | N/A | AGAAGCTACCATGACC | 66 | 9158 | 9173 | 1000 |
| 827080 | N/A | N/A | GGCCTATCAACTAGGC | 154 | 9783 | 9798 | 1001 |
| 827081 | N/A | N/A | CACAATTCCATCGGGC | 22 | 9837 | 9852 | 1002 |
| 827082 | N/A | N/A | CCCTACATTGGAGGGT | 188 | 9866 | 9881 | 1003 |
| 827083 | N/A | N/A | AGGGATAAAGAATGCC | 57 | 9978 | 9993 | 1004 |
| 827084 | N/A | N/A | GACCAGCGGCTGGAGG | 46 | 9996 | 10011 | 1005 |
| 827085 | N/A | N/A | AGACATCCGATCTTGT | 42 | 10020 | 10035 | 1006 |
| 827086 | N/A | N/A | TGACACCTAGAGCTAA | 60 | 10068 | 10083 | 1007 |
| 827087 | N/A | N/A | GGCAGAGCCTTTGAGT | 58 | 10084 | 10099 | 1008 |
| 827100 | N/A | N/A | TACGCACCTCCCTCCT | 41 | 10649 | 10664 | 1009 |
| | | | | | 10672 | 10687 | |
| 827101 | N/A | N/A | CTACGCACCTCCCTCC | 128 | 10650 | 10665 | 1010 |
| | | | | | 10673 | 10688 | |
| 827102 | N/A | N/A | CCCTACGCACCTCCCT | 88 | 10652 | 10667 | 1011 |
| | | | | | 10675 | 10690 | |
| 827103 | N/A | N/A | ACCCTACGCACCTCCC | 34 | 10653 | 10668 | 1012 |
| | | | | | 10676 | 10691 | |
| 827104 | N/A | N/A | CCACCCTACGCACCTC | 36 | 10655 | 10670 | 1013 |
| | | | | | 10678 | 10693 | |
| 827105 | N/A | N/A | GCCACCCTACGCACCT | 40 | 10656 | 10671 | 1014 |
| | | | | | 10679 | 10694 | |
| 827106 | N/A | N/A | CTGCCACCCTACGCAC | 47 | 10658 | 10673 | 1015 |
| | | | | | 10681 | 10696 | |
| 827107 | N/A | N/A | CCTGCCACCCTACGCA | 40 | 10659 | 10674 | 1016 |
| | | | | | 10682 | 10697 | |
| 827120 | N/A | N/A | CCACATGGTGCCCCAG | 61 | 11248 | 11263 | 1017 |
| 827121 | N/A | N/A | TTTTAGGAGGGCCACA | 126 | 11259 | 11274 | 1018 |
| 827122 | N/A | N/A | GCCCTCTGGTCCGTCC | 87 | 11291 | 11306 | 1019 |
| 827123 | N/A | N/A | GGTCAGACAGCACTCC | 33 | 11319 | 11334 | 1020 |
| 827124 | N/A | N/A | AGCTAGCAAATGGGTC | 82 | 11331 | 11346 | 1021 |
| 827125 | N/A | N/A | TTCCAGTTGGCACAGC | 42 | 11344 | 11359 | 1022 |
| 827126 | N/A | N/A | CACAATTGTCATTCCC | 22 | 11395 | 11410 | 1023 |
| 827127 | N/A | N/A | TGTTAGCTAACACAAT | 48 | 11405 | 11420 | 1024 |
| 827140 | N/A | N/A | CCCACAGAAAACGGAA | 40 | 11690 | 11705 | 1025 |
| 827141 | N/A | N/A | GGCTGCTGCATGATTC | 24 | 11738 | 11753 | 1026 |
| 827142 | N/A | N/A | ACCAGAATAGATTCAC | 108 | 11766 | 11781 | 1027 |
| 827143 | N/A | N/A | TCGAATCGAGTGCCCC | 35 | 11791 | 11806 | 1028 |
| 827144 | N/A | N/A | AACAATGAACCTCGAA | 98 | 11802 | 11817 | 1029 |
| 827145 | N/A | N/A | TGGTATTAGAATGTAC | 29 | 11881 | 11896 | 1030 |
| 827146 | N/A | N/A | GTGGTATTAGAATGTA | 41 | 11882 | 11897 | 1031 |
| 827147 | N/A | N/A | TGTGGTATTAGAATGT | 23 | 11883 | 11898 | 1032 |
| 827160 | N/A | N/A | GTGCAGGGTCTTACTT | 55 | 12230 | 12245 | 1033 |
| 827161 | N/A | N/A | AAATACCAGTGCAGGG | 73 | 12238 | 12253 | 1034 |
| 827162 | N/A | N/A | GTACATCAATTATGCC | 47 | 12268 | 12283 | 1035 |
| 827163 | N/A | N/A | GGGCACTCAAGATTTG | 52 | 12295 | 12310 | 1036 |
| 827164 | N/A | N/A | CAAACCTGAGTGGGCA | 43 | 12306 | 12321 | 1037 |
| 827165 | N/A | N/A | CTCGACTGTCAAACCT | 50 | 12315 | 12330 | 1038 |
| 827166 | N/A | N/A | GTTCAACATCTCGACT | 44 | 12324 | 12339 | 1039 |
| 827167 | N/A | N/A | GTAATGGGAGTGTTCA | 18 | 12335 | 12350 | 1040 |
| 827180 | N/A | N/A | GTTGAAGGTGTGTGTT | 35 | 13095 | 13110 | 1041 |
| 827181 | N/A | N/A | AGCAACTCAAAGGTGT | 38 | 13111 | 13126 | 1042 |
| 827182 | N/A | N/A | AGATTTGTACATGAGG | 78 | 13481 | 13496 | 1043 |
| 827183 | N/A | N/A | ACCCGAAACACATTAG | 66 | 13504 | 13519 | 1044 |
| 827184 | N/A | N/A | GTTTAGGCCGCACCCG | 27 | 13515 | 13530 | 1045 |
| 827185 | N/A | N/A | ATTTACGGTGTTTAGG | 61 | 13524 | 13539 | 1046 |
| 827186 | N/A | N/A | GGGATTTACAGTGAGC | 40 | 13548 | 13563 | 1047 |
| 827187 | N/A | N/A | AAAGCATATGCCCCCA | 26 | 13678 | 13693 | 1048 |
| 827200 | N/A | N/A | AACCGTATGTAGTAGG | 27 | 14153 | 14168 | 1049 |
| 827201 | N/A | N/A | CAACCGTATGTAGTAG | 53 | 14154 | 14169 | 1050 |
| 827202 | N/A | N/A | ACAACCGTATGTAGTA | 39 | 14155 | 14170 | 1051 |
| 827203 | N/A | N/A | AACAACCGTATGTAGT | 50 | 14156 | 14171 | 1052 |
| 827204 | N/A | N/A | GAACAACCGTATGTAG | 53 | 14157 | 14172 | 1053 |
| 827205 | N/A | N/A | AGAACAACCGTATGTA | 43 | 14158 | 14173 | 1054 |
| 827206 | N/A | N/A | TAGAACAACCGTATGT | 46 | 14159 | 14174 | 1055 |
| 827219 | N/A | N/A | TGACATACTGCTTCTA | 54 | 14642 | 14657 | 1056 |
| 827220 | N/A | N/A | CCCCAGCAGGTATTTT | 156 | 14667 | 14682 | 1057 |
| 827221 | N/A | N/A | CCCAAGCAATCACCAG | 120 | 14737 | 14752 | 1058 |
| 827222 | N/A | N/A | GACCAAAAGTGTGCCA | 45 | 14831 | 14846 | 1059 |
| 827223 | N/A | N/A | GACACAATCGCCGCTC | 114 | 14905 | 14920 | 1060 |
| 827224 | N/A | N/A | GAATAAGTGGAGATAT | 55 | 15017 | 15032 | 1061 |
| 827225 | N/A | N/A | TGCATTTCCGTCTCAA | 21 | 15204 | 15219 | 1062 |
| 827226 | N/A | N/A | GGGTATCGAGACCACC | 116 | 15441 | 15456 | 1063 |
| 827239 | N/A | N/A | CCGGACCTAGAAGGGA | 117 | 15987 | 16002 | 1064 |
| 827240 | N/A | N/A | GGCCACGGCGAGCCCA | 153 | 16080 | 16095 | 1065 |
| 827241 | N/A | N/A | GTAAACAGGTGTGTCC | 63 | 16110 | 16125 | 1066 |
| 827242 | N/A | N/A | CTGGAGCGAGTGTCTG | 165 | 16238 | 16253 | 1067 |
| 827243 | N/A | N/A | GCGGAGCCCATGGGTG | 73 | 16616 | 16631 | 1068 |
| 827244 | N/A | N/A | TGTCACTGGGCTGCGC | 60 | 16650 | 16665 | 1069 |
| 827245 | N/A | N/A | CCGCGAGCCCACGAGG | 54 | 16676 | 16691 | 1070 |
| 827246 | N/A | N/A | CACCAAGAGGTGTTAT | 54 | 16738 | 16753 | 1071 |
| 827259 | N/A | N/A | ATTTATACCTCCCCTG | 101 | 17495 | 17510 | 1072 |
| 827260 | N/A | N/A | CACACACGGTTTTGGT | 18 | 17513 | 17528 | 1073 |
| 827261 | N/A | N/A | GACCAGTAGCTGCACA | 72 | 17527 | 17542 | 1074 |
| 827262 | N/A | N/A | ATTAAGGGAGTTGCAG | 106 | 17555 | 17570 | 1075 |
| 827263 | N/A | N/A | CCCTAGGAGCATGGAC | 56 | 17585 | 17600 | 1076 |
| 827264 | N/A | N/A | GCAGAAGTCCCTAGGA | 92 | 17593 | 17608 | 1077 |
| 827265 | N/A | N/A | ACAGGAGTCGGAAGCC | 48 | 17640 | 17655 | 1078 |
| 827266 | N/A | N/A | GGGATAAGCCCCTTGG | 87 | 17705 | 17720 | 1079 |
| 827279 | N/A | N/A | AGATCCATGCTTCCAG | 17 | 18216 | 18231 | 1080 |
| 827280 | N/A | N/A | AAGATCCATGCTTCCA | 11 | 18217 | 18232 | 1081 |
| 827281 | N/A | N/A | CCAAGATCCATGCTTC | 22 | 18219 | 18234 | 1082 |
| 827282 | N/A | N/A | ACCAAGATCCATGCTT | 55 | 18220 | 18235 | 1083 |
| 827283 | N/A | N/A | GACCAAGATCCATGCT | 17 | 18221 | 18236 | 1084 |
| 827284 | N/A | N/A | AGACCAAGATCCATGC | 12 | 18222 | 18237 | 1085 |
| 827285 | N/A | N/A | AAGACCAAGATCCATG | 19 | 18223 | 18238 | 1086 |
| 827298 | N/A | N/A | AGGATGATGTGATACA | 30 | 18735 | 18750 | 1087 |
| 827299 | N/A | N/A | AAGGATGATGTGATAC | 74 | 18736 | 18751 | 1088 |
| 827300 | N/A | N/A | ATCTAAGAAATAGGCT | 35 | 18755 | 18770 | 1089 |
| 827301 | N/A | N/A | CACATAGCCCAGATAG | 31 | 18834 | 18849 | 1090 |
| 827302 | N/A | N/A | TGCCAAAGGAGCATGG | 61 | 18901 | 18916 | 1091 |
| 827303 | N/A | N/A | CTTGAGTAAAAGTGCC | 30 | 18913 | 18928 | 1092 |
| 827304 | N/A | N/A | GTACAGCTCTTGAGAT | 46 | 18955 | 18970 | 1093 |
| 827317 | N/A | N/A | GGTAAGAAGTGACACC | 57 | 19364 | 19379 | 1094 |
| 827318 | N/A | N/A | GTGTACTGGGCAGAGT | 20 | 19390 | 19405 | 1095 |
| 827319 | N/A | N/A | TGCTACCATCTTACTT | 52 | 19463 | 19478 | 1096 |
| 827320 | N/A | N/A | GGCTTAGGTGTTGCTA | 45 | 19474 | 19489 | 1097 |
| 827321 | N/A | N/A | GCGGACTCAGGCTTAG | 51 | 19483 | 19498 | 1098 |
| 827322 | N/A | N/A | TGACAGGTGTGGGCGG | 48 | 19495 | 19510 | 1099 |
| 827323 | N/A | N/A | GTGACAGGTGTGGGCG | 66 | 19496 | 19511 | 1100 |
| 827324 | N/A | N/A | GTCCAGGTGACAGTTA | 38 | 19522 | 19537 | 1101 |
| 827337 | N/A | N/A | CCCAGGCGAGCAATGA | 20 | 19746 | 19761 | 1102 |
| 827338 | N/A | N/A | GGTATAACAACCCAGG | 25 | 19756 | 19771 | 1103 |
| 827339 | N/A | N/A | CAGTAGGGTGGAGTGG | 74 | 19774 | 19789 | 1104 |
| 827340 | N/A | N/A | GTACAAAGGTTCCTGT | 74 | 19829 | 19844 | 1105 |
| 827341 | N/A | N/A | CGTGAAGTAAGGTTGA | 31 | 19846 | 19861 | 1106 |
| 827342 | N/A | N/A | GTTACATGTGGTGACG | 53 | 19860 | 19875 | 1107 |
| 827343 | N/A | N/A | AGTTACATGTGGTGAC | 45 | 19861 | 19876 | 1108 |
| 827344 | N/A | N/A | TAGTTACATGTGGTGA | 31 | 19862 | 19877 | 1109 |
| 827356 | N/A | N/A | AGGACTAACTGGAATA | 27 | 19876 | 19891 | 1110 |
| 827357 | N/A | N/A | CAGGACTAACTGGAAT | 31 | 19877 | 19892 | 1111 |
| 827358 | N/A | N/A | GCCCGGTGAGATATTC | 80 | 19923 | 19938 | 1112 |
| 827359 | N/A | N/A | CCCGATAGCTGGTTGT | 18 | 20415 | 20430 | 1113 |
| 827360 | N/A | N/A | TTAATTAGTTCACCCG | 12 | 20427 | 20442 | 1114 |
| 827361 | N/A | N/A | AGTGAATCCTCACACT | 161 | 20444 | 20459 | 1115 |
| 827362 | N/A | N/A | CCTAGCTGGGAGGTGT | 56 | 20516 | 20531 | 1116 |
| 827363 | N/A | N/A | CATGTTGGAGGTGATC | 58 | 20531 | 20546 | 1117 |
| 827376 | N/A | N/A | TCATAGGTAAACACCC | 31 | 21565 | 21580 | 1118 |
| 827377 | N/A | N/A | GAAAAGTCTGGTAGCT | 23 | 21628 | 21643 | 1119 |
| 827378 | N/A | N/A | TGGTGTGACCATTTGG | 13 | 21643 | 21658 | 1120 |
| 827379 | N/A | N/A | ATGGTGTGACCATTTG | 7 | 21644 | 21659 | 1121 |
| 827380 | N/A | N/A | AAATGGTGTGACCATT | 72 | 21646 | 21661 | 1122 |
| 827381 | N/A | N/A | TAAATGGTGTGACCAT | 41 | 21647 | 21662 | 1123 |
| 827382 | N/A | N/A | GTTAAATGGTGTGACC | 14 | 21649 | 21664 | 1124 |
| 827394 | N/A | N/A | GTACGATTACAGGGAT | 31 | 21753 | 21768 | 1125 |
| 827395 | N/A | N/A | AGTACGATTACAGGGA | 5 | 21754 | 21769 | 1126 |
| 827396 | N/A | N/A | TAGTACGATTACAGGG | 90 | 21755 | 21770 | 1127 |
| 827397 | N/A | N/A | CTAGTACGATTACAGG | 24 | 21756 | 21771 | 1128 |
| 827398 | N/A | N/A | ACTAGTACGATTACAG | 29 | 21757 | 21772 | 1129 |
| 827399 | N/A | N/A | CACTAGTACGATTACA | 50 | 21758 | 21773 | 1130 |
| 827400 | N/A | N/A | TCACTAGTACGATTAC | 78 | 21759 | 21774 | 1131 |
| 827401 | N/A | N/A | CTCACTAGTACGATTA | 34 | 21760 | 21775 | 1132 |
| 827413 | N/A | N/A | CCTATGAGAATCAGTA | 16 | 22313 | 22328 | 1133 |
| 827414 | N/A | N/A | GCCTATGAGAATCAGT | 19 | 22314 | 22329 | 1134 |
| 827415 | N/A | N/A | CTATAGTGGCCTATGA | 111 | 22322 | 22337 | 1135 |
| 827416 | N/A | N/A | GATACACACTAAGCAC | 23 | 22342 | 22357 | 1136 |
| 827417 | N/A | N/A | AGATACACACTAAGCA | 11 | 22343 | 22358 | 1137 |
| 827418 | N/A | N/A | GCACACTACAGCGAGA | 55 | 22455 | 22470 | 1138 |
| 827419 | N/A | N/A | AAACATAGAGCTTCGA | 7 | 22721 | 22736 | 1139 |
| 827432 | N/A | N/A | GGTGAGCCCTTCGCAC | 11 | 22828 | 22843 | 1140 |
| 827433 | N/A | N/A | TGAAGGAGAGGCTACA | 39 | 22866 | 22881 | 1141 |
| 827434 | N/A | N/A | ATTCTAGGATGTACTG | 53 | 22926 | 22941 | 1142 |
| 827435 | N/A | N/A | GTGACATACTGGTGCA | 17 | 22943 | 22958 | 1143 |
| 827436 | N/A | N/A | GGGATATTCCACTGGC | 37 | 22983 | 22998 | 1144 |
| 827437 | N/A | N/A | AACTAGGTGATCCGGG | 9 | 22996 | 23011 | 1145 |
| 827438 | N/A | N/A | AATGTAGGATGATTCT | 29 | 23030 | 23045 | 1146 |
| 827439 | N/A | N/A | TTCTAAGCTTATTCTC | 34 | 23057 | 23072 | 1147 |
| 827451 | N/A | N/A | GGTGAGCACGGAGCTG | 35 | 23471 | 23486 | 1148 |
| 827452 | N/A | N/A | GGAGAAAGTGTGACCA | 57 | 23489 | 23504 | 1149 |
| 827453 | N/A | N/A | GAGCAGGGTTAAAGGA | 110 | 23502 | 23517 | 1150 |
| 827454 | N/A | N/A | TGTCATCTAGGAGATA | 123 | 23597 | 23612 | 1151 |
| 827455 | N/A | N/A | TTGCATAGATCCTGTC | 47 | 23609 | 23624 | 1152 |
| 827456 | N/A | N/A | CTTGATGACAGGAGCC | 67 | 23660 | 23675 | 1153 |
| 827457 | N/A | N/A | TTGAATCATGAGCTCC | 35 | 23675 | 23690 | 1154 |
| 827458 | N/A | N/A | GCCCATGCATCTAAGT | 38 | 23703 | 23718 | 1155 |
| 827471 | N/A | N/A | GGACTATGTGGCACCT | 43 | 24342 | 24357 | 1156 |
| 827472 | N/A | N/A | TGGCAACCCCTGAGCT | 80 | 24412 | 24427 | 1157 |
| 827473 | N/A | N/A | GTTCAGGAAGACCCGC | 166 | 24437 | 24452 | 1158 |
| 827474 | N/A | N/A | GCAGAGGCGGGAATCC | 78 | 24524 | 24539 | 1159 |
| 827475 | N/A | N/A | CATCAGGGACAGACCT | 78 | 24564 | 24579 | 1160 |
| 827476 | N/A | N/A | CTGCAATCTGAGGCGC | 85 | 24761 | 24776 | 1161 |
| 827477 | N/A | N/A | CCCTAAGCATGCCTTG | 46 | 24939 | 24954 | 1162 |
| 827478 | N/A | N/A | TTTCAAGGCCACTAGG | 109 | 24974 | 24989 | 1163 |
| 827491 | N/A | N/A | ACCTTAGGAGCCATTG | 27 | 26493 | 26508 | 1164 |
| 827492 | N/A | N/A | ACCCATGTATCTTCTA | 46 | 26627 | 26642 | 1165 |
| 827493 | N/A | N/A | AATGAGACAGACCCAT | 62 | 26637 | 26652 | 1166 |
| 827494 | N/A | N/A | GGATACAGTATGTCCA | 78 | 26685 | 26700 | 1167 |
| 827495 | N/A | N/A | CTCTACTATTGAATGG | 46 | 26699 | 26714 | 1168 |
| 827496 | N/A | N/A | ATTATATACCTCTACT | 58 | 26708 | 26723 | 1169 |
| 827497 | N/A | N/A | ATCTTAAACAGGTCCA | 16 | 26745 | 26760 | 1170 |
| 827498 | N/A | N/A | ACTGATTGTGCCCTTG | 17 | 26777 | 26792 | 1171 |
| 827511 | N/A | N/A | CCAGGAGGCCACGACT | 30 | 27241 | 27256 | 1172 |
| 827512 | N/A | N/A | TACAATCCTCTAAGGT | 43 | 27271 | 27286 | 1173 |
| 827513 | N/A | N/A | CTGTATACCCTGGGAC | 59 | 27378 | 27393 | 1174 |
| 827514 | N/A | N/A | TCTCAGCAATCAATAT | 62 | 27490 | 27505 | 1175 |
| 827515 | N/A | N/A | GGGAAGTAAGCCCTAG | 47 | 27559 | 27574 | 1176 |
| 827516 | N/A | N/A | GGCTGGAGATCTTTAG | 34 | 27607 | 27622 | 1177 |
| 827517 | N/A | N/A | CCCAAATCCCTACCAG | 61 | 27623 | 27638 | 1178 |
| 827518 | N/A | N/A | GTCATTATTGCTACTT | 17 | 27675 | 27690 | 1179 |
| 827531 | N/A | N/A | CAGAATAGCCGGGCGC | 46 | 28650 | 28665 | 1180 |
| 827532 | N/A | N/A | GGCAGACACGAGGGTC | 40 | 28699 | 28714 | 1181 |
| 827533 | N/A | N/A | CCATACGGATGAACCT | 35 | 28741 | 28756 | 1182 |
| 827534 | N/A | N/A | TACCATACGGATGAAC | 20 | 28743 | 28758 | 1183 |
| 827535 | N/A | N/A | CTACCATACGGATGAA | 49 | 28744 | 28759 | 1184 |
| 827536 | N/A | N/A | GCTACCATACGGATGA | 23 | 28745 | 28760 | 1185 |
| 827537 | N/A | N/A | TGCTACCATACGGATG | 59 | 28746 | 28761 | 1186 |
| 827550 | N/A | N/A | AGGGAATTAAGCCACA | 13 | 29501 | 29516 | 1187 |
| 827551 | N/A | N/A | GGATACACCAGTGTAA | 32 | 29904 | 29919 | 1188 |
| 827552 | N/A | N/A | AGCTAAGTCAGGCGAA | 67 | 29930 | 29945 | 1189 |
| 827553 | N/A | N/A | TATGAGTGTGCCTTTG | 25 | 30329 | 30344 | 1190 |
| 827554 | N/A | N/A | TTCAAGGTTGCAAGTG | 41 | 30348 | 30363 | 1191 |
| 827555 | N/A | N/A | AGCTAAGCCAGGGACA | 67 | 30416 | 30431 | 1192 |
| 827556 | N/A | N/A | GCCTTATGAGTGGCAG | 54 | 30456 | 30471 | 1193 |
| 827557 | N/A | N/A | CCACTTTACAAGAGCA | 22 | 30492 | 30507 | 1194 |
| 827570 | N/A | N/A | ATCAAGGTCACTCCCA | 48 | 30959 | 30974 | 1195 |
| 827571 | N/A | N/A | GAAGACCCATTCCTAG | 78 | 30992 | 31007 | 1196 |
| 827572 | N/A | N/A | CCATATCGATCCCTCT | 132 | 31115 | 31130 | 1197 |
| 827573 | N/A | N/A | GAATTTCCTGGACCTT | 84 | 31142 | 31157 | 1198 |
| 827574 | N/A | N/A | GAAATGGTAGAGGATG | 46 | 31157 | 31172 | 1199 |
| 827575 | N/A | N/A | AGGCACGACCTACCGT | 139 | 31272 | 31287 | 1200 |
| 827576 | N/A | N/A | CTCCATCCAGGCACGA | 55 | 31280 | 31295 | 1201 |
| 827577 | N/A | N/A | AAGTAAGACCCCCAGA | 79 | 31306 | 31321 | 1202 |

TABLE 4
Percent level of human α-ENaC mRNA

| | SEQ | SEQ | | | | | |
|---|---|---|---|---|---|---|---|
| | ID: | ID: | | | SEQ | SEQ | |
| Com- | 1 | 1 | | α-ENaC | ID: 2 | ID 2: | SEQ |
| pound | Start | Stop | | (% | Start | Stop | ID |
| Number | Site | Site | Sequence | control) | Site | Site | NO |
| 668182 | 535 | 550 | CCAGAAGGCCGTCTTC | 34 | 5463 | 5478 | 1203 |
| 668208 | 764 | 779 | ATTTGTACAGGTCAAA | 35 | 16349 | 16364 | 1204 |
| 668218 | 974 | 989 | ATTTGTTCTGGTTGCA | 19 | 17760 | 17775 | 1205 |
| 826072 | 7 | 22 | GCTTTAGACGCAGACA | 102 | 4268 | 4283 | 1206 |
| 826073 | 9 | 24 | GGGCTTTAGACGCAGA | 68 | 4270 | 4285 | 1207 |
| 826082 | 37 | 52 | TGGACCTGAGAAGGCG | 58 | 4298 | 4313 | 1208 |
| 826083 | 40 | 55 | TACTGGACCTGAGAAG | 66 | 4301 | 4316 | 1209 |
| 826084 | 42 | 57 | AGTACTGGACCTGAGA | 55 | 4303 | 4318 | 1210 |
| 826085 | 44 | 59 | GGAGTACTGGACCTGA | 55 | 4305 | 4320 | 1211 |
| 826086 | 45 | 60 | GGGAGTACTGGACCTG | 62 | 4306 | 4321 | 1212 |
| 826087 | 46 | 61 | TGGGAGTACTGGACCT | 81 | 4307 | 4322 | 1213 |
| 826088 | 47 | 62 | CTGGGAGTACTGGACC | 59 | 4308 | 4323 | 1214 |
| 826089 | 50 | 65 | GAACTGGGAGTACTGG | 81 | 4311 | 4326 | 1215 |
| 826092 | 62 | 77 | CCGAGGGCAGGTGAAC | 99 | 4323 | 4338 | 1216 |
| 826093 | 72 | 87 | AGGAGGGCTCCCGAGG | 81 | 4333 | 4348 | 1217 |
| 826102 | 94 | 109 | GAGCCGGGAGTTTTCC | 45 | 4355 | 4370 | 1218 |
| 826103 | 95 | 110 | AGAGCCGGGAGTTTTC | 71 | 4356 | 4371 | 1219 |
| 826104 | 98 | 113 | GTCAGAGCCGGGAGTT | 73 | 4359 | 4374 | 1220 |
| 826105 | 99 | 114 | AGTCAGAGCCGGGAGT | 73 | 4360 | 4375 | 1221 |
| 826106 | 100 | 115 | GAGTCAGAGCCGGGAG | 51 | 4361 | 4376 | 1222 |
| 826107 | 101 | 116 | GGAGTCAGAGCCGGGA | 53 | 4362 | 4377 | 1223 |
| 826108 | 138 | 153 | AATTAAAGGTGAGCAG | 67 | 4399 | 4414 | 1224 |
| 826109 | 143 | 158 | ATCTCAATTAAAGGTG | 91 | 4404 | 4419 | 1225 |
| 826112 | 164 | 179 | AGCGACAGGAATCTCA | 79 | 4425 | 4440 | 1226 |
| 826113 | 166 | 181 | GAAGCGACAGGAATCT | 68 | 4427 | 4442 | 1227 |
| 826122 | 181 | 196 | GGCCGGCCAGGGATGG | 41 | 4442 | 4457 | 1228 |
| 826123 | 182 | 197 | TGGCCGGCCAGGGATG | 55 | 4443 | 4458 | 1229 |
| 826124 | 183 | 198 | CTGGCCGGCCAGGGAT | 69 | 4444 | 4459 | 1230 |
| 826125 | 187 | 202 | CCCGCTGGCCGGCCAG | 32 | 4448 | 4463 | 1231 |
| 826126 | 188 | 203 | GCCCGCTGGCCGGCCA | 90 | 4449 | 4464 | 1232 |
| 826127 | 190 | 205 | CCGCCCGCTGGCCGGC | 58 | 4451 | 4466 | 1233 |
| 826128 | 192 | 207 | GCCCGCCCGCTGGCCG | 81 | 4453 | 4468 | 1234 |
| 826129 | 194 | 209 | GAGCCCGCCCGCTGGC | 57 | 4455 | 4470 | 1235 |
| 826132 | 217 | 232 | ACAGGTGCAGCGGCCT | 76 | 4478 | 4493 | 1236 |
| 826133 | 224 | 239 | TCCCCTGACAGGTGCA | 78 | N/A | N/A | 1237 |
| 826142 | 256 | 271 | CAGAGGTCTAGGGTCC | 26 | 5184 | 5199 | 1238 |
| 826143 | 259 | 274 | CTGCAGAGGTCTAGGG | 36 | 5187 | 5202 | 1239 |
| 826144 | 267 | 282 | GGTATGGGCTGCAGAG | 24 | 5195 | 5210 | 1240 |
| 826145 | 269 | 284 | CTGGTATGGGCTGCAG | 31 | 5197 | 5212 | 1241 |
| 826146 | 272 | 287 | GACCTGGTATGGGCTG | 30 | 5200 | 5215 | 1242 |
| 826147 | 275 | 290 | TGAGACCTGGTATGGG | 38 | 5203 | 5218 | 1243 |
| 826148 | 278 | 293 | CCATGAGACCTGGTAT | 50 | 5206 | 5221 | 1244 |
| 826149 | 280 | 295 | CTCCATGAGACCTGGT | 42 | 5208 | 5223 | 1245 |
| 826152 | 284 | 299 | TCCCCTCCATGAGACC | 50 | 5212 | 5227 | 1246 |
| 826153 | 286 | 301 | GTTCCCCTCCATGAGA | 65 | 5214 | 5229 | 1247 |
| 826162 | 332 | 347 | GCCCTGGAGTGGACTG | 46 | 5260 | 5275 | 1248 |
| 826163 | 333 | 348 | AGCCCTGGAGTGGACT | 41 | 5261 | 5276 | 1249 |
| 826164 | 343 | 358 | CCCCTTCATGAGCCCT | 30 | 5271 | 5286 | 1250 |
| 826165 | 344 | 359 | TCCCCTTCATGAGCCC | 30 | 5152 | 5167 | 1251 |
| | | | | | 5272 | 5287 | |
| 826166 | 345 | 360 | TTCCCCTTCATGAGCC | 29 | 5153 | 5168 | 1252 |
| | | | | | 5273 | 5288 | |
| 826167 | 350 | 365 | GCTTGTTCCCCTTCAT | 12 | 5158 | 5173 | 1253 |
| | | | | | 5278 | 5293 | |
| 826168 | 351 | 366 | CGCTTGTTCCCCTTCA | 19 | 5279 | 5294 | 1254 |
| 826169 | 352 | 367 | ACGCTTGTTCCCCTTC | 20 | 5280 | 5295 | 1255 |
| 826172 | 356 | 37 | CCTCACGCTTGTTCCC | 41 | 5284 | 5299 | 1256 |
| 826173 | 359 | 374 | GCTCCTCACGCTTGTT | 48 | 5287 | 5302 | 1257 |
| 826182 | 425 | 440 | ACTCGATCAGGGCCTC | 34 | 5353 | 5368 | 1258 |
| 826183 | 427 | 442 | GAACTCGATCAGGGCC | 21 | 5355 | 5370 | 1259 |
| 826184 | 429 | 444 | TGGAACTCGATCAGGG | 45 | 5357 | 5372 | 1260 |
| 826185 | 431 | 446 | GGTGGAACTCGATCAG | 26 | 5359 | 5374 | 1261 |
| 826186 | 432 | 447 | CGGTGGAACTCGATCA | 36 | 5360 | 5375 | 1262 |
| 826187 | 433 | 448 | GCGGTGGAACTCGATC | 47 | 5361 | 5376 | 1263 |
| 826188 | 435 | 450 | GAGCGGTGGAACTCGA | 39 | 5363 | 5378 | 1264 |
| 826189 | 436 | 451 | GGAGCGGTGGAACTCG | 53 | 5364 | 5379 | 1265 |
| 826192 | 443 | 458 | CTCGGTAGGAGCGGTG | 50 | 5371 | 5386 | 1266 |
| 826193 | 445 | 460 | CTCTCGGTAGGAGCGG | 42 | 5373 | 5388 | 1267 |
| 826202 | 516 | 531 | CGGTTGTGCTGGGAGC | 19 | 5444 | 5459 | 1268 |
| 826203 | 517 | 532 | GCGGTTGTGCTGGGAG | 24 | 5445 | 5460 | 1269 |
| 826204 | 519 | 534 | ATGCGGTTGTGCTGGG | 23 | 5447 | 5462 | 1270 |
| 826205 | 524 | 539 | TCTTCATGCGGTTGTG | 31 | 5452 | 5467 | 1271 |
| 826206 | 529 | 544 | GGCCGTCTTCATGCGG | 39 | 5457 | 5472 | 1272 |
| 826207 | 532 | 547 | GAAGGCCGTCTTCATG | 27 | 5460 | 5475 | 1273 |
| 826208 | 564 | 579 | ATGCCAAAGGTGCAGA | 51 | 5492 | 5507 | 1274 |
| 826211 | 569 | 584 | ACATCATGCCAAAGGT | 33 | 5497 | 5512 | 1275 |
| 826212 | 573 | 588 | CAGTACATCATGCCAA | 42 | 5501 | 5516 | 1276 |
| 826221 | 590 | 605 | AAAGCAGGCCGAATTG | 38 | 5518 | 5533 | 1277 |
| 826222 | 594 | 609 | CCGAAAAGCAGGCCGA | 27 | 5522 | 5537 | 1278 |
| 826223 | 596 | 611 | CTCCGAAAAGCAGGCC | 31 | 5524 | 5539 | 1279 |
| 826224 | 598 | 613 | CTCTCCGAAAAGCAGG | 37 | 5526 | 5541 | 1280 |
| 826225 | 599 | 614 | ACTCTCCGAAAAGCAG | 27 | 5527 | 5542 | 1281 |
| 826226 | 601 | 616 | GTACTCTCCGAAAAGC | 20 | 5529 | 5544 | 1282 |
| 826227 | 602 | 617 | AGTACTCTCCGAAAAG | 43 | 5530 | 5545 | 1283 |
| 826228 | 605 | 620 | TGAAGTACTCTCCGAA | 44 | 5533 | 5548 | 1284 |
| 826231 | 610 | 625 | GTAGCTGAAGTACTCT | 35 | 5538 | 5553 | 1285 |
| 826232 | 611 | 626 | GGTAGCTGAAGTACTC | 23 | 5539 | 5554 | 1286 |
| 826241 | 650 | 665 | CGAGCTTGTCCGAGTT | 17 | 5578 | 5593 | 1287 |
| 826242 | 652 | 667 | GACGAGCTTGTCCGAG | 22 | 5580 | 5595 | 1288 |
| 826243 | 653 | 668 | AGACGAGCTTGTCCGA | 23 | 5581 | 5596 | 1289 |
| 826244 | 655 | 670 | GAAGACGAGCTTGTCC | 30 | 5583 | 5598 | 1290 |
| 826245 | 656 | 671 | GGAAGACGAGCTTGTC | 27 | 5584 | 5599 | 1291 |
| 826246 | 657 | 672 | GGGAAGACGAGCTTGT | 21 | 5585 | 5600 | 1292 |
| 826247 | 699 | 714 | TCCGGGTACCTGTAGG | 32 | N/A | N/A | 1293 |
| 826248 | 700 | 715 | TTCCGGGTACCTGTAG | 38 | N/A | N/A | 1294 |
| 826251 | 705 | 720 | TTAATTTCCGGGTACC | 27 | 16290 | 16305 | 1295 |
| 826252 | 709 | 724 | CTCTTTAATTTCCGGG | 31 | 16294 | 16309 | 1296 |
| 826261 | 763 | 778 | TTTGTACAGGTCAAAG | 43 | 16348 | 16363 | 1297 |
| 826262 | 766 | 781 | GTATTTGTACAGGTCA | 12 | 16351 | 16366 | 1298 |
| 826263 | 77 | 792 | GTGAAGGAGCTGTATT | 93 | 16362 | 16377 | 1299 |
| 826264 | 786 | 801 | ACGAGAGTGGTGAAGG | 43 | 16371 | 16386 | 1300 |
| 826265 | 788 | 803 | CCACGAGAGTGGTGAA | 56 | 16373 | 16388 | 1301 |
| 826266 | 789 | 804 | GCCACGAGAGTGGTGA | 77 | 16374 | 16389 | 1302 |
| 826269 | 794 | 809 | AGCCGGCCACGAGAGT | 43 | 16379 | 16394 | 1303 |
| 826270 | 795 | 810 | GAGCCGGCCACGAGAG | 42 | 16380 | 16395 | 1304 |
| 826279 | 812 | 827 | GGTCGCGACGGCTGCG | 31 | 16397 | 16412 | 1305 |
| 826280 | 815 | 830 | GCAGGTCGCGACGGCT | 48 | 16400 | 16415 | 1306 |
| 826281 | 816 | 831 | CGCAGGTCGCGACGGC | 35 | 16401 | 16416 | 1307 |
| 826282 | 819 | 834 | CCCCGCAGGTCGCGAC | 49 | 16404 | 16419 | 1308 |
| 826283 | 820 | 835 | CCCCCGCAGGTCGCGA | 43 | 16405 | 16420 | 1309 |
| 826284 | 821 | 836 | TCCCCCGCAGGTCGCG | 35 | 16406 | 16421 | 1310 |
| 826285 | 822 | 837 | GTCCCCCGCAGGTCGC | 49 | 16407 | 16422 | 1311 |
| 826286 | 824 | 839 | GAGTCCCCCGCAGGTC | 67 | 16409 | 16424 | 1312 |
| 826289 | 831 | 846 | TGCGGCAGAGTCCCCC | 37 | 16416 | 16431 | 1313 |
| 826290 | 833 | 848 | GGTGCGGCAGAGTCCC | 38 | 16418 | 16433 | 1314 |
| 826299 | 893 | 908 | CCACGCTACGGGCTCG | 33 | 16478 | 16493 | 1315 |
| 826300 | 895 | 910 | GGCCACGCTACGGGCT | 56 | 16480 | 16495 | 1316 |
| 826301 | 897 | 912 | GAGGCCACGCTACGGG | 36 | 16482 | 16497 | 1317 |
| 826302 | 898 | 913 | GGAGGCCACGCTACGG | 34 | 16483 | 16498 | 1318 |
| 826303 | 900 | 915 | CTGGAGGCCACGCTAC | 49 | 16485 | 16500 | 1319 |
| 826304 | 902 | 917 | AGCTGGAGGCCACGCT | 28 | 16487 | 16502 | 1320 |
| 826305 | 908 | 923 | CCCGCAAGCTGGAGGC | 41 | 16493 | 16508 | 1321 |
| 826306 | 913 | 928 | GTTGTCCCGCAAGCTG | 23 | 16498 | 16513 | 1322 |
| 826309 | 917 | 932 | GGTTGTTGTCCCGCAA | 33 | 16502 | 16517 | 1323 |
| 826310 | 918 | 933 | GGGTTGTTGTCCCGCA | 43 | 16503 | 16518 | 1324 |
| 826319 | 971 | 986 | TGTTCTGGTTGCACAG | 35 | N/A | N/A | 1325 |
| 826320 | 972 | 987 | TTGTTCTGGTTGCACA | 38 | N/A | N/A | 1326 |
| 826321 | 973 | 988 | TTTGTTCTGGTTGCAC | 23 | 17759 | 17774 | 1327 |
| 826322 | 975 | 990 | GATTTGTTCTGGTTGC | 36 | 17761 | 17776 | 1328 |
| 826323 | 976 | 991 | CGATTTGTTCTGGTTG | 22 | 17762 | 17777 | 1329 |
| 826324 | 978 | 993 | TCCGATTTGTTCTGGT | 38 | 17764 | 17779 | 1330 |
| 826327 | 981 | 996 | CAGTCCGATTTGTTCT | 36 | 17767 | 17782 | 1331 |
| 826328 | 982 | 997 | GCAGTCCGATTTGTTC | 34 | 17768 | 17783 | 1332 |
| 826337 | 1000 | 1015 | TGAGTATGTCTGGTAG | 19 | 17786 | 17801 | 1333 |
| 826338 | 1001 | 1016 | ATGAGTATGTCTGGTA | 15 | 17787 | 17802 | 1334 |
| 826339 | 1003 | 1018 | TGATGAGTATGTCTGG | 19 | 17789 | 17804 | 1335 |
| 826340 | 1007 | 1022 | CCCCTGATGAGTATGT | 41 | 17793 | 17808 | 1336 |
| 826341 | 1009 | 1024 | CACCCCTGATGAGTAT | 61 | 17795 | 17810 | 1337 |
| 826342 | 1011 | 1026 | TCCACCCCTGATGAGT | 36 | 17797 | 17812 | 1338 |
| 826343 | 1014 | 1029 | GCATCCACCCCTGATG | 56 | 17800 | 17815 | 1339 |
| 826344 | 1015 | 1030 | CGCATCCACCCCTGAT | 70 | 17801 | 17816 | 1340 |
| 826347 | 1019 | 1034 | TCACCGCATCCACCCC | 35 | 17805 | 17820 | 1341 |
| 826348 | 1021 | 1036 | CCTCACCGCATCCACC | 72 | 17807 | 17822 | 1342 |
| 826357 | 1047 | 1062 | TTGATGTAGTGGAAGC | 39 | 17833 | 17848 | 1343 |
| 826358 | 1058 | 1073 | TCGACAGGATGTTGAT | 56 | 17844 | 17859 | 1344 |
| 826359 | 1060 | 1075 | CCTCGACAGGATGTTG | 28 | 17846 | 17861 | 1345 |
| 826360 | 1061 | 1076 | GCCTCGACAGGATGTT | 37 | 17847 | 17862 | 1346 |
| 826361 | 1064 | 1079 | GCAGCCTCGACAGGAT | 45 | 17850 | 17865 | 1347 |
| 826362 | 1065 | 1080 | GGCAGCCTCGACAGGA | 22 | 17851 | 17866 | 1348 |
| 826363 | 1074 | 1089 | AGAGTCTCTGGCAGCC | 25 | 17860 | 17875 | 1349 |
| 826364 | 1110 | 1125 | ATGAAGTTGCCCAGCG | 64 | 17896 | 17911 | 1350 |
| 826367 | 1117 | 1132 | GGCGAAGATGAAGTTG | 47 | 17903 | 17918 | 1351 |
| 826368 | 1121 | 1136 | GGCAGGCGAAGATGAA | 46 | 17907 | 17922 | 1352 |
| 826377 | 1148 | 1163 | CCTGGTTGCAGGAGAC | 24 | 17934 | 17949 | 1353 |
| 826378 | 1150 | 1165 | CGCCTGGTTGCAGGAG | 38 | 17936 | 17951 | 1354 |
| 826379 | 1152 | 1167 | TTCGCCTGGTTGCAGG | 54 | N/A | N/A | 1355 |
| 826380 | 1153 | 1168 | ATTCGCCTGGTTGCAG | 52 | N/A | N/A | 1356 |
| 826381 | 1155 | 1170 | TAATTCGCCTGGTTGC | 86 | N/A | N/A | 1357 |
| 826382 | 1157 | 1172 | AGTAATTCGCCTGGTT | 73 | N/A | N/A | 1358 |
| 826383 | 1158 | 1173 | GAGTAATTCGCCTGGT | 49 | N/A | N/A | 1359 |
| 826384 | 1160 | 1175 | GAGAGTAATTCGCCTG | 63 | N/A | N/A | 1360 |
| 826387 | 1168 | 1183 | GTGGAAGTGAGAGTAA | 48 | 24124 | 24139 | 1361 |
| 826388 | 1230 | 1245 | GACATCCAGAGGTTGG | 47 | 24186 | 24201 | 1362 |
| 826397 | 1261 | 1276 | GGACAGACCGTTGTTG | 31 | N/A | N/A | 1363 |
| 826398 | 1267 | 1282 | CATCAGGGACAGACCG | 40 | N/A | N/A | 1364 |
| 826399 | 1268 | 1283 | GCATCAGGGACAGACC | 24 | 24565 | 24580 | 1365 |
| 826400 | 1273 | 1288 | GCGCAGCATCAGGGAC | 28 | 24570 | 24585 | 1366 |
| 826401 | 1275 | 1290 | GCGCGCAGCATCAGGG | 37 | 24572 | 24587 | 1367 |
| 826402 | 1277 | 1292 | CTGCGCGCAGCATCAG | 35 | 24574 | 24589 | 1368 |
| 826403 | 1279 | 1294 | CTCTGCGCGCAGCATC | 20 | 24576 | 24591 | 1369 |
| 826404 | 1280 | 1295 | GCTCTGCGCGCAGCAT | 49 | 24577 | 24592 | 1370 |
| 826407 | 1283 | 1298 | TCTGCTCTGCGCGCAG | 65 | 24580 | 24595 | 1371 |
| 826408 | 1284 | 1299 | TTCTGCTCTGCGCGCA | 49 | 24581 | 24596 | 1372 |
| 826417 | 1333 | 1348 | CACCATTACCCGGGCC | 34 | 24630 | 24645 | 1373 |
| 826418 | 1341 | 1356 | TGCCCGTGCACCATTA | 30 | 24638 | 24653 | 1374 |
| 826419 | 1343 | 1358 | CCTGCCCGTGCACCAT | 25 | 24640 | 24655 | 1375 |
| 826420 | 1344 | 1359 | TCCTGCCCGTGCACCA | 18 | 24641 | 24656 | 1376 |
| 826421 | 1345 | 1360 | ATCCTGCCCGTGCACC | 31 | 24642 | 24657 | 1377 |
| 826422 | 1348 | 1363 | TTCATCCTGCCCGTGC | 20 | 24645 | 24660 | 1378 |
| 826423 | 1350 | 1365 | GGTTCATCCTGCCCGT | 53 | 24647 | 24662 | 1379 |
| 826424 | 1351 | 1366 | AGGTTCATCCTGCCCG | 78 | 24648 | 24663 | 1380 |
| 826427 | 1358 | 1373 | TAAAGGCAGGTTCATC | 89 | 24655 | 24670 | 1381 |
| 826428 | 1361 | 1376 | CCATAAAGGCAGGTTC | 73 | 24658 | 24673 | 1382 |
| 826437 | 1393 | 1408 | CACGCCAGGCCGCAAG | 36 | 24690 | 24705 | 1383 |
| 826438 | 1394 | 1409 | CCACGCCAGGCCGCAA | 45 | 24691 | 24706 | 1384 |
| 826439 | 1395 | 1410 | TCCACGCCAGGCCGCA | 28 | 24692 | 24707 | 1385 |
| 826440 | 1396 | 1411 | CTCCACGCCAGGCCGC | 38 | 24693 | 24708 | 1386 |
| 826441 | 1399 | 1414 | GGTCTCCACGCCAGGC | 38 | 24696 | 24711 | 1387 |
| 826442 | 1401 | 1416 | GAGGTCTCCACGCCAG | 25 | 24698 | 24713 | 1388 |
| 826443 | 1402 | 1417 | GGAGGTCTCCACGCCA | 55 | 24699 | 24714 | 1389 |
| 826444 | 1404 | 1419 | ATGGAGGTCTCCACGC | 45 | 24701 | 24716 | 1390 |
| 826447 | 1442 | 1457 | CGCCCCCAAGTCTGTC | 38 | 25162 | 25177 | 1391 |
| 826448 | 1443 | 1458 | TCGCCCCCAAGTCTGT | 32 | 25163 | 25178 | 1392 |
| 826457 | 1461 | 1476 | TTGGTGCAGTCGCCAT | 25 | 25181 | 25196 | 1393 |
| 826458 | 1462 | 1477 | CTTGGTGCAGTCGCCA | 43 | 25182 | 25197 | 1394 |
| 826459 | 1463 | 1478 | TCTTGGTGCAGTCGCC | 24 | 25183 | 25198 | 1395 |
| 826460 | 1466 | 1481 | CATTCTTGGTGCAGTC | 40 | 25186 | 25201 | 1396 |
| 826461 | 1468 | 1483 | GCCATTCTTGGTGCAG | 45 | 25188 | 25203 | 1397 |
| 826462 | 1470 | 1485 | CTGCCATTCTTGGTGC | 30 | 25190 | 25205 | 1398 |
| 826463 | 1480 | 1495 | AGGAACATCACTGCCA | 29 | 25200 | 25215 | 1399 |
| 826464 | 1496 | 1511 | GGTAAAGGTTCTCAAC | 68 | 25216 | 25231 | 1400 |
| 826467 | 1509 | 1524 | GTGTACTTTGAAGGGT | 49 | 25229 | 25244 | 1401 |
| 826468 | 1526 | 1541 | GAATACACACCTGCTG | 54 | N/A | N/A | 1402 |
| 826477 | 1558 | 1573 | CTCCTTGATCATGCTC | 31 | 25472 | 25487 | 1403 |
| 826478 | 1559 | 1574 | ACTCCTTGATCATGCT | 36 | 25473 | 25488 | 1404 |
| 826479 | 1560 | 1575 | CACTCCTTGATCATGC | 29 | 25474 | 25489 | 1405 |
| 826480 | 1561 | 1576 | ACACTCCTTGATCATG | 29 | 25475 | 25490 | 1406 |
| 826481 | 1562 | 1577 | CACACTCCTTGATCAT | 38 | 25476 | 25491 | 1407 |
| 826482 | 1564 | 1579 | GCCACACTCCTTGATC | 32 | 25478 | 25493 | 1408 |
| 826483 | 1576 | 1591 | GATGTAGGCACAGCCA | 25 | 25490 | 25505 | 1409 |
| 826484 | 1577 | 1592 | AGATGTAGGCACAGCC | 22 | 25491 | 25506 | 1410 |
| 826486 | 1581 | 1596 | TAGAAGATGTAGGCAC | 47 | 25495 | 25510 | 1411 |
| 826487 | 1582 | 1597 | ATAGAAGATGTAGGCA | 49 | 25496 | 25511 | 1412 |
| 826496 | 1638 | 1653 | TACCCCCAGGAACTGT | 27 | N/A | N/A | 1413 |
| 826497 | 1639 | 1654 | GTACCCCCAGGAACTG | 49 | N/A | N/A | 1414 |
| 826498 | 1640 | 1655 | AGTACCCCCAGGAACT | 65 | N/A | N/A | 1415 |
| 826499 | 1641 | 1656 | CAGTACCCCCAGGAAC | 44 | N/A | N/A | 1416 |
| 826500 | 1648 | 1663 | ATAGTAGCAGTACCCC | 36 | N/A | N/A | 1417 |
| 826501 | 1650 | 1665 | TTATAGTAGCAGTACC | 38 | 30596 | 30611 | 1418 |
| 826502 | 1654 | 1669 | GAGCTTATAGTAGCAG | 59 | 30600 | 30615 | 1419 |
| 826503 | 1655 | 1670 | GGAGCTTATAGTAGCA | 55 | 30601 | 30616 | 1420 |
| 826506 | 1662 | 1677 | TCAACCTGGAGCTTAT | 39 | 30608 | 30623 | 1421 |
| 826507 | 1664 | 1679 | AGTCAACCTGGAGCTT | 41 | 30610 | 30625 | 1422 |
| 826516 | 1717 | 1732 | CACGCTGCATGGCTTC | 21 | N/A | N/A | 1423 |
| 826517 | 1720 | 1735 | GGTCACGCTGCATGGC | 38 | N/A | N/A | 1424 |
| 826518 | 1722 | 1737 | CTGGTCACGCTGCATG | 35 | N/A | N/A | 1425 |
| 826519 | 1723 | 1738 | GCTGGTCACGCTGCAT | 30 | N/A | N/A | 1426 |
| 826520 | 1724 | 1739 | AGCTGGTCACGCTGCA | 41 | N/A | N/A | 1427 |
| 826521 | 1727 | 1742 | GGTAGCTGGTCACGCT | 26 | 30778 | 30793 | 1428 |
| 826522 | 1729 | 1744 | CTGGTAGCTGGTCACG | 46 | 30780 | 30795 | 1429 |
| 826523 | 1732 | 1747 | GAGCTGGTAGCTGGTC | 48 | 30783 | 30798 | 1430 |
| 826526 | 1737 | 1752 | GCAGAGAGCTGGTAGC | 67 | 30788 | 30803 | 1431 |
| 826527 | 1749 | 1764 | CGTGAGTAACCAGCAG | 48 | 30800 | 30815 | 1432 |
| 826556 | 1890 | 1905 | GACTCAGAATTGGTTT | 61 | 31246 | 31261 | 1433 |
| 826557 | 1961 | 1976 | CCGAGGAGCCGAACCA | 50 | 31790 | 31805 | 1434 |
| 826558 | 1965 | 1980 | AACACCGAGGAGCCGA | 14 | 31794 | 31809 | 1435 |
| 826559 | 1966 | 1981 | CAACACCGAGGAGCCG | 36 | 31795 | 31810 | 1436 |
| 826560 | 1968 | 1983 | GACAACACCGAGGAGC | 56 | 31797 | 31812 | 1437 |
| 826561 | 1970 | 1985 | CAGACAACACCGAGGA | 30 | 31799 | 31814 | 1438 |
| 826562 | 1972 | 1987 | CACAGACAACACCGAG | 42 | 31801 | 31816 | 1439 |
| 826563 | 1973 | 1988 | CCACAGACAACACCGA | 60 | 31802 | 31817 | 1440 |
| 826566 | 1998 | 2013 | TCAAAGACGAGCTCAG | 61 | 31827 | 31842 | 1441 |
| 826567 | 1999 | 2014 | GTCAAAGACGAGCTCA | 62 | 31828 | 31843 | 1442 |
| 826576 | 2036 | 2051 | ACCTTCGGAGCAGCAT | 17 | 31865 | 31880 | 1443 |
| 826577 | 2038 | 2053 | GAACCTTCGGAGCAGC | 28 | 31867 | 31882 | 1444 |
| 826578 | 2039 | 2054 | GGAACCTTCGGAGCAG | 17 | 31868 | 31883 | 1445 |
| 826579 | 2040 | 2055 | CGGAACCTTCGGAGCA | 20 | 31869 | 31884 | 1446 |
| 826580 | 2041 | 2056 | TCGGAACCTTCGGAGC | 32 | 31870 | 31885 | 1447 |
| 826581 | 2042 | 2057 | TTCGGAACCTTCGGAG | 30 | 31871 | 31886 | 1448 |
| 826582 | 2043 | 2058 | CTTCGGAACCTTCGGA | 47 | 31872 | 31887 | 1449 |
| 826583 | 2044 | 2059 | GCTTCGGAACCTTCGG | 30 | 31873 | 31888 | 1450 |
| 826585 | 2047 | 2062 | TCGGCTTCGGAACCTT | 32 | 31876 | 31891 | 1451 |
| 826586 | 2048 | 2063 | ATCGGCTTCGGAACCT | 33 | 31877 | 31892 | 1452 |
| 826595 | 2059 | 2074 | TGGAGACCAGTATCGG | 20 | 31888 | 31903 | 1453 |
| 826596 | 2061 | 2076 | CCTGGAGACCAGTATC | 30 | 31890 | 31905 | 1454 |
| 826597 | 2066 | 2081 | CTCGGCCTGGAGACCA | 35 | 31895 | 31910 | 1455 |
| 826598 | 2069 | 2084 | CCCCTCGGCCTGGAGA | 42 | 31898 | 31913 | 1456 |
| 826599 | 2071 | 2086 | GCCCCCTCGGCCTGGA | 64 | 31900 | 31915 | 1457 |
| 826600 | 2072 | 2087 | TGCCCCCTCGGCCTGG | 46 | 31901 | 31916 | 1458 |
| 826601 | 2093 | 2108 | AGGCTACCTCCTGAGC | 48 | 31922 | 31937 | 1459 |
| 826602 | 2098 | 2113 | GGTGGAGGCTACCTCC | 62 | 31927 | 31942 | 1460 |
| 826605 | 2279 | 2294 | GCCCCCCCAGAGGACA | 90 | 32108 | 32123 | 1461 |
| 826606 | 2280 | 2295 | GGCCCCCCCAGAGGAC | 75 | 32109 | 32124 | 1462 |
| 826615 | 2320 | 2335 | GCATCTGCCTTGGTGT | 24 | 32149 | 32164 | 1463 |
| 826616 | 2322 | 2337 | GAGCATCTGCCTTGGT | 24 | 32151 | 32166 | 1464 |
| 826617 | 2324 | 2339 | AGGAGCATCTGCCTTG | 32 | 32153 | 32168 | 1465 |
| 826618 | 2330 | 2345 | CACCAGAGGAGCATCT | 46 | 32159 | 32174 | 1466 |
| 826619 | 2331 | 2346 | CCACCAGAGGAGCATC | 34 | 32160 | 32175 | 1467 |
| 826620 | 2355 | 2370 | AATCTTGCCAGGGCCA | 21 | 32184 | 32199 | 1468 |
| 826621 | 2356 | 2371 | CAATCTTGCCAGGGCC | 37 | 32185 | 32200 | 1469 |
| 826622 | 2359 | 2374 | CTTCAATCTTGCCAGG | 41 | 32188 | 32203 | 1470 |
| 826625 | 2393 | 2408 | GTTTGGGCGGCTCTGA | 36 | 32222 | 32237 | 1471 |
| 826626 | 2395 | 2410 | CAGTTTGGGCGGCTCT | 22 | 32224 | 32239 | 1472 |
| 826635 | 2412 | 2427 | CCTCCACACATCAACG | 38 | 32241 | 32256 | 1473 |
| 826636 | 2435 | 2450 | GAGCCCTTACCCATCT | 50 | 32264 | 32279 | 1474 |
| 826637 | 2436 | 2451 | TGAGCCCTTACCCATC | 49 | 32265 | 32280 | 1475 |
| 826638 | 2439 | 2454 | TCCTGAGCCCTTACCC | 33 | 32268 | 32283 | 1476 |
| 826639 | 2447 | 2462 | GAGCAACTTCCTGAGC | 28 | 32276 | 32291 | 1477 |
| 826640 | 2449 | 2464 | TGGAGCAACTTCCTGA | 39 | 32278 | 32293 | 1478 |
| 826641 | 2459 | 2474 | CTACTGTTCTTGGAGC | 28 | 32288 | 32303 | 1479 |
| 826642 | 2462 | 2477 | CAGCTACTGTTCTTGG | 22 | 32291 | 32306 | 1480 |
| 826645 | 2477 | 2492 | TCTGGGCAGCTTCATC | 37 | 32306 | 32321 | 1481 |
| 826646 | 2490 | 2505 | GAGCCAAGGCACTTCT | 31 | 32319 | 32334 | 1482 |
| 826655 | 2553 | 2568 | CTTAGCCGCAGTTGGG | 13 | 32382 | 32397 | 1483 |
| 826656 | 2554 | 2569 | ACTTAGCCGCAGTTGG | 35 | 32383 | 32398 | 1484 |
| 826657 | 2555 | 2570 | GACTTAGCCGCAGTTG | 40 | 32384 | 32399 | 1485 |
| 826658 | 2556 | 2571 | AGACTTAGCCGCAGTT | 24 | 32385 | 32400 | 1486 |
| 826659 | 2557 | 2572 | GAGACTTAGCCGCAGT | 19 | 32386 | 32401 | 1487 |
| 826660 | 2559 | 2574 | AAGAGACTTAGCCGCA | 18 | 32388 | 32403 | 1488 |
| 826661 | 2561 | 2576 | AAAAGAGACTTAGCCG | 42 | 32390 | 32405 | 1489 |
| 826663 | 2577 | 2592 | TGGCTGATCCAAGGGA | 46 | 32406 | 32421 | 1490 |
| 826664 | 2578 | 2593 | TTGGCTGATCCAAGGG | 48 | 32407 | 32422 | 1491 |
| 826673 | 2587 | 2602 | AAGTTTCGCTTGGCTG | 18 | 32416 | 32431 | 1492 |
| 826674 | 2589 | 2604 | CCAAGTTTCGCTTGGC | 63 | 32418 | 32433 | 1493 |
| 826675 | 2591 | 2606 | CTCCAAGTTTCGCTTG | 33 | 32420 | 32435 | 1494 |
| 826676 | 2593 | 2608 | AGCTCCAAGTTTCGCT | 48 | 32422 | 32437 | 1495 |
| 826677 | 2600 | 2615 | TTGTCAAAGCTCCAAG | 19 | 32429 | 32444 | 1496 |
| 826678 | 2602 | 2617 | CCTTGTCAAAGCTCCA | 8 | 32431 | 32446 | 1497 |
| 826679 | 2604 | 2619 | TTCCTTGTCAAAGCTC | 24 | 32433 | 32448 | 1498 |
| 826680 | 2607 | 2622 | AAGTTCCTTGTCAAAG | 39 | 32436 | 32451 | 1499 |
| 826683 | 2621 | 2636 | GCGGTTTCTTAGGAAA | 22 | 32450 | 32465 | 1500 |
| 826684 | 2622 | 2637 | AGCGGTTTCTTAGGAA | 26 | 32451 | 32466 | 1501 |
| 826693 | 2653 | 2668 | GTACCCTTGGTTGTGT | 21 | 32482 | 32497 | 1502 |
| 826694 | 2655 | 2670 | GTGTACCCTTGGTTGT | 31 | 32484 | 32499 | 1503 |
| 826695 | 2656 | 2671 | CGTGTACCCTTGGTTG | 22 | 32485 | 32500 | 1504 |
| 826696 | 2670 | 2685 | CCCGTGCATGCCTGCG | 23 | 32499 | 32514 | 1505 |
| 826697 | 2674 | 2689 | GAAACCCGTGCATGCC | 28 | 32503 | 32518 | 1506 |
| 826698 | 2676 | 2691 | AGGAAACCCGTGCATG | 21 | 32505 | 32520 | 1507 |
| 826699 | 2677 | 2692 | CAGGAAACCCGTGCAT | 31 | 32506 | 32521 | 1508 |
| 826700 | 2678 | 2693 | GCAGGAAACCCGTGCA | 58 | 32507 | 32522 | 1509 |
| 826703 | 2685 | 2700 | TCGCTGGGCAGGAAAC | 31 | 32514 | 32529 | 1510 |
| 826704 | 2687 | 2702 | CGTCGCTGGGCAGGAA | 29 | 32516 | 32531 | 1511 |
| 826713 | 2736 | 2751 | GTGCTACTGGAGAGCA | 34 | 32565 | 32580 | 1512 |
| 826714 | 2737 | 2752 | TGTGCTACTGGAGAGC | 35 | 32566 | 32581 | 1513 |
| 826715 | 2738 | 2753 | CTGTGCTACTGGAGAG | 19 | 32567 | 32582 | 1514 |
| 826716 | 2739 | 2754 | TCTGTGCTACTGGAGA | 23 | 32568 | 32583 | 1515 |
| 826717 | 2740 | 2755 | ATCTGTGCTACTGGAG | 19 | 32569 | 32584 | 1516 |
| 826718 | 2750 | 2765 | AGGAGCAGACATCTGT | 17 | 32579 | 32594 | 1517 |
| 826719 | 2775 | 2790 | GGGTTTCCCACCCAAG | 91 | 32604 | 32619 | 1518 |
| 826720 | 2810 | 2825 | GGAATTGCCTAAGTAA | 29 | 32639 | 32654 | 1519 |
| 826723 | 2837 | 2852 | GCCCTAGCCCTCGGGA | 66 | 32666 | 32681 | 1520 |
| 826724 | 2839 | 2854 | TAGCCCTAGCCCTCGG | 60 | 32668 | 32683 | 1521 |
| 826733 | 2860 | 2875 | TTTACTTACCCGGGTC | 40 | 32689 | 32704 | 1522 |
| 826734 | 2862 | 2877 | CCTTTACTTACCCGGG | 47 | 32691 | 32706 | 1523 |
| 826735 | 2865 | 2880 | CTGCCTTTACTTACCC | 37 | 32694 | 32709 | 1524 |
| 826736 | 2884 | 2899 | GGCTAGAGGAGCCCTG | 48 | 32713 | 32728 | 1525 |
| 826737 | 2886 | 2901 | GAGGCTAGAGGAGCCC | 46 | 32715 | 32730 | 1526 |
| 826738 | 2891 | 2906 | GGTATGAGGCTAGAGG | 27 | 32720 | 32735 | 1527 |
| 826739 | 2893 | 2908 | CGGGTATGAGGCTAGA | 34 | 32722 | 32737 | 1528 |
| 826740 | 2895 | 2910 | CACGGGTATGAGGCTA | 55 | 32724 | 32739 | 1529 |
| 826743 | 2951 | 2966 | CATGTAGAGGTATGAA | 26 | 32780 | 32795 | 1530 |
| 826744 | 2953 | 2968 | GACATGTAGAGGTATG | 31 | 32782 | 32797 | 1531 |
| 826753 | 2966 | 2981 | AATATCTCAAGCAGAC | 18 | 32795 | 32810 | 1532 |
| 826754 | 2986 | 3001 | GGAAACTTTCAGGCTG | 22 | 32815 | 32830 | 1533 |
| 826755 | 2987 | 3002 | GGGAAACTTTCAGGCT | 25 | 32816 | 32831 | 1534 |
| 826756 | 3001 | 3016 | CTGGCAGATGGTTGGG | 31 | 32830 | 32845 | 1535 |
| 826757 | 3003 | 3018 | CTCTGGCAGATGGTTG | 30 | 32832 | 32847 | 1536 |
| 826758 | 3008 | 3023 | GAGTTCTCTGGCAGAT | 20 | 32837 | 32852 | 1537 |
| 826759 | 3010 | 3025 | AGGAGTTCTCTGGCAG | 22 | 32839 | 32854 | 1538 |
| 826760 | 3011 | 3026 | TAGGAGTTCTCTGGCA | 31 | 32840 | 32855 | 1539 |
| 826762 | 3014 | 3029 | GCATAGGAGTTCTCTG | 30 | 32843 | 32858 | 1540 |
| 826763 | 3015 | 3030 | TGCATAGGAGTTCTCT | 18 | 32844 | 32859 | 1541 |
| 826772 | 3035 | 3050 | CTGAGCAGGGTTCTAA | 20 | 32864 | 32879 | 1542 |
| 826773 | 3036 | 3051 | TCTGAGCAGGGTTCTA | 24 | 32865 | 32880 | 1543 |
| 826774 | 3039 | 3054 | GTGTCTGAGCAGGGTT | 13 | 32868 | 32883 | 1544 |
| 826775 | 3044 | 3059 | TAATGGTGTCTGAGCA | 19 | 32873 | 32888 | 1545 |
| 826776 | 3045 | 3060 | GTAATGGTGTCTGAGC | 11 | 32874 | 32889 | 1546 |
| 826777 | 3074 | 3089 | GACAAGATGTGGCAGA | 22 | 32903 | 32918 | 1547 |
| 826778 | 3097 | 3112 | GCGGAGTGATCAATTT | 50 | 32926 | 32941 | 1548 |
| 826779 | 3099 | 3114 | AGGCGGAGTGATCAAT | 55 | 32928 | 32943 | 1549 |
| 826782 | 3119 | 3134 | TGCTACGGGAGCCCAG | 46 | 32948 | 32963 | 1550 |
| 826783 | 3120 | 3135 | GTGCTACGGGAGCCCA | 24 | 32949 | 32964 | 1551 |
| 826791 | 3129 | 3144 | TGTTATAGTGTGCTAC | 27 | 32958 | 32973 | 1552 |
| 826792 | 3130 | 3145 | ATGTTATAGTGTGCTA | 34 | 32959 | 32974 | 1553 |
| 826793 | 3131 | 3146 | GATGTTATAGTGTGCT | 12 | 32960 | 32975 | 1554 |
| 826794 | 3132 | 3147 | AGATGTTATAGTGTGC | 13 | 32961 | 32976 | 1555 |
| 826795 | 3133 | 3148 | CAGATGTTATAGTGTG | 21 | 32962 | 32977 | 1556 |
| 826796 | 3135 | 3150 | AGCAGATGTTATAGTG | 14 | 32964 | 32979 | 1557 |
| 826797 | 3136 | 3151 | CAGCAGATGTTATAGT | 37 | 32965 | 32980 | 1558 |
| 826798 | 3137 | 3152 | CCAGCAGATGTTATAG | 50 | 32966 | 32981 | 1559 |
| 826801 | 3146 | 3161 | AGCAACACTCCAGCAG | 46 | 32975 | 32990 | 1560 |
| 826802 | 3147 | 3162 | CAGCAACACTCCAGCA | 35 | 32976 | 32991 | 1561 |
| 826810 | 3161 | 3176 | AAAGTATGGTGCAACA | 22 | 32990 | 33005 | 1562 |
| 826811 | 3162 | 3177 | GAAAGTATGGTGCAAC | 19 | 32991 | 33006 | 1563 |
| 826812 | 3163 | 3178 | AGAAAGTATGGTGCAA | 24 | 32992 | 33007 | 1564 |
| 826813 | 3204 | 3219 | GGCACTTACAGTCTAG | 16 | 33033 | 33048 | 1565 |
| 826814 | 3208 | 3223 | GCAAGGCACTTACAGT | 31 | 33037 | 33052 | 1566 |
| 826815 | 3210 | 3225 | CCGCAAGGCACTTACA | 37 | 33039 | 33054 | 1567 |
| 826816 | 3211 | 3226 | ACCGCAAGGCACTTAC | 21 | 33040 | 33055 | 1568 |
| 826819 | 3214 | 3229 | CTGACCGCAAGGCACT | 23 | 33043 | 33058 | 1569 |
| 826820 | 3215 | 3230 | CCTGACCGCAAGGCAC | 38 | 33044 | 33059 | 1570 |
| 826829 | 3226 | 3241 | AAGATTCAGTCCCTGA | 27 | 33055 | 33070 | 1571 |
| 826830 | 3228 | 3243 | GCAAGATTCAGTCCCT | 25 | 33057 | 33072 | 1572 |
| 826831 | 3229 | 3244 | GGCAAGATTCAGTCCC | 21 | 33058 | 33073 | 1573 |
| 826832 | 3230 | 3245 | GGGCAAGATTCAGTCC | 41 | 33059 | 33074 | 1574 |
| 826833 | 3231 | 3246 | CGGGCAAGATTCAGTC | 29 | 33060 | 33075 | 1575 |
| 826834 | 3232 | 3247 | ACGGGCAAGATTCAGT | 29 | 33061 | 33076 | 1576 |
| 826835 | 3233 | 3248 | AACGGGCAAGATTCAG | 23 | 33062 | 33077 | 1577 |
| 826838 | 3237 | 3252 | CATAAACGGGCAAGAT | 47 | 33066 | 33081 | 1578 |
| 826839 | 3238 | 3253 | ACATAAACGGGCAAGA | 42 | 33067 | 33082 | 1579 |
| 826848 | 3256 | 3271 | GGGCTAGACATGGAGC | 20 | 33085 | 33100 | 1580 |
| 826849 | 3259 | 3274 | GATGGGCTAGACATGG | 26 | 33088 | 33103 | 1581 |
| 826850 | 3261 | 3276 | ATGATGGGCTAGACAT | 34 | 33090 | 33105 | 1582 |
| 826851 | 3275 | 3290 | TTGCTCCAAGCAGGAT | 39 | 33104 | 33119 | 1583 |
| 826852 | 3276 | 3291 | CTTGCTCCAAGCAGGA | 75 | 33105 | 33120 | 1584 |
| 826853 | 3279 | 3294 | CTACTTGCTCCAAGCA | 66 | 33108 | 33123 | 1585 |
| 826854 | 3281 | 3296 | GCCTACTTGCTCCAAG | 53 | 33110 | 33125 | 1586 |
| 826855 | 3292 | 3307 | ATTGAGCTCCTGCCTA | 42 | 33121 | 33136 | 1587 |
| 826858 | N/A | N/A | TACCTCCCCTTGGAAG | 95 | 2717 | 2732 | 1588 |
| 826859 | N/A | N/A | GATACCTCCCCTTGGA | 43 | 2719 | 2734 | 1589 |
| 826868 | N/A | N/A | CCCTTGATACTGCTCA | 48 | N/A | N/A | 1590 |
| 826869 | N/A | N/A | CCCCTTGATACTGCTC | 89 | N/A | N/A | 1591 |
| 826870 | N/A | N/A | TGTTCCCCTTGATACT | 61 | N/A | N/A | 1592 |
| 826871 | N/A | N/A | TTGTTCCCCTTGATAC | 70 | N/A | N/A | 1593 |
| 826872 | N/A | N/A | CTTGTTCCCCTTGATA | 27 | N/A | N/A | 1594 |
| 826873 | N/A | N/A | AGCTTGTTCCCCTTGA | 25 | N/A | N/A | 1595 |
| 826874 | N/A | N/A | CAGCTTGTTCCCCTTG | 40 | N/A | N/A | 1596 |
| 826875 | N/A | N/A | CCAGCTTGTTCCCCTT | 51 | 5161 | 5176 | 1597 |
| 826878 | N/A | N/A | TTGTCCTAGCACCTCC | 25 | 4870 | 4885 | 1598 |
| 826879 | N/A | N/A | GTTTTGTCCTAGCACC | 21 | 4873 | 4888 | 1599 |
| 826888 | N/A | N/A | GATAGGGCCACCTTTC | 31 | 4891 | 4906 | 1600 |
| 826889 | N/A | N/A | CTGATAGGGCCACCTT | 38 | 4893 | 4908 | 1601 |
| 826890 | N/A | N/A | TCCCTGATAGGGCCAC | 24 | 4896 | 4911 | 1602 |
| 826891 | N/A | N/A | CTTCCCTGATAGGGCC | 15 | 4898 | 4913 | 1603 |
| 826892 | N/A | N/A | TGCTTCCCTGATAGGG | 32 | 4900 | 4915 | 1604 |
| 826893 | N/A | N/A | AACGGCCTCTCCTCTG | 18 | 4914 | 4929 | 1605 |
| 826894 | N/A | N/A | GAACGGCCTCTCCTCT | 27 | 4915 | 4930 | 1606 |
| 826895 | N/A | N/A | TAGAACGGCCTCTCCT | 50 | 4917 | 4932 | 1607 |
| 826898 | N/A | N/A | GGCTTCCCTAGAACGG | 44 | 4925 | 4940 | 1608 |
| 826899 | N/A | N/A | CTGGGCTTCCCTAGAA | 47 | 4928 | 4943 | 1609 |
| 826908 | N/A | N/A | GGGCCAAAAGTGCCGG | 49 | 4946 | 4961 | 1610 |
| 826909 | N/A | N/A | GACCTGCGGGAGTTGG | 42 | 4961 | 4976 | 1611 |
| 826910 | N/A | N/A | CAGACCTGCGGGAGTT | 18 | 4963 | 4978 | 1612 |
| 826911 | N/A | N/A | GCAGACCTGCGGGAGT | 26 | 4964 | 4979 | 1613 |
| 826912 | N/A | N/A | GCGCCCACATTCTCCC | 48 | 5015 | 5030 | 1614 |
| 826913 | N/A | N/A | CCTGCGCCCACATTCT | 55 | 5018 | 5033 | 1615 |
| 826914 | N/A | N/A | CCCTGCGCCCACATTC | 68 | 5019 | 5034 | 1616 |
| 826915 | N/A | N/A | CCACCCTGCGCCCACA | 48 | 5022 | 5037 | 1617 |
| 826918 | N/A | N/A | GGAACCCGAGTGAGGC | 30 | 5060 | 5075 | 1618 |
| 826919 | N/A | N/A | TGGAACCCGAGTGAGG | 41 | 5061 | 5076 | 1619 |
| 826928 | N/A | N/A | CCCTTCATGAGCCCCG | 23 | 5150 | 5165 | 1620 |
| 826929 | N/A | N/A | CCCCTTCATGAGCCCC | 42 | 5151 | 5166 | 1621 |
| 826930 | N/A | N/A | AGCTTGTTCCCCTTCA | 23 | 5159 | 5174 | 1622 |
| 826931 | N/A | N/A | CAGCTTGTTCCCCTTC | 35 | 5160 | 5175 | 1623 |
| 826932 | N/A | N/A | CTGCAATAAGGTGCTC | 106 | 2302 | 2317 | 1624 |
| 826933 | N/A | N/A | GGGCATGGTCCTCCCT | 54 | 2316 | 2331 | 1625 |
| 826934 | N/A | N/A | GTCCTTACATTGGGCA | 71 | 2327 | 2342 | 1626 |
| 826935 | N/A | N/A | CCTAGAAACTCCAGTC | 75 | 2356 | 2371 | 1627 |
| 826938 | N/A | N/A | GGCTATCTACTTAGCG | 86 | 2568 | 2583 | 1628 |
| 826939 | N/A | N/A | ATAGGAGCAGAGCTAT | 101 | 2616 | 2631 | 1629 |
| 826948 | N/A | N/A | GTGCAGATCTCAGATT | 58 | 3013 | 3028 | 1630 |
| 826949 | N/A | N/A | ACGGACTTCTAACAAA | 73 | 3030 | 3045 | 1631 |
| 826950 | N/A | N/A | AGGAGATAGGCCTGCA | 73 | 3097 | 3112 | 1632 |
| 826951 | N/A | N/A | ATTGATACACACCGGG | 59 | 3115 | 3130 | 1633 |
| 826952 | N/A | N/A | GTGCAGGAATGTGGTC | 90 | 3185 | 3200 | 1634 |
| 826953 | N/A | N/A | GGGAAGGCTGCCGCTT | 41 | 3231 | 3246 | 1635 |
| 826954 | N/A | N/A | CTGCACGCGGCAGGGA | 62 | 3243 | 3258 | 1636 |
| 826955 | N/A | N/A | AGGAGACTCGGGAGAG | 90 | 3279 | 3294 | 1637 |
| 826958 | N/A | N/A | AAGGAGTGGAGTGCCA | 90 | 3350 | 3365 | 1638 |
| 826959 | N/A | N/A | CCAAACTTAATGCAGC | 54 | 3374 | 3389 | 1639 |
| 826968 | N/A | N/A | CCAAAGGGAGTCTGTC | 59 | 3981 | 3996 | 1640 |
| 826969 | N/A | N/A | GCAAATAGAAGGAGCC | 76 | 4001 | 4016 | 1641 |
| 826970 | N/A | N/A | ACTGAGTGAGTAGAGG | 67 | 4039 | 4054 | 1642 |
| 826971 | N/A | N/A | GGGACAGCGAAGGACA | 54 | 4079 | 4094 | 1643 |
| 826972 | N/A | N/A | ACAATAGAGAGGGACA | 99 | 4089 | 4104 | 1644 |
| 826973 | N/A | N/A | GCCCACAGCTAGGAGG | 66 | 4185 | 4200 | 1645 |
| 826974 | N/A | N/A | AGTAGAAGGATCCTGA | 50 | 4201 | 4216 | 1646 |
| 826975 | N/A | N/A | TGGCAGCCAAACCTCT | 69 | 4509 | 4524 | 1647 |
| 826978 | N/A | N/A | TCCCAGGTTGCGGCTG | 42 | 4555 | 4570 | 1648 |
| 826979 | N/A | N/A | CCTGACCTCGAGCTGT | 38 | 4639 | 4654 | 1649 |
| 826988 | N/A | N/A | CTTATACTTTGCTGGC | 25 | 5994 | 6009 | 1650 |
| 826989 | N/A | N/A | GGTGGACGAGGTCTTA | 27 | 6006 | 6021 | 1651 |
| 826990 | N/A | N/A | CTGCACGGTCTCGCCT | 43 | 6064 | 6079 | 1652 |
| 826991 | N/A | N/A | CCCTAACCTCCACGAT | 73 | 6150 | 6165 | 1653 |
| 826992 | N/A | N/A | CTGTAAGGCCCCTGCC | 43 | 6190 | 6205 | 1654 |
| 826993 | N/A | N/A | GGGCAGGTCAACAGTG | 30 | 6282 | 6297 | 1655 |
| 826994 | N/A | N/A | GTAAAGGTCAGGCACC | 42 | 6299 | 6314 | 1656 |
| 826995 | N/A | N/A | CCGATGAAACCCAAAA | 59 | 6336 | 6351 | 1657 |
| 826998 | N/A | N/A | CGCTTACCACCTGCTC | 38 | 6383 | 6398 | 1658 |
| 826999 | N/A | N/A | AGGTATACAAAAGCAC | 98 | 6425 | 6440 | 1659 |
| 827008 | N/A | N/A | GTACACTAACTCACCA | 53 | 6657 | 6672 | 1660 |
| 827009 | N/A | N/A | AAAGATTTTGCACTCC | 42 | 6679 | 6694 | 1661 |
| 827010 | N/A | N/A | ACCCACACCCATAAAG | 98 | 6691 | 6706 | 1662 |
| 827011 | N/A | N/A | TACCAATATGTGCACA | 44 | 6718 | 6733 | 1663 |
| 827012 | N/A | N/A | TCGCACACATACCAAT | 78 | 6727 | 6742 | 1664 |
| 827013 | N/A | N/A | CAGCATCCAAAATCGC | 44 | 6739 | 6754 | 1665 |
| 827014 | N/A | N/A | ACCCACAGCATGACCA | 68 | 6760 | 6775 | 1666 |
| 827015 | N/A | N/A | ATGGAATATACGAAGG | 35 | 6783 | 6798 | 1667 |
| 827018 | N/A | N/A | ACGAAATGACCTGGCT | 35 | 6870 | 6885 | 1668 |
| 827019 | N/A | N/A | GGACATTATACAGACG | 30 | 6893 | 6908 | 1669 |
| 827028 | N/A | N/A | CACCAAGGCCTAAAGG | 62 | 7268 | 7283 | 1670 |
| 827029 | N/A | N/A | GGCCTCACCCGATTCA | 47 | 7395 | 7410 | 1671 |
| 827030 | N/A | N/A | TAAGAACTGTGCAGGC | 5 | 7408 | 7423 | 1672 |
| 827031 | N/A | N/A | GTCTAGGGCCCCGCAT | 47 | 7435 | 7450 | 1673 |
| 827032 | N/A | N/A | GGGCTTATGGCTTCCT | 42 | 7473 | 7488 | 1674 |
| 827033 | N/A | N/A | CTCCTAGGTGTTCTCT | 38 | 7576 | 7591 | 1675 |
| 827034 | N/A | N/A | TGCTTGGTGGGTGTTG | 53 | 7638 | 7653 | 1676 |
| 827035 | N/A | N/A | GTCAAGTGTGTAGTGC | 14 | 7651 | 7666 | 1677 |
| 827038 | N/A | N/A | GCTTTAGGGTGTAGCA | 76 | 7684 | 7699 | 1678 |
| 827039 | N/A | N/A | GTCTATAAAGCACCCA | 36 | 7700 | 7715 | 1679 |
| 827048 | N/A | N/A | GCTAACCTGAGATGCC | 64 | 8501 | 8516 | 1680 |
| 827049 | N/A | N/A | CACTAGGTTTGTGACT | 82 | 8522 | 8537 | 1681 |
| 827050 | N/A | N/A | AATCAAACACACTAGG | 46 | 8531 | 8546 | 1682 |
| 827051 | N/A | N/A | GGGTAAAAGAGCTTTG | 25 | 8548 | 8563 | 1683 |
| 827052 | N/A | N/A | CCCTACTTAAAGTGTA | 83 | 8566 | 8581 | 1684 |
| 827053 | N/A | N/A | CCTGACAAATTGTCCT | 26 | 8651 | 8666 | 1685 |
| 827054 | N/A | N/A | TTAACCTGTTTACCTC | 38 | 8717 | 8732 | 1686 |
| 827055 | N/A | N/A | TCCCGGTGATTCACTC | 61 | 8744 | 8759 | 1687 |
| 827058 | N/A | N/A | AACCGATCTCCTCGGT | 110 | 8778 | 8793 | 1688 |
| 827059 | N/A | N/A | CTCTAGCTCATCAACC | 69 | 8790 | 8805 | 1689 |
| 827068 | N/A | N/A | CCCTAGCTCAGGGCTT | 48 | 9259 | 9274 | 1690 |
| 827069 | N/A | N/A | ACAGGCAGGGACGGCC | 32 | 9276 | 9291 | 1691 |
| 827070 | N/A | N/A | ATGCAGGGTCTGCCCG | 42 | 9307 | 9322 | 1692 |
| 827071 | N/A | N/A | CTCAACAGTCCAGGCT | 29 | 9376 | 9391 | 1693 |
| 827072 | N/A | N/A | GGTTAGAGGGATGTCA | 48 | 9395 | 9410 | 1694 |
| 827073 | N/A | N/A | CACCATGGCAGGGTTA | 45 | 9406 | 9421 | 1695 |
| 827074 | N/A | N/A | AGCCGCCTAGGCCCCA | 40 | 9449 | 9464 | 1696 |
| 827075 | N/A | N/A | GCCCATGCTCATCCTA | 62 | 9476 | 9491 | 1697 |
| 827078 | N/A | N/A | CCAGGACGGAGCAGCA | 60 | 9585 | 9600 | 1698 |
| 827079 | N/A | N/A | AACTAGGCAAATTCCC | 41 | 9775 | 9790 | 1699 |
| 827088 | N/A | N/A | TAGAAATTCCTATAGC | 46 | 10104 | 10119 | 1700 |
| 827089 | N/A | N/A | CATGACCCCGTGATAC | 50 | 10120 | 10135 | 1701 |
| 827090 | N/A | N/A | TATGATAAATTAGCCG | 57 | 10310 | 10325 | 1702 |
| 827091 | N/A | N/A | GACGGAATGGCCGGGC | 24 | 10443 | 10458 | 1703 |
| 827092 | N/A | N/A | TGGCATAAGATAAGAC | 34 | 10456 | 10471 | 1704 |
| 827093 | N/A | N/A | CCAAAAGGTCTACTGC | 30 | 10482 | 10497 | 1705 |
| 827094 | N/A | N/A | TTCCATAGGGCCCCAC | 50 | 10508 | 10523 | 1706 |
| 827095 | N/A | N/A | CACTGATGAGCCCCCC | 82 | 10601 | 10616 | 1707 |
| 827098 | N/A | N/A | CCTGCCACCCTACGCG | 44 | 10636 | 10651 | 1708 |
| 827099 | N/A | N/A | TCCTGCCACCCTACGC | 46 | 10637 | 10652 | 1709 |
| | | | | | 10660 | 10675 | |
| | | | | | 10683 | 10698 | |
| 827108 | N/A | N/A | CCCTACACGCCTCCCT | 56 | 10721 | 10736 | 1710 |
| 827109 | N/A | N/A | TCAATCTGGTTGTCAC | 55 | 10812 | 10827 | 1711 |
| 827110 | N/A | N/A | TCTCATCACCAACTTC | 42 | 10826 | 10841 | 1712 |
| 827111 | N/A | N/A | AAGAATCCAGATCCCC | 36 | 10943 | 10958 | 1713 |
| 827112 | N/A | N/A | AGCGAATTTGCCTTTC | 34 | 10974 | 10989 | 1714 |
| 827113 | N/A | N/A | GCCCAGGAAAGCGAAT | 55 | 10983 | 10998 | 1715 |
| 827114 | N/A | N/A | TCGACTATCAGGAAGA | 42 | 11015 | 11030 | 1716 |
| 827115 | N/A | N/A | GTGAAGAGACCATCGA | 41 | 11027 | 11042 | 1717 |
| 827118 | N/A | N/A | GGGTAAGTGACCCAGC | 48 | 11154 | 11169 | 1718 |
| 827119 | N/A | N/A | GAAAGAGCACCCAAGC | 51 | 11233 | 11248 | 1719 |
| 827128 | N/A | N/A | TACCGTTAGCCACTGT | 30 | 11418 | 11433 | 1720 |
| 827129 | N/A | N/A | CTTCAGTGTAACACAG | 41 | 11436 | 11451 | 1721 |
| 827130 | N/A | N/A | CTTTAGGACAAACTTT | 62 | 11503 | 11518 | 1722 |
| 827131 | N/A | N/A | TCACTATGCATGAAGA | 14 | 11522 | 11537 | 1723 |
| 827132 | N/A | N/A | ATCTAGTTAGGTGGCA | 32 | 11540 | 11555 | 1724 |
| 827133 | N/A | N/A | AGGTAGGTTATAGTGT | 22 | 11564 | 11579 | 1725 |
| 827134 | N/A | N/A | TGCTATAAAGGTAGGT | 18 | 11572 | 11587 | 1726 |
| 827135 | N/A | N/A | TGACAAGTGGGCTGCC | 44 | 11612 | 11627 | 1727 |
| 827138 | N/A | N/A | GTACAGAAACACCCGG | 63 | 11669 | 11684 | 1728 |
| 827139 | N/A | N/A | AACGGAAGTAAGGTAC | 47 | 11681 | 11696 | 1729 |
| 827148 | N/A | N/A | CGTTTATCGAGCACTT | 13 | 11915 | 11930 | 1730 |
| 827149 | N/A | N/A | GGCAAGCATAGCTAGC | 18 | 11930 | 11945 | 1731 |
| 827150 | N/A | N/A | GTGTGTTTGGCATTCT | 7 | 11980 | 11995 | 1732 |
| | | | | | 13086 | 13101 | |
| 827151 | N/A | N/A | GGTGTGTTTGGCATTC | 12 | 11981 | 11996 | 1733 |
| 827152 | N/A | N/A | AGGTGTGTTTGGCATT | 29 | 11982 | 11997 | 1734 |
| 827153 | N/A | N/A | GCCTTAGGCATCAGCT | 25 | 12045 | 12060 | 1735 |
| 827154 | N/A | N/A | GTCTAGCTGGCTGGGC | 42 | 12059 | 12074 | 1736 |
| 827155 | N/A | N/A | AACCACCGTCTAGTCC | 42 | 12126 | 12141 | 1737 |
| 827158 | N/A | N/A | CCAGAACAAGGTTGTT | 56 | 12181 | 12196 | 1738 |
| 827159 | N/A | N/A | TCAGATTTAATGGGTC | 43 | 12207 | 12222 | 1739 |
| 827168 | N/A | N/A | AACCAGTTGATAGAGA | 44 | 12409 | 12424 | 1740 |
| 827169 | N/A | N/A | ATACGAATTCTATGAA | 80 | 12432 | 12447 | 1741 |
| 827170 | N/A | N/A | TCCCATTTATACGAAT | 57 | 12440 | 12455 | 1742 |
| 827171 | N/A | N/A | CCAGAATAGGCTCATC | 33 | 12570 | 12585 | 1743 |
| 827172 | N/A | N/A | GCTCAAATCAGGCAGC | 89 | 12593 | 12608 | 1744 |
| 827173 | N/A | N/A | GCAAAGAACGATGCTC | 46 | 12605 | 12620 | 1745 |
| 827174 | N/A | N/A | TAACAGAGTTGACTTG | 100 | 12622 | 12637 | 1746 |
| 827175 | N/A | N/A | TGGTATTAGAATGTGC | 18 | 12681 | 12696 | 1747 |
| 827178 | N/A | N/A | ACGACGAAACCTTGTA | 48 | 12932 | 12947 | 1748 |
| 827179 | N/A | N/A | GTGTTTGGCATTCTAG | 19 | 13084 | 13099 | 1749 |
| 827188 | N/A | N/A | ACCATATAACCCATCC | 30 | 13715 | 13730 | 1750 |
| 827189 | N/A | N/A | ATGATACGATCATTTT | 34 | 13774 | 13789 | 1751 |
| 827190 | N/A | N/A | CTGTACACAGCTAGTG | 30 | 13800 | 13815 | 1752 |
| 827191 | N/A | N/A | CGAACAGACCTACATT | 41 | 13833 | 13848 | 1753 |
| 827192 | N/A | N/A | CCGAACAGACCTACAT | 32 | 13834 | 13849 | 1754 |
| 827193 | N/A | N/A | AGCCGAACAGACCTAC | 42 | 13836 | 13851 | 1755 |
| 827194 | N/A | N/A | TGAACAGACCTACATT | 52 | 14130 | 14145 | 1756 |
| 827195 | N/A | N/A | ATGAACAGACCTACAT | 60 | 14131 | 14146 | 1757 |
| 827198 | N/A | N/A | AGTAGGCACTTTATGA | 55 | 14143 | 14158 | 1758 |
| 827199 | N/A | N/A | CCGTATGTAGTAGGCA | 24 | 14151 | 14166 | 1759 |
| 827207 | N/A | N/A | CTAGAACAACCGTATG | 29 | 14160 | 14175 | 1760 |
| 827208 | N/A | N/A | CCTAGAACAACCGTAT | 34 | 14161 | 14176 | 1761 |
| 827209 | N/A | N/A | CCCTAGAACAACCGTA | 30 | 14162 | 14177 | 1762 |
| 827210 | N/A | N/A | TCCCTAGAACAACCGT | 21 | 14163 | 14178 | 1763 |
| 827211 | N/A | N/A | TGGAAGATATCTTCCT | 90 | 14229 | 14244 | 1764 |
| 827212 | N/A | N/A | CCTTATGCTATACAGG | 43 | 14311 | 14326 | 1765 |
| 827213 | N/A | N/A | GAATACTGTATTGGAA | 43 | 14349 | 14364 | 1766 |
| 827214 | N/A | N/A | TGTTAGCAGGTTCTGC | 48 | 14375 | 14390 | 1767 |
| 827217 | N/A | N/A | ACAATGCGGTTCTTGG | 38 | 14507 | 14522 | 1768 |
| 827218 | N/A | N/A | CTAAGACTTATCTGGA | 48 | 14629 | 14644 | 1769 |
| 827227 | N/A | N/A | TGCATTTAGGCCGGGT | 46 | 15520 | 15535 | 1770 |
| 827228 | N/A | N/A | TTGCAGGGTACACAAC | 51 | 15557 | 15572 | 1771 |
| 827229 | N/A | N/A | CTGAACAAGGTTGCAG | 46 | 15567 | 15582 | 1772 |
| 827230 | N/A | N/A | TAGAACTAACAAACTG | 53 | 15580 | 15595 | 1773 |
| 827231 | N/A | N/A | GGCCTGAGGGATGTCA | 52 | 15617 | 15632 | 1774 |
| 827232 | N/A | N/A | CATCATGAAAGTCCAG | 39 | 15641 | 15656 | 1775 |
| 827233 | N/A | N/A | CACCGAAATCAAGAGT | 58 | 15834 | 15849 | 1776 |
| 827234 | N/A | N/A | TGCCGCTTGGCACCGA | 96 | 15844 | 15859 | 1777 |
| 827237 | N/A | N/A | ACCCAGGTCATCCCGC | 106 | 15902 | 15917 | 1778 |
| 827238 | N/A | N/A | ACCCGGAACTTGTCTG | 45 | 15971 | 15986 | 1779 |
| 827247 | N/A | N/A | CCCAAAAGCTTGGGCA | 40 | 16752 | 16767 | 1780 |
| 827248 | N/A | N/A | ACATAGGACCCCAGGG | 37 | 16775 | 16790 | 1781 |
| 827249 | N/A | N/A | TCCCACTAGTGGGCAC | 53 | 16919 | 16934 | 1782 |
| 827250 | N/A | N/A | TCCTAACTGAGTCCCA | 32 | 16930 | 16945 | 1783 |
| 827251 | N/A | N/A | TCACGCTGGAGGGTCC | 31 | 16943 | 16958 | 1784 |
| 827252 | N/A | N/A | TGTTAGCCCAGTTCTC | 44 | 16961 | 16976 | 1785 |
| 827253 | N/A | N/A | CTATCTTGGGCTGTTA | 93 | 16972 | 16987 | 1786 |
| 827254 | N/A | N/A | GGCAGACGAGCTCACT | 11 | 17288 | 17303 | 1787 |
| 827257 | N/A | N/A | GACTGAGGGATCAAGA | 44 | 17431 | 17446 | 1788 |
| 827258 | N/A | N/A | TGCCTAGGGTGGAAGG | 42 | 17481 | 17496 | 1789 |
| 827267 | N/A | N/A | GACTAGAGTCAGAGGG | 43 | 17733 | 17748 | 1790 |
| 827268 | N/A | N/A | TCTGGTTGCACTGGAC | 28 | 17754 | 17769 | 1791 |
| 827269 | N/A | N/A | TTCTGGTTGCACTGGA | 38 | 17755 | 17770 | 1792 |
| 827270 | N/A | N/A | GTTCTGGTTGCACTGG | 15 | 17756 | 17771 | 1793 |
| 827271 | N/A | N/A | TGTTCTGGTTGCACTG | 37 | 17757 | 17772 | 1794 |
| 827272 | N/A | N/A | TTGTTCTGGTTGCACT | 32 | 17758 | 17773 | 1795 |
| 827273 | N/A | N/A | GCGGACCCCGCGGAGA | 39 | 17978 | 17993 | 1796 |
| 827274 | N/A | N/A | ACCCAGGGAAGCGGAC | 60 | 17988 | 18003 | 1797 |
| 827277 | N/A | N/A | ATCCATGCTTCCAGCC | 14 | 18214 | 18229 | 1798 |
| 827278 | N/A | N/A | GATCCATGCTTCCAGC | 19 | 18215 | 18230 | 1799 |
| 827286 | N/A | N/A | AAAGACCAAGATCCAT | 19 | 18224 | 18239 | 1800 |
| 827287 | N/A | N/A | GGCCTTAGAAAGACCA | 86 | 18551 | 18566 | 1801 |
| 827288 | N/A | N/A | AGCTTTGATGCTAGGG | 16 | 18574 | 18589 | 1802 |
| 827289 | N/A | N/A | GACAGATGATCTCCTA | 11 | 18600 | 18615 | 1803 |
| 827290 | N/A | N/A | CCTCACTACTACTGCC | 21 | 18643 | 18658 | 1804 |
| 827291 | N/A | N/A | CCTCAACCCATGCCAC | 51 | 18663 | 18678 | 1805 |
| 827292 | N/A | N/A | ACTTAGGTTTAGTCCC | 36 | 18677 | 18692 | 1806 |
| 827293 | N/A | N/A | AGCTAGAGTGGGAACT | 44 | 18690 | 18705 | 1807 |
| 827296 | N/A | N/A | TGATACATCCAGAGTC | 42 | 18726 | 18741 | 1808 |
| 827297 | N/A | N/A | ATGATGTGATACATCC | 58 | 18732 | 18747 | 1809 |
| 827305 | N/A | N/A | ACACACTTGGTACAGC | 23 | 18964 | 18979 | 1810 |
| 827306 | N/A | N/A | GGTCTATAAAGTGCCC | 23 | 18995 | 19010 | 1811 |
| 827307 | N/A | N/A | AGAGTAATGAAACCCA | 5 | 19022 | 19037 | 1812 |
| 827308 | N/A | N/A | CTTCACCTGTTTGAGT | 32 | 19041 | 19056 | 1813 |
| 827309 | N/A | N/A | TCCTTAGCCAGGGCCG | 15 | 19079 | 19094 | 1814 |
| 827310 | N/A | N/A | AATGAATACCCGAGGG | 25 | 19113 | 19128 | 1815 |
| 827311 | N/A | N/A | GGACATTATAACAGGG | 28 | 19139 | 19154 | 1816 |
| 827312 | N/A | N/A | CTGCTATGAGCTGCTT | 71 | 19159 | 19174 | 1817 |
| 827315 | N/A | N/A | CTGTAGAGTGGAGCCA | 44 | 19305 | 19320 | 1818 |
| 827316 | N/A | N/A | AGGGAATGCCCCCTGT | 95 | 19317 | 19332 | 1819 |
| 827325 | N/A | N/A | GGCATAGGGAAAGCAC | 30 | 19542 | 19557 | 1820 |
| | | | | | 19624 | 19639 | |
| 827326 | N/A | N/A | GAGGCATCGGGTGAGG | 47 | 19557 | 19572 | 1821 |
| 827327 | N/A | N/A | GGACTTTCTGTTGATG | 21 | 19598 | 19613 | 1822 |
| 827328 | N/A | N/A | TGGACTTTCTGTTGAT | 29 | 19599 | 19614 | 1823 |
| 827329 | N/A | N/A | CCATGGACTTTCTGTT | 32 | 19602 | 19617 | 1824 |
| | | | | | 19685 | 19700 | |
| 827330 | N/A | N/A | TCCATGGACTTTCTGT | 28 | 19603 | 19618 | 1825 |
| 827331 | N/A | N/A | GTCCATGGACTTTCTG | 41 | 19604 | 19619 | 1826 |
| 827332 | N/A | N/A | GGACTTTCTGTTGAGG | 30 | 19681 | 19696 | 1827 |
| 827335 | N/A | N/A | CGCCTAAGTGCCAAGA | 26 | 19711 | 19726 | 1828 |
| 827336 | N/A | N/A | AGCAATGAGGCTCTGA | 30 | 19738 | 19753 | 1829 |
| 827345 | N/A | N/A | ATAGTTACATGTGGTG | 25 | 19863 | 19878 | 1830 |
| 827346 | N/A | N/A | AATAGTTACATGTGGT | 30 | 19864 | 19879 | 1831 |
| 827347 | N/A | N/A | GAATAGTTACATGTGG | 13 | 19865 | 19880 | 1832 |
| 827348 | N/A | N/A | TGGAATAGTTACATGT | 18 | 19867 | 19882 | 1833 |
| 827349 | N/A | N/A | CTGGAATAGTTACATG | 25 | 19868 | 19883 | 1834 |
| 827350 | N/A | N/A | ACTGGAATAGTTACAT | 47 | 19869 | 19884 | 1835 |
| 827351 | N/A | N/A | TAACTGGAATAGTTAC | 93 | 19871 | 19886 | 1836 |
| 827354 | N/A | N/A | GACTAACTGGAATAGT | 73 | 19874 | 19889 | 1837 |
| 827355 | N/A | N/A | GGACTAACTGGAATAG | 33 | 19875 | 19890 | 1838 |
| 827364 | N/A | N/A | GGAGGATACAGTTTGG | 32 | 20588 | 20603 | 1839 |
| 827365 | N/A | N/A | ACACTGAACGATTTTA | 32 | 20608 | 20623 | 1840 |
| 827366 | N/A | N/A | CTGGAGGCCGTGAGAG | 45 | 20624 | 20639 | 1841 |
| 827367 | N/A | N/A | ACCAACTTGATGCTGG | 51 | 20636 | 20651 | 1842 |
| 827368 | N/A | N/A | GGTGAGAAAGCCATGC | 17 | 20660 | 20675 | 1843 |
| 827369 | N/A | N/A | GAAAAGGGTGTAGTTA | 35 | 20690 | 20705 | 1844 |
| 827370 | N/A | N/A | AAACAGGTAGTGGTAA | 27 | 20826 | 20841 | 1845 |
| 827371 | N/A | N/A | GTGAAATGTCCACCAC | 38 | 20962 | 20977 | 1846 |
| 827374 | N/A | N/A | GCAAAAATGTGGGCCG | 50 | 21420 | 21435 | 1847 |
| 827375 | N/A | N/A | TCCATGTACAGGATCC | 38 | 21529 | 21544 | 1848 |
| 827383 | N/A | N/A | TAAGATGGCTAAAGTC | 13 | 21733 | 21748 | 1849 |
| 827384 | N/A | N/A | GGATTCATTAAGATGG | 23 | 21741 | 21756 | 1850 |
| 827385 | N/A | N/A | GGGATTCATTAAGATG | 31 | 21742 | 21757 | 1851 |
| 827386 | N/A | N/A | AGGGATTCATTAAGAT | 14 | 21743 | 21758 | 1852 |
| 827387 | N/A | N/A | CAGGGATTCATTAAGA | 30 | 21744 | 21759 | 1853 |
| 827388 | N/A | N/A | ACAGGGATTCATTAAG | 38 | 21745 | 21760 | 1854 |
| 827389 | N/A | N/A | TACAGGGATTCATTAA | 42 | 21746 | 21761 | 1855 |
| 827390 | N/A | N/A | TTACAGGGATTCATTA | 55 | 21747 | 21762 | 1856 |
| 827393 | N/A | N/A | TACGATTACAGGGATT | 11 | 21752 | 21767 | 1857 |
| 827402 | N/A | N/A | GCTCACTAGTACGATT | 43 | 21761 | 21776 | 1858 |
| 827403 | N/A | N/A | AGCTCACTAGTACGAT | 46 | 21762 | 21777 | 1859 |
| 827404 | N/A | N/A | GCCTTAGTAAGAGCTG | 29 | 21782 | 21797 | 1860 |
| 827405 | N/A | N/A | AGTTACTTACTTAATC | 28 | 21896 | 21911 | 1861 |
| 827406 | N/A | N/A | GACCAAACAAGTTACT | 21 | 21905 | 21920 | 1862 |
| 827407 | N/A | N/A | GGACCAAACAAGTTAC | 27 | 21906 | 21921 | 1863 |
| 827408 | N/A | N/A | ATTAGATGTGGGACCA | 17 | 21916 | 21931 | 1864 |
| 827411 | N/A | N/A | TATGAGAATCAGTATA | 56 | 22311 | 22326 | 1865 |
| 827412 | N/A | N/A | CTATGAGAATCAGTAT | 44 | 22312 | 22327 | 1866 |
| 827420 | N/A | N/A | GGAGAAACACGGATGG | 10 | 22743 | 22758 | 1867 |
| 827421 | N/A | N/A | TTCCATCAGCGGTGGA | 52 | 22756 | 22771 | 1868 |
| 827422 | N/A | N/A | GGCACAAGTTCCATCA | 23 | 22764 | 22779 | 1869 |
| 827423 | N/A | N/A | AGGCACAAGTTCCATC | 34 | 22765 | 22780 | 1870 |
| 827424 | N/A | N/A | CAGGCACAAGTTCCAT | 32 | 22766 | 22781 | 1871 |
| 827425 | N/A | N/A | GCAGGCACAAGTTCCA | 19 | 22767 | 22782 | 1872 |
| 827426 | N/A | N/A | AGCAGGCACAAGTTCC | 14 | 22768 | 22783 | 1873 |
| 827427 | N/A | N/A | AAGCAGGCACAAGTTC | 44 | 22769 | 22784 | 1874 |
| 827430 | N/A | N/A | GTGCTGCCCCCATGGA | 37 | 22785 | 22800 | 1875 |
| 827431 | N/A | N/A | GGGACAAGTATAATGG | 58 | 22804 | 22819 | 1876 |
| 827440 | N/A | N/A | AAGCAGGTCATTGTTT | 28 | 23071 | 23086 | 1877 |
| 827441 | N/A | N/A | TGGTTGTACGGTCTCA | 19 | 23086 | 23101 | 1878 |
| 827442 | N/A | N/A | GCAAAGACGGAAAGGG | 34 | 23180 | 23195 | 1879 |
| 827443 | N/A | N/A | GCGACGGGAGCCAGGC | 36 | 23194 | 23209 | 1880 |
| 827444 | N/A | N/A | CTTGGAGCTAGCGACG | 20 | 23204 | 23219 | 1881 |
| 827445 | N/A | N/A | TGCTACCCTGCCATCT | 24 | 23218 | 23233 | 1882 |
| 827446 | N/A | N/A | TGCCACACGGCACAGA | 64 | 23248 | 23263 | 1883 |
| 827447 | N/A | N/A | GCAAATCACAGGTTCC | 19 | 23302 | 23317 | 1884 |
| 827449 | N/A | N/A | CATCAGTATGTCTCAG | 18 | 23412 | 23427 | 1885 |
| 827450 | N/A | N/A | GAGGAAGATCAGTACC | 39 | 23451 | 23466 | 1886 |
| 827459 | N/A | N/A | CCCTAACTGCCCATGC | 20 | 23711 | 23726 | 1887 |
| 827460 | N/A | N/A | CGGCATTGACTTCCGT | 48 | 23775 | 23790 | 1888 |
| 827461 | N/A | N/A | TCACACATCTACCTTC | 34 | 23828 | 23843 | 1889 |
| 827462 | N/A | N/A | TTTACTCACACTCCCT | 24 | 23936 | 23951 | 1890 |
| 827463 | N/A | N/A | CCTACAGGACTTGTGC | 26 | 24009 | 24024 | 1891 |
| 827464 | N/A | N/A | AGAGAGAGTAGGGTCA | 64 | 24094 | 24109 | 1892 |
| 827465 | N/A | N/A | TGAGAGTAATTCCTTA | 50 | 24117 | 24132 | 1893 |
| 827466 | N/A | N/A | CACCGTTGTTGATTCC | 39 | 24212 | 24227 | 1894 |
| 827469 | N/A | N/A | GCTCAAGGTAAGTACA | 55 | 24276 | 24291 | 1895 |
| 827470 | N/A | N/A | GCTCTAGGAGGTGAGC | 83 | 24290 | 24305 | 1896 |
| 827479 | N/A | N/A | TCCTACTGGCCTCGCC | 49 | 25036 | 25051 | 1897 |
| 827480 | N/A | N/A | TTCCAGGTTGTATCTC | 53 | 25067 | 25082 | 1898 |
| 827481 | N/A | N/A | ACATACACCAAGAGAT | 16 | 25127 | 25142 | 1899 |
| 827482 | N/A | N/A | CCTATGAACCCACATA | 107 | 25138 | 25153 | 1900 |
| 827483 | N/A | N/A | GGCTGCCACGGAATCA | 50 | 25265 | 25280 | 1901 |
| 827484 | N/A | N/A | ATACACAACCCCTCCA | 104 | 25316 | 25331 | 1902 |
| 827485 | N/A | N/A | GTGAATACACACCTGG | 48 | 25442 | 25457 | 1903 |
| 827486 | N/A | N/A | CCTCAGTGAGTACTGG | 52 | 25602 | 25617 | 1904 |
| 827489 | N/A | N/A | GCCTGCAGGTTGTTTT | 56 | 26100 | 26115 | 1905 |
| 827490 | N/A | N/A | TGAGGAACCGCTGGAG | 41 | 26479 | 26494 | 1906 |
| 827499 | N/A | N/A | TCCAAACTTTACTGAT | 41 | 26787 | 26802 | 1907 |
| 827500 | N/A | N/A | TAAGGAGGAGATTCCA | 38 | 26809 | 26824 | 1908 |
| 827501 | N/A | N/A | GTCCTATACCAGGATA | 47 | 26823 | 26838 | 1909 |
| 827502 | N/A | N/A | AGAGATTTGTCTAGTC | 12 | 26836 | 26851 | 1910 |
| 827503 | N/A | N/A | ACTCAACTGTAGTCAA | 36 | 26858 | 26873 | 1911 |
| 827504 | N/A | N/A | TGGCACACGACTTCCC | 28 | 26883 | 26898 | 1912 |
| 827505 | N/A | N/A | TGCAAACCCTTGCAGC | 80 | 26942 | 26957 | 1913 |
| 827506 | N/A | N/A | CACTACCATGTCCCCT | 60 | 27075 | 27090 | 1914 |
| 827509 | N/A | N/A | TGCGGAGCCAGCCCAG | 43 | 27202 | 27217 | 1915 |
| 827510 | N/A | N/A | CACGACTGGAAAGTCC | 42 | 27232 | 27247 | 1916 |
| 827519 | N/A | N/A | TAATGGAACTGTAGAT | 31 | 27734 | 27749 | 1917 |
| 827520 | N/A | N/A | TATATGATGATTGCAC | 35 | 27883 | 27898 | 1918 |
| 827521 | N/A | N/A | TGCCTGGCTTGAGTGA | 47 | 27944 | 27959 | 1919 |
| 827522 | N/A | N/A | GAGTACAAGGTTTATT | 22 | 28237 | 28252 | 1920 |
| 827523 | N/A | N/A | TGAGTACAAGGTTTAT | 26 | 28238 | 28253 | 1921 |
| 827524 | N/A | N/A | ATGAGTACAAGGTTTA | 7 | 28239 | 28254 | 1922 |
| 827525 | N/A | N/A | GACTTGCTAATGAGTA | 36 | 28248 | 28263 | 1923 |
| 827526 | N/A | N/A | GGACTTGCTAATGAGT | 58 | 28249 | 28264 | 1924 |
| 827529 | N/A | N/A | TTGGGACTTGCTAATG | 53 | 28252 | 28267 | 1925 |
| 827530 | N/A | N/A | GCTTGGGACTTGCTAA | 43 | 28254 | 28269 | 1926 |
| 827538 | N/A | N/A | TTGCTACCATACGGAT | 23 | 28747 | 28762 | 1927 |
| 827539 | N/A | N/A | ATTGCTACCATACGGA | 38 | 28748 | 28763 | 1928 |
| 827540 | N/A | N/A | TATTGCTACCATACGG | 31 | 28749 | 28764 | 1929 |
| 827541 | N/A | N/A | CTATTGCTACCATACG | 17 | 28750 | 28765 | 1930 |
| 827542 | N/A | N/A | GCTATTGCTACCATAC | 31 | 28751 | 28766 | 1931 |
| 827543 | N/A | N/A | CTGCTATTGCTACCAT | 25 | 28753 | 28768 | 1932 |
| 827544 | N/A | N/A | AGGCACTGCTATTGCT | 42 | 28758 | 28773 | 1933 |
| 827545 | N/A | N/A | CCATACAAGGGAGTGT | 66 | 28799 | 28814 | 1934 |
| 827548 | N/A | N/A | TGCTGCTAGGGATGTA | 45 | 29051 | 29066 | 1935 |
| 827549 | N/A | N/A | ACCCATTAAGATGTGT | 92 | 29468 | 29483 | 1936 |
| 827558 | N/A | N/A | CCACACCCAAGAGGTC | 42 | 30527 | 30542 | 1937 |
| 827559 | N/A | N/A | TCCTAGGGCACCTCAG | 100 | 30554 | 30569 | 1938 |
| 827560 | N/A | N/A | TAGCAGTACCCTGTGG | 56 | 30590 | 30605 | 1939 |
| 827561 | N/A | N/A | GGACACTAACCTGCAT | 47 | 30669 | 30684 | 1940 |
| 827562 | N/A | N/A | ACCCAACCTGTACCCG | 57 | 30692 | 30707 | 1941 |
| 827563 | N/A | N/A | GGCTCGGTAACCTGTA | 56 | 30712 | 30727 | 1942 |
| 827564 | N/A | N/A | TCCCAAATGCTTGGCT | 58 | 30724 | 30739 | 1943 |
| 827565 | N/A | N/A | GGCCACAGCATTACAT | 100 | 30879 | 30894 | 1944 |
| 827568 | N/A | N/A | GGCTTACAGGGATAGG | 61 | 30934 | 30949 | 1945 |
| 827569 | N/A | N/A | CCCATATGCTTCAGGC | 61 | 30947 | 30962 | 1946 |
| 827578 | N/A | N/A | CCGACAGCCGCCCTGC | 43 | 31323 | 31338 | 1947 |
| 827579 | N/A | N/A | GGCCGAGCTCCTTCTT | 71 | 31435 | 31450 | 1948 |
| 827580 | N/A | N/A | ACCTTATGCCCCGGCC | 56 | 31447 | 31462 | 1949 |
| 827581 | N/A | N/A | CCCCAGAGACCTTATG | 69 | 31455 | 31470 | 1950 |
| 827582 | N/A | N/A | ACTGATAACTGGCCCA | 58 | 31549 | 31564 | 1951 |
| 827583 | N/A | N/A | CACCAAGCTGTCTCCC | 50 | 31655 | 31670 | 1952 |
| 827584 | N/A | N/A | GACGATGGGACAGAGG | 76 | 31711 | 31726 | 1953 |
| 827585 | N/A | N/A | GAGGAGAGGTACATTG | 60 | 31726 | 31741 | 1954 |

TABLE 5
Percent level of human α-ENaC mRNA

| | SEQ | SEQ | | | SEQ | SEQ | |
|---|---|---|---|---|---|---|---|
| Compound | ID: 1 | ID: 1 | | α-ENaC | ID: 2 | ID 2: | SEQ |
| Number | Start Site | Stop Site | Sequence | (% control) | Start Site | Stop Site | ID NO |
| 797469 | N/A | N/A | GGATGATGTGATACAT | 5 | 18734 | 18749 | 400 |
| 797524 | N/A | N/A | ACCATACGGATGAACC | 23 | 28742 | 28757 | 455 |
| 826071 | 5 | 20 | TTTAGACGCAGACAGG | 85 | 4266 | 4281 | 466 |
| 826074 | 25 | 40 | GGCGGACTCTGGGCAG | 74 | 4286 | 4301 | 611 |
| 826075 | 28 | 43 | GAAGGCGGACTCTGGG | 81 | 4289 | 4304 | 612 |
| 826076 | 29 | 44 | AGAAGGCGGACTCTGG | 82 | 4290 | 4305 | 613 |
| 826077 | 31 | 46 | TGAGAAGGCGGACTCT | 85 | 4292 | 4307 | 614 |
| 826078 | 32 | 47 | CTGAGAAGGCGGACTC | 70 | 4293 | 4308 | 615 |
| 826079 | 33 | 48 | CCTGAGAAGGCGGACT | 79 | 4294 | 4309 | 616 |
| 826091 | 53 | 68 | GGTGAACTGGGAGTAC | 111 | 4314 | 4329 | 468 |
| 826094 | 73 | 88 | AAGGAGGGCTCCCGAG | 66 | 4334 | 4349 | 618 |
| 826095 | 74 | 89 | GAAGGAGGGCTCCCGA | 90 | 4335 | 4350 | 619 |
| 826096 | 81 | 96 | TCCGAAGGAAGGAGGG | 81 | 4342 | 4357 | 620 |
| 826097 | 83 | 98 | TTTCCGAAGGAAGGAG | 95 | 4344 | 4359 | 621 |
| 826098 | 85 | 100 | GTTTTCCGAAGGAAGG | 69 | 4346 | 4361 | 622 |
| 826099 | 88 | 103 | GGAGTTTTCCGAAGGA | 105 | 4349 | 4364 | 623 |
| 826111 | 163 | 178 | GCGACAGGAATCTCAT | 90 | 4424 | 4439 | 470 |
| 826114 | 167 | 182 | GGAAGCGACAGGAATC | 60 | 4428 | 4443 | 626 |
| 826115 | 169 | 184 | ATGGAAGCGACAGGAA | 63 | 4430 | 4445 | 627 |
| 826116 | 170 | 185 | GATGGAAGCGACAGGA | 53 | 4431 | 4446 | 628 |
| 826117 | 171 | 186 | GGATGGAAGCGACAGG | 78 | 4432 | 4447 | 629 |
| 826118 | 172 | 187 | GGGATGGAAGCGACAG | 38 | 4433 | 4448 | 630 |
| 826119 | 175 | 190 | CCAGGGATGGAAGCGA | 56 | 4436 | 4451 | 631 |
| 826131 | 216 | 231 | CAGGTGCAGCGGCCTG | 116 | 4477 | 4492 | 472 |
| 826134 | 226 | 241 | GTTCCCCTGACAGGTG | 84 | N/A | N/A | 634 |
| 826135 | 228 | 243 | TTGTTCCCCTGACAGG | 70 | N/A | N/A | 635 |
| 826136 | 229 | 244 | CTTGTTCCCCTGACAG | 60 | N/A | N/A | 636 |
| 826137 | 230 | 245 | GCTTGTTCCCCTGACA | 16 | N/A | N/A | 637 |
| 826138 | 232 | 247 | CAGCTTGTTCCCCTGA | 52 | N/A | N/A | 638 |
| 826139 | 233 | 248 | CCAGCTTGTTCCCCTG | 61 | N/A | N/A | 639 |
| 826151 | 283 | 298 | CCCCTCCATGAGACCT | 26 | 5211 | 5226 | 474 |
| 826154 | 292 | 307 | CAGCTTGTTCCCCTCC | 14 | 5220 | 5235 | 642 |
| 826155 | 310 | 325 | GCTAGAGTCCTGCTCC | 43 | 5238 | 5253 | 643 |
| 826156 | 312 | 327 | GGGCTAGAGTCCTGCT | 78 | 5240 | 5255 | 644 |
| 826157 | 315 | 330 | GGAGGGCTAGAGTCCT | 94 | 5243 | 5258 | 645 |
| 826158 | 320 | 335 | ACTGTGGAGGGCTAGA | 67 | 5248 | 5263 | 646 |
| 826159 | 321 | 336 | GACTGTGGAGGGCTAG | 92 | 5249 | 5264 | 647 |
| 826171 | 355 | 370 | CTCACGCTTGTTCCCC | 34 | 5283 | 5298 | 476 |
| 826174 | 360 | 375 | TGCTCCTCACGCTTGT | 13 | 5288 | 5303 | 650 |
| 826175 | 362 | 377 | CCTGCTCCTCACGCTT | 40 | 5290 | 5305 | 651 |
| 826176 | 363 | 378 | CCCTGCTCCTCACGCT | 20 | 5291 | 5306 | 652 |
| 826177 | 386 | 401 | GCGCCGCAGGTTCGGG | 49 | 5314 | 5329 | 653 |
| 826178 | 405 | 420 | TCCGCCGTGGGCTGCT | 44 | 5333 | 5348 | 654 |
| 826179 | 407 | 422 | CCTCCGCCGTGGGCTG | 36 | 5335 | 5350 | 655 |
| 826191 | 441 | 456 | CGGTAGGAGCGGTGGA | 55 | 5369 | 5384 | 478 |
| 826194 | 446 | 461 | GCTCTCGGTAGGAGCG | 60 | 5374 | 5389 | 658 |
| 826195 | 448 | 463 | GAGCTCTCGGTAGGAG | 61 | 5376 | 5391 | 959 |
| 826196 | 451 | 466 | GAAGAGCTCTCGGTAG | 48 | 5379 | 5394 | 660 |
| 826197 | 453 | 468 | TCGAAGAGCTCTCGGT | 36 | 5381 | 5396 | 661 |
| 826198 | 456 | 471 | AACTCGAAGAGCTCTC | 36 | 5384 | 5399 | 662 |
| 826199 | 457 | 472 | GAACTCGAAGAGCTCT | 49 | 5385 | 5400 | 663 |
| 826210 | 566 | 581 | TCATGCCAAAGGTGCA | 27 | 5494 | 5509 | 480 |
| 826213 | 575 | 590 | GCCAGTACATCATGCC | 9 | 5503 | 5518 | 666 |
| 826214 | 577 | 592 | TTGCCAGTACATCATG | 40 | 5505 | 5520 | 667 |
| 826215 | 580 | 595 | GAATTGCCAGTACATC | 36 | 5508 | 5523 | 668 |
| 826216 | 581 | 596 | CGAATTGCCAGTACAT | 26 | 5509 | 5524 | 669 |
| 826217 | 582 | 597 | CCGAATTGCCAGTACA | 24 | 5510 | 5525 | 670 |
| 826218 | 585 | 600 | AGGCCGAATTGCCAGT | 54 | 5513 | 5528 | 671 |
| 826230 | 607 | 622 | GCTGAAGTACTCTCCG | 47 | 5535 | 5550 | 482 |
| 826233 | 626 | 641 | TGTTGAGGCTGACGGG | 5 | 5554 | 5569 | 674 |
| 826234 | 628 | 643 | GATGTTGAGGCTGACG | 31 | 5556 | 5571 | 675 |
| 826235 | 639 | 654 | GAGTTGAGGTTGATGT | 34 | 5567 | 5582 | 676 |
| 826236 | 641 | 656 | CCGAGTTGAGGTTGAT | 22 | 5569 | 5584 | 677 |
| 826237 | 643 | 658 | GTCCGAGTTGAGGTTG | 30 | 5571 | 5586 | 678 |
| 826238 | 644 | 659 | TGTCCGAGTTGAGGTT | 35 | 5572 | 5587 | 679 |
| 826250 | 703 | 718 | AATTTCCGGGTACCTG | 29 | 16288 | 16303 | 484 |
| 826253 | 731 | 746 | TGCGGTCCAGCTCCTC | 17 | 16316 | 16331 | 682 |
| 826254 | 734 | 749 | TGATGCGGTCCAGCTC | 41 | 16319 | 16334 | 683 |
| 826255 | 737 | 752 | CTGTGATGCGGTCCAG | 49 | 16322 | 16337 | 684 |
| 826256 | 739 | 754 | CTCTGTGATGCGGTCC | 21 | 16324 | 16339 | 685 |
| 826257 | 740 | 755 | GCTCTGTGATGCGGTC | 26 | 16325 | 16340 | 686 |
| 826258 | 759 | 774 | TACAGGTCAAAGAGCG | 24 | 16344 | 16359 | 687 |
| 826268 | 792 | 807 | CCGGCCACGAGAGTGG | 93 | 16377 | 16392 | 486 |
| 826271 | 798 | 813 | CGGGAGCCGGCCACGA | 36 | 16383 | 16398 | 690 |
| 826272 | 800 | 815 | TGCGGGAGCCGGCCAC | 39 | 16385 | 16400 | 691 |
| 826273 | 803 | 818 | GGCTGCGGGAGCCGGC | 61 | 16388 | 16403 | 692 |
| 826274 | 804 | 819 | CGGCTGCGGGAGCCGG | 71 | 16389 | 16404 | 693 |
| 826275 | 805 | 820 | ACGGCTGCGGGAGCCG | 90 | 16390 | 16405 | 694 |
| 826276 | 807 | 822 | CGACGGCTGCGGGAGC | 68 | 16392 | 16407 | 695 |
| 826288 | 830 | 845 | GCGGCAGAGTCCCCCG | 62 | 16415 | 16430 | 488 |
| 826291 | 834 | 849 | GGGTGCGGCAGAGTCC | 22 | 16419 | 16434 | 698 |
| 826292 | 858 | 873 | GGCGGGACCCTCAGGC | 38 | 16443 | 16458 | 699 |
| 826293 | 877 | 892 | ACGGGCCCCGTGAGGC | 46 | 16462 | 16477 | 700 |
| 826294 | 879 | 894 | CGACGGGCCCCGTGAG | 54 | 16464 | 16479 | 701 |
| 826295 | 882 | 897 | GCTCGACGGGCCCCGT | 37 | 16467 | 16482 | 702 |
| 826296 | 883 | 898 | GGCTCGACGGGCCCCG | 46 | 16468 | 16483 | 703 |
| 826308 | 916 | 931 | GTTGTTGTCCCGCAAG | 24 | 16501 | 16516 | 490 |
| 826311 | 952 | 967 | GAAGCCGATCTTCCAG | 22 | 16537 | 16552 | 706 |
| 826312 | 953 | 968 | GGAAGCCGATCTTCCA | 72 | 16538 | 16553 | 707 |
| 826313 | 954 | 969 | TGGAAGCCGATCTTCC | 71 | 16539 | 16554 | 708 |
| 826314 | 956 | 971 | GCTGGAAGCCGATCTT | 37 | 16541 | 16556 | 709 |
| 826315 | 958 | 973 | CAGCTGGAAGCCGATC | 59 | 16543 | 16558 | 710 |
| 826316 | 968 | 983 | TCTGGTTGCACAGCTG | 30 | N/A | N/A | 711 |
| 826326 | 980 | 995 | AGTCCGATTTGTTCTG | 36 | 17766 | 17781 | 492 |
| 826329 | 983 | 998 | AGCAGTCCGATTTGTT | 29 | 17769 | 17784 | 714 |
| 826330 | 985 | 1000 | GAAGCAGTCCGATTTG | 51 | 17771 | 17786 | 715 |
| 826331 | 986 | 1001 | AGAAGCAGTCCGATTT | 33 | 17772 | 17787 | 716 |
| 826332 | 987 | 1002 | TAGAAGCAGTCCGATT | 61 | 17773 | 17788 | 717 |
| 826333 | 988 | 1003 | GTAGAAGCAGTCCGAT | 44 | 17774 | 17789 | 718 |
| 826334 | 989 | 1004 | GGTAGAAGCAGTCCGA | 18 | 17775 | 17790 | 719 |
| 826346 | 1018 | 1033 | CACCGCATCCACCCCT | 42 | 17804 | 17819 | 494 |
| 826349 | 1022 | 1037 | CCCTCACCGCATCCAC | 30 | 17808 | 17823 | 722 |
| 826350 | 1025 | 1040 | ACTCCCTCACCGCATC | 39 | 17811 | 17826 | 723 |
| 826351 | 1026 | 1041 | CACTCCCTCACCGCAT | 48 | 17812 | 17827 | 724 |
| 826352 | 1028 | 1043 | ACCACTCCCTCACCGC | 32 | 17814 | 17829 | 725 |
| 826353 | 1032 | 1047 | CGGTACCACTCCCTCA | 28 | 17818 | 17833 | 726 |
| 826354 | 1033 | 1048 | GCGGTACCACTCCCTC | 46 | 17819 | 17834 | 727 |
| 826366 | 1115 | 1130 | CGAAGATGAAGTTGCC | 67 | 17901 | 17916 | 496 |
| 826369 | 1123 | 1138 | GCGGCAGGCGAAGATG | 62 | 17909 | 17924 | 730 |
| 826370 | 1126 | 1141 | GAAGCGGCAGGCGAAG | 51 | 17912 | 17927 | 731 |
| 826371 | 1129 | 1144 | GTTGAAGCGGCAGGCG | 43 | 17915 | 17930 | 732 |
| 826372 | 1130 | 1145 | GGTTGAAGCGGCAGGC | 20 | 17916 | 17931 | 733 |
| 826373 | 1134 | 1149 | ACCTGGTTGAAGCGGC | 28 | 17920 | 17935 | 734 |
| 826374 | 1136 | 1151 | AGACCTGGTTGAAGCG | 31 | 17922 | 17937 | 735 |
| 826386 | 1164 | 1179 | AAGTGAGAGTAATTCG | 42 | N/A | N/A | 498 |
| 826389 | 1232 | 1247 | AAGACATCCAGAGGTT | 79 | 24188 | 24203 | 738 |
| 826390 | 1250 | 1265 | TGTTGATTCCAGGCAT | 27 | 24206 | 24221 | 739 |
| 826391 | 1251 | 1266 | TTGTTGATTCCAGGCA | 29 | 24207 | 24222 | 740 |
| 826392 | 1252 | 1267 | GTTGTTGATTCCAGGC | 16 | 24208 | 24223 | 741 |
| 826393 | 1254 | 1269 | CCGTTGTTGATTCCAG | 11 | 24210 | 24225 | 742 |
| 826394 | 1255 | 1270 | ACCGTTGTTGATTCCA | 9 | 24211 | 24226 | 743 |
| 826406 | 1282 | 1297 | CTGCTCTGCGCGCAGC | 56 | 24579 | 24594 | 500 |
| 826409 | 1285 | 1300 | ATTCTGCTCTGCGCGC | 16 | 24582 | 24597 | 746 |
| 826410 | 1286 | 1301 | CATTCTGCTCTGCGCG | 30 | 24583 | 24598 | 747 |
| 826411 | 1287 | 1302 | TCATTCTGCTCTGCGC | 20 | 24584 | 24599 | 748 |
| 826412 | 1323 | 1338 | CGGGCCCCAGTCACTG | 55 | 24620 | 24635 | 749 |
| 826413 | 1325 | 1340 | CCCGGGCCCCAGTCAC | 51 | 24622 | 24637 | 750 |
| 826414 | 1327 | 1342 | TACCCGGGCCCCAGTC | 38 | 24624 | 24639 | 751 |
| 826426 | 1356 | 1371 | AAGGCAGGTTCATCCT | 122 | 24653 | 24668 | 502 |
| 826429 | 1366 | 1381 | ATCATCCATAAAGGCA | 32 | 24663 | 24678 | 754 |
| 826430 | 1379 | 1394 | AGTTAAAGCCACCATC | 37 | 24676 | 24691 | 755 |
| 826431 | 1383 | 1398 | CGCAAGTTAAAGCCAC | 33 | 24680 | 24695 | 756 |
| 826432 | 1385 | 1400 | GCCGCAAGTTAAAGCC | 43 | 24682 | 24697 | 757 |
| 826433 | 1387 | 1402 | AGGCCGCAAGTTAAAG | 56 | 24684 | 24699 | 758 |
| 826434 | 1388 | 1403 | CAGGCCGCAAGTTAAA | 42 | 24685 | 24700 | 759 |
| 826446 | 1416 | 1431 | TTCCTCATGCTGATGG | 33 | 24713 | 24728 | 504 |
| 826449 | 1446 | 1461 | TAATCGCCCCCAAGTC | 22 | 25166 | 25181 | 762 |
| 826450 | 1447 | 1462 | ATAATCGCCCCCAAGT | 61 | 25167 | 25182 | 763 |
| 826451 | 1448 | 1463 | CATAATCGCCCCCAAG | 17 | 25168 | 25183 | 764 |
| 826452 | 1450 | 1465 | GCCATAATCGCCCCCA | 24 | 25170 | 25185 | 765 |
| 826453 | 1451 | 1466 | CGCCATAATCGCCCCC | 25 | 25171 | 25186 | 766 |
| 826454 | 1453 | 1468 | GTCGCCATAATCGCCC | 47 | 25173 | 25188 | 767 |
| 826466 | 1500 | 1515 | GAAGGGTAAAGGTTCT | 60 | 25220 | 25235 | 506 |
| 826469 | 1528 | 1543 | GTGAATACACACCTGC | 45 | N/A | N/A | 770 |
| 826470 | 1530 | 1545 | GAGTGAATACACACCT | 58 | 25444 | 25459 | 771 |
| 826471 | 1531 | 1546 | GGAGTGAATACACACC | 69 | 25445 | 25460 | 772 |
| 826472 | 1534 | 1549 | GCAGGAGTGAATACAC | 73 | 25448 | 25463 | 773 |
| 826473 | 1553 | 1568 | TGATCATGCTCTCCTG | 29 | 25467 | 25482 | 774 |
| 826474 | 1554 | 1569 | TTGATCATGCTCTCCT | 19 | 25468 | 25483 | 775 |
| 826485 | 1579 | 1594 | GAAGATGTAGGCACAG | 56 | 25493 | 25508 | 507 |
| 826488 | 1583 | 1598 | GATAGAAGATGTAGGC | 26 | 25497 | 25512 | 778 |
| 826489 | 1584 | 1599 | GGATAGAAGATGTAGG | 35 | 25498 | 25513 | 779 |
| 826490 | 1585 | 1600 | CGGATAGAAGATGTAG | 47 | 25499 | 25514 | 780 |
| 826491 | 1587 | 1602 | CGCGGATAGAAGATGT | 47 | 25501 | 25516 | 781 |
| 826492 | 1588 | 1603 | CCGCGGATAGAAGATG | 71 | 25502 | 25517 | 782 |
| 826493 | 1589 | 1604 | GCCGCGGATAGAAGAT | 62 | 25503 | 25518 | 783 |
| 826505 | 1661 | 1676 | CAACCTGGAGCTTATA | 54 | 30607 | 30622 | 509 |
| 826508 | 1669 | 1684 | GGAGAAGTCAACCTGG | 10 | 30615 | 30630 | 786 |
| 826509 | 1675 | 1690 | GTCTGAGGAGAAGTCA | 32 | 30621 | 30636 | 787 |
| 826510 | 1696 | 1711 | CTTGGTGAAACAGCCC | 53 | 30642 | 30657 | 788 |
| 826511 | 1702 | 1717 | CCGGCACTTGGTGAAA | 92 | 30648 | 30663 | 789 |
| 826512 | 1708 | 1723 | TGGCTTCCGGCACTTG | 25 | 30654 | 30669 | 790 |
| 826513 | 1709 | 1724 | ATGGCTTCCGGCACTT | 34 | 30655 | 30670 | 791 |
| 826525 | 1736 | 1751 | CAGAGAGCTGGTAGCT | 72 | 30787 | 30802 | 511 |
| 826565 | 1993 | 2008 | GACGAGCTCAGCCATC | 39 | 31822 | 31837 | 513 |
| 826568 | 2001 | 2016 | AGGTCAAAGACGAGCT | 29 | 31830 | 31845 | 795 |
| 826569 | 2002 | 2017 | CAGGTCAAAGACGAGC | 28 | 31831 | 31846 | 796 |
| 826570 | 2003 | 2018 | GCAGGTCAAAGACGAG | 42 | 31832 | 31847 | 797 |
| 826571 | 2009 | 2024 | TGACCAGCAGGTCAAA | 106 | 31838 | 31853 | 798 |
| 826572 | 2011 | 2026 | GATGACCAGCAGGTCA | 71 | 31840 | 31855 | 799 |
| 826573 | 2032 | 2047 | TCGGAGCAGCATGAGG | 37 | 31861 | 31876 | 800 |
| 826584 | 2046 | 2061 | CGGCTTCGGAACCTTC | 41 | 31875 | 31890 | 514 |
| 826587 | 2049 | 2064 | TATCGGCTTCGGAACC | 43 | 31878 | 31893 | 803 |
| 826588 | 2050 | 2065 | GTATCGGCTTCGGAAC | 30 | 31879 | 31894 | 804 |
| 826589 | 2051 | 2066 | AGTATCGGCTTCGGAA | 27 | 31880 | 31895 | 805 |
| 826590 | 2053 | 2068 | CCAGTATCGGCTTCGG | 23 | 31882 | 31897 | 806 |
| 826591 | 2054 | 2069 | ACCAGTATCGGCTTCG | 12 | 31883 | 31898 | 807 |
| 826592 | 2055 | 2070 | GACCAGTATCGGCTTC | 36 | 31884 | 31899 | 808 |
| 826604 | 2217 | 2232 | CCCAGGGTGGCATAGG | 46 | 32046 | 32061 | 516 |
| 826607 | 2282 | 2297 | AGGGCCCCCCCAGAGG | 97 | 32111 | 32126 | 811 |
| 826608 | 2284 | 2299 | TCAGGGCCCCCCCAGA | 70 | 32113 | 32128 | 812 |
| 826609 | 2308 | 2323 | GTGTGAGAAACCTCTC | 34 | 32137 | 32152 | 813 |
| 826610 | 2310 | 2325 | TGGTGTGAGAAACCTC | 58 | 32139 | 32154 | 814 |
| 826611 | 2313 | 2328 | CCTTGGTGTGAGAAAC | 46 | 32142 | 32157 | 815 |
| 826612 | 2314 | 2329 | GCCTTGGTGTGAGAAA | 37 | 32143 | 32158 | 816 |
| 826624 | 2390 | 2405 | TGGGCGGCTCTGAGAG | 52 | 32219 | 32234 | 518 |
| 826627 | 2399 | 2414 | ACGGCAGTTTGGGCGG | 30 | 32228 | 32243 | 819 |
| 826628 | 2400 | 2415 | AACGGCAGTTTGGGCG | 37 | 32229 | 32244 | 820 |
| 826629 | 2401 | 2416 | CAACGGCAGTTTGGGC | 36 | 32230 | 32245 | 821 |
| 826630 | 2403 | 2418 | ATCAACGGCAGTTTGG | 35 | 32232 | 32247 | 822 |
| 826631 | 2405 | 2420 | ACATCAACGGCAGTTT | 16 | 32234 | 32249 | 823 |
| 826632 | 2407 | 2422 | ACACATCAACGGCAGT | 15 | 32236 | 32251 | 824 |
| 826644 | 2476 | 2491 | CTGGGCAGCTTCATCA | 42 | 32305 | 32320 | 520 |
| 826647 | 2491 | 2506 | GGAGCCAAGGCACTTC | 13 | 32320 | 32335 | 827 |
| 826648 | 2492 | 2507 | TGGAGCCAAGGCACTT | 29 | 32321 | 32336 | 828 |
| 826649 | 2502 | 2517 | GGTACAGGGCTGGAGC | 52 | 32331 | 32346 | 829 |
| 826650 | 2520 | 2535 | TCAGAGGCAGTACCAA | 30 | 32349 | 32364 | 830 |
| 826651 | 2523 | 2538 | TGTTCAGAGGCAGTAC | 33 | 32352 | 32367 | 831 |
| 826652 | 2533 | 2548 | GAAACCAGAGTGTTCA | 48 | 32362 | 32377 | 832 |
| 826665 | 2579 | 2594 | CTTGGCTGATCCAAGG | 61 | 32408 | 32423 | 835 |
| 826666 | 2580 | 2595 | GCTTGGCTGATCCAAG | 45 | 32409 | 32424 | 836 |
| 826667 | 2581 | 2596 | CGCTTGGCTGATCCAA | 27 | 32410 | 32425 | 837 |
| 826668 | 2582 | 2597 | TCGCTTGGCTGATCCA | 6 | 32411 | 32426 | 838 |
| 826669 | 2583 | 2598 | TTCGCTTGGCTGATCC | 34 | 32412 | 32427 | 839 |
| 826670 | 2584 | 2599 | TTTCGCTTGGCTGATC | 30 | 32413 | 32428 | 840 |
| 826682 | 2612 | 2627 | TAGGAAAGTTCCTTGT | 83 | 32441 | 32456 | 523 |
| 826685 | 2623 | 2638 | CAGCGGTTTCTTAGGA | 16 | 32452 | 32467 | 843 |
| 826686 | 2625 | 2640 | ATCAGCGGTTTCTTAG | 42 | 32454 | 32469 | 844 |
| 826687 | 2627 | 2642 | TTATCAGCGGTTTCTT | 22 | 32456 | 32471 | 845 |
| 826688 | 2629 | 2644 | GGTTATCAGCGGTTTC | 9 | 32458 | 32473 | 846 |
| 826689 | 2632 | 2647 | CCTGGTTATCAGCGGT | 17 | 32461 | 32476 | 847 |
| 826690 | 2634 | 2649 | GTCCTGGTTATCAGCG | 25 | 32463 | 32478 | 848 |
| 826702 | 2681 | 2696 | TGGGCAGGAAACCCGT | 58 | 32510 | 32525 | 525 |
| 826705 | 2692 | 2707 | TAAGCCGTCGCTGGGC | 34 | 32521 | 32536 | 851 |
| 826706 | 2693 | 2708 | TTAAGCCGTCGCTGGG | 47 | 32522 | 32537 | 852 |
| 826707 | 2696 | 2711 | GGCTTAAGCCGTCGCT | 24 | 32525 | 32540 | 853 |
| 826708 | 2698 | 2713 | CTGGCTTAAGCCGTCG | 42 | 32527 | 32542 | 854 |
| 826709 | 2700 | 2715 | GGCTGGCTTAAGCCGT | 90 | 32529 | 32544 | 855 |
| 826710 | 2701 | 2716 | GGGCTGGCTTAAGCCG | 83 | 32530 | 32545 | 856 |
| 826722 | 2835 | 2850 | CCTAGCCCTCGGGAGT | 71 | 32664 | 32679 | 527 |
| 826725 | 2846 | 2861 | TCTGCTCTAGCCCTAG | 52 | 32675 | 32690 | 859 |
| 826726 | 2847 | 2862 | GTCTGCTCTAGCCCTA | 35 | 32676 | 32691 | 860 |
| 826727 | 2850 | 2865 | CGGGTCTGCTCTAGCC | 61 | 32679 | 32694 | 861 |
| 826728 | 2852 | 2867 | CCCGGGTCTGCTCTAG | 84 | 32681 | 32696 | 862 |
| 826729 | 2854 | 2869 | TACCCGGGTCTGCTCT | 63 | 32683 | 32698 | 863 |
| 826730 | 2855 | 2870 | TTACCCGGGTCTGCTC | 54 | 32684 | 32699 | 864 |
| 826742 | 2949 | 2964 | TGTAGAGGTATGAAAG | 55 | 32778 | 32793 | 529 |
| 826745 | 2954 | 2969 | AGACATGTAGAGGTAT | 17 | 32783 | 32798 | 867 |
| 826746 | 2955 | 2970 | CAGACATGTAGAGGTA | 7 | 32784 | 32799 | 868 |
| 826747 | 2959 | 2974 | CAAGCAGACATGTAGA | 31 | 32788 | 32803 | 869 |
| 826748 | 2960 | 2975 | TCAAGCAGACATGTAG | 30 | 32789 | 32804 | 870 |
| 826749 | 2961 | 2976 | CTCAAGCAGACATGTA | 24 | 32790 | 32805 | 871 |
| 826750 | 2963 | 2978 | ATCTCAAGCAGACATG | 39 | 32792 | 32807 | 872 |
| 826764 | 3016 | 3031 | ATGCATAGGAGTTCTC | 8 | 32845 | 32860 | 875 |
| 826765 | 3017 | 3032 | GATGCATAGGAGTTCT | 30 | 32846 | 32861 | 876 |
| 826766 | 3019 | 3034 | GGGATGCATAGGAGTT | 32 | 32848 | 32863 | 877 |
| 826767 | 3021 | 3036 | AAGGGATGCATAGGAG | 31 | 32850 | 32865 | 878 |
| 826768 | 3022 | 3037 | TAAGGGATGCATAGGA | 25 | 32851 | 32866 | 879 |
| 826769 | 3023 | 3038 | CTAAGGGATGCATAGG | 35 | 32852 | 32867 | 880 |
| 826781 | 3117 | 3132 | CTACGGGAGCCCAGGA | 32 | 32946 | 32961 | 532 |
| 826784 | 3121 | 3136 | TGTGCTACGGGAGCCC | 8 | 32950 | 32965 | 883 |
| 826785 | 3122 | 3137 | GTGTGCTACGGGAGCC | 20 | 32951 | 32966 | 884 |
| 826786 | 3123 | 3138 | AGTGTGCTACGGGAGC | 15 | 32952 | 32967 | 885 |
| 826787 | 3124 | 3139 | TAGTGTGCTACGGGAG | 21 | 32953 | 32968 | 886 |
| 826788 | 3125 | 3140 | ATAGTGTGCTACGGGA | 29 | 32954 | 32969 | 887 |
| 826789 | 3126 | 3141 | TATAGTGTGCTACGGG | 30 | 32955 | 32970 | 888 |
| 826800 | 3145 | 3160 | GCAACACTCCAGCAGA | 17 | 32974 | 32989 | 534 |
| 826803 | 3153 | 3168 | GTGCAACAGCAACACT | 36 | 32982 | 32997 | 890 |
| 826804 | 3154 | 3169 | GGTGCAACAGCAACAC | 33 | 32983 | 32998 | 891 |
| 826805 | 3155 | 3170 | TGGTGCAACAGCAACA | 30 | 32984 | 32999 | 892 |
| 826806 | 3156 | 3171 | ATGGTGCAACAGCAAC | 11 | 32985 | 33000 | 893 |
| 826807 | 3157 | 3172 | TATGGTGCAACAGCAA | 21 | 32986 | 33001 | 894 |
| 826808 | 3158 | 3173 | GTATGGTGCAACAGCA | 20 | 32987 | 33002 | 895 |
| 826818 | 3213 | 3228 | TGACCGCAAGGCACTT | 30 | 33042 | 33057 | 536 |
| 826821 | 3216 | 3231 | CCCTGACCGCAAGGCA | 7 | 33045 | 33060 | 897 |
| 826822 | 3217 | 3232 | TCCCTGACCGCAAGGC | 28 | 33046 | 33061 | 898 |
| 826823 | 3218 | 3233 | GTCCCTGACCGCAAGG | 25 | 33047 | 33062 | 899 |
| 826824 | 3219 | 3234 | AGTCCCTGACCGCAAG | 33 | 33048 | 33063 | 900 |
| 826825 | 3220 | 3235 | CAGTCCCTGACCGCAA | 17 | 33049 | 33064 | 901 |
| 826826 | 3222 | 3237 | TTCAGTCCCTGACCGC | 19 | 33051 | 33066 | 902 |
| 826837 | 3236 | 3251 | ATAAACGGGCAAGATT | 57 | 33065 | 33080 | 538 |
| 826840 | 3239 | 3254 | TACATAAACGGGCAAG | 40 | 33068 | 33083 | 905 |
| 826841 | 3242 | 3257 | GCATACATAAACGGGC | 35 | 33071 | 33086 | 906 |
| 826842 | 3244 | 3259 | GAGCATACATAAACGG | 27 | 33073 | 33088 | 907 |
| 826843 | 3249 | 3264 | ACATGGAGCATACATA | 69 | 33078 | 33093 | 908 |
| 826844 | 3250 | 3265 | GACATGGAGCATACAT | 42 | 33079 | 33094 | 909 |
| 826845 | 3251 | 3266 | AGACATGGAGCATACA | 48 | 33080 | 33095 | 910 |
| 826857 | N/A | N/A | TCCCCTTGGAAGGGAC | 109 | 2713 | 2728 | 540 |
| 826860 | N/A | N/A | TGATACCTCCCCTTGG | 90 | 2720 | 2735 | 913 |
| 826861 | N/A | N/A | ATGATACCTCCCCTTG | 84 | 2721 | 2736 | 914 |
| 826862 | N/A | N/A | GCTCATGATACCTCCC | 68 | 2725 | 2740 | 915 |
| 826863 | N/A | N/A | ACTGCTCATGATACCT | 89 | 2728 | 2743 | 916 |
| 826864 | N/A | N/A | ATACTGCTCATGATAC | 70 | 2730 | 2745 | 917 |
| 826865 | N/A | N/A | GATACTGCTCATGATA | 87 | 2731 | 2746 | 918 |
| 826877 | N/A | N/A | GTCCTAGCACCTCCCT | 15 | 4868 | 4883 | 542 |
| 826880 | N/A | N/A | CGAGTTTTGTCCTAGC | 7 | 4876 | 4891 | 921 |
| 826881 | N/A | N/A | TCGAGTTTTGTCCTAG | 25 | 4877 | 4892 | 922 |
| 826882 | N/A | N/A | CTTTCGAGTTTTGTCC | 36 | 4880 | 4895 | 923 |
| 826883 | N/A | N/A | CACCTTTCGAGTTTTG | 26 | 4883 | 4898 | 924 |
| 826884 | N/A | N/A | GCCACCTTTCGAGTTT | 30 | 4885 | 4900 | 925 |
| 826885 | N/A | N/A | GGGCCACCTTTCGAGT | 27 | 4887 | 4902 | 926 |
| 826897 | N/A | N/A | CTTCCCTAGAACGGCC | 37 | 4923 | 4938 | 544 |
| 826900 | N/A | N/A | GCCGGAGCTGGGCTTC | 36 | 4935 | 4950 | 929 |
| 826901 | N/A | N/A | TGCCGGAGCTGGGCTT | 80 | 4936 | 4951 | 930 |
| 826902 | N/A | N/A | GTGCCGGAGCTGGGCT | 67 | 4937 | 4952 | 931 |
| 826903 | N/A | N/A | AAGTGCCGGAGCTGGG | 30 | 4939 | 4954 | 932 |
| 826904 | N/A | N/A | AAAAGTGCCGGAGCTG | 45 | 4941 | 4956 | 933 |
| 826905 | N/A | N/A | CCAAAAGTGCCGGAGC | 28 | 4943 | 4958 | 934 |
| 826917 | N/A | N/A | GAACCCGAGTGAGGCT | 39 | 5059 | 5074 | 546 |
| 826920 | N/A | N/A | CCCCTGGAACCCGAGT | 25 | 5065 | 5080 | 937 |
| 826921 | N/A | N/A | CACCCCTGGAACCCGA | 50 | 5067 | 5082 | 938 |
| 826922 | N/A | N/A | CCCGGAGTGGATTGGG | 100 | 5138 | 5153 | 939 |
| 826923 | N/A | N/A | GCCCCGGAGTGGATTG | 50 | 5140 | 5155 | 940 |
| 826924 | N/A | N/A | GAGCCCCGGAGTGGAT | 46 | 5142 | 5157 | 941 |
| 826925 | N/A | N/A | ATGAGCCCCGGAGTGG | 38 | 5144 | 5159 | 942 |
| 826937 | N/A | N/A | AGCCGGGAAGGCCTCC | 124 | 2486 | 2501 | 548 |
| 826940 | N/A | N/A | TGCTTACCTTGATACT | 113 | 2741 | 2756 | 945 |
| 826941 | N/A | N/A | CCAAACCAGGTTCCCT | 125 | 2757 | 2772 | 946 |
| 826942 | N/A | N/A | AGCCGGTGTCAACCAG | 103 | 2777 | 2792 | 947 |
| 826943 | N/A | N/A | AAAGTGAAAGCCGGTG | 112 | 2785 | 2800 | 948 |
| 826944 | N/A | N/A | TGCGACTTCTTAAAGT | 84 | 2796 | 2811 | 949 |
| 826945 | N/A | N/A | GCTCAGGGTCCAACCT | 109 | 2844 | 2859 | 950 |
| 826957 | N/A | N/A | GCCAAGTGGTGAGCAA | 42 | 3338 | 3353 | 550 |
| 826960 | N/A | N/A | CGTTGATGGGCTATAT | 99 | 3408 | 3423 | 953 |
| 826961 | N/A | N/A | CGCCTAGACAGGCCCT | 44 | 3440 | 3455 | 954 |
| 826962 | N/A | N/A | ACGCAGGACACTGTGG | 80 | 3555 | 3570 | 955 |
| 826963 | N/A | N/A | AGGCAGCGCGAGGGCC | 101 | 3571 | 3586 | 956 |
| 826964 | N/A | N/A | GTGTAATCGCCCCTGC | 90 | 3622 | 3637 | 957 |
| 826965 | N/A | N/A | GGCCCTAGGACATTCT | 79 | 3674 | 3689 | 958 |
| 826977 | N/A | N/A | TGGGACTGGTTCCTTT | 94 | 4536 | 4551 | 552 |
| 826980 | N/A | N/A | GGGACTAACCGACCTG | 98 | 5631 | 5646 | 961 |
| 826981 | N/A | N/A | TTCCAGGCGCAGGCAC | 39 | 5662 | 5677 | 862 |
| 826982 | N/A | N/A | CAGTAAGCTGGAGGCT | 92 | 5785 | 5800 | 963 |
| 826983 | N/A | N/A | CGCCAGTCCAGTAAGC | 85 | 5793 | 5808 | 964 |
| 826984 | N/A | N/A | GCTAGGATGGCTCCAC | 27 | 5819 | 5834 | 965 |
| 826985 | N/A | N/A | CCACACTCTGGGTGAG | 36 | 5843 | 5858 | 966 |
| 826997 | N/A | N/A | CCAGACCCAACATTGG | 84 | 6361 | 6376 | 554 |
| 827000 | N/A | N/A | TCCCAAGGTGTGGCAT | 15 | 6462 | 6477 | 969 |
| 827001 | N/A | N/A | TTGAAGCAGGTGTTCC | 57 | 6475 | 6490 | 970 |
| 827002 | N/A | N/A | TGCCAGGTGCCTAGCC | 44 | 6502 | 6517 | 971 |
| 827003 | N/A | N/A | CAATAAAGGGCTTATG | 64 | 6538 | 6553 | 972 |
| 827004 | N/A | N/A | AACTACCTGGCCTTCA | 69 | 6552 | 6567 | 973 |
| 827005 | N/A | N/A | GGCTTATATGCCTGTC | 68 | 6605 | 6620 | 974 |
| 827017 | N/A | N/A | CTTTCTTAGTCCGTAA | 48 | 6815 | 6830 | 556 |
| 827020 | N/A | N/A | AGGAAATGGTCCCTAC | 70 | 6912 | 6927 | 977 |
| 827021 | N/A | N/A | GTGCACACGGCAGCTT | 52 | 6932 | 6947 | 978 |
| 827022 | N/A | N/A | CCCAAGACACCTTCGC | 33 | 6955 | 6970 | 979 |
| 827023 | N/A | N/A | TAGCACCGGGCTTGTA | 51 | 6994 | 7009 | 980 |
| 827024 | N/A | N/A | AACAGGATGAGTCACA | 31 | 7088 | 7103 | 981 |
| 827025 | N/A | N/A | AGTTTTGGGATTAGGC | 30 | 7107 | 7122 | 982 |
| 827040 | N/A | N/A | GGGAATAATACTGCCC | 91 | 7751 | 7766 | 985 |
| 827041 | N/A | N/A | AATGTATGTTCCCTTG | 27 | 7816 | 7831 | 986 |
| 827042 | N/A | N/A | GTAAAAAGTCTGGCCC | 16 | 8222 | 8237 | 987 |
| 827043 | N/A | N/A | TCCAAGGTGTGTTGTG | 22 | 8283 | 8298 | 988 |
| 827044 | N/A | N/A | CATGAGACCTACTTCC | 26 | 8296 | 8311 | 989 |
| 827045 | N/A | N/A | ATAAGAGTCATCATGA | 46 | 8307 | 8322 | 990 |
| 827057 | N/A | N/A | TCGGTAGGAGTCATTC | 39 | 8767 | 8782 | 559 |
| 827060 | N/A | N/A | CCTCAGCAGGTAGGCA | 60 | 8836 | 8851 | 993 |
| 827061 | N/A | N/A | TCGGACTCAGCACTTC | 64 | 8961 | 8976 | 994 |
| 827062 | N/A | N/A | CTGCAGTGGCCAACCC | 51 | 8983 | 8998 | 995 |
| 827063 | N/A | N/A | CTGTAGGTATGACTGG | 18 | 9047 | 9062 | 996 |
| 827064 | N/A | N/A | TTCCATGACTGTAGGT | 32 | 9055 | 9070 | 997 |
| 827065 | N/A | N/A | GCCTAAACCGTTCCTG | 36 | 9105 | 9120 | 998 |
| 827077 | N/A | N/A | TCTTACCCCGGTGGCC | 69 | 9507 | 9522 | 561 |
| 827080 | N/A | N/A | GGCCTATCAACTAGGC | 103 | 9783 | 9798 | 1001 |
| 827081 | N/A | N/A | CACAATTCCATCGGGC | 17 | 9837 | 9852 | 1002 |
| 827082 | N/A | N/A | CCCTACATTGGAGGGT | 91 | 9866 | 9881 | 1003 |
| 827083 | N/A | N/A | AGGGATAAAGAATGCC | 38 | 9978 | 9993 | 1004 |
| 827084 | N/A | N/A | GACCAGCGGCTGGAGG | 54 | 9996 | 10011 | 1005 |
| 827085 | N/A | N/A | AGACATCCGATCTTGT | 41 | 10020 | 10035 | 1006 |
| 827097 | N/A | N/A | CTGCCACCCTACGCGC | 54 | 10635 | 10650 | 563 |
| 827100 | N/A | N/A | TACGCACCTCCCTCCT | 35 | 10649 | 10664 | 1009 |
| | | | | | 10672 | 10687 | |
| 827101 | N/A | N/A | CTACGCACCTCCCTCC | 64 | 10650 | 10665 | 1010 |
| | | | | | 10673 | 10688 | |
| 827102 | N/A | N/A | CCCTACGCACCTCCCT | 60 | 10652 | 10667 | 1011 |
| | | | | | 10675 | 10690 | |
| 827103 | N/A | N/A | ACCCTACGCACCTCCC | 34 | 10653 | 10668 | 1012 |
| | | | | | 10676 | 10691 | |
| 827104 | N/A | N/A | CCACCCTACGCACCTC | 36 | 10655 | 10670 | 1013 |
| | | | | | 10678 | 10693 | |
| 827105 | N/A | N/A | GCCACCCTACGCACCT | 49 | 10656 | 10671 | 1014 |
| | | | | | 10679 | 10694 | |
| 827117 | N/A | N/A | GGGCATAACACTAGAT | 53 | 11100 | 11115 | 565 |
| 827120 | N/A | N/A | CCACATGGTGCCCCAG | 40 | 11248 | 11263 | 1017 |
| 827121 | N/A | N/A | TTTTAGGAGGGCCACA | 60 | 11259 | 11274 | 1018 |
| 827122 | N/A | N/A | GCCCTCTGGTCCGTCC | 40 | 11291 | 11306 | 1019 |
| 827123 | N/A | N/A | GGTCAGACAGCACTCC | 36 | 11319 | 11334 | 1020 |
| 827124 | N/A | N/A | AGCTAGCAAATGGGTC | 56 | 11331 | 11346 | 1021 |
| 827125 | N/A | N/A | TTCCAGTTGGCACAGC | 26 | 11344 | 11359 | 1022 |
| 827137 | N/A | N/A | GGTTACACCCCCGGCG | 44 | 11650 | 11665 | 567 |
| 827140 | N/A | N/A | CCCACAGAAAACGGAA | 74 | 11690 | 11705 | 1025 |
| 827141 | N/A | N/A | GGCTGCTGCATGATTC | 33 | 11738 | 11753 | 1026 |
| 827142 | N/A | N/A | ACCAGAATAGATTCAC | 63 | 11766 | 11781 | 1027 |
| 827143 | N/A | N/A | TCGAATCGAGTGCCCC | 40 | 11791 | 11806 | 1028 |
| 827144 | N/A | N/A | AACAATGAACCTCGAA | 51 | 11802 | 11817 | 1029 |
| 827145 | N/A | N/A | TGGTATTAGAATGTAC | 19 | 11881 | 11896 | 1030 |
| 827157 | N/A | N/A | TTGAAAGAGCCCCCAC | 65 | 12166 | 12181 | 569 |
| 827160 | N/A | N/A | GTGCAGGGTCTTACTT | 19 | 12230 | 12245 | 1033 |
| 827161 | N/A | N/A | AAATACCAGTGCAGGG | 41 | 12238 | 12253 | 1034 |
| 827162 | N/A | N/A | GTACATCAATTATGCC | 36 | 12268 | 12283 | 1035 |
| 827163 | N/A | N/A | GGGCACTCAAGATTTG | 62 | 12295 | 12310 | 1036 |
| 827164 | N/A | N/A | CAAACCTGAGTGGGCA | 36 | 12306 | 12321 | 1037 |
| 827165 | N/A | N/A | CTCGACTGTCAAACCT | 30 | 12315 | 12330 | 1038 |
| 827177 | N/A | N/A | GCAATCATAGCTAGCA | 57 | 12729 | 12744 | 571 |
| 827180 | N/A | N/A | GTTGAAGGTGTGTGTT | 35 | 13095 | 13110 | 1041 |
| 827181 | N/A | N/A | AGCAACTCAAAGGTGT | 22 | 13111 | 13126 | 1042 |
| 827182 | N/A | N/A | AGATTTGTACATGAGG | 30 | 13481 | 13496 | 1043 |
| 827183 | N/A | N/A | ACCCGAAACACATTAG | 61 | 13504 | 13519 | 1044 |
| 827184 | N/A | N/A | GTTTAGGCCGCACCCG | 36 | 13515 | 13530 | 1045 |
| 827185 | N/A | N/A | ATTTACGGTGTTTAGG | 42 | 13524 | 13539 | 1046 |
| 827197 | N/A | N/A | GTAGGCACTTTATGAA | 68 | 14142 | 14157 | 573 |
| 827200 | N/A | N/A | AACCGTATGTAGTAGG | 18 | 14153 | 14168 | 1049 |
| 827201 | N/A | N/A | CAACCGTATGTAGTAG | 25 | 14154 | 14169 | 1050 |
| 827202 | N/A | N/A | ACAACCGTATGTAGTA | 40 | 14155 | 14170 | 1051 |
| 827203 | N/A | N/A | AACAACCGTATGTAGT | 36 | 14156 | 14171 | 1052 |
| 827204 | N/A | N/A | GAACAACCGTATGTAG | 39 | 14157 | 14172 | 1053 |
| 827216 | N/A | N/A | GGAGAGACAATAGATC | 50 | 14488 | 14503 | 575 |
| 827219 | N/A | N/A | TGACATACTGCTTCTA | 33 | 14642 | 14657 | 1056 |
| 827220 | N/A | N/A | CCCCAGCAGGTATTTT | 98 | 14667 | 14682 | 1057 |
| 827221 | N/A | N/A | CCCAAGCAATCACCAG | 61 | 14737 | 14752 | 1058 |
| 827222 | N/A | N/A | GACCAAAAGTGTGCCA | 54 | 14831 | 14846 | 1059 |
| 827223 | N/A | N/A | GACACAATCGCCGCTC | 64 | 14905 | 14920 | 1060 |
| 827224 | N/A | N/A | GAATAAGTGGAGATAT | 84 | 15017 | 15032 | 1061 |
| 827236 | N/A | N/A | CCGCAGGCGAGTGTCG | 61 | 15878 | 15893 | 577 |
| 827239 | N/A | N/A | CCGGACCTAGAAGGGA | 89 | 15987 | 16002 | 1064 |
| 827240 | N/A | N/A | GGCCACGGCGAGCCCA | 82 | 16080 | 16095 | 1065 |
| 827241 | N/A | N/A | GTAAACAGGTGTGTCC | 48 | 16110 | 16125 | 1066 |
| 827242 | N/A | N/A | CTGGAGCGAGTGTCTG | 93 | 16238 | 16253 | 1067 |
| 827243 | N/A | N/A | GCGGAGCCCATGGGTG | 52 | 16616 | 16631 | 1068 |
| 827244 | N/A | N/A | TGTCACTGGGCTGCGC | 41 | 16650 | 16665 | 1069 |
| 827256 | N/A | N/A | CAAGAGATTTGTCCCA | 67 | 17420 | 17435 | 579 |
| 827259 | N/A | N/A | ATTTATACCTCCCCTG | 60 | 17495 | 17510 | 1072 |
| 827260 | N/A | N/A | CACACACGGTTTTGGT | 26 | 17513 | 17528 | 1073 |
| 827261 | N/A | N/A | GACCAGTAGCTGCACA | 34 | 17527 | 17542 | 1074 |
| 827262 | N/A | N/A | ATTAAGGGAGTTGCAG | 61 | 17555 | 17570 | 1075 |
| 827263 | N/A | N/A | CCCTAGGAGCATGGAC | 47 | 17585 | 17600 | 1076 |
| 827264 | N/A | N/A | GCAGAAGTCCCTAGGA | 61 | 17593 | 17608 | 1077 |
| 827276 | N/A | N/A | CCTCAGATCCAGCAGT | 49 | 18147 | 18162 | 581 |
| 827279 | N/A | N/A | AGATCCATGCTTCCAG | 11 | 18216 | 18231 | 1080 |
| 827280 | N/A | N/A | AAGATCCATGCTTCCA | 17 | 18217 | 18232 | 1081 |
| 827281 | N/A | N/A | CCAAGATCCATGCTTC | 20 | 18219 | 18234 | 1082 |
| 827282 | N/A | N/A | ACCAAGATCCATGCTT | 35 | 18220 | 18235 | 1083 |
| 827283 | N/A | N/A | GACCAAGATCCATGCT | 13 | 18221 | 18236 | 1084 |
| 827295 | N/A | N/A | GATACATCCAGAGTCA | 36 | 18725 | 18740 | 583 |
| 827298 | N/A | N/A | AGGATGATGTGATACA | 24 | 18735 | 18750 | 1087 |
| 827299 | N/A | N/A | AAGGATGATGTGATAC | 36 | 18736 | 18751 | 1088 |
| 827300 | N/A | N/A | ATCTAAGAAATAGGCT | 29 | 18755 | 18770 | 1089 |
| 827301 | N/A | N/A | CACATAGCCCAGATAG | 21 | 18834 | 18849 | 1090 |
| 827302 | N/A | N/A | TGCCAAAGGAGCATGG | 52 | 18901 | 18916 | 1091 |
| 827314 | N/A | N/A | GGAGATGGCTCCGGAA | 63 | 19278 | 19293 | 585 |
| 827317 | N/A | N/A | GGTAAGAAGTGACACC | 62 | 19364 | 19379 | 1094 |
| 827318 | N/A | N/A | GTGTACTGGGCAGAGT | 16 | 19390 | 19405 | 1095 |
| 827319 | N/A | N/A | TGCTACCATCTTACTT | 26 | 19463 | 19478 | 1096 |
| 827320 | N/A | N/A | GGCTTAGGTGTTGCTA | 28 | 19474 | 19489 | 1097 |
| 827321 | N/A | N/A | GCGGACTCAGGCTTAG | 50 | 19483 | 19498 | 1098 |
| 827322 | N/A | N/A | TGACAGGTGTGGGCGG | 46 | 19495 | 19510 | 1099 |
| 827334 | N/A | N/A | AACCATGGACTTTCTG | 46 | 19687 | 19702 | 587 |
| 827337 | N/A | N/A | CCCAGGCGAGCAATGA | 49 | 19746 | 19761 | 1102 |
| 827338 | N/A | N/A | GGTATAACAACCCAGG | 34 | 19756 | 19771 | 1103 |
| 827339 | N/A | N/A | CAGTAGGGTGGAGTGG | 41 | 19774 | 19789 | 1104 |
| 827340 | N/A | N/A | GTACAAAGGTTCCTGT | 48 | 19829 | 19844 | 1105 |
| 827341 | N/A | N/A | CGTGAAGTAAGGTTGA | 25 | 19846 | 19861 | 1106 |
| 827342 | N/A | N/A | GTTACATGTGGTGACG | 43 | 19860 | 19875 | 1107 |
| 827353 | N/A | N/A | ACTAACTGGAATAGTT | 99 | 19873 | 19888 | 589 |
| 827356 | N/A | N/A | AGGACTAACTGGAATA | 15 | 19876 | 19891 | 1110 |
| 827357 | N/A | N/A | CAGGACTAACTGGAAT | 31 | 19877 | 19892 | 1111 |
| 827358 | N/A | N/A | GCCCGGTGAGATATTC | 55 | 19923 | 19938 | 1112 |
| 827359 | N/A | N/A | CCCGATAGCTGGTTGT | 11 | 20415 | 20430 | 1113 |
| 827360 | N/A | N/A | TTAATTAGTTCACCCG | 4 | 20427 | 20442 | 1114 |
| 827361 | N/A | N/A | AGTGAATCCTCACACT | 88 | 20444 | 20459 | 1115 |
| 827373 | N/A | N/A | AAAAAGGTGGTGTATC | 80 | 21111 | 21126 | 591 |
| 827376 | N/A | N/A | TCATAGGTAAACACCC | 14 | 21565 | 21580 | 1118 |
| 827377 | N/A | N/A | GAAAAGTCTGGTAGCT | 23 | 21628 | 21643 | 1119 |
| 827378 | N/A | N/A | TGGTGTGACCATTTGG | 9 | 21643 | 21658 | 1120 |
| 827379 | N/A | N/A | ATGGTGTGACCATTTG | 15 | 21644 | 21659 | 1121 |
| 827380 | N/A | N/A | AAATGGTGTGACCATT | 46 | 21646 | 21661 | 1122 |
| 827381 | N/A | N/A | TAAATGGTGTGACCAT | 25 | 21647 | 21662 | 1123 |
| 827392 | N/A | N/A | CGATTACAGGGATTCA | 15 | 21750 | 21765 | 593 |
| 827394 | N/A | N/A | GTACGATTACAGGGAT | 11 | 21753 | 21768 | 1125 |
| 827395 | N/A | N/A | AGTACGATTACAGGGA | 8 | 21754 | 21769 | 1126 |
| 827396 | N/A | N/A | TAGTACGATTACAGGG | 29 | 21755 | 21770 | 1127 |
| 827397 | N/A | N/A | CTAGTACGATTACAGG | 23 | 21756 | 21771 | 1128 |
| 827398 | N/A | N/A | ACTAGTACGATTACAG | 15 | 21757 | 21772 | 1129 |
| 827399 | N/A | N/A | CACTAGTACGATTACA | 39 | 21758 | 21773 | 1130 |
| 827410 | N/A | N/A | GAATCAGTATAATGTG | 16 | 22306 | 22321 | 595 |
| 827413 | N/A | N/A | CCTATGAGAATCAGTA | 14 | 22313 | 22328 | 1133 |
| 827414 | N/A | N/A | GCCTATGAGAATCAGT | 13 | 22314 | 22329 | 1134 |
| 827415 | N/A | N/A | CTATAGTGGCCTATGA | 33 | 22322 | 22337 | 1135 |
| 827416 | N/A | N/A | GATACACACTAAGCAC | 22 | 22342 | 22357 | 1136 |
| 827417 | N/A | N/A | AGATACACACTAAGCA | 29 | 22343 | 22358 | 1137 |
| 827429 | N/A | N/A | CTGCCCCCATGGAAAG | 70 | 22782 | 22797 | 597 |
| 827432 | N/A | N/A | GGTGAGCCCTTCGCAC | 3 | 22828 | 22843 | 1140 |
| 827433 | N/A | N/A | TGAAGGAGAGGCTACA | 45 | 22866 | 22881 | 1141 |
| 827434 | N/A | N/A | ATTCTAGGATGTACTG | 36 | 22926 | 22941 | 1142 |
| 827435 | N/A | N/A | GTGACATACTGGTGCA | 3 | 22943 | 22958 | 1143 |
| 827436 | N/A | N/A | GGGATATTCCACTGGC | 29 | 22983 | 22998 | 1144 |
| 827437 | N/A | N/A | AACTAGGTGATCCGGG | 11 | 22996 | 23011 | 1145 |
| 827448 | N/A | N/A | CTGCAGTAGGACTGCA | 111 | 23326 | 23341 | 598 |
| 827451 | N/A | N/A | GGTGAGCACGGAGCTG | 14 | 23471 | 23486 | 1148 |
| 827452 | N/A | N/A | GGAGAAAGTGTGACCA | 56 | 23489 | 23504 | 1149 |
| 827453 | N/A | N/A | GAGCAGGGTTAAAGGA | 49 | 23502 | 23517 | 1150 |
| 827454 | N/A | N/A | TGTCATCTAGGAGATA | 70 | 23597 | 23612 | 1151 |
| 827455 | N/A | N/A | TTGCATAGATCCTGTC | 35 | 23609 | 23624 | 1152 |
| 827456 | N/A | N/A | CTTGATGACAGGAGCC | 38 | 23660 | 23675 | 1153 |
| 827468 | N/A | N/A | CGCCATGGAGCAAGCA | 67 | 24238 | 24253 | 600 |
| 827471 | N/A | N/A | GGACTATGTGGCACCT | 47 | 24342 | 24357 | 1156 |
| 827472 | N/A | N/A | TGGCAACCCCTGAGCT | 59 | 24412 | 24427 | 1157 |
| 827473 | N/A | N/A | GTTCAGGAAGACCCGC | 65 | 24437 | 24452 | 1158 |
| 827474 | N/A | N/A | GCAGAGGCGGGAATCC | 49 | 24524 | 24539 | 1159 |
| 827475 | N/A | N/A | CATCAGGGACAGACCT | 43 | 24564 | 24579 | 1160 |
| 827476 | N/A | N/A | CTGCAATCTGAGGCGC | 58 | 24761 | 24776 | 1161 |
| 827488 | N/A | N/A | GGACAATTCCTTGACA | 36 | 26078 | 26093 | 602 |
| 827491 | N/A | N/A | ACCTTAGGAGCCATTG | 18 | 26493 | 26508 | 1164 |
| 827492 | N/A | N/A | ACCCATGTATCTTCTA | 44 | 26627 | 26642 | 1165 |
| 827493 | N/A | N/A | AATGAGACAGACCCAT | 42 | 26637 | 26652 | 1166 |
| 827494 | N/A | N/A | GGATACAGTATGTCCA | 52 | 26685 | 26700 | 1167 |
| 827495 | N/A | N/A | CTCTACTATTGAATGG | 45 | 26699 | 26714 | 1168 |
| 827496 | N/A | N/A | ATTATATACCTCTACT | 58 | 26708 | 26723 | 1169 |
| 827508 | N/A | N/A | AGGTAGGGATGGACGC | 38 | 27147 | 27162 | 604 |
| 827511 | N/A | N/A | CCAGGAGGCCACGACT | 32 | 27241 | 27256 | 1172 |
| 827512 | N/A | N/A | TACAATCCTCTAAGGT | 47 | 27271 | 27286 | 1173 |
| 827513 | N/A | N/A | CTGTATACCCTGGGAC | 41 | 27378 | 27393 | 1174 |
| 827514 | N/A | N/A | TCTCAGCAATCAATAT | 75 | 27490 | 27505 | 1175 |
| 827515 | N/A | N/A | GGGAAGTAAGCCCTAG | 22 | 27559 | 27574 | 1176 |
| 827516 | N/A | N/A | GGCTGGAGATCTTTAG | 36 | 27607 | 27622 | 1177 |
| 827528 | N/A | N/A | TGGGACTTGCTAATGA | 42 | 28251 | 28266 | 606 |
| 827531 | N/A | N/A | CAGAATAGCCGGGCGC | 36 | 28650 | 28665 | 1180 |
| 827532 | N/A | N/A | GGCAGACACGAGGGTC | 31 | 28699 | 28714 | 1181 |
| 827533 | N/A | N/A | CCATACGGATGAACCT | 24 | 28741 | 28756 | 1182 |
| 827534 | N/A | N/A | TACCATACGGATGAAC | 31 | 28743 | 28758 | 1183 |
| 827535 | N/A | N/A | CTACCATACGGATGAA | 38 | 28744 | 28759 | 1184 |
| 827547 | N/A | N/A | GGTGATGTCACTTCGG | 8 | 29031 | 29046 | 608 |
| 827550 | N/A | N/A | AGGGAATTAAGCCACA | 8 | 29501 | 29516 | 1187 |
| 827551 | N/A | N/A | GGATACACCAGTGTAA | 43 | 29904 | 29919 | 1188 |
| 827552 | N/A | N/A | AGCTAAGTCAGGCGAA | 38 | 29930 | 29945 | 1189 |
| 827553 | N/A | N/A | TATGAGTGTGCCTTTG | 42 | 30329 | 30344 | 1190 |
| 827554 | N/A | N/A | TTCAAGGTTGCAAGTG | 24 | 30348 | 30363 | 1191 |
| 827555 | N/A | N/A | AGCTAAGCCAGGGACA | 59 | 30416 | 30431 | 1192 |
| 827567 | N/A | N/A | GGATAGGGTTGTGTCA | 96 | 30925 | 30940 | 610 |
| 827570 | N/A | N/A | ATCAAGGTCACTCCCA | 65 | 30959 | 30974 | 1195 |
| 827571 | N/A | N/A | GAAGACCCATTCCTAG | 69 | 30992 | 31007 | 1196 |
| 827572 | N/A | N/A | CCATATCGATCCCTCT | 67 | 31115 | 31130 | 1197 |
| 827573 | N/A | N/A | GAATTTCCTGGACCTT | 64 | 31142 | 31157 | 1198 |
| 827574 | N/A | N/A | GAAATGGTAGAGGATG | 82 | 31157 | 31172 | 1199 |
| 827575 | N/A | N/A | AGGCACGACCTACCGT | 124 | 31272 | 31287 | 1200 |

### Example 2: Effect of Modified Oligonucleotides Complementary to α-ENaC in Hep3B Cells at Various Doses

Selected oligonucleotides listed in Example 1 were tested at various doses in Hep3B cells. Cells were plated at a density of 20,000 cells per well and transfected using electroporation with 148, 444, 1,333, or 4,000 nM of modified oligonucleotide, as specified in the tables below. After a treatment period of approximately 24 hours, total RNA was isolated and analyzed as described in Example 1. As illustrated in the tables below, α-ENaC mRNA levels were reduced in a dose-dependent manner in cells treated with a modified oligonucleotide complementary to an α-ENaC nucleic acid.

TABLE 6
Percent level of human α-ENaC mRNA

| Compound | α-ENaC expression (% control) | | | |
|---|---|---|---|---|
| Number | 148 nM | 444 nM | 1,333 nM | 4,000 nM |
| 797192 | 39 | 30 | 13 | 8 |
| 797235 | 68 | 13 | 6 | 5 |
| 797294 | 41 | 25 | 13 | 5 |
| 797495 | 16 | 17 | 23 | 12 |
| 797501 | 34 | 21 | 20 | 3 |
| 797507 | 25 | 14 | 5 | 8 |
| 826229 | 74 | 17 | 8 | 4 |
| 826249 | 86 | 24 | 8 | 6 |
| 826683 | 80 | 64 | 40 | 15 |
| 826761 | 53 | 11 | 9 | 5 |
| 826799 | 40 | 26 | 12 | 7 |
| 826800 | 51 | 40 | 24 | 11 |
| 826877 | 63 | 49 | 27 | 8 |
| 827277 | 36 | 42 | 35 | 10 |
| 827372 | 9 | 14 | 1 | 2 |
| 827392 | 17 | 13 | 7 | 3 |
| 827410 | 66 | 39 | 23 | 7 |
| 827449 | 35 | 22 | 18 | 5 |
| 827547 | 11 | 5 | 4 | 1 |

TABLE 7
Percent level of human α-ENaC mRNA

| Compound | α-ENaC expression (% control) | | | |
|---|---|---|---|---|
| Number | 148 nM | 444 nM | 1,333 nM | 4,000 nM |
| 797469 | 78 | 49 | 24 | 16 |
| 797501 | 46 | 29 | 40 | 20 |
| 826232 | 40 | 43 | 16 | 7 |
| 826233 | 74 | 49 | 19 | 28 |
| 826626 | 70 | 51 | 38 | 9 |
| 826743 | 75 | 43 | 25 | 12 |
| 826763 | 65 | 25 | 24 | 7 |
| 826764 | 58 | 43 | 34 | 25 |
| 826784 | 81 | 50 | 16 | 15 |
| 826819 | 42 | 26 | 15 | 3 |
| 826821 | 73 | 51 | 29 | 10 |
| 826878 | 38 | 32 | 22 | 2 |
| 826880 | 35 | 30 | 11 | 17 |
| 827179 | 50 | 28 | 8 | 5 |
| 827199 | 39 | 14 | 18 | 9 |
| 827278 | 33 | 22 | 10 | 9 |
| 827393 | 48 | 23 | 9 | 6 |
| 827432 | 59 | 49 | 18 | 7 |
| 827550 | 77 | 53 | 38 | 14 |

TABLE 8
Percent level of human α-ENaC mRNA

| Compound | α-ENaC expression (% control) | | | |
|---|---|---|---|---|
| Number | 148 nM | 444 nM | 1,333 nM | 4,000 nM |
| 797501 | 27 | 17 | 10 | 5 |
| 826213 | 35 | 23 | 16 | 9 |
| 826411 | 57 | 30 | 23 | 7 |
| 826451 | 80 | 59 | 30 | 27 |
| 826508 | 55 | 42 | 25 | 11 |
| 826668 | 34 | 27 | 13 | 16 |
| 826687 | 69 | 34 | 29 | 6 |
| 826688 | 43 | 22 | 6 | 4 |
| 826746 | 34 | 21 | 21 | 9 |
| 826785 | 39 | 23 | 9 | 4 |
| 827042 | 57 | 30 | 18 | 15 |
| 827081 | 37 | 11 | 6 | 3 |
| 827200 | 35 | 19 | 11 | 7 |
| 827280 | 19 | 15 | 7 | 4 |
| 827318 | 51 | 21 | 11 | 11 |
| 827378 | 44 | 29 | 8 | 12 |
| 827395 | 44 | 22 | 19 | 4 |
| 827414 | 54 | 27 | 15 | 14 |
| 827435 | 28 | 19 | 7 | 3 |

TABLE 9
Percent level of human α-ENaC mRNA

| Compound | α-ENaC expression (% control) | | | |
|---|---|---|---|---|
| Number | 148 nM | 444 nM | 1,333 nM | 4,000 nM |
| 797309 | 53 | 15 | 12 | 8 |
| 797501 | 24 | 20 | 16 | 11 |
| 826334 | 65 | 43 | 26 | 23 |
| 826392 | 46 | 26 | 25 | 5 |
| 826393 | 57 | 12 | 12 | 7 |
| 826394 | 51 | 32 | 16 | 17 |
| 826591 | 44 | 14 | 13 | 6 |
| 826631 | 22 | 10 | 9 | 2 |
| 826632 | 38 | 22 | 13 | 14 |
| 826689 | 54 | 33 | 17 | 10 |
| 826809 | 21 | 12 | 10 | 2 |
| 826825 | 41 | 23 | 18 | 3 |
| 827283 | 46 | 28 | 20 | 14 |
| 827301 | 65 | 49 | 24 | 13 |
| 827359 | 18 | 15 | 6 | 3 |
| 827360 | 33 | 10 | 15 | 2 |
| 827379 | 28 | 22 | 13 | 8 |
| 827398 | 34 | 25 | 16 | 4 |
| 827437 | 37 | 24 | 8 | 11 |

TABLE 10
Percent level of human α-ENaC mRNA

| Compound | α-ENaC expression (% control) | | | |
|---|---|---|---|---|
| Number | 148 nM | 444 nM | 1,333 nM | 4,000 nM |
| 797304 | 55 | 31 | 17 | 7 |
| 797308 | 28 | 19 | 7 | 14 |
| 797494 | 44 | 35 | 14 | 11 |
| 797501 | 56 | 26 | 12 | 17 |
| 826259 | 79 | 40 | 19 | 8 |
| 826514 | 54 | 53 | 32 | 25 |
| 826655 | 65 | 46 | 32 | 18 |
| 826711 | 51 | 28 | 30 | 12 |
| 826828 | 57 | 35 | 18 | 4 |
| 826906 | 72 | 20 | 22 | 24 |
| 827148 | 74 | 47 | 34 | 23 |
| 827284 | 34 | 22 | 13 | 5 |
| 827382 | 53 | 31 | 27 | 18 |
| 827383 | 69 | 60 | 37 | 23 |
| 827419 | 33 | 18 | 13 | 5 |
| 827420 | 71 | 34 | 20 | 12 |
| 827497 | 46 | 14 | 11 | 9 |
| 827498 | 56 | 34 | 24 | 13 |
| 827518 | 65 | 33 | 15 | 14 |

TABLE 11
Percent level of human α-ENaC mRNA

| Compound | α-ENaC expression (% control) | | | |
|---|---|---|---|---|
| Number | 148 nM | 444 nM | 1,333 nM | 4,000 nM |
| 797501 | 44 | 33 | 16 | 6 |
| 826183 | 57 | 38 | 13 | 10 |
| 826202 | 77 | 35 | 25 | 5 |
| 826241 | 43 | 36 | 25 | 18 |
| 826262 | 81 | 50 | 21 | 12 |
| 826338 | 79 | 35 | 13 | 5 |
| 826558 | 80 | 53 | 24 | 16 |
| 826576 | 70 | 45 | 27 | 12 |
| 826673 | 88 | 40 | 29 | 12 |
| 826753 | 64 | 39 | 21 | 23 |
| 826754 | 67 | 47 | 22 | 11 |
| 826793 | 62 | 35 | 13 | 6 |
| 826811 | 85 | 42 | 18 | 3 |
| 827030 | 43 | 23 | 9 | 4 |
| 827149 | 42 | 38 | 23 | 11 |
| 827150 | 54 | 41 | 20 | 10 |
| 827307 | 46 | 23 | 20 | 6 |
| 827347 | 66 | 41 | 26 | 16 |
| 827441 | 30 | 13 | 11 | 1 |

TABLE 12
Percent level of human α-ENaC

| Compound | α-ENaC expression (% control) | | | |
|---|---|---|---|---|
| Number | 148 nM | 444 nM | 1,333 nM | 4,000 nM |
| 797131 | 55 | 27 | 15 | 7 |
| 797497 | 56 | 31 | 28 | 13 |
| 797501 | 34 | 23 | 24 | 5 |
| 826659 | 50 | 40 | 13 | 5 |
| 826678 | 44 | 25 | 23 | 9 |
| 826776 | 71 | 34 | 24 | 9 |
| 826794 | 52 | 25 | 18 | 9 |
| 826891 | 50 | 32 | 18 | 9 |
| 827131 | 100 | 63 | 44 | 10 |
| 827151 | 32 | 29 | 12 | 10 |
| 827270 | 54 | 33 | 23 | 11 |
| 827288 | 42 | 35 | 20 | 5 |
| 827289 | 65 | 33 | 21 | 7 |
| 827309 | 79 | 45 | 30 | 7 |
| 827348 | 69 | 54 | 33 | 10 |
| 827368 | 63 | 35 | 22 | 7 |
| 827386 | 85 | 46 | 19 | 6 |
| 827502 | 55 | 21 | 12 | 11 |
| 827524 | 78 | 39 | 26 | 14 |

TABLE 13
Percent level of human α-ENaC

| Compound | α-ENaC expression (% control) | | | |
|---|---|---|---|---|
| Number | 148 nM | 444 nM | 1,333 nM | 4,000 nM |
| 797264 | 59 | 30 | 28 | 5 |
| 797312 | 63 | 44 | 28 | 11 |
| 797501 | 47 | 12 | 6 | 3 |
| 826168 | 87 | 58 | 16 | 11 |
| 826169 | 65 | 29 | 15 | 13 |
| 826403 | 66 | 35 | 18 | 13 |
| 826484 | 65 | 46 | 23 | 11 |
| 826660 | 60 | 53 | 22 | 7 |
| 826679 | 60 | 46 | 35 | 14 |
| 826718 | 68 | 51 | 40 | 6 |
| 826796 | 66 | 61 | 36 | 9 |
| 826816 | 104 | 53 | 26 | 10 |
| 827035 | 57 | 28 | 25 | 9 |
| 827134 | 112 | 57 | 31 | 33 |
| 827175 | 43 | 39 | 13 | 7 |
| 827254 | 53 | 36 | 24 | 6 |
| 827408 | 53 | 28 | 21 | 15 |
| 827426 | 79 | 34 | 23 | 9 |
| 827447 | 35 | 18 | 13 | 10 |

TABLE 14
Percent level of human α-ENaC

| Compound | α-ENaC expression (% control) | | | |
|---|---|---|---|---|
| Number | 148 nM | 444 nM | 1,333 nM | 4,000 nM |
| 797469 | 81 | 55 | 24 | 17 |
| 797501 | 26 | 25 | 41 | 21 |
| 826232 | 39 | 32 | 14 | 5 |
| 826233 | 88 | 58 | 23 | 29 |
| 826626 | 59 | 53 | 39 | 8 |
| 826743 | 83 | 47 | 23 | 10 |
| 826763 | 77 | 24 | 20 | 9 |
| 826764 | 33 | 45 | 37 | 26 |
| 826784 | 44 | 47 | 16 | 15 |
| 826819 | 40 | 24 | 10 | 2 |
| 826821 | 81 | 52 | 31 | 11 |
| 826878 | 39 | 31 | 19 | 2 |
| 826880 | 41 | 28 | 13 | 17 |
| 827179 | 62 | 24 | 7 | 5 |
| 827199 | 44 | 14 | 20 | 10 |
| 827278 | 31 | 23 | 10 | 9 |
| 827393 | 55 | 22 | 9 | 6 |
| 827432 | 66 | 52 | 24 | 7 |
| 827550 | 62 | 43 | 36 | 16 |

TABLE 15
Percent level of human α-ENaC

| Compound | α-ENaC expression (% control) | | | |
|---|---|---|---|---|
| Number | 148 nM | 444 nM | 1,333 nM | 4,000 nM |
| 797131 | 58 | 28 | 16 | 7 |
| 797497 | 61 | 34 | 30 | 14 |
| 797501 | 37 | 24 | 26 | 5 |
| 826659 | 54 | 42 | 14 | 5 |
| 826678 | 47 | 26 | 25 | 9 |
| 826776 | 76 | 36 | 26 | 10 |
| 826794 | 57 | 28 | 19 | 10 |
| 826891 | 54 | 34 | 19 | 10 |
| 827131 | 109 | 69 | 48 | 10 |
| 827151 | 34 | 32 | 13 | 11 |
| 827270 | 57 | 35 | 25 | 11 |
| 827288 | 45 | 37 | 21 | 6 |
| 827289 | 71 | 36 | 23 | 8 |
| 827309 | 84 | 48 | 32 | 8 |
| 827348 | 73 | 58 | 35 | 11 |
| 827368 | 67 | 38 | 23 | 7 |
| 827386 | 92 | 50 | 21 | 7 |
| 827502 | 60 | 24 | 13 | 12 |
| 827524 | 83 | 41 | 28 | 15 |

### Example 3: Effect of Various Doses of Modified Oligonucleotides Complementary to Human α-ENaC In Vitro Via Free Uptake

Selected oligonucleotides were tested at various doses in A431 cells by free uptake. Cells were plated at a density of 10,000 cells per well with 16, 49, 148, 1,333, or 4,000 nM of modified oligonucleotide, as specified in the tables below. After a treatment period of approximately 24 hours, total RNA was isolated and analyzed as in Example 1. As illustrated in the tables below, α-ENaC mRNA levels were reduced in a dose-dependent manner in cells treated with a modified oligonucleotide complementary to an α-ENaC nucleic acid.

TABLE 16
Level of α-ENaC mRNA in A431 cells

| | α-ENaC expression (% control) | | | | | | |
|---|---|---|---|---|---|---|---|
| Compound | 16 | 49 | 148 | 444 | 1,333 | 4,000 | IC50 |
| Number | nM | nM | nM | nM | nM | nM | (μM) |
| 797236 | 129 | 92 | 50 | 31 | 18 | 6 | 0.23 |
| 797308 | 89 | 66 | 27 | 13 | 9 | 4 | 0.08 |
| 797313 | 94 | 82 | 47 | 25 | 15 | 9 | 0.17 |
| 797468 | 90 | 77 | 55 | 30 | 19 | 11 | 0.19 |
| 797495 | 50 | 26 | 11 | 3 | 8 | 7 | 0.01 |
| 826632 | 76 | 75 | 61 | 28 | 22 | 11 | 0.22 |
| 826743 | 85 | 81 | 57 | 28 | 23 | 19 | 0.22 |
| 826763 | 73 | 55 | 35 | 16 | 14 | 8 | 0.06 |
| 826819 | 85 | 87 | 73 | 58 | 44 | 38 | 1.06 |
| 826906 | 85 | 75 | 52 | 30 | 17 | 9 | 0.16 |

TABLE 17
Level of α-ENaC mRNA in A431 cells

| | α-ENaC expression (% control) | | | | | | |
|---|---|---|---|---|---|---|---|
| Compound | 16 | 49 | 148 | 444 | 1,333 | 4,000 | IC50 |
| Number | nM | nM | nM | nM | nM | nM | (μM) |
| 827030 | 109 | 98 | 78 | 55 | 41 | 35 | 0.97 |
| 827200 | 100 | 85 | 79 | 55 | 50 | 38 | 1.23 |
| 827288 | 85 | 71 | 53 | 33 | 13 | 21 | 0.16 |
| 827307 | 68 | 58 | 33 | 14 | 5 | 2 | 0.06 |
| 827347 | 66 | 47 | 18 | 5 | 2 | 1 | 0.04 |
| 827359 | 56 | 45 | 27 | 11 | 6 | 4 | 0.03 |
| 827372 | 47 | 22 | 7 | 3 | 1 | 1 | 0.01 |
| 827392 | 76 | 44 | 18 | 7 | 4 | 3 | 0.04 |
| 827414 | 79 | 60 | 38 | 17 | 8 | 5 | 0.08 |
| 827497 | 64 | 49 | 24 | 8 | 6 | 5 | 0.04 |

### Example 4: Tolerability of Modified Oligonucleotides Complementary to Human α-ENaC in CD1 Mice Following Systemic Delivery

CD1 ® mice (Charles River, MA) are a multipurpose mice model, frequently utilized for safety and efficacy testing. The mice were treated with modified oligonucleotides selected from studies described above and evaluated for changes in the levels of various plasma chemistry markers.

### Treatment

Groups of 6-8 week old male CD1 mice were injected subcutaneously once a week for 6 weeks with 50 mg/kg of a modified oligonucleotide listed in the tables below (50 mg/kg/week dose). Each group contained 4 mice. One group of male CD1 mice was injected subcutaneously once a week for 6 weeks with PBS. Mice were sacrificed 48 hours after the last dose, and organs and plasma were harvested for further analysis.

### Plasma Chemistry Markers

To evaluate the effect of modified oligonucleotides on liver and kidney function, plasma levels of transaminases, albumin, BUN, and billirubin were measured using an automated clinical chemistry analyzer (Hitachi Olympus AU400e, Melville, NY). The results in the tables below show that most of the tested modified oligonucleotides were well tolerated when delivered systemically, with ALT and AST levels under approximately 200 IU/L and albumin, BUN, creatine, and total bilirubin within acceptable ranges.

TABLE 18
Levels of plasma chemistry markers

| Compound | ALT | AST | | | | |
|---|---|---|---|---|---|---|
| No. | (IU/L) | (IU/L) | Albumin | BUN | Creatine | T. Bil. |
| PBS | 34.8 | 39.8 | 2.83 | 25.0 | .065 | .195 |
| 797131 | 167.8 | 162.0 | 2.39 | 21.6 | .055 | .188 |
| 797236 | 70.5 | 93.8 | 2.78 | 24.8 | .057 | .183 |
| 797258 | 1061.3 | 1077.8 | 2.91 | 25.8 | .060 | .250 |
| 797262 | 244.5 | 324.0 | 2.54 | 27.9 | .045 | .165 |
| 797264 | 484.0 | 247.3 | 2.92 | 26.1 | .080 | .178 |
| 797266 | 641.3 | 330.3 | 3.05 | 24.7 | .070 | .180 |
| 797289 | 218.8 | 175.5 | 2.66 | 21.8 | .065 | .148 |
| 797293 | 921.5 | 638.5 | 2.85 | 27.1 | .080 | .208 |
| 797294 | 248.5 | 226.0 | 2.99 | 23.1 | .043 | .268 |
| 797295 | 1262.8 | 954.3 | 3.01 | 22.7 | .063 | .408 |
| 797304 | 252.3 | 208.8 | 2.70 | 23.0 | .060 | .208 |
| 797307 | 151.3 | 123.8 | 2.97 | 25.6 | .093 | .198 |
| 797308 | 65.8 | 114.3 | 2.71 | 22.8 | .070 | .158 |
| 797312 | 1630.5 | 862.8 | 3.40 | 25.2 | .135 | .315 |
| 797313 | 46.3 | 77.0 | 3.15 | 23.4 | .140 | .203 |
| 797340 | 558.3 | 316.0 | 3.26 | 28.6 | .143 | .263 |
| 797444 | 224.0 | 285.8 | 2.23 | 27.6 | .048 | .205 |
| 797466 | 433.5 | 446.3 | 2.79 | 22.4 | .093 | .138 |
| 797468 | 87.0 | 101.8 | 2.94 | 23.4 | .120 | .145 |
| 797495 | 45.8 | 121.8 | 3.62 | 24.0 | .148 | .263 |
| 797497 | 61.0 | 78.0 | 2.95 | 24.3 | .125 | .148 |
| 797500 | 43.8 | 59.3 | 3.11 | 24.2 | .118 | .193 |
| 797501 | 546.0 | 500.8 | 2.82 | 25.6 | .115 | .303 |
| 797507 | 65.5 | 89.8 | 3.03 | 22.8 | .090 | .168 |
| 797508 | 120.5 | 188.3 | 2.41 | 23.9 | .055 | .155 |
| 797523 | 237.3 | 168.8 | 3.48 | 25.4 | .110 | .170 |

TABLE 19
Levels of plasma chemistry markers

| Compound | ALT | AST | | | | |
|---|---|---|---|---|---|---|
| No. | (IU/L) | (IU/L) | Albumin | BUN | Creatine | T. Bil. |
| PBS | 54.0 | 59.5 | 2.81 | 25.8 | .080 | .193 |
| 797192 | 2079.8 | 889.5 | 3.26 | 25.2 | .125 | .793 |
| 797309 | 112.5 | 91.5 | 2.91 | 22.5 | .078 | .165 |
| 797469 | 3698.3 | 3053.3 | 3.80 | 20.1 | .148 | .188 |
| 826183 | n.d. | n.d. | n.d. | n.d. | n.d. | n.d. |
| 826262 | 236.8 | 196.8 | 2.45 | 21.9 | .058 | .195 |
| 826393 | 116.8 | 89.8 | 2.85 | 20.5 | .070 | .168 |
| 826394 | 466.0 | 196.3 | 2.81 | 23.1 | .068 | .218 |
| 826558 | 126.8 | 117.3 | 2.97 | 21.6 | .080 | .155 |
| 826631 | 1073.5 | 1267.0 | 2.61 | 19.0 | .040 | .275 |
| 826632 | 113.3 | 129.5 | 2.94 | 22.4 | .085 | .173 |
| 826655 | 1349.8 | 754.5 | 3.13 | 21.1 | .123 | 6.315 |
| 826687 | 1174.0 | 647.0 | 2.85 | 20.0 | .075 | .178 |
| 826688 | 430.0 | 362.0 | 3.38 | 22.5 | .100 | .215 |
| 826743 | 115.0 | 111.8 | 2.94 | 21.6 | .085 | .160 |
| 826753 | 58.0 | 70.0 | 2.68 | 24.5 | .070 | .138 |
| 826763 | 77.0 | 89.3 | 2.97 | 22.9 | .083 | .203 |
| 826776 | 1685.8 | 1027.0 | 2.55 | 20.7 | .058 | .248 |
| 826793 | 1306.3 | 563.5 | 2.85 | 18.8 | .065 | .250 |
| 826796 | 1552.5 | 740.8 | 3.29 | 19.5 | .088 | .453 |
| 826811 | 439.3 | 347.5 | 3.00 | 19.5 | .085 | .438 |
| 826819 | 297.0 | 146.8 | 2.88 | 26.6 | .125 | .163 |
| 826828 | 559.0 | 314.0 | 2.71 | 17.4 | .060 | .170 |
| 826906 | 41.0 | 56.5 | 2.98 | 22.5 | .093 | .163 |
| 827030 | 44.0 | 55.8 | 3.04 | 20.0 | .093 | .180 |
| 827035 | n.d. | n.d. | n.d. | n.d. | n.d. | n.d. |

TABLE 20
Levels of plasma chemistry markers

| Compound | ALT | AST | | | | |
|---|---|---|---|---|---|---|
| No. | (IU/L) | (IU/L) | Albumin | BUN | Creatine | T. Bil. |
| PBS | 45.0 | 53.0 | 2.80 | 25.5 | .065 | .255 |
| 827148 | 33.5 | 52.3 | 2.63 | 23.8 | .080 | .223 |
| 827150 | n.d. | n.d. | n.d. | n.d. | n.d. | n.d. |
| 827175 | 123.8 | 246.8 | 2.50 | 26.1 | .078 | .218 |
| 827200 | 61.5 | 51.0 | 2.79 | 27.1 | .058 | .195 |
| 827254 | 155.3 | 161.5 | 2.77 | 26.9 | .068 | .265 |
| 827288 | 65.8 | 59.5 | 2.77 | 22.5 | .068 | .280 |
| 827307 | 55.5 | 61.0 | 2.70 | 26.3 | .053 | .203 |
| 827347 | 52.5 | 60.0 | 2.72 | 25.9 | .063 | .140 |
| 827348 | 284.5 | 197.5 | 2.99 | 21.9 | .070 | .330 |
| 827359 | 65.8 | 72.8 | 2.60 | 22.7 | .063 | .210 |
| 827360 | 45.8 | 52.8 | 2.68 | 24.1 | .088 | .188 |
| 827372 | 49.0 | 51.5 | 2.73 | 24.7 | .053 | .213 |
| 827382 | 33.3 | 45.8 | 2.69 | 22.0 | .048 | .183 |
| 827392 | 39.5 | 57.0 | 2.77 | 21.3 | .063 | .163 |
| 827393 | 207.5 | 118.8 | 2.82 | 24.2 | .050 | .220 |
| 827398 | 121.3 | 181.3 | 2.66 | 22.8 | .045 | .178 |
| 827408 | 339.8 | 292.5 | 2.53 | 28.0 | .048 | .143 |
| 827410 | 205.8 | 436.8 | 2.66 | 19.7 | .040 | .163 |
| 827414 | 145.8 | 123.0 | 2.59 | 23.2 | .055 | .148 |
| 827419 | 62.0 | 73.3 | 2.68 | 27.5 | .065 | .178 |
| 827437 | 63.8 | 103.8 | 2.56 | 19.5 | .050 | .243 |
| 827449 | 300.0 | 211.3 | 2.49 | 22.0 | .040 | .168 |
| 827497 | 100.5 | 112.8 | 2.19 | 22.0 | .043 | .170 |
| 827502 | 126.3 | 106.3 | 2.36 | 22.9 | .043 | .108 |

### Organ Weights

Organ weights were measured at the end of the study, and kidney, liver, and spleen weights are presented in the table below. The results provide additional evidence that most of the modified oligonucleotides were well tolerated when delivered systemically.

TABLE 21
Organ weights

| Compound No. | Kidney (g) | Liver (g) | Spleen (g) |
|---|---|---|---|
| PBS | 0.678 | 2.542 | 0.133 |
| 797131 | 0.598 | 2.263 | 0.151 |
| 797236 | 0.621 | 2.281 | 0.195 |
| 797258 | 0.719 | 3.663 | 0.212 |
| 797262 | 0.625 | 3.214 | 0.127 |
| 797264 | 0.496 | 2.485 | 0.169 |
| 797266 | 0.547 | 2.633 | 0.169 |
| 797289 | 0.618 | 3.064 | 0.165 |
| 797293 | 0.647 | 2.197 | 0.200 |
| 797294 | 0.566 | 1.675 | 0.154 |
| 797295 | 0.558 | 2.147 | 0.170 |
| 797304 | 0.691 | 3.263 | 0.173 |
| 797307 | 0.607 | 2.546 | 0.203 |
| 797308 | 0.616 | 2.113 | 0.143 |
| 797312 | 0.562 | 2.941 | 0.123 |
| 797313 | 0.536 | 2.236 | 0.138 |
| 797340 | 0.529 | 2.325 | 0.144 |
| 797444 | 0.580 | 6.846 | 0.551 |
| 797466 | 0.491 | 2.289 | 0.222 |
| 797468 | 0.617 | 2.275 | 0.145 |
| 797495 | 0.602 | 2.300 | 0.128 |
| 797497 | 0.651 | 2.483 | 0.151 |
| 797500 | 0.563 | 2.031 | 0.119 |
| 797501 | 0.479 | 2.978 | 0.140 |
| 797507 | 0.551 | 2.359 | 0.115 |
| 797508 | 0.469 | 1.322 | 0.148 |
| 797523 | 0.560 | 2.614 | 0.172 |

TABLE 22
Organ weights

| Compound No. | Kidney (g) | Liver (g) | Spleen (g) |
|---|---|---|---|
| PBS | 0.610 | 2.212 | 0.138 |
| 797192 | 0.530 | 4.632 | 0.157 |
| 797309 | 0.586 | 2.160 | 0.115 |
| 797469 | 0.633 | 4.636 | 0.238 |
| 826183 | n.d. | n.d. | n.d. |
| 826262 | 0.609 | 2.197 | 0.227 |
| 826393 | 0.508 | 3.084 | 0.210 |
| 826394 | 0.530 | 2.914 | 0.206 |
| 826558 | 0.567 | 2.048 | 0.149 |
| 826631 | 0.587 | 2.361 | 0.169 |
| 826632 | 0.595 | 2.442 | 0.136 |
| 826655 | 0.588 | 3.511 | 0.113 |
| 826687 | 0.619 | 2.750 | 0.261 |
| 826688 | 0.546 | 2.418 | 0.214 |
| 826743 | 0.608 | 2.162 | 0.110 |
| 826753 | 0.538 | 2.364 | 0.140 |
| 826763 | 0.542 | 2.478 | 0.144 |
| 826776 | 0.574 | 4.112 | 0.386 |
| 826793 | 0.555 | 2.295 | 0.173 |
| 826796 | 0.605 | 2.566 | 0.151 |
| 826811 | 0.557 | 2.085 | 0.133 |
| 826819 | 0.517 | 2.559 | 0.144 |
| 826828 | 0.590 | 2.046 | 0.191 |
| 826906 | 0.561 | 2.121 | 0.123 |
| 827030 | 0.564 | 1.974 | 0.114 |
| 827035 | n.d. | n.d. | n.d. |

TABLE 23
Organ weights

| Compound No. | Kidney (g) | Liver (g) | Spleen (g) |
|---|---|---|---|
| PBS | 0.615 | 2.172 | 0.108 |
| 827148 | 0.623 | 2.413 | 0.142 |
| 827150 | n.d. | n.d. | n.d. |
| 827175 | 0.683 | 2.521 | 0.139 |
| 827200 | 0.640 | 2.682 | 0.127 |
| 827254 | 0.631 | 2.589 | 0.139 |
| 827288 | 0.579 | 2.341 | 0.138 |
| 827307 | 0.614 | 2.391 | 0.133 |
| 827347 | 0.596 | 2.235 | 0.152 |
| 827348 | 0.678 | 2.832 | 0.251 |
| 827359 | 0.647 | 2.316 | 0.146 |
| 827360 | 0.517 | 2.098 | 0.147 |
| 827372 | 0.657 | 2.120 | 0.140 |
| 827382 | 0.574 | 2.089 | 0.142 |
| 827392 | 0.595 | 2.208 | 0.124 |
| 827393 | 0.603 | 2.307 | 0.137 |
| 827398 | 0.590 | 2.249 | 0.141 |
| 827408 | 0.751 | 2.399 | 0.290 |
| 827410 | 0.653 | 3.247 | 0.174 |
| 827414 | 0.663 | 2.787 | 0.185 |
| 827419 | 0.682 | 2.327 | 0.150 |
| 827437 | 0.674 | 2.523 | 0.544 |
| 827449 | 0.619 | 2.798 | 0.155 |
| 827497 | 0.630 | 2.368 | 0.189 |
| 827502 | 0.674 | 3.082 | 0.183 |

### Example 5: Establishment of a Transgenic Mouse Line Expressing Human α-ENaC

A transgenic mouse was developed to analyze knockdown of human α-ENaC in a mouse model. A 41,279 bp portion of the gene for human α-ENaC ABC14-50929300K14 (digested with NotI) was microinjected into embryos of C57BL/6 WT mice. Five transgene positive F0 mouse pups were obtained, and one founder was used to generate a C57BL/6 hα-ENaC mouse line. The line was evaluated for expression of hα-ENaC in tongue, brain, heart, colon, trachea, pancreas, kidney, liver, spleen, skeletal muscle, fat, uterus, and both total lung and lung fractions. The mouse model exhibits hα-ENaC expression in a variety of tissues, and, importantly, high levels of expression in all fractions of the lung.

### Example 6: Effect of Modified Oligonucleotides on Human α-ENaC Expression in a Transgenic Mouse

### Treatment

Transgenic mice were maintained on a 12-hour light/dark cycle and were fed ad libitum normal diet. Animals were acclimated for at least 7 days in the research facility before initiation of the experiment. Modified oligonucleotides were prepared in buffered saline (PBS) and sterilized by filtering through a 0.2 micron filter. Oligonucleotides were dissolved in 0.9% PBS for injection.

The C57Bl/6-TG(hα-ENaC) mice weighing ˜20 g were divided into groups of 2-4 mice. Groups of mice were administered 2.5 mg/kg of modified oligonucleotide twice a week for two weeks (5 mg/kg/week) via oropharyngeal aspiration. A control group of 6 mice was given PBS twice per week for two weeks. The PBS group served as the control group to which animals dosed with modified oligonucleotide were compared. Mice were sacrificed 48 hrs after the last dose and organs were harvested for further analysis.

### Human α-ENaC Expression Levels

Total RNA was isolated from the whole lung and human α-ENaC mRNA levels were measured as described in Example 1. Results are presented in the table below as percent reduction of the amount of α-ENaC mRNA relative to untreated control. As illustrated in the table below, α-ENaC mRNA levels were reduced in lung of modified oligonucleotide-treated animals.

TABLE 24
Percent level of human α-ENaC mRNA

| | Tissue | | | |
|---|---|---|---|---|
| Compound No. | Lung | Liver | Colon | Kidney |
| 797236 | 41 | 62 | 85 | 87 |
| 797308* | 36 | 41 | 99 | 87 |
| 797313 | 41 | 54 | 87 | 89 |
| 797468 | 45 | 64 | 77 | 102 |
| 797495 | 27 | 30 | 69 | 66 |
| 826632** | 40 | 81 | 77 | 87 |
| 826743 | 46 | 73 | 106 | 101 |
| 826763 | 32 | 51 | 94 | 96 |
| 826819 | 45 | 50 | 93 | 86 |
| 826906 | 42 | 64 | 107 | 101 |
| 827030 | 45 | 59 | 90 | 73 |
| 827200 | 51 | 72 | 101 | 129 |
| 827288 | 54 | 66 | 105 | 75 |
| 827307 | 34 | 68 | 85 | 91 |
| 827347 | 28 | 66 | 97 | 103 |
| 827359* | 34 | 37 | 82 | 90 |
| 827372 | 21 | 29 | 50 | 70 |
| 827392 | 28 | 50 | 73 | 72 |
| 827414 | 36 | 45 | 84 | 93 |
| 827497 | 34 | 61 | 90 | 86 |

*Group contained 3 mice
**Group contained 2 mice
All other groups contained 4 mice

### Example 7: Dose-Response of Compound 827359 on Human α-ENaC Expression in a Transgenic Mouse

### Treatment

Transgenic mice were maintained on a 12-hour light/dark cycle and were fed ad libitum normal diet. Animals were acclimated for at least 7 days in the research facility before initiation of the experiment. Modified oligonucleotides were prepared in buffered saline (PBS) and sterilized by filtering through a 0.2 micron filter. Oligonucleotides were dissolved in 0.9% PBS.

The C57Bl/6-TG(hα-ENaC) mice weighing ˜20 g were divided into groups of 12 mice. Groups of 12 mice were administered 0.033, 0.1, 0.33 or 1.0 mg/kg of modified oligonucleotide twice a week for three weeks (5 mg/kg/week) via aerosol dosing. A control group of 12 mice was given aerosol saline twice per week for 3 weeks. The PBS group served as the control group to which animals dosed with modified oligonucleotide were compared. Mice were sacrificed 3 days after the last dose and organs were harvested for further analysis.

### Human α-ENaC Expression Levels

Total RNA was isolated from the whole lung and human α-ENaC mRNA levels were measured by quantitative real-time PCR as described in Example 1. Results are presented in the table below as percent reduction of the amount of α-ENaC mRNA relative to untreated control. As illustrated in the table below, α-ENaC mRNA levels were reduced in a dose-dependent manner in modified oligonucleotide-treated animals.

TABLE 25
Dose Response of 827359 in transgenic mouse

| Conc. 827359 | % |
|---|---|
| (mg/kg/dose) | Control |
| 0 [Saline] | 100.0 |
| 0.033 | 73.4 |
| 0.100 | 50.4 |
| 0.330 | 38.1 |
| 1.000 | 33.3 |

### Example 8: Human Peripheral Blood Mononuclear Cells (hPBMC) Assay

The hPBMC assay was performed using BD Vautainer CPT tube method. A sample of whole blood from volunteered donors with informed consent at US HealthWorks clinic (Faraday & El Camino Real, Carlsbad) was obtained and collected in 4-15 BD Vacutainer CPT 8 ml tubes (VWR Cat. #BD362753). The approximate starting total whole blood volume in the CPT tubes for each donor was recorded using the PBMC assay data sheet.

The blood sample was remixed immediately prior to centrifugation by gently inverting tubes 8-10 times. CPT tubes were centrifuged at rt (18-25° C.) in a horizontal (swing-out) rotor for 30 min. at 1500-1800 RCF with brake off (2700 RPM Beckman Allegra 6R). The cells were retrieved from the buffy coat interface (between Ficoll and polymer gel layers); transferred to a sterile 50 ml conical tube and pooled up to 5 CPT tubes/50 ml conical tube/donor. The cells were then washed twice with PBS (Ca⁺⁺, Mg⁺⁺ free; GIBCO). The tubes were topped up to 50 ml and mixed by inverting several times. The sample was then centrifuged at 330 × g for 15 minutes at rt (1215 RPM in Beckman Allegra 6R) and aspirated as much supernatant as possible without disturbing pellet. The cell pellet was dislodged by gently swirling tube and resuspended cells in RPMI+10% FBS+pen/strep (˜1 ml/10 ml starting whole blood volume). A 60 μl sample was pipette into a sample vial (Beckman Coulter) with 600 μl VersaLyse reagent (Beckman Coulter Cat #A09777) and was gently vortexed for 10-15 sec. The sample was allowed to incubate for 10 min. at rt and being mixed again before counting. The cell suspension was counted on Vicell XR cell viability analyzer (Beckman Coulter) using PBMC cell type (dilution factor of 1:11 was stored with other parameters). The live cell/ml and viability were recorded. The cell suspension was diluted to 1×10⁷ live PBMC/ml in RPMI+10% FBS+pen/strep.

The cells were plated at 5×10⁵ in 50 μl/well of 96-well tissue culture plate (Falcon Microtest). 50 μl/well of 2×concentration oligos/controls diluted in RPMI+10% FBS+pen/strep. was added according to experiment template (100 μl/well total). Plates were placed on the shaker and allowed to mix for approx. 1 min. After being incubated for 24 hrs at 37° C.; 5% CO₂, the plates were centrifuged at 400×g for 10 minutes before removing the supernatant for MSD cytokine assay (i.e. human IL-6, IL-10, and TNF-α).

Compound 353512 is an internal standard known to be a high responder for IL-6 release in the assay, while compound 104838 is a negative control. The hPBMCs were isolated from fresh, volunteered donors and were treated with modified oligonucleotide at 0.064, 0.32, and 1.6 200 μM concentrations. After a 24 hr treatment, the cytokine levels were measured and averaged across two donors. The results presented in the table below show that selected modified oligonucleotides targeting human α-ENaC have low proinflammatory responses in human peripheral mononuclear blood cells.

TABLE 26
Modified oligonucleotides tested as
controls in hPBMC assay

| Compound | | | SEQ |
|---|---|---|---|
| No. | Sequence (5′ to 3′) | Target | ID No. |
| 104838 | Gₑₛ^{m}CₑₛTₑₛGₑₛAₑₛT_{ds}T_{ds}A_{ds}G_{ds}A_{ds}G_{ds} | TNFα | 1969 |
| | A_{ds}G_{ds}A_{ds}G_{ds}GₑₛTₑₛ^{m}Cₑₛ^{m}Cₑₛ^{m}Cₑ | | |
| 353512 | Tₑₛ^{m}Cₑₛ^{m}Cₑₛ^{m}C_{ds}A_{ds}T_{ds}T_{ds}T_{ds}^{m}C_{ds}A_{ds}G_{ds} | CRP | 1970 |
| | G_{ds}A_{ds}G_{ds}A_{ds}^{m}C_{ds}^{m}C_{ds}TₑₛGₑₛGₑ | | |

TABLE 27
Results of hPBMC Assay for Selected Modified Oligonucleotides

| Compound | | IL-10 | IL-6 | TNF-α |
|---|---|---|---|---|
| No. | Dose | (pg/mL) | (pg/mL) | (pg/mL) |
| 104838 | 0.064 | 7.4 | 63.4 | 10.5 |
| (− control) | 0.32 | 8.9 | 75.2 | 11.6 |
| | 1.6 | 12.7 | 118.9 | 19.2 |
| 353512 | 0.064 | 26.7 | 130.0 | 14.6 |
| (+ control) | 0.32 | 39.9 | 199.9 | 17.0 |
| | 1.6 | 33.0 | 230.4 | 27.7 |
| 797236 | 0.064 | 9.4 | 59.2 | 10.4 |
| | 0.32 | 20.1 | 105.5 | 13.4 |
| | 1.6 | 27.3 | 173.1 | 19.5 |
| 797308 | 0.064 | 5.6 | 55.9 | 9.3 |
| | 0.32 | 7.6 | 60.7 | 10.8 |
| | 1.6 | 9.5 | 83.6 | 13.4 |
| 797313 | 0.064 | 4.6 | 56.0 | 8.8 |
| | 0.32 | 8.9 | 55.5 | 10.7 |
| | 1.6 | 14.2 | 95.8 | 14.7 |
| 797468 | 0.064 | 7.1 | 94.0 | 9.9 |
| | 0.32 | 6.7 | 53.4 | 9.7 |
| | 1.6 | 11.8 | 103.5 | 15.0 |
| 797495 | 0.064 | 5.5 | 63.1 | 9.6 |
| | 0.32 | 8.5 | 58.9 | 10.8 |
| | 1.6 | 8.9 | 83.1 | 15.2 |
| 826262 | 0.064 | 6.1 | 50.8 | 9.7 |
| | 0.32 | 13.0 | 81.5 | 12.2 |
| | 1.6 | 10.4 | 98.2 | 14.2 |
| 826632 | 0.064 | 4.1 | 55.0 | 9.4 |
| | 0.32 | 6.6 | 65.8 | 10.8 |
| | 1.6 | 7.5 | 111.3 | 15.5 |
| 826743 | 0.064 | 4.4 | 60.1 | 9.2 |
| | 0.32 | 7.7 | 63.8 | 11.1 |
| | 1.6 | 6.0 | 81.8 | 16.1 |
| 826763 | 0.064 | 4.5 | 58.2 | 9.6 |
| | 0.32 | 8.9 | 63.1 | 10.8 |
| | 1.6 | 11.6 | 116.7 | 20.9 |
| 826819 | 0.064 | 4.7 | 51.6 | 8.2 |
| | 0.32 | 4.3 | 52.5 | 7.9 |
| | 1.6 | 7.3 | 62.8 | 11.3 |
| 826906 | 0.064 | 4.4 | 48.3 | 7.6 |
| | 0.32 | 4.8 | 68.9 | 9.2 |
| | 1.6 | 6.3 | 60.4 | 13.9 |
| 827030 | 0.064 | 3.7 | 40.8 | 7.9 |
| | 0.32 | 5.4 | 42.4 | 7.5 |
| | 1.6 | 4.5 | 54.1 | 8.4 |
| 827200 | 0.064 | 4.2 | 49.4 | 8.9 |
| | 0.32 | 5.3 | 67.6 | 9.5 |
| | 1.6 | 5.4 | 55.2 | 9.5 |
| 827288 | 0.064 | 4.5 | 44.1 | 7.7 |
| | 0.32 | 6.0 | 50.2 | 8.7 |
| | 1.6 | 7.6 | 76.3 | 14.9 |
| 827307 | 0.064 | 4.6 | 62.2 | 9.9 |
| | 0.32 | 5.3 | 52.3 | 9.0 |
| | 1.6 | 5.0 | 54.1 | 10.6 |
| 827347 | 0.064 | 8.3 | 53.6 | 10.9 |
| | 0.32 | 20.7 | 115.2 | 12.8 |
| | 1.6 | 33.9 | 163.3 | 21.1 |
| 827359 | 0.064 | 5.8 | 61.8 | 9.4 |
| | 0.32 | 6.2 | 52.7 | 10.3 |
| | 1.6 | 11.0 | 75.2 | 11.8 |
| 827372 | 0.064 | 4.7 | 56.5 | 8.8 |
| | 0.32 | 7.3 | 65.4 | 9.6 |
| | 1.6 | 13.1 | 81.3 | 13.1 |
| 827392 | 0.064 | 4.5 | 45.5 | 7.7 |
| | 0.32 | 5.1 | 48.0 | 8.8 |
| | 1.6 | 5.4 | 50.9 | 9.9 |
| 827414 | 0.064 | 5.5 | 51.6 | 8.7 |
| | 0.32 | 7.6 | 58.1 | 10.3 |
| | 1.6 | 16.6 | 102.4 | 16.3 |
| 827419 | 0.064 | 4.2 | 52.5 | 7.9 |
| | 0.32 | 7.5 | 62.0 | 11.2 |
| | 1.6 | 8.0 | 93.8 | 16.5 |
| 827497 | 0.064 | 4.5 | 50.5 | 8.3 |
| | 0.32 | 5.1 | 56.9 | 9.5 |
| | 1.6 | 5.8 | 73.7 | 13.0 |

### Example 9: Effects of a Modified Oligonucleotide Complementary to α-ENaC in a Mouse Model of Cystic Fibrosis

A modified oligonucleotide complementary to mouse α-ENaC was tested for its effects on preventing and treating airway restriction in a mouse model of cystic fibrosis. Treatment of wild type mice with a modified oligonucleotide complementary to Nedd4L induced a cystic fibrosis-like phenotype (See Crosby et al. J. of Cystic Fibrosis, 2017). Compound 668395 has a 3-10-3 phosphothiorate cEt gapmer motif. It is 16 nucleobases in length, wherein the central gap segment contains ten 2′-deoxynucleosides and is flanked by wing segments on the 3′ and 5′ ends, each containing three cEt nucleosides. All cytosine residues throughout the modified oligonucletoide are 5-methyl cytosines. The internucleoside linkages are all phosphorothioate internucleoside linkages. The sequence is GAGCATCTAATACAGC (SEQ ID NO: 1958), which is 100% complementary to mouse α-ENaC.

Adult mice were treated twice a week for 2 weeks with compound 668395 or vehicle (control) at 0.33 mg/kg/dose via aerosol dosing. Then, mice were treated with an antisense oligonucleotide that reduces Nedd4L (Nedd 4L ASO) via oropharyngeal dosing at 10 mg/kg/dose once a week for 6 weeks. After 8 weeks, airway restriction was tested with a methacholine challenge. Lung function was measured using the Penh score obtained through unrestrained plethysmography. A higher Penh score indicates more lung constriction. Each group contained 8 mice. The results, shown in the table below, indicate that pre-treatment with a modified oligonucleotide complementary to α-ENaC prevented the decrease in lung function observed in the cycstic fibrosis mouse model.

TABLE 28
Penh scores

| | Methacholine (mg/mL) | | | | |
|---|---|---|---|---|---|
| | 0 | 3 | 6 | 12 | 25 |
| Treatment | Penh score | | | | |
| Naïve (saline) | 0.7 | 0.9 | 1.1 | 1.3 | 1.7 |
| Vehicle + Nedd4L ASO | 1.2 | 1.7 | 2.1 | 3.7 | 5.4 |
| Compound No. 668395 + | 0.9 | 0.8 | 1.0 | 1.2 | 2.1 |
| Nedd4L ASO | | | | | |

In order to test the effect of a modified oligonucleotide complementary to mouse α-ENaC on reversal of airway restriction in a mouse model of cystic fibrosis, adult mice were treated with Nedd4L ASO via oropharyngeal dosing at 10 mg/kg/dose once a week for a total of 9 weeks; and compound 668395 was not administered until week 6. Starting at 6 weeks, mice were administered compound 668395, vehicle, or a control 3-10-3 cEt modified oligonucleotide (control compound) via aerosol dosing three times per week for three weeks. Lung function was tested with a methacholine challenge prior to the first treatment at 6 weeks and at 9 weeks, and Penh scores were obtained through unrestrained plethysmography. Each group contained 12 mice. The results, shown in the tables below, indicate that treatment with a modified oligonucleotide complementary to α-ENaC restored lung function in a mouse model of cystic fibrosis.

TABLE 29
Penh scores at 6 weeks

| | Methacholine (mg/mL) | | | | |
|---|---|---|---|---|---|
| | 0 | 3 | 6 | 12 | 25 |
| Treatment | Penh score | | | | |
| Naïve (no treatment) | 0.7 | 0.8 | 0.9 | 1.3 | 2.4 |
| Nedd4L ASO (baseline scores for | 0.9 | 1.3 | 1.7 | 3.1 | 4.5 |
| vehicle group) | | | | | |
| Nedd4L ASO (baseline scores for | 0.8 | 1.2 | 1.6 | 2.5 | 4.7 |
| compound 668395 group) | | | | | |
| Nedd4L ASO (baseline scores for | 1.0 | 1.3 | 1.8 | 2.9 | 5.1 |
| control compound group) | | | | | |

TABLE 30
Penh scores at 9 weeks

| | Methacholine (mg/mL) | | | | |
|---|---|---|---|---|---|
| | 0 | 3 | 6 | 12 | 25 |
| Treatment | Penh score | | | | |
| Naïve (saline) | 0.7 | 0.9 | 0.9 | 1.0 | 1.8 |
| Nedd4L ASO + vehicle | 1.1 | 1.2 | 1.5 | 2.3 | 4.0 |
| Nedd4L ASO + compound 668395 | 0.9 | 1.0 | 1.1 | 1.3 | 1.9 |
| Nedd4L ASO + control compound | 1.1 | 1.1 | 1.6 | 2.4 | 4.2 |

### Example 10: Effect of Modified Oligonucleotides Complementary to Human α-ENaC on Cystic Fibrosis Patient Derived Primary Human Bronchial Epithelial Cells

Primary human bronchial epithelial cells from cystic fibrosis patients were obtained from Epithelix. Cells were cultured at an Air-Liquid Interface (ALI) on transwell membrane inserts (Corning®) with PneumaCult™-ALI Medium (StemCell Technologies) on the basolateral side of the membrane. At 6 weeks post seeding, cells were treated either with ION No. 827359, or with ION No. 549148 (3-10-3 cET gapmer, GGCTACTACGCCGTCA, designated herein as SEQ ID NO: 1959), that served as a negative control that does not target α-ENaC. Both modified oligonucleotides were treated using free uptake at a concentration of 1 μM on the basolateral side. Cells were lysed 72 hours post treatment.

### Human α-ENaC Expression Levels

Total RNA was isolated from the cells 72 hours post treatment. α-ENaC mRNA levels were measured using human primer probe set hSCNN1A_LTS01170. α-ENaC mRNA levels were normalized to cyclophilin A. Cyclophilin A was amplified using primer-probe set HTS3936 (forward sequence, GCCATGGAGCGCTTTGG, designated herein as SEQ ID NO: 1960; reverse sequence, TCCACAGTCAGCAATGGTGATC, designated herein as SEQ ID NO: 1961; probe sequence, TCCAGGAATGGCAAGACCAGCAAGA, designated herein as SEQ ID NO: 1962). Results are presented in the tables below as percent control of the amount of α-ENaC mRNA relative to control cells (% control).

TABLE 31
Inhibition of α-ENaC mRNA by in cystic fibrosis patient
derived primary human bronchial epithelial cells

| ION | % |
|---|---|
| No. | control |
| 549148 | 100 |
| 827359 | 7 |

### Measurement of Amiloride Sensitive Current

72 hours post treatment with modified oligonucleotide, the transwell inserts were mounted in Ussing chambers (Physiologic Instruments, San Diego, CA). Short-circuit current (I_{sc}) was measured. Data were analyzed using ACQUIRE & ANALYZE 2.3 (Physiologic Instruments). The basolateral solution contained (in mM) 145 NaCl, 3.3 K2HPO4, 0.8 KH2PO4, 1.2 MgCl2, 1.2 CaCl₂), 10 glucose, 10 Hepes (adjusted to pH 7.35 with NaOH) and the apical solution contained (in mM) 145 sodium gluconate, 3.3 K2HPO4, 0.8 KH2PO4, 1.2 MgCl2, 1.2 CaCl₂), 10 glucose, 10 Hepes (adjusted to pH 7.35 with NaOH)_Amiloride was added to apical side at 100 μM. Amiloride-sensitive currents were measured in order to assess ENaC functional activity.

TABLE 32
Amiloride response in cystic fibrosis patient derived
primary human bronchial epithelial cells

| ION | ΔIsc |
|---|---|
| No. | (μA/cm²) |
| 549148 | −26 |
| 827359 | −9 |

### Measurement of Airway Surface Liquid (ASL)

72 hours post start of treatment, the effect of modified oligonucleotide on Airway Surface Liquid (ASL) was measured. Immediately before measuring the ASL, cultures were washed three times with PBS to remove excess mucus. 150 μL of KBR buffer (89 mM NaCl, 4 mM KCl, 1.2 mM MgCl2, 1.2 mM CaCl₂), 1 mM Hepes, 16 mM Na-gluconate, 10 mM glucose) was added to the apical surface of the cells as the absorption volume. ASL volume was then measured 24 hours, 48 hours and 72 hours post additional of KBR buffer.

TABLE 33
ASL volume in cystic fibrosis patient derived
primary human bronchial epithelial cells

| Time | ASL volume (μL) | |
|---|---|---|
| (hr) | 549148 | 827359 |
| 0 | 150 | 150 |
| 24 hr | 62 | 84 |
| 48 hr | 20 | 67 |
| 72 hr | 18 | 38 |

### Example 11: Effect of Combination Treatment of Modified Oligonucleotides with VX-661 (Tezacaftor) and VX-770 (Ivacaftor)

Primary human bronchial epithelial cells from cystic fibrosis patients were obtained from Epithelix. Cells were cultured at an Air-Liquid Interface (ALI) on transwell membrane inserts (Corning®) with PneumaCult™-ALI Medium (Stemcell Technologies) on the basolateral side of the membrane for 6 weeks before treatment. At Day 0, Day 4 and Day 8 of treatment, cells were treated either with ION Nos. 827359, or 549148 at 10 μM on the basolateral side of the membrane (a total of 3 doses of each ASO). One set of cells was left untreated with modified oligonucleotide. At Day 11, VX-661 (Tezacaftor) (Medchem Express) was added at 18 μM to both the previously untreated well and to one of the wells treated with ION No. 827359. On Day 14, VX-770 (Ivacaftor) (Medchem Express) was added at 10 μM to the cells previously treated with VX-661. On the same day (Day 14), cultures were washed three times on the apical side with PBS to remove excess mucus. 150 μL of PBS (absorption volume) was added to the apical surface of the cells. ASL volume was measured the next day (Day 15). Combination treatment was found to further increase ASL volume compared to control.

TABLE 34
ASL volume in cystic fibrosis patient derived
primary human bronchial epithelial cells

| | ASL volume |
|---|---|
| Treatment | (μL) |
| 549148 | 23 |
| Vx-661 + Vx-770 | 38 |
| 827359 | 59 |
| Vx-661+ Vx-770 + 827359 | 66 |

1-116. (canceled)

117. An oligomeric compound comprising a modified oligonucleotide consisting of 12 to 30 linked nucleosides, wherein the modified oligonucleotide has a nucleobase sequence comprising at least 8, at least 9, at least 10, at least 11, at least 12, at least 13, at least 14, at least 15, or 16 contiguous nucleobases of any of the nucleobase sequences of SEQ ID NOs: 6-1954, wherein the modified oligonucleotide comprises at least one modification selected from a modified sugar moiety and a modified internucleoside linkage.

118. An oligomeric compound comprising a modified oligonucleotide consisting of 12 to 30 linked nucleosides, wherein the modified oligonucleotide has a nucleobase sequence at least 85%, at least 90%, at least 95% or 100% complementary to an equal length portion within nucleobases 17,951-24,120; 19,022-19,037; 20,415-20,430; 21,750-21,766; 32,129-33,174; 32,844-32,859; or 32,989-33,004 of SEQ ID NO: 2.

119. The oligomeric compound of claim 117, wherein the modified oligonucleotide has a nucleobase sequence that is at least 85% complementary to an equal length portion of any of SEQ ID NO: 1, SEQ ID NO: 2, or SEQ ID NO: 1957.

120. The oligomeric compound of claim 117, wherein the modified oligonucleotide is single-stranded.

121. The oligomeric compound of claim 117, wherein the modified oligonucleotide comprises at least one modified internucleoside linkage.

122. The oligomeric compound of claim 121, wherein the modified internucleoside linkage is a phosphorothioate internucleoside linkage.

123. The oligomeric compound of claim 117, wherein at least one nucleoside comprises at least one modified nucleobase.

124. The oligomeric compound of claim 123, wherein the modified nucleobase is a 5-methylcytosine.

125. The oligomeric compound of claim 117, wherein the modified oligonucleotide comprises at least one nucleoside comprising a modified sugar moiety.

126. The oligomeric compound of claim 125, wherein the at least one modified sugar moiety is a bicyclic sugar moiety.

127. The oligomeric compound of claim 126, wherein the bicyclic sugar moiety comprises a 4′-2′ bridge selected from 4′-CH₂—O-2′ and 4′-CH(CH₃)—O-2′.

128. The oligomeric compound of claim 125, wherein the at least one modified sugar moiety is a non-bicyclic-modified sugar moiety.

129. The oligomeric compound of claim 128, wherein the non-bicyclic sugar moiety is a 2′-O-methoxyethyl sugar moiety, or a 2′-O-methyl sugar moiety.

130. The oligomeric compound of claim 117, wherein the modified oligonucleotide comprises:
a gap segment consisting of linked deoxynucleosides;
a 5′ wing segment consisting of linked nucleosides; and
a 3′ wing segment consisting of linked nucleosides;
wherein the gap segment is positioned between the 5′ wing segment and the 3′ wing segment and wherein each nucleoside of each wing segment comprises a modified sugar.

131. The oligomeric compound of claim 130, wherein the modified oligonucleotide comprises:
a gap segment consisting of 10 linked deoxynucleosides;
a 5′ wing segment consisting of 3 linked nucleosides; and
a 3′ wing segment consisting of 3 linked nucleosides;
wherein the gap segment is positioned between the 5′ wing segment and the 3′ wing segment and wherein each nucleoside of each wing segment comprises a modified sugar.

132. The oligomeric compound of claim 131, wherein the modified oligonucleotide comprises:
a gap segment consisting of 10 linked deoxynucleosides;
a 5′ wing segment consisting of 3 linked nucleosides; and
a 3′ wing segment consisting of 3 linked nucleosides;
wherein the gap segment is positioned between the 5′ wing segment and the 3′ wing segment and wherein each nucleoside of each wing segment comprises a cEt sugar moiety.

133. A pharmaceutical composition comprising the oligomeric compound of claim 117, and a pharmaceutically acceptable diluent.

134. The pharmaceutical composition of claim 133, wherein the pharmaceutically acceptable diluent is phosphate-buffered saline.

135. A method comprising administering to an individual the pharmaceutical composition of claim 133.

136. The method of claim 135, wherein the individual is a human.

137. A method of treating cystic fibrosis, COPD, asthma, or chronic bronchitis in an individual comprising administering to the individual the pharmaceutical composition of claim 133.
